(19) United States
(12) Patent Application Publication
(10) Pub. No.: US 2013/0323242 A1
(43) Pub. Date: Dec. 5, 2013

(54) COMPOSITIONS COMPRISING AN ANTI-PDGF APTAMER AND A VEGF ANTAGONIST

(71) Applicant: OPHTHOTECH CORP., Princeton, NJ (US)

(72) Inventors: Richard Everett, Princeton, NJ (US); Byeong Seon Chang, Sherman Oaks, CA (US)

(73) Assignee: OPHTHOTECH CORP., Princeton, NJ (US)

(21) Appl. No.: 13/797,821

(22) Filed: Mar. 12, 2013

Related U.S. Application Data
(60) Provisional application No. 61/654,672, filed on Jun. 1, 2012.

(51) Int. Cl. A61K 39/395; A61K 31/711

(52) U.S. Cl. CPC A61K 39/3955; A61K 31/711
USPC 424/134.1; 424/133.1

(57) ABSTRACT

The present invention is directed to compositions comprising an anti-PDGF aptamer and a VEGF antagonist. In certain embodiments, the compositions of the invention are useful for treating or preventing an ophthalmological disease.

### CROSS REFERENCE TO RELATED APPLICATION

This application claims the benefit of U.S. provisional application No. 61/654,672, filed Jun. 1, 2012, the disclosure of which is incorporated by reference herein in its entirety.

### SEQUENCE LISTING

The Sequence Listing associated with this application is provided in text format in lieu of a paper copy, and is hereby incorporated by reference into the specification. The name of the text file containing the Sequence Listing is OPHT_{—}010_{—}02US_ST25.txt. The text file is about 35 KB, was created on Mar. 12, 2013, and is being submitted electronically via EFS-Web.

### FIELD OF THE INVENTION

This invention relates to compositions comprising an anti-platelet derived growth factor (anti-PDGF) aptamer and a vascular endothelial growth factor (VEGF) antagonist. This invention also relates to methods for inhibiting hyperproliferation of cells or aberrant angiogenesis, as well as to methods for treating or preventing an ophthalmological disease, comprising administering a composition comprising an anti-PDGF aptamer and a VEGF antagonist. Furthermore, this invention relates to compositions and drug delivery devices that provide extended delivery of anti-PDGF aptamers and VEGF antagonists.

### BACKGROUND OF THE INVENTION

Various disorders of the eye are characterized by, caused by, or result in choroidal, retinal or iris neovascularization, or retinal edema. These disorders include macular degeneration, diabetic retinopathy, hypertensive retinopathy, central serous chorioretinopathy, cystoid macular edema, Coats disease, and ocular or adnexal neoplasms, such as choroidal hemangioma, retinal pigment epithelial carcinoma, and intraocular lymphoma. Age-related macular degeneration (AMD) is a disease that affects approximately one in ten Americans over the age of 65. One type of AMD, “wet AMD,” also known as “neovascular AMD” and “exudative AMD,” accounts for only 10% of AMD cases but results in 90% of cases of legal blindness from macular degeneration in the elderly. Diabetic retinopathy can affect up to 80% of all patients having diabetes for 10 years or more and is the third leading cause of adult blindness, accounting for almost 7% of blindness in the USA.

Advances have been made in understanding the molecular events accompanying or leading to ocular neovascularization, including the role of growth factors such as platelet derived growth factor (PDGF) and vascular endothelial growth factor (VEGF). Therapeutic agents that inhibit the activity of these growth factors have been shown to provide a therapeutic benefit to patients suffering from vascular disorders of the eye such as AMD and diabetic retinopathy, including aptamers composed of synthetic oligonucleotides. More recently, the combined use of therapeutic agents that target either PDGF or VEGF is being explored.

Combined inhibition of both PDGF and VEGF may lead to a greater benefit in treating various disorders of the eye that are characterized by, caused by, or result in choroidal, retinal or iris neovascularization, or retinal edema. Combined inhibition of both PDGF and VEGF by individual agents specific to each growth factor may be accomplished by simultaneous coadministration of both agents.

Unfortunately, polypeptide therapeutic agents can be susceptible to physical and chemical degradation. The stability of polypeptide therapeutic agents can be influenced by a variety of factors, including the polypeptide itself, e.g., its amino acid sequence. Thus, the development of stable pharmaceutical compositions comprising polypeptide therapeutics poses a significant challenge. The challenge is even greater for the development of compositions comprising a polypeptide therapeutic and another therapeutic agent, such as a polynucleotide therapeutic agent, since it requires the identification of excipients and conditions that stabilize two different therapeutic agents with acceptable compatability.

There is clearly a need in the art for stable compositions comprising multiple therapeutic agents, including those comprising an anti-PDGF aptamer and a VEGF antagonist.

### BRIEF SUMMARY OF THE INVENTION

The present invention provides compositions comprising an effective amount of: (a) an anti-PDGF aptamer or a pharmaceutically acceptable salt thereof; and (b) a VEGF antagonist or a pharmaceutically acceptable salt thereof. A composition comprising an effective amount of (a) an anti-PDGF aptamer or a pharmaceutically acceptable salt thereof and (b) a VEGF antagonist or a pharmaceutically acceptable salt thereof is a “composition of the invention.”

In certain embodiments, a composition of the invention comprises an effective amount of: (a) about 0.3 mg/mL to about 30 mg/mL Antagonist A or a pharmaceutically acceptable salt thereof; (b) about 0.5 mg/mL to about 20 mg/mL ranibizumab or a pharmaceutically acceptable salt thereof; and one or both of: (c) a buffer capable of achieving or maintaining the pH of the composition at about pH 5.0 to about pH 8.0; and (d) a tonicity modifier. In certain embodiments, the buffer is about 1 mM to about 20 mM L-histidine or about 1 mM to about 20 mM sodium phosphate, and the tonicity modifier is about 10 mM to about 200 mM NaCl, about 1% to about 20% (w/v) sorbitol, or about 1% to about 20% (w/v) trehalose. In particular embodiments, the composition of the invention further comprises: (e) about 0.001% (w/v) to about 0.05% (w/v) surfactant.

In certain embodiments, a composition of the invention comprises an effective amount of: (a) about 0.3 mg/mL to about 30 mg/mL Antagonist A or a pharmaceutically acceptable salt thereof; and (b) about 0.5 mg/mL to about 25 mg/mL bevacizumab or a pharmaceutically acceptable salt thereof; and one or both of: (c) a buffer capable of achieving or maintaining the pH of the composition at about pH 5.0 to about pH 8.0; and (d) a tonicity modifier. In certain embodiments, the buffer is about 5 mM to about 200 mM sodium phosphate or about 5 mM to about 200 mM Tris.HCl, and the tonicity modifier is about 10 mM to about 200 mM NaCl, about 1% to about 20% (w/v) sorbitol, or about 1% to about 20% (w/v) trehalose. In particular embodiments, the composition of the invention further comprises: (e) about 0.001% (w/v) to about 0.05% (w/v) surfactant.

In certain embodiments, a composition of the invention comprises an effective amount of: (a) about 0.3 mg/mL to about 30 mg/mL Antagonist A or a pharmaceutically acceptable salt thereof; (b) about 5 mg/mL to about 40 mg/mL aflibercept or a pharmaceutically acceptable salt thereof; and one or more of: (c) a buffer capable of achieving or maintaining the pH of the composition at about pH 5.0 to about pH 8.0; (d) a tonicity modifier; and (e) 0 to about 10% (w/v) sucrose. In certain embodiments, the buffer is about 5 mM to about 50 mM phosphate, and the tonicity modifier is about 10 mM to about 200 mM NaCl. In particular embodiments, the composition of the invention further comprises: (f) about 0.001% (w/v) to about 0.05% (w/v) surfactant.

In certain embodiments, a composition of the invention comprises an effective amount of: (a) about 3 mg/mL to about 90 mg/mL Antagonist A or a pharmaceutically acceptable salt thereof; (b) about 1.0 mg/mL to about 30 mg/mL ranibizumab or a pharmaceutically acceptable salt thereof; and one or both of: (c) a buffer capable of achieving or maintaining the pH of the composition at about pH 5.0 to about pH 8.0; and (d) a tonicity modifier. In certain embodiments, the buffer comprises about 1 mM to about 100 mM sodium phosphate or about 1.0 mM to about 10 mM histidine.HCl, and the tonicity modifier is about 0.5% (w/v) to about 10% (w/v) trehalose.

The present invention further provides methods for treating or preventing an ophthalmological disease, comprising administering to a mammal in need thereof a composition of the invention.

### BRIEF DESCRIPTION OF THE DRAWINGS

FIG. 1 shows AEX-HPLC chromatograms of selected compositions of the invention stored for 8 weeks at 37° C.

FIG. 2 shows WCX-HPLC chromatograms of selected compositions of the invention stored for 8 weeks at 37° C.

FIG. 3 shows SE-HPLC chromatograms of selected compositions of the invention stored for 8 weeks at 37° C.

FIG. 4 shows an AEX-HPLC trend graph of Antagonist A stability in selected compositions of the invention stored at 37° C.

FIG. 5 shows a WCX-HPLC trend graph of ranibizumab stability in selected compositions of the invention stored at 37° C.

FIG. 6 shows a SE-HPLC trend graph of Antagonist A stability in selected compositions of the invention stored at 37° C.

FIG. 7 shows a SE-HPLC trend graph of ranibizumab stability in selected compositions of the invention stored at 37° C.

FIG. 8 shows an AEX-HPLC trend graph of Antagonist A stability in selected compositions of the invention stored at 25° C.

FIG. 9 shows a WCX-HPLC trend graph of ranibizumab stability in selected compositions of the invention stored at 25° C.

FIG. 10 shows a SE-HPLC trend graph of Antagonist A stability in selected compositions of the invention stored at 25° C.

FIG. 11 shows a SE-HPLC trend graph of ranibizumab stability in selected compositions of the invention stored at 25° C.

FIG. 12 shows an AEX-HPLC trend graph of Antagonist A stability in selected compositions of the invention stored at 4° C.

FIG. 13 shows a WCX-HPLC trend graph of ranibizumab stability in selected compositions of the invention stored at 4° C.

FIG. 14 shows a SE-HPLC trend graph of Antagonist A stability in selected compositions of the invention stored at 4° C.

FIG. 15 shows a SE-HPLC trend graph of ranibizumab stability in selected compositions of the invention stored at 4° C.

FIGS. 16A and 16B show AEX-HPLC trend graphs of Antagonist A stability in selected compositions of the invention having various pHs stored at 37° C. FIG. 16A shows the percent purity of Antagonist A in compositions comprising 5% sorbitol over time at various pHs, and FIG. 16B shows the percent purity of Antagonist A in compositions comprising 130 mM NaCl over time at various pHs.

FIGS. 17A and 17B show WCX-HPLC trend graphs of ranibizumab stability in selected compositions having various pHs stored at 37° C. FIG. 17A shows the percent purity of ranibizumab in compositions comprising 5% sorbitol over time at various pHs, and FIG. 17B shows the percent purity of ranibizumab in compositions comprising 130 mM NaCl over time at various pHs.

FIG. 18 shows a SE-HPLC trend graph of Antagonist A stability in selected compositions having various pHs stored at 37° C.

FIGS. 19A and 19B show SE-HPLC trend graphs of ranibizumab stability in selected compositions of the invention having various pHs stored 37° C. FIG. 19A shows the percent purity of ranibizumab in compositions comprising 5% sorbitol, and FIG. 19B shows the percent purity of ranibizumab in compositions comprising 130 mM NaCl.

FIG. 20 shows an AEX-HPLC trend graph of Antagonist A stability in selected compositions of the invention comprising various tonicity modifiers at various pHs stored at 37° C.

FIG. 21 shows an AEX-HPLC trend graph of Antagonist A stability in selected compositions of the invention comprising various tonicity modifiers at pH 8.0 stored at 37° C.

FIG. 22 shows a WCX-HPLC trend graph of ranibizumab stability in selected compositions of the invention comprising various tonicity modifiers at various pHs stored at 37° C.

FIG. 23 shows a SE-HPLC trend graph of ranibizumab stability in selected compositions of the invention comprising various tonicity modifiers at various pHs stored at 37° C.

FIG. 24 shows a SE-HPLC trend graph of Antagonist A stability in selected compositions of the invention stored at 37° C.

FIGS. 25A and 25B show AEX-HPLC trend graphs of Antagonist A stability in selected composition of the invention stored at 25° C. (FIG. 25A) and 37° C. (FIG. 25B).

FIGS. 26A and 26B show WCX-HPLC trend graphs of Antagonist A stability in selected compositions of the invention stored at 25° C. (FIG. 26A) and 37° C. (FIG. 26B).

FIGS. 27A, 27B, and 27C show SE-HPLC chromatograms of selected compositions of the invention stored for 8 weeks at 37° C. (FIG. 27A), 25° C. (FIG. 27B) and 4° C. (FIG. 27C).

FIG. 28 shows an AEX-HPLC trend graph of Antagonist A stability in composition F6 stored at 4° C., 25° C. and 37° C.

FIG. 29 shows a WCX-HPLC trend graph of ranibizumab stability in composition F6 stored at 4° C., 25° C. and 37° C.

FIG. 30 shows a SE-HPLC trend graph of Antagonist A stability in composition F6 stored at 4° C., 25° C. and 37° C.

FIG. 31 shows a SE-HPLC trend graph of ranibizumab stability in selected compositions of the invention stored at 4° C., 25° C. and 37° C.

FIG. 32 shows AEX-HPLC chromatograms of selected compositions of the invention stored for two weeks at 37° C.

FIG. 33 shows WCX-HPLC chromatograms of selected compositions of the invention stored for 8 weeks at 25° C.

FIG. 34 shows SE-HPLC chromatograms of selected compositions of the invention stored for 8 weeks at 37° C.

FIG. 35 shows an AEX-HPLC trend graph of Antagonist A stability in selected compositions of the invention stored at 37° C.

FIG. 36 shows an AEX-HPLC trend graph of Antagonist A stability in selected compositions of the invention stored at 37° C.

FIG. 37 shows a WCX-HPLC trend graph of bevacizumab stability in selected compositions of the invention stored at 37° C.

FIG. 38 shows a WCX-HPLC trend graph of bevacizumab stability in selected compositions of the invention stored at 37° C.

FIG. 39 shows a SE-HPLC trend graph of Antagonist A stability in selected compositions of the invention stored at 37° C.

FIG. 40 shows a SE-HPLC trend graph of Antagonist A stability in selected compositions of the invention stored at 37° C.

FIG. 41 shows a SE-HPLC trend graph of bevacizumab stability in selected compositions of the invention stored at 37° C.

FIG. 42 shows a SE-HPLC trend graph of bevacizumab stability in selected compositions of the invention stored at 37° C.

FIG. 43 shows an AEX-HPLC trend graph of Antagonist A stability in selected compositions of the invention stored at 25° C.

FIG. 44 shows an AEX-HPLC trend graph of Antagonist A stability in selected compositions of the invention stored at 25° C.

FIG. 45 shows a WCX-HPLC trend graph of bevacizumab stability in selected compositions of the invention stored at 25° C.

FIG. 46 shows a WCX-HPLC trend graph of bevacizumab stability in selected compositions of the invention stored at 25° C.

FIG. 47 shows a SE-HPLC trend graph of Antagonist A stability in selected compositions of the invention stored at 25° C.

FIG. 48 shows a SE-HPLC trend graph of Antagonist A stability in selected compositions of the invention stored at 25° C.

FIG. 49 shows a SE-HPLC trend graph of bevacizumab stability in selected compositions of the invention stored at 25° C.

FIG. 50 shows a SE-HPLC trend graph of bevacizumab stability in selected compositions of the invention stored at 25° C.

FIG. 51 shows an AEX-HPLC trend graph of Antagonist A stability in selected compositions of the invention stored at 4° C.

FIG. 52 shows a WCX-HPLC trend graph of bevacizumab stability in selected compositions of the invention stored at 4° C.

FIG. 53 shows a SE-HPLC trend graph of Antagonist A stability in selected compositions of the invention stored at 4° C.

FIG. 54 shows a SE-HPLC trend graph of Antagonist A stability in selected compositions of the invention stored at 4° C.

FIG. 55 shows a SE-HPLC trend graph of bevacizumab stability in selected compositions of the invention stored at 4° C.

FIG. 56 shows an AEX-HPLC trend graph of Antagonist A stability in selected sorbitol-containing compositions of the invention having various pHs stored at 37° C.

FIG. 57 shows a WCX-HPLC trend graph of bevacizumab stability in selected sorbitol-containing compositions of the invention having various pHs stored at 37° C.

FIG. 58 shows a SE-HPLC trend graph of Antagonist A stability in selected sorbitol-containing compositions of the invention having various pHs stored at 37° C.

FIG. 59 shows a SE-HPLC trend graph of bevacizumab stability in selected sorbitol-containing compositions of the invention having various pHs stored at 37° C.

FIGS. 60A and 60B show AEX-HPLC trend graphs of Antagonist A stability in selected compositions of the invention having various pHs stored at 37° C. FIG. 60A shows the percent purity of Antagonist A in compositions comprising 5% sorbitol over time at various pHs, and FIG. 60B shows the percent purity of Antagonist A in compositions comprising 130 mM NaCl or 150 mM NaCl over time at various pHs.

FIGS. 61A and 61B show WCX-HPLC trend graphs of bevacizumab stability in selected compositions of the invention having various pHs stored at 37° C. FIG. 61A shows the percent purity of bevacizumab in compositions comprising 5% sorbitol, and FIG. 61B shows the percent purity of bevacizumab in compositions comprising 130 mM NaCl or 150 mM NaCl over time at various pHs.

FIGS. 62A and 62B show SE-HPLC trend graphs of Antagonist A stability in selected compositions of the invention having various pHs stored at 37° C. FIG. 62A shows the percent purity of Antagonist A in compositions comprising 5% sorbitol, and FIG. 62B shows the percent purity of Antagonist A in compositions comprising 130 mM NaCl or 150 mM NaCl over time at various pHs.

FIGS. 63A and 63B show SE-HPLC trend graphs of bevacizumab stability in selected compositions of the invention having various pHs stored at 37° C. FIG. 63A shows the percent purity of Antagonist A in compositions comprising 5% sorbitol, and FIG. 63B shows the percent purity of Antagonist A in compositions comprising 130 mM NaCl or 150 mM NaCl over time at various pHs.

FIG. 64 shows an AEX-HPLC trend graph of Antagonist A stability in selected compositions of the invention comprising various concentrations of Antagonist A stored for 8 weeks at 37° C.

FIG. 65 shows a WCX-HPLC trend graph of bevacizumab stability in selected compositions of the invention comprising various concentrations of Antagonist A stored for 8 weeks at 37° C.

FIG. 66 shows a SE-HPLC trend graph of Antagonist A stability in selected compositions of the invention comprising various concentrations of Antagonist A stored at 37° C.

FIG. 67 shows a SE-HPLC trend graph of bevacizumab stability in selected compositions of the invention comprising various concentrations of Antagonist A stored for 8 weeks at 37° C.

FIG. 68 shows an AEX-HPLC trend graph of Antagonist A stability in composition F19 at various storage temperatures.

FIG. 69 shows a WCX-HPLC trend graph of bevacizumab stability in composition F19 at various storage temperatures.

FIG. 70 shows a SE-HPLC trend graph of Antagonist A stability in composition F19 at various storage temperatures.

FIG. 71 shows a SE-HPLC trend graph of bevacizumab stability in composition F19 at various storage temperatures.

FIG. 72 shows an AEX-HPLC trend graph of Antagonist A stability in composition F19 as compared to composition F25 at various storage temperatures.

FIG. 73 shows a SE-HPLC trend graph of Antagonist A stability in composition F19 as compared to composition F25 at various storage conditions.

FIG. 74 shows a WCX-HPLC trend graph of bevacizumab stability in composition F19 as compared to composition F 18 at various storage temperatures.

FIG. 75 shows a SE-HPLC trend graph of bevacizumab stability in composition F19 as compared to composition F18 at various storage conditions.

FIG. 76 shows a graph depicting suppression of VEGF-induced TF expression by various compositions of the invention.

FIG. 77 shows a graph depicting suppression of PDGF-induced BTG2 expression by various compositions of the invention.

FIG. 78 shows the structure of Antagonist A (panels A-F), where designation \textcircled{B}-\textcircled{F} indicate a continuation from a previous panel.

FIGS. 79A and 79B show graphs depicting the subtracted micro-flow imaging (MFI) results for Composition F27 under varying storage conditions. The graphs provide the particle count (number of particles/mL) determined for each of the listed equivalent circular diameter ranges when stored at either 5° C. or 30° C. in either a vial or a syringe. FIG. 79A provides the particle counts within various ranges spanning 1 m to 100 m equivalent circular diameter, and FIG. 79B provides the particle counts within selected ranges spanning 10 m to 100 m equivalent circular diameter. The legends from top to bottom correspond to the bars from left to right for each particle diameter range.

FIGS. 80A and 80B show graphs depicting the subtracted MFI results for Composition F28 under varying storage conditions. The graphs provides the particle count (number of particles/mL) determined for each of the listed equivalent circular diameter ranges when stored at either 5° C. or 30° C. in either a vial or a syringe. FIG. 80A provides the particle counts within various ranges spanning 1 m to 100 m equivalent circular diameter, and FIG. 80B provides the particle counts within selected ranges spanning 10 m to 100 m equivalent circular diameter. The legends from top to bottom correspond to the bars from left to right for each particle diameter range.

FIGS. 81A and 81B show graphs depicting the subtracted MFI results for Composition F29 under varying storage conditions. The graphs provides the particle count (number of particles/mL) determined for each of the listed equivalent circular diameter ranges when stored at either 5° C. or 30° C. in either a vial or a syringe. FIG. 81A provides the particle counts within various ranges spanning 1 m to 100 m equivalent circular diameter, and FIG. 81B provides the particle counts within selected ranges spanning 10 m to 100 m equivalent circular diameter. The legends from top to bottom correspond to the bars from left to right for each particle diameter range.

FIGS. 82A and 82B show graphs depicting the subtracted MFI results for Composition F30 under varying storage conditions. The graphs provide the particle count (number of particles/mL) determined for each of the listed equivalent circular diameter ranges when stored at either 5° C. or 30° C. in either a vial or a syringe. FIG. 82A provides the particle counts within various ranges spanning 1 m to 100 m equivalent circular diameter, and FIG. 82B provides the particle counts within selected ranges spanning 10 m to 100 m equivalent circular diameter. The legends from top to bottom correspond to the bars from left to right for each particle diameter range.

FIGS. 83A and 83B show graphs depicting the subtracted MFI results for Composition F31 under varying storage conditions. The graphs provide the particle count (number of particles/mL) determined for each of the listed equivalent circular diameter ranges when stored at either 5° C. or 30° C. in either a vial or a syringe. FIG. 83A provides the particle counts within various ranges spanning 1 m to 100 m equivalent circular diameter, and FIG. 83B provides the particle counts within selected ranges spanning 10 m to 100 m equivalent circular diameter. The legends from top to bottom correspond to the bars from left to right for each particle diameter range.

FIGS. 84A and 84B show graphs comparing the subtracted MFI results for Compositions F27 to F31 under varying storage conditions. The graphs provide the particle count (number of particles/mL) determined for each of the listed equivalent circular diameter ranges when stored at either 5° C. or 30° C. in either a vial or a syringe. FIG. 84A provides the particle counts within various ranges spanning 1 m to 100 m equivalent circular diameter, and FIG. 84B provides the particle counts within selected ranges spanning 10 m to 75 m equivalent circular diameter. In FIG. 84A, the particle count within the range of <1 m to <2 m equivalent circular diameter obtained for Composition F31 stored at 30° C. in a vial was 217,404, which exceeded the values depicted in the y-axis of the graph, so this value is indicated above the corresponding bar. In FIG. 84B, the particle count within the range of <10 m to <25 m equivalent circular diameter obtained for Composition F31 stored at 30° C. in a vial was 3,044, which exceeded the values depicted in the y-axis of the graph, so this value is indicated above the corresponding bar. The legends from top to bottom correspond to the bars from left to right for each particle diameter range.

### DETAILED DESCRIPTION OF THE INVENTION

### Definitions and Abbreviations

As used herein, the following terms and phrases shall have the meanings set forth herein.

The term “about” when used in connection with a referenced numeric indication means the referenced numeric indication plus or minus up to 10% of that referenced numeric indication. For example, “about 100” means from 90 to 110.

The term “antagonist” refers to an agent that inhibits, either partially or fully, the activity or production of a target molecule. In particular, the term “antagonist,” as applied selectively herein, means an agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of one or both of ligand synthesis and stability (e.g., using, antisense, ribozymes or RNAi compositions targeting the ligand gene/nucleic acid), blocking of binding of the ligand to its cognate receptor (e.g., using anti-ligand aptamers, antibodies, anti-receptor antibodies, or a soluble, decoy cognate receptor or fragment thereof), repression of one or both of receptor synthesis and stability (e.g., using antisense, ribozymes or RNAi compositions targeting the ligand receptor gene/nucleic acid), blocking of the binding of the receptor to its cognate response element (e.g., using anti-receptor antibodies) and blocking of the activation of the receptor by its cognate ligand (e.g., using receptor tyrosine kinase inhibitors). In addition, the antagonist may directly or indirectly inhibit the target molecule.

As used herein, an “antibody” includes whole antibodies and any antigen binding fragment or a single chain thereof. Thus the term “antibody” includes any protein or peptide containing molecule that comprises at least a portion of an immunoglobulin molecule having biological activity of binding to the antigen. Examples of such may comprise a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. Antibodies include monoclonal antibodies and polyclonal antibodies.

The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include: (i) a Fab fragment, a monovalent fragment consisting of the V_{L}, V_{H}, C_{L} and C_{H1} domains, (ii) a F(ab′)₂ fragment, a bivalent fragment comprising two Fab fragments linked by a disulfide bridge at the hinge region, (iii) a Fd fragment consisting of the V_{H} and C_{H1} domains, (iv) a Fv fragment consisting of the V_{L} and V_{H} domains of a single arm of an antibody, (v) a dAb fragment (Ward et al., (1989) Nature 341 544-546), which consists of a V_{H} domain, and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv), see e.g., Bird et al. (1988) Science 242 423-426, and Huston et al. (1988) Proc Natl Acad Sci USA 85 5879-5883). Such single chain antibodies are also intended to be encompassed within the term “antigen-binding fragment” of an antibody. These antibody fragments are obtained using conventional techniques known to those in the art, and the fragments can be screened for utility in the same manner as whole antibodies.

The term “aptamer” refers to a peptide or nucleic acid that has an inhibitory effect on a target. Inhibition of the target by the aptamer can occur by binding of the target, by catalytically altering the target, by reacting with the target in a way which modifies the target or the functional activity of the target, by ionically or covalently attaching to the target as in a suicide inhibitor or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein. Aptamers can be pegylated or unpegylated. For example, one or more polyethylene glycol chains can be linked to the 5′ end of a nucleic acid aptamer via a linker.

A “composition” can comprise an active agent and a carrier, inert or active. The compositions are useful for diagnostic or therapeutic use in vitro, in vivo or ex vivo. In particular embodiments, the compositions are sterile, substantially free of endotoxins or non-toxic to recipients at the dosage or concentration employed.

The term “label” includes, but is not limited to, a radioactive isotope, a fluorophore, a chemiluminescent moiety, an enzyme, an enzyme substrate, an enzyme cofactor, an enzyme inhibitor, a dye, a metal ion, a ligand (e.g., biotin or a hapten) and the like. Examples of fluorophore labels include fluorescein, rhodamine, dansyl, umbelliferone, Texas red, and luminol. Other examples of labels include NADPH, alpha-beta-galactosidase and horseradish peroxidase.

The term “nucleic acid” refers to a polynucleotide such as deoxyribonucleic acid (DNA) or ribonucleic acid (RNA). The term also includes analogs of RNA or DNA made from nucleotide analogs, and, as applicable to the embodiment being described, single (sense or antisense) and double-stranded polynucleotides, expressed sequence tags (ESTs), chromosomes, cDNAs, mRNAs, and rRNAs. Nucleic acids include modified forms of nucleic acids that deviate structurally from naturally occurring nucleic acid structures based on the standard building blocks (adenosine, cytidine, guanosine, thymidine and uridine). Modifications may be to the backbone, sugar or nucleobase and can be naturally occurring or artificially introduced. For example, nucleic acids may be modified within their backbone. Illustrative modifications are disclosed herein. Nucleic acids can include nucleic acid aptamers and spiegelmers.

In some embodiments, Antagonist A exists in a modified form. A modified form of Antagonist A is that which comprises a nucleotide in a modified form as described herein, where the nucleotide is present in an unmodified form in Antagonist A.

The terms “RNA interference,” “RNAi,” “miRNA,” and “siRNA” refer to any method by which expression of a gene or gene product is decreased by introducing into a target cell one or more double-stranded RNAs, which are homologous to a gene of interest (particularly to the messenger RNA of the gene of interest, e.g., PDGF or VEGF).

The term “neovascularization” refers to new blood vessel formation in abnormal tissue or in abnormal positions.

The term “angiogenesis” refers to formation of new blood vessels in normal or in abnormal tissue or positions.

The term “ophthalmological disease” includes diseases of the eye and diseases of the ocular adnexa.

The term “ocular neovascular disorder” refers to an ocular disorder characterized by neovascularization. Certain cancers are ocular neovascular disorders. In one embodiment, the ocular neovascular disorder is a disorder other than cancer. Examples of ocular neovascular disorders other than cancer include diabetic retinopathy and age-related macular degeneration.

The term “mammal” includes human and non-human mammals, such as, e.g., a human, mouse, rat, rabbit, monkey, cow, hog, sheep, horse, dog, and cat.

The term “protein” and “polypeptide” are used interchangeably and in their broadest sense refer to a compound of two or more subunit amino acids, amino acid analogs or peptidomimetics. The subunits may be linked by peptide bonds. In another embodiment, the subunit may be linked by other bonds, e.g., ester, ether, etc. No limitation is placed on the maximum number of amino acids which may comprise a protein's or peptide's sequence.

As used herein the term “amino acid” refers to natural or unnatural or synthetic amino acids, including glycine and both the D and L optical isomers, amino acid analogs and peptidomimetics.

The term “PDGF” refers to a platelet-derived growth factor that regulates cell growth or division. As used herein, the term “PDGF” includes the various subtypes of PDGF including PDGF-B (e.g., GenBank Accession Nos. X02811 and CAa26579), PDGF-A (GenBank Accession nos. X06374 and CAA29677), PDGF-C (GenBank Accession Nos. NM 016205 and NP 057289), PDGF-D, variants 1 and 2 (GenBank Accession Nos. NM 025208, NP 079484, NM 033135, NP 149126), and dimerized forms thereof, including PDGF-AA, PDGF-AB, PDGF-BB, PDGF-CC, and PDGF— DD. Platelet derived growth factors includes homo- or heterodimers of A-chain (PDGF-A) and B-chain (PDGF-B) that exert their action via binding to and dimerization of two related receptor tyrosine kinase platelet-derived growth factor cell surface receptors (i.e., PDGFRs), PDGFR-α (see GenBank Accession Nos. NM 006206 and NP 006197) and PDGFR-β (see GenBank Accession Nos. NM 002609 and NP 002600). See, also, PCT Application Publication No. 2010/127029, which is incorporated herein in its entirety, for PDGF sequences. In addition, PDGF-C and PDGF-D, two additional protease-activated ligands for the PDGFR complexes, have been identified (Li et al., (2000) Nat. Cell. Biol. 2: 302-9; Bergsten et al., (2001) Nat. Cell. Biol. 3: 512-6; and Uutele et al., (2001) Circulation 103: 2242-47). Due to the different ligand binding specificities of the PDGFRs, it is known that PDGFR-α/α binds PDGF-AA, PDGF-BB, PDGF-AB, and PDGF-CC; PDGFR-β/β binds PDGF-BB and PDGF-DD; whereas PDGFR-α/β binds PDGF-AB, PDGF-BB, PDGF-CC, and PDGF-DD (Betsholtz et al., (2001) BioEssays 23: 494-507). As used herein, the term “PDGF” also refers to those members of the class of growth factors that induce DNA synthesis and mitogenesis through the binding and activation of a PDGFR on a responsive cell type. PDGFs can effect, for example: directed cell migration (chemotaxis) and cell activation; phospholipase activation; increased phosphatidylinositol turnover and prostaglandin metabolism; stimulation of both collagen and collagenase synthesis by responsive cells; alteration of cellular metabolic activities, including matrix synthesis, cytokine production, and lipoprotein uptake; induction, indirectly, of a proliferative response in cells lacking PDGF receptors; and potent vasoconstrictor activity. The term “PDGF” can be used to refer to a “PDGF” polypeptide, a “PDGF” encoding gene or nucleic acid, or a dimerized form thereof. The term “PDGF-A” refers to an A chain polypeptide of PDGF or its corresponding encoding gene or nucleic acid. The term “PDGF-B” refers to a B chain polypeptide of PDGF or its corresponding encoding gene or nucleic acid. The term “PDGF-C” refers to a C chain polypeptide of PDGF or its corresponding encoding gene or nucleic acid. The term “PDGF-D” refers to a D chain polypeptide of PDGF or its corresponding encoding gene or nucleic acid, including variants 1 and 2 of the D chain polypeptide of PDGF. The term “PDGF-AA” refers to a dimer having two PDGF-A chain polypeptides. The term “PDGF-AB” refers to a dimer having one PDGF-A chain polypeptide and one PDGF-B chain polypeptide. The term “PDGF-BB” refers to a dimer having two PDGF-B chain polypeptides. The term “PDGF-CC” refers to a dimer having two PDGF-C chain polypeptides. The term “PDGF-DD” refers to a dimer having two PDGF-D chain polypeptides.

The term “VEGF” refers to a vascular endothelial growth factor that induces angiogenesis or an angiogenic process. As used herein, the term “VEGF” includes the various subtypes of VEGF (also known as vascular permeability factor (VPF) and VEGF-A) (see GenBank Accession Nos. NM 003376 and NP 003367) that arise by, e.g., alternative splicing of the VEGF— A/VPF gene including VEGF₁₂₁, VEGF₁₆₅ and VEGF₁₈₉. See, also, PCT Application Publication No. 2010/127029, which is incorporated herein in its entirety, for VEGF sequences. Further, as used herein, the term “VEGF” includes VEGF-related angiogenic factors such as P1GF (placenta growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-I (FIt-I) (see GenBank Accession No. AF063657 and SID NO:8 of PCT Application Publication No. WO 2010/127029), VEGFR-2 (KDR/Flk-1) (see GenBank Accession Nos. AF035121 and AAB88005), or VEGFR-3 (FLT-4). The term “VEGF” can be used to refer to a “VEGF” polypeptide or a “VEGF” encoding gene or nucleic acid.

The term “PDGF antagonist” refers generally to an agent that reduces, or inhibits, either partially or fully, the activity or production of a PDGF. A PDGF antagonist can directly or indirectly reduce or inhibit the activity or production of a specific PDGF such as PDGF-B. Furthermore, “PDGF antagonists,” consistent with the above definition of “antagonist,” include agents that act on a PDGF ligand or its cognate receptor so as to reduce or inhibit a PDGF-associated receptor signal. Examples of “PDGF antagonists” include antisense molecules, ribozymes or RNAi that target a PDGF nucleic acid; anti-PDGF aptamers, anti-PDGF antibodies to PDGF itself or its receptor, or soluble PDGF receptor decoys that prevent binding of a PDGF to its cognate receptor; antisense molecules, ribozymes or RNAi that target a cognate PDGF receptor (PDGFR) nucleic acid; anti-PDGFR aptamers or anti-PDGFR antibodies that bind to a cognate PDGFR receptor; and PDGFR tyrosine kinase inhibitors.

The term “VEGF antagonist” refers generally to an agent that reduces, or inhibits, either partially or fully, the activity or production of a VEGF. A VEGF antagonist can directly or indirectly reduce or inhibit the activity or production of a specific VEGF such as VEGF₁₆₅. Furthermore, “VEGF antagonists,” consistent with the above definition of “antagonist,” include agents that act on either a VEGF ligand or its cognate receptor so as to reduce or inhibit a VEGF-associated receptor signal. Examples of “VEGF antagonists” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors. As used herein, the term “VEGF antagonist” is used to refer collectively to ranibizumab, bevacizumab, and aflibercept.

“Pharmaceutically acceptable salts” include sulfate, citrate, acetate, oxalate, chloride, bromide, iodide, nitrate, bisulfate, phosphate, acid phosphate, lsomcotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dimtrobenzoate, hydroxybenzoate, methoxybenzoate, methylbenzoate, o-acetoxybenzoate, naphthalene-2-benzoate, isobutyrate, phenylbutyrate, alpha-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, mcotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” also refers to a salt of an antagonist of the present invention having an acidic functional group, such as a carboxylic acid functional group, and a base. Suitable bases include, but are not limited to, hydroxides of alkali metals such as sodium, potassium, and lithium, hydroxides of alkaline earth metal such as calcium and magnesium, hydroxides of other metals, such as aluminum and zinc, ammonia, and organic amines, such as unsubstituted or hydroxy-substituted mono-, di-, or tri-alkylamines, dicyclohexylamine, tributylamine, pyridine, N-methyl, N-ethylamine, diethylamine, triethylamine, mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine, N-methyl-D-glucamine, and amino acids such as arginine, lysine, and the like. The term “pharmaceutically acceptable salt” also includes a hydrate of a compound of the invention.

The term “effective amount,” when used in connection with a composition of the invention or treatment or prevention of an ophthalmological disease, refers to a combined amount of a PDGF antagonist and a VEGF antagonist that is useful to treat or prevent an ophthalmological disease. The “effective amount” can vary depending upon the mode of administration, specific locus of the ophthalmological disease, the age, body weight, and general health of the mammal. The effective amount of each antagonist of a composition of the invention is the amount of each that is useful for treating or preventing an ophthalmological disease with the composition, even if the amount of the PDGF antagonist in the absense of the VEGF antagonist, or the VEGF antagonist in the absense of the PDGF antagonist, is ineffective to treat or prevent the ophthalmological disease.

A “variant” of polypeptide X refers to a polypeptide having the amino acid sequence of polypeptide X that is altered in one or more amino acid residues. The variant can have “conservative” changes, wherein a substituted amino acid has similar structural or chemical properties (e.g., replacement of leucine with isoleucine). More rarely, a variant can have “nonconservative” changes (e.g., replacement of glycine with tryptophan). Analogous minor variations may also include amino acid deletions or insertions, or both. Guidance in determining which amino acid residues may be substituted, inserted, or deleted without eliminating biological or immunological activity can be determined using computer programs well known in the art, for example, LASERGENE software (DNASTAR).

The term “variant,” when used in the context of a polynucleotide sequence, can encompass a polynucleotide sequence related to that of a gene, coding sequence thereof, aptamer, or other polynucleotide sequence. The variant may include one or more nucleotide or nucleoside substitutions, additions or insertions as compared to the reference gene, coding sequence, aptamer or other polynucleotide sequence. This definition also includes, for example, “allelic,” “splice,” “species,” or “polymorphic” variants. A splice variant can have significant identity to a reference molecule, but will generally have a greater or lesser number of polynucleotides due to alternative splicing of exons during mRNA processing. Species variants are polynucleotide sequences that vary from one species to another. A polymorphic variant is a variation in the polynucleotide sequence of a particular gene between individuals of a given species.

As used herein, the term “excipient” refers to a typically inert substance that is commonly used as a diluent, vehicle, preservative, binder, or stabilizing agent for active agents and includes, but is not limited to, proteins (e.g., serum albumin, etc.), amino acids (e.g., aspartic acid, glutamic acid, lysine, arginine, glycine, histidine, alanine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, Pa.) and Handbook of Pharmaceutical Excipients (by Raymond C. Rowe, 5th ed., APhA Publications, Washington, D.C.) which are hereby incorporated in its entirety. In certain embodiments, the excipient(s) imparts a beneficial physical property to the composition, such as increased protein, polynucleotide, aptamer or small molecule stability, increased protein, polynucleotidem aptamer or small molecule solubility, or decreased viscosity. In some embodiments, the compositions comprise a plurality of active agents, and the excipient(s) help stabilize the active agents.

The term “buffer” as used herein denotes a pharmaceutically acceptable excipient, which stabilizes the pH of a pharmaceutical preparation. Suitable buffers are well known in the art. Suitable pharmaceutically acceptable buffers include but are not limited to acetate-buffers, histidine-buffers, citrate-buffers, succinate-buffers, tris-buffers and phosphate-buffers. Methods for preparing such buffers are known in the art. Independently from the buffer used, the pH can be adjusted at a value from about 4.5 to about 7.0 or alternatively from about 5.5 to about 6.5 or alternatively about 6.0 with an acid or a base known in the art, e.g., succinic acid, hydrochloric acid, acetic acid, phosphoric acid, sulfuric acid and citric acid, sodium hydroxide and potassium hydroxide. Suitable buffers include, without limitation, histidine buffer, 2-morpholinoethanesulfonic acid (MES), cacodylate, phosphate, acetate, succinate, and citrate buffers. Additional examples of phosphate buffers also include, without limitation, sodium phosphate buffers and potassium phosphate buffers. Sodium phosphate buffer may be prepared, e.g., by combining a solution of NaH₂PO₄ (monobasic) with a solution of Na₂HPO₄ (dibasic) and then adjusting the pH of the combined solutions with either phosphoric acid or sodium hydroxide to achieve the desired pH. 2-amino-2-hydroxymethyl-propane-1,3-diol (Tris) buffers may be prepared, e.g., by adjusting the pH of a solution of Tris using HCl to achieve a desired pH, e.g., a pH in the range of about pH 7.0 to about pH 9.0. L-histidine may also be used as a buffer according to the invention. In certain embodiments, a buffer is capable of achieving or maintaining the pH of a composition of the invention within a desired range or at or near a desired pH, e.g., during storage, e.g., during storage at room temperature or 4° C. for at least one week, at least one month, at least two months, at least four months, at least six months, at least one year, or at least two years. In certain embodiments, the concentration of the buffer is from about 0.01 mM to about 1000 mM, about 0.1 mM to about 1000 mM, about 0.1 mM to about 500 mM, about 0.1 to about 200 mM, about 0.1 to about 100 mM, about 1 mM to about 1000 mM, about 1 mM to about 500 mM, about 1 mM to about 200 mM, about 1 mM to about 100 mM, about 1 mM to about 50 mM, about 2 mM to about 60 mM, about 4 mM to about 60 mM, or about 4 mM to about 40 mM, about 5 mM to about 20 mM, or about 5 mM to about 25 mM.

Pharmaceutically acceptable “cryoprotectants” are known in the art and include without limitation, e.g., sucrose, trehalose, and glycerol. Pharmaceutically acceptable cryoprotectants provide stability protection of compositions, or one or more active ingredients therein, from the effects of freezing or lyophilization.

The term “tonicity agent” or “tonicity modifier” as used herein denotes pharmaceutically acceptable agents used to modulate the tonicity of a composition. Suitable tonicity agents include, but are not limited to, sodium chloride, sorbitol, trehalose, potassium chloride, glycerin and any component from the group of amino acids, sugars, as defined herein as well as combinations thereof. In certain embodiments, tonicity agents may be used in an amount of about 1 mM to about 1000 mM, about 1 mM to about 500 mM, about 5 mM to about 500 mM, about 10 mM to about 450 mM, about 20 mM to about 400 mM, about 50 mM to about 300 mM, about 100 mM to about 200 mM, or about 125 mM to about 175 mM. In certain embodiments, a tonicity agent comprises an amino acid present in a composition at about 5 mM to about 500 mM.

The term “stabilizer” indicates a pharmaceutical acceptable excipient, which protects the active pharmaceutical ingredient(s) or agents(s) or the composition from chemical or physical degradation during manufacturing, storage and application. Stabilizers include, but are not limited to, sugars, amino acids, polyols, surfactants, antioxidants, preservatives, cyclodextrines, e.g. hydroxypropyl-β-cyclodextrine, sulfobutylethyl-β-cyclodextrin, β-cyclodextrin, polyethyleneglycols, e.g. PEG 3000, PEG 3350, PEG 4000, PEG 6000, albumin, e.g. human serum albumin (HSA), bovine serum albumin (BSA), salts, e.g. sodium chloride, magnesium chloride, calcium chloride, and chelators, e.g. EDTA. Stabilizers may be present in the composition in an amount of about 0.1 mM to about 1000 mM, about 1 mM to about 500 mM, about 10 to about 300 mM, or about 100 mM to about 300 mM.

As used herein, the term “surfactant” refers to a pharmaceutically acceptable organic substance having amphipathic structures; namely, it is composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants may be used as wetting, emulsifying, solubilizing, and dispersing agents for pharmaceutical compositions and preparations of biological materials. In some embodiments of the compositions described herein, the amount of surfactant is described as a percentage expressed in weight/volume percent (w/v %). Suitable pharmaceutically acceptable surfactants include, but are not limited to, the group of polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), or sodium dodecyl sulphate (SDS). Polyoxyethylenesorbitan-fatty acid esters include polysorbate 20, (sold under the trademark Tween 20™) and polysorbate 80 (sold under the trademark Tween 80™). Polyethylene-polypropylene copolymers include those sold under the names Pluronic® F68 or Poloxamer 188™. Polyoxyethylene alkyl ethers include those sold under the trademark Brij™. Alkylphenolpolyoxyethylene ethers include those sold under the tradename Triton-X. Polysorbate 20 (Tween 20™) and polysorbate 80 (Tween 80™) are generally used in a concentration range of about 0.001% w/v to about 1% w/v or about 0.002% w/v to about 0.1% w/v of the total volume of the composition, or alternatively of about 0.003% w/v to about 0.007% w/v. In some embodiments, Tween 80™ is used at about 0.003% w/v, about 0.004% w/v, about 0.0045% w/v, about 0.005% w/v, about 0.0055% w/v, about 0.006% w/v or about 0.007% w/v. In some embodiments, Tween 80™ is used at about 0.005% w/v. In this aspect, “w/v” intends the weight of surfactant per total volume of the composition.

A “lyoprotectant” refers to a pharmaceutically acceptable substance that stabilizes a protein, nucleic acid or other active pharmaceutical ingredient(s) or agent(s) during lyophilization. Examples of lyoprotectants include, without limitation, sucrose, trehalose or mannitol.

A “polyol” refers to an alcohol containing multiple hydroxyl groups, or a sugar alcohol. A sugar alcohol is a hydrogenated form of carbohydrate, whose carbonyl group (aldehyde or ketone, reducing sugar) has been reduced to a primary or secondary hydroxyl group (hence the alcohol). Sugar alcohols have the general formula H(HCHO)ₙ₊₁H, whereas sugars have H(HCHO)ₙHCO.

An “antioxidant” refers to a molecule capable of slowing or preventing the oxidation of other molecules. Antioxidants are often reducing agents, chelating agents and oxygen scavengers such as thiols, ascorbic acid or polyphenols. Non-limiting examples of antioxidants include ascorbic acid (AA, E300), thiosulfate, methionine, tocopherols (E306), propyl gallate (PG, E310), tertiary butylhydroquinone (TBHQ), butylated hydroxyanisole (BHA, E320) and butylated hydroxytoluene (BHT, E321).

A “preservative” is a natural or synthetic chemical that is added to products such as foods, pharmaceutical compositions, paints, biological samples, wood, etc. to prevent decomposition by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Examples of antimicrobial preservatives include benzalkonium chloride, benzoic acid, cholorohexidine, glycerin, phenol, potassium sorbate, thimerosal, sulfites (sulfur dioxide, sodium bisulfate, potassium hydrogen sulfite, etc.) and disodium EDTA. Other preservatives include those commonly used in patenteral protein compositions such as benzyl alcohol, phenol, m-cresol, chlorobutanol or methylparaben.

The present invention provides compositions comprising at least one anti-PDGF aptamer and at least one VEGF antagonist, as well as related methods of manufacture and use thereof.

In one embodiment, the present invention provides a composition comprising an effective amount of: (a) an anti-PDGF aptamer or a pharmaceutically acceptable salt thereof; and (b) a VEGF antagonist or a pharmaceutically acceptable salt thereof. In particular embodiments, at least about 90% of one or both of the anti-PDGF aptamer and the VEGF antagonist is chemically stable when the composition is stored at a temperature from about 2.0° C. to about 8.0° C. for at least about twelve weeks.

In particular embodiments of various compositions and methods of the present invention, the anti-PDGF aptamer is Antagonist A or a modified form thereof. In particular embodiments of various compositions and methods of the present invention, the VEGF antagonist is ranibizumab, bevacizumab, or aflibercept, or pharmaceutically acceptable salts thereof.

In another embodiment, the present invention provides methods for treating or preventing an ophthalmological disease, comprising administering to a mammal in need thereof a composition of the invention. The composition is administered in an amount effective to treat or prevent the ophthalmological disease. In various embodiments, the ophthalmological disease is age-related macular degeneration, polypoidal choroidal vasculopathy, condition associated with choroidal neovascularization, hypertensive retinopathy, diabetic retinopathy, sickle cell retinopathy, condition associated with peripheral retinal neovascularization, retinopathy of prematurity, venous occlusive disease, arterial occlusive disease, central serous chorioretinopathy, cystoid macular edema, retinal telangiectasia, arterial macroaneurysm, retinal angiomatosis, radiation-induced retinopathy, rubeosis iridis, or a neoplasm. In particular embodiments, the ophthalmological disease is age-related macular degeneration, and the age-related macular degeneration is wet age-related macular degeneration or dry age-related macular degeneration. In certain embodiments, the composition is present in a drug-delivery device. In certain embodiments, the composition is administered intraocularly. In specific embodiments, the intraocular administration is intravitreal administration or anterior chamber administration. In other embodiments, the mammal is a human.

### PDGF Aptamers and VEGF Antagonists

The present invention provides compositions, including pharmaceutical compositions, comprising an anti-PDGF aptamer and a VEGF antagonist. In particular embodiments, the anti-PDGF aptamer is Antagonist A or a modified form thereof (or a pharmaceutically acceptable salt thereof), and the VEGF antagonist is ranibizumab, bevacizumab, or aflibercept (or a pharmaceutically acceptable salt thereof). The present invention further provides compositions comprising an effective amount of an anti-PDGF aptamer and a VEGF antagonist.

Anti-PDGF Aptamers

In certain embodiments, anti-PDGF aptamers include, but are not limited to, those described in U.S. Pat. No. 8,039,443, incorporated by reference herein in its entirety, which include both PDGF-specific and PDGF-VEGF-specific aptamers. Examples of anti-PDGF aptamers include aptamers whose oligonucleotide sequence comprises, consists essentially of or consists of one of the following sequences: ARC126: 5′-(5′—NH2 dC dA dG dG dC fU dA fC mG-3′, SEQ ID NO:1)-HEG-(5′-dC-dG-T-dA-mG-dA-mG dC dA fU fC mA-3′, SEQ ID NO:2)-HEG-(5′-T-dG-dA-T-fC-fC-fU-mG-3′ dT-3′, SEQ ID NO:3)-3′ wherein HEG=hexaethylene glycol amidite; ARC127: 5′-[40K PEG]-(5′—NH2 dC dA dG dG dC fU dA fC mG-3′, SEQ ID NO:1)-HEG-(5′-dC-dG-T-dA-mG-dA-mG dC dA fU fC mA-3′, SEQ ID NO:2)-HEG-(5′-T-dG-dA-T-fC-fC— fU-mG-3′ dT-3′, SEQ ID NO:3)-3′ wherein HEG=hexaethylene glycol amidite; ARC240: 5′-[20K PEG]-(5′—NH2 dC dA dG dG dC fU dA fC mG-3′, SEQ ID NO:1)-HEG-(5′-dC-dG-T-dA-mG-dA-mG dC dA fU fC mA-3′, SEQ ID NO:2)-HEG-(5′-T-dG-dA-T-fC-fC-fU-mG-3′ dT-3′, SEQ ID NO:3)-3′ wherein HEG=hexaethylene glycol amidite; ARC308: 5′-[30K PEG]-(5′—NH2 dC dA dG dG dC fU dA fC mG-3′, SEQ ID NO:1)-HEG-(5′-dC-dG-T-dA-mG-dA-mG dC dA fU fC mA-3′, SEQ ID NO:2)-HEG-(5′-T-dG-dA-T-fC-fC-fU-mG-3′ dT-3′, SEQ ID NO:3)-3′ wherein HEG=hexaethylene glycol amidite; deoxyARC126: 5′-dCdAdGdGdCdTdAdCdGdCdGdTdAdGdAdGdCdAdTdCdAdTdGdAdTdCdCdTd G-[3T]-3′ (SEQ ID NO:75) wherein “d” indicates unmodified deoxynucleotides and “[3T]” refers to an inverted thymidine nucleotide that is attached to the 3′ end of the oligonucleotide at the 3′ position on the ribose sugar, thus the oligonucleotide has two 5′ ends and is thus resistant to nucleases acting on the 3′ hydroxyl end; and ARC124: 5′CACAGGCTACGGCACGTAGAGCATCACCATGATCCTGTG 3′InvdT (SEQ ID NO:6).

Examples of PDGF/VEGF binding multivalent aptamers include the PDGF-B-VEGF aptamer chimeras TK.131.12. A and TK.131.12.B, which allow for the simultaneous targeting of PDGF-B and VEGF. These aptamera chimeras are described in PCT Patent Application Publication Nos. WO2006/050498 and WO2004/094164. The sequence of TK.131.012.A is: 5′ dCdAdGdGdCdTdAdCdGmAmUmGmCmAmGmUmUmUmGmAmGmAmAmGm UmCmGmCmGmCmAmUdCdGdTdAdGdAdGdCdAdTdCdAdGdAdAdAdTdGdAdT dCdCdTdG[3T]-3′ (SEQ ID NO:4), wherein “m” indicates 2′-OMe nucleotides, “d” and “[3T]” are as defined above; and the sequence of TK.131.012.B is: 5′ dCdAdGdGdCdTdAdCdGmUmGmCmAmGmUmUmUmGmAmGmAmAmGmUmC mGmCmGmCmAdCdGdTdAdGdAdGdCdAdTdCdAdGdAdAdAdTdGdAdTdCdCdTd G-[3T] (SEQ ID NO:5) wherein “m” and “[3T]” are as defined above.

In particular embodiments, an anti-PDGF aptamer binds PDGF. Examples of anti-PDGF aptamers include a series of nucleic acid aptamers of 31-35 nucleotides in length (SEQ ID NO:1 to SEQ ID NO:3, SEQ ID NO:4 to SEQ ID NO:30, SEQ ID NO:31 to SEQ ID NO:68, SEQ ID NO:69, and SEQ ID NO:70 to SEQ ID NO:74 of U.S. Pat. No. 8,039,443), that bind specifically to PDGF-B protein in vitro and which functionally block the activity of PDGF-BB in in vivo and cell-based assays. In particular embodiments, the anti-PDGF-B aptamers are derived from a parent molecule ARC126 (5′-(SEQ ID NO:1)-HEG-(SEQ ID NO:2)-HEG-(SEQ ID NO:3)-3′-dT-3′) which contains seven individual 2′F containing residues. The 2′F containing residues can increase the in vitro serum and in vivo stability of the aptamer by blocking its degradation by serum endonucleases or exonucleases. In particular embodiments, the anti-PDGF aptamers are fully 2′F-free aptamers that retain potent in vitro binding and anti-proliferative activity and contain naturally occurring 2′ deoxy or 2′OMe substituted nucleotides. In addition, in particular embodiments, these aptamers retain substantial serum stability as determined through resistance to nuclease degradation in an in vitro stability assay.

In certain embodiments, the anti-PDGF aptamer is Antagonist A or a pharmaceutically acceptable salt thereof. The chemical name of Antagonist A is [(monomethoxy 20K polyethylene glycol carbamoyl-N2-) (monomethoxy 20K polyethylene glycol carbamoyl-N-6-)]-lysine-amido-6-hexandilyl-(1-5′)-2′-deoxycytidylyl-(3′-5′)-2′-deoxyadenylyl-(3′-5′)-2′-deoxyguanylyl-(3′-5′)-2′-deoxyguanylyl-(3′-5′)-2′-deoxycytidylyl-(3′-5′)-2′-deoxy-2′-fluorouridylyl-(3′-5′)-2′-deoxyadenylyl-(3′-5′)-2′-deoxy-2′-fluorocytidylyl-(3′-5′)-2′-deoxy-2′-methoxyguanylyl-(3′-1)-PO₃-hexa(ethyloxy)-(18-5′)-2′-deoxycytidylyl-(3′-5′)-2′-deoxyguanylyl-(3′-5′)-thymidylyl-(3′-5′)-2′-deoxyadenylyl-(3′-5′)-2′-deoxy-2′-methoxyguanylyl-(3′-5′)-2′-deoxyadenylyl-(3′-5′)-2′-deoxy-2′-methoxyguanylyl-(3′-5′)-2′-deoxycytidylyl-(3′-5′)-2′-deoxyadenylyl-(3′-5′)-2′-deoxy-2′-fluorouridylyl-(3′-5′)-2′-deoxy-2′-fluorocytidylyl-(3′-5′)-2′-deoxy-2′-methoxyadenylyl-(3′-1)-PO₃-hexa(ethyloxy)-(18-5′)-thymidylyl-(3′-5′)-2′-deoxyguanylyl-(3′-5′)-2′-deoxyadenylyl-(3′-5′)-thymidylyl-(3′-5′)-2′-deoxy-2′-fluorocytidylyl-(3′-5′)-2′-deoxy-2′-fluorocytidylyl-(3′-5′)-2′-deoxy-2′-fluorouridylyl-(3′-5′)-2′-methoxyguanylyl-(3′-3′)-thymidine.

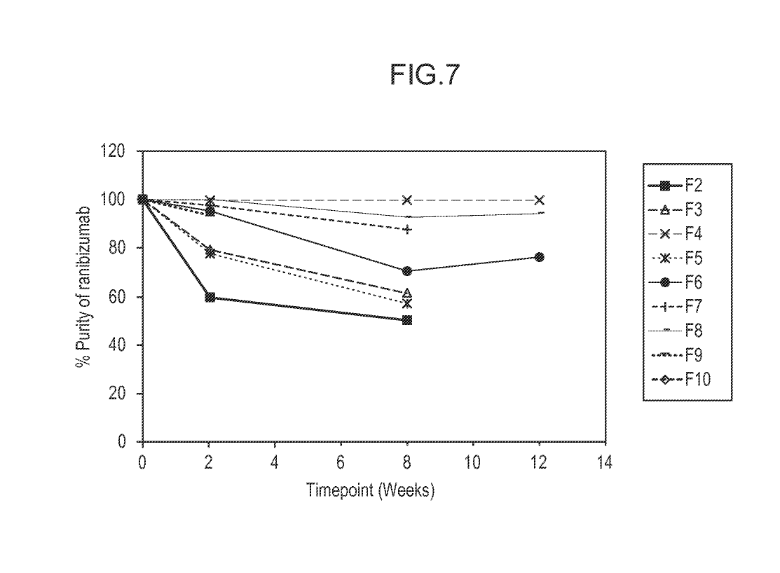

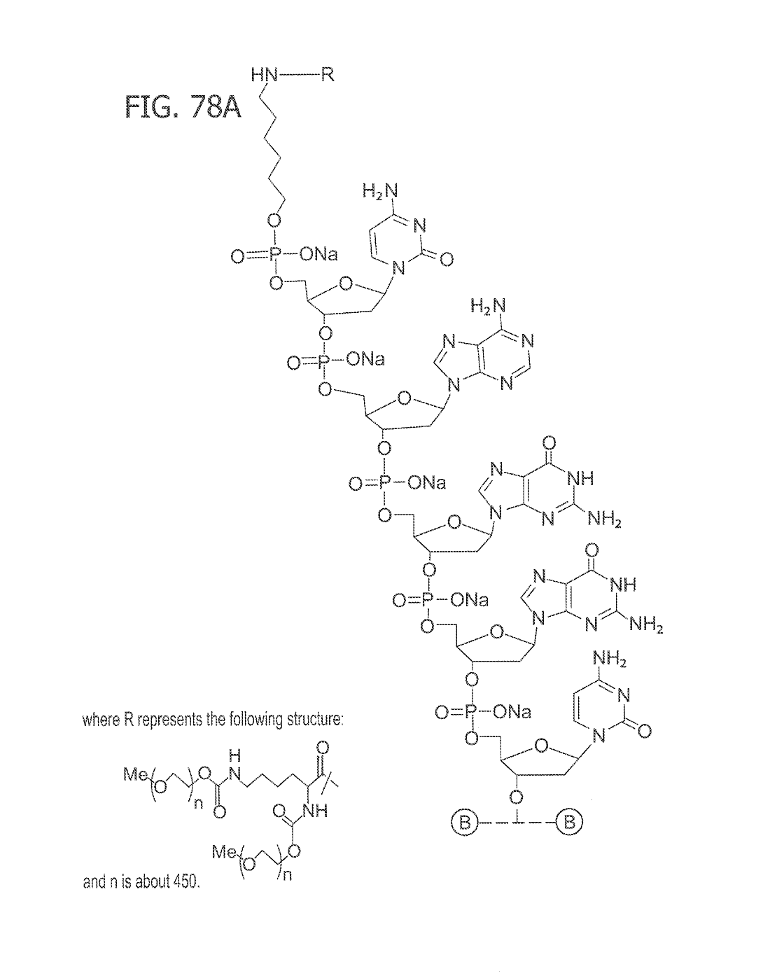

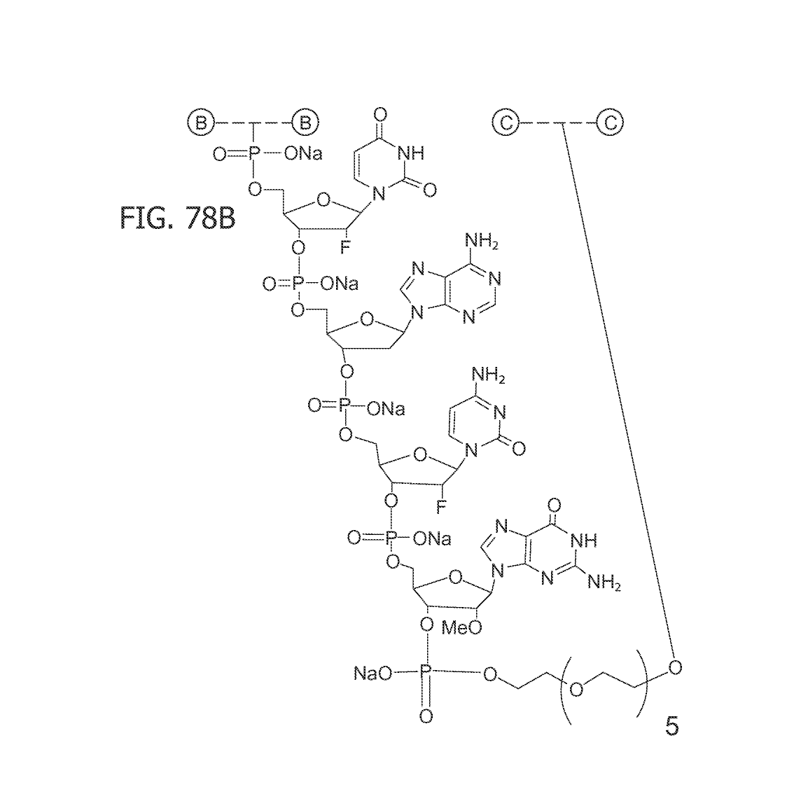

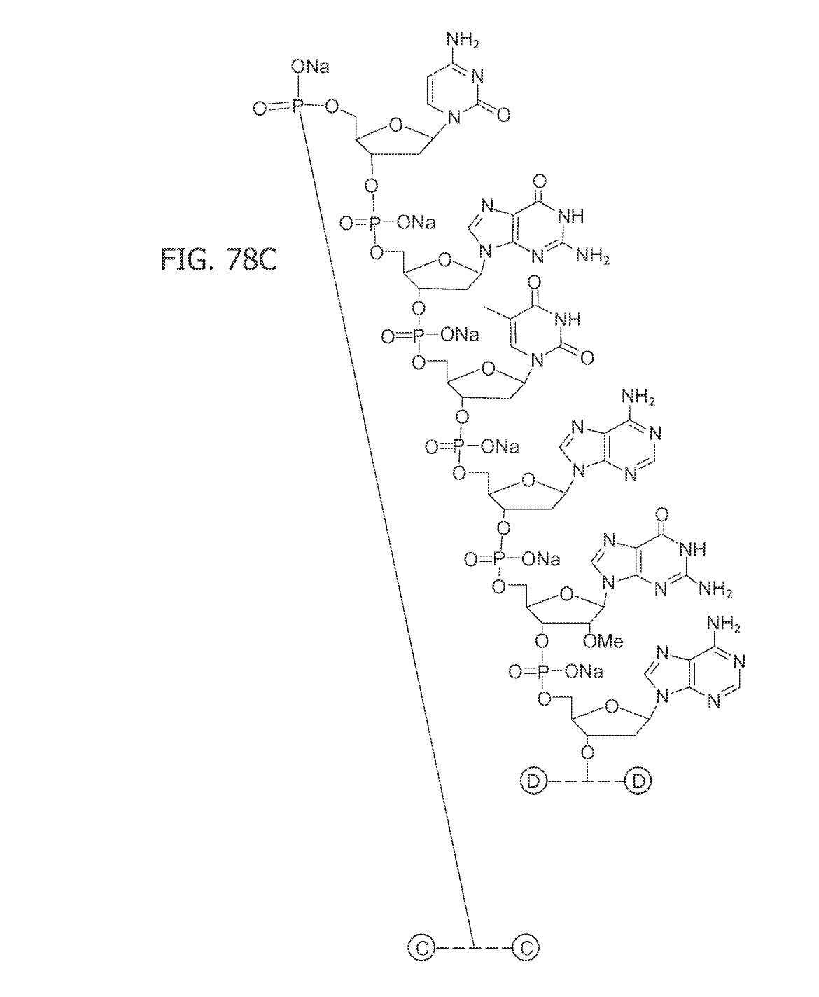

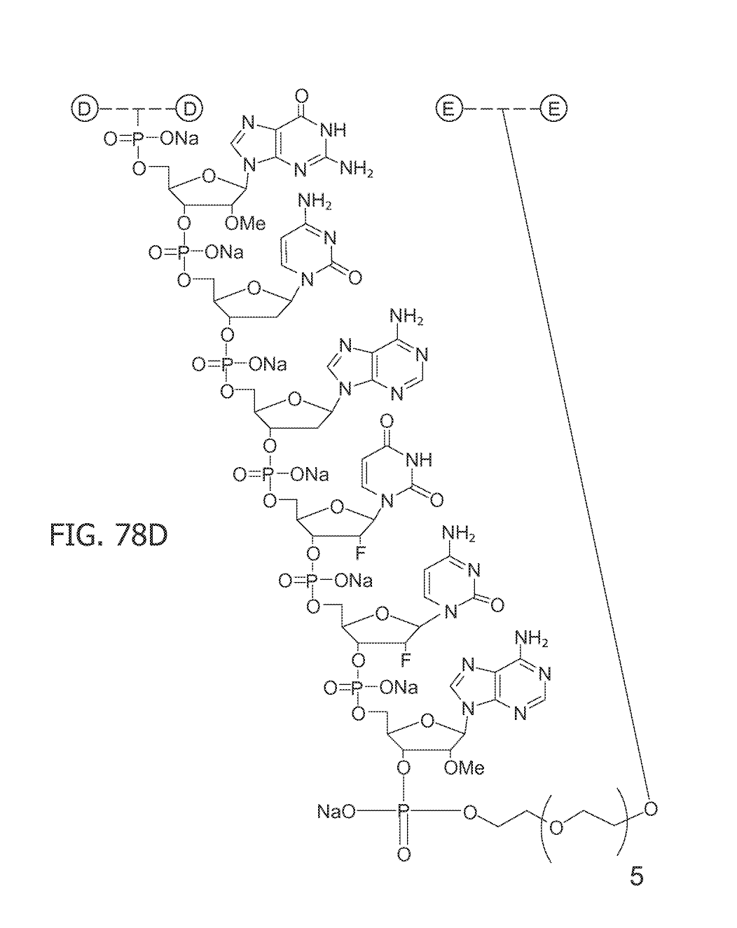

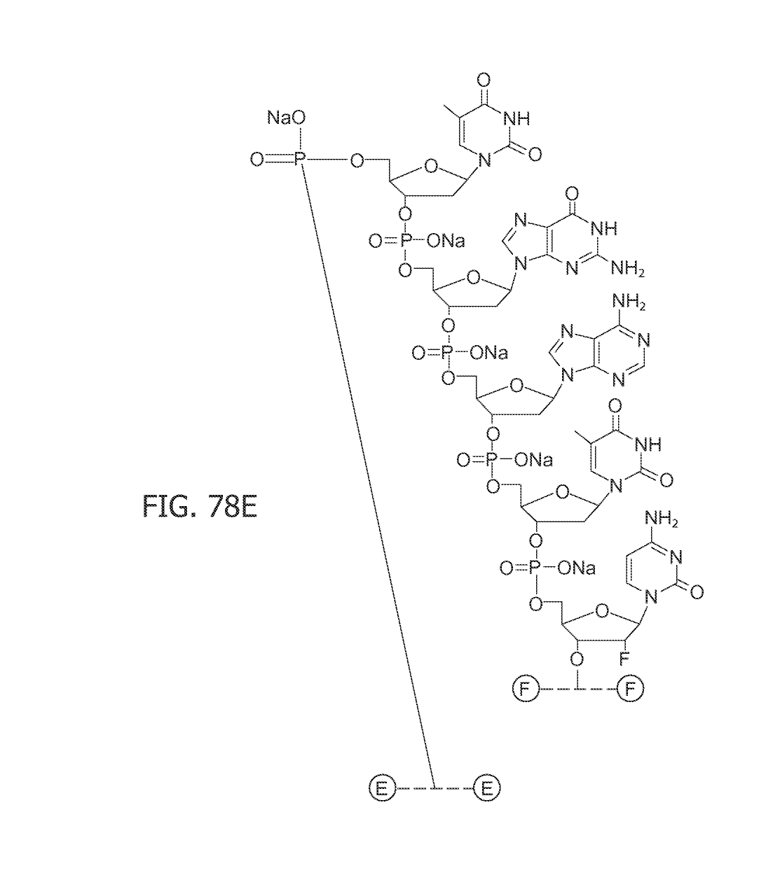

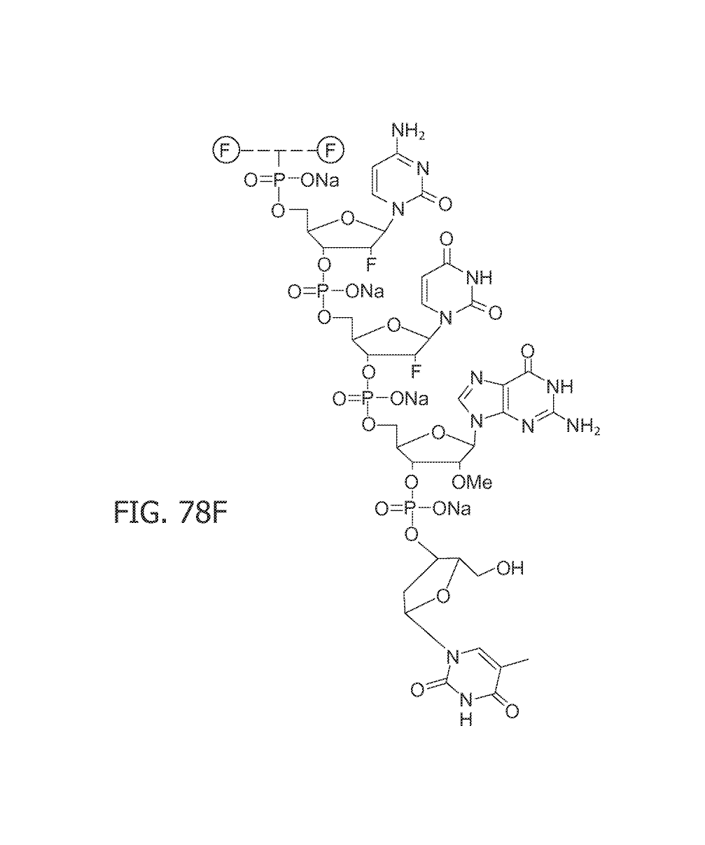

The structure of Antagonist A is shown in FIGS. 78A-F, and it is also described in FIG. 7 of PCT Application Publication No. WO 2010/127029, which is incorporated herein its entirety.

The sequence of Antagonist A is:

5′-[mPEG2 40 kD]-[HN—(CH₂)₆O] CAGGCU_{f}AC_{f}Gₘ [PO₃(CH₂CH₂O)₆] CGTAGₘAGₘCAU_{f}C_{f}Aₘ [PO₃(CH₂CH₂O)₆] TGATC_{f}C_{f}U_{f}Gₘ-iT-3′ (SEQ ID Nos. 1-3), where

[mPEG2 40 kD] represents two 20 kD polyethylene glycol (PEG) polymer chains, in one embodiment two about 20 kD PEG polymer chains, that are covalently attached to the two amino groups of a lysine residue via carbamate linkages. This moiety is in turn linked with the oligonucleotide via the amino linker described below.

[HN—(CH₂)₆O] represents a bifunctional α-hydroxy-w-amino linker that is covalently attached to the PEG polymer via an amide bond. The linker is attached to the oligonucleotide at the 5′-end of Antagonist A by a phosphodiester linkage.

[PO₃(CH₂CH₂O)₆] represents the hexaethylene glycol (HEX) moieties that join segments of the oligonucleotide via phosphodiester linkages. Antagonist A has two HEX linkages that join together the 9^{th} and 10^{th} nucleotides and 21^{st} and 22^{nd} nucleotides via phosphodiester linkages between the linker and the respective nucleotides.

C, A, G, and T represent the single letter code for the 2′-deoxy derivatives of cytosine, adenosine, guanosine, and thymidine nucleic acids, respectively. Antagonist A has four T-deoxyribocytosine, six T-deoxyriboadenosine, four 2′-deoxyriboguanosine, and four 2′-deoxyribothymidine.

Gₘ and Aₘ represent 2′-methoxy substituted forms of guanosine and adenosine, respectively. Antagonist A has four 2′-methoxyguanosines and one 2′-methoxyadenosine. C_{f} and U_{f} represent the 2′-fluoro substituted forms of cytosine and uridine, respectively. Antagonist A has four 2′-fluorocytosines and three 2′-fluorouridines.

The phosphodiester linkages in the oligonucleotide, with the exception of the 3′-terminus, connect the 5′- and 3′-oxygens of the ribose ring with standard nucleoside or nucleotide phosphodiester linkages. The phosphodiester linkage between the 3′-terminal thymidine and the penultimate Gₘ links their respective 3′-oxygens, which is referred to as the 3′,3′-cap.

Antagonist A has a molecular weight from about 40,000 to about 60,000 Daltons for the entire molecule (including the nucleic acid, amino linker and polyethylene glycol moieties), in one embodiment from 40,000 to 60,000 Daltons, and can be colorless to slightly yellow in solution. Antagonist A can be present in a solution of monobasic sodium phosphate monohydrate and dibasic sodium phosphate heptahydrate as buffering agents and sodium chloride as a tonicity adjuster. Antagonist A is a hydrophilic polymer. The Antagonist A sodium salt is soluble in water and in phosphate-buffered saline (PBS), as assessed by visual inspection, to at least about 50 mg (based on oligonucleotide weight)/mL solution.

In one embodiment, Antagonist A is manufactured using an iterative chemical synthesis procedure to produce the oligonucleotide portion and amino linker, which is then covalently bonded to a pegylation reagent, as shown in Example 4 and as described in Example 4 of PCT Application Publication No. WO 2010/127029, which is hereby incorporated by reference in its entirety.

Antagonist A can possess a sufficiently basic functional group, which can react with any of a number of inorganic and organic acids, to form a pharmaceutically acceptable salt. A pharmaceutically-acceptable acid addition salt is formed from a pharmaceutically-acceptable acid, as is well known in the art. Such salts include those described herein and the pharmaceutically acceptable salts listed in Journal of Pharmaceutical Science, 66, 2-19 (1977) and The Handbook of Pharmaceutical Salts, Properties, Selection, and Use, P H Stahl and C G Wermuth (ED s), Verlag, Zurich (Switzerland) 2002, which are hereby incorporated by reference in their entirety.

In other embodiments, the anti-PDGF aptamer is a modified form of an aptamer, such as Antagonist A, or another aptamer described herein, which may include one or more of the modifications described herein. Although discussed specifically with respect to Antagonist A, it is understood that any of the modifications described herein may be present in a modified form of any other anti-PDGF aptamer described herein, each of which may be useful in the present invention. In particular embodiments, a modified form of an aptamer, e.g., a modified form of Antagonist A, comprises or consists of the same nucleotide sequence and nucleic acids as the aptamer, but comprises one or more different polyethylene glycol polymer chains as compared to the aptamer, comprises one or more different linkers coupling one or more of the polyethylene glycol polymer chains to the nucleic acid portion of the aptamer.

In some embodiments, a modified form of an aptamer, e.g., a modified form of Antagonist A, can have chemically modified nucleotides as compared to the aptamer, including 5-X or 2′-Y substitutions in pyrimidine bases and 8-X or 2′-Y substitutions in purine bases. 2′-Modifications, such as 2′-fluoro and 2′-O-Me, can be utilized for stabilization against nucleases without compromising the aptamer binding interaction with the target. See, e.g., Lin et al., Nucleic Acids Res., 22, 5229-5234 (1994); Jellinek et al., Biochemistry, 34, 11363-1137 (1995); Lin et al., Nucleic Acids Res., 22, 5229-5234 (1994); Kubik et al., J. Immunol., 159(1), 259-267 (1997); Pagratis et al., Nat. Biotechnol., 1, 68-73 (1997); and Wilson et al., Curr Opin Chem Biol, 10(6), 607-614 (2006). In some embodiments, the chemical substitution can be a chemical substitution at a sugar position, a chemical substitution at a base position, or a chemical substitution at a phosphate position.

Modifications that may be present in modified forms of an aptamer, e.g., Antagonist A, include, but are not limited to, those which provide other chemical groups that incorporate additional charge, polarizability, hydrophobicity, hydrogen bonding, electrostatic interaction, or fluxionality to the aptamer bases or to the aptamer as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping or modification with sugar moieties. In some embodiments of the invention, the modified forms of an aptamer, e.g., modified forms of Antagonist A, are RNA molecules that are 2′-fluoro (2′-F) modified on the sugar moiety of pyrimidine residues. Examples of modifications that may be present in modified forms of an aptamer, e.g., modified forms of Antagonist A, as well as stabilized aptamers that may be used according to the present invention, are described in U.S. Pat. No. 8,039,443, which is hereby incorporated by reference in its entirety. In certain embodiments, the anti-PDGF aptamer is an anti-PDGF-B aptamer, including but not limited to those described in U.S. Pat. No. 8,039,443.

In some embodiments, the stability of the aptamer can be increased by the introduction of such modifications and as well as by modifications and substitutions along the phosphate backbone of the RNA, which may also be present in modified forms of the aptamer, e.g., modified forms of Antagonist A. In addition, a variety of modifications can be made on the nucleobases themselves which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art. Any such modifications may be present in a modified form of Antagonist A.

Other modifications that may be present in a modified form of an aptamer, e.g., modified form of Antagonist A, include the incorporation of modified bases (or modified nucleoside or modified nucleotides) that are variations of standard bases, sugars or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). A modified form of Antagonist A can include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. A modified form of Antagonist A can include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. A modified form of an aptamer, e.g., a modified form of Antagonist A, may include adenine or any adenine-related base including 6-methyladenine, N6-isopentenyladenine, N6-methyladenine, 1-methyladenine, 2-methyladenine, 2-methylthio-N-6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N2-carboxypropyl)uracil, 5-methylaminomethyluracil, 5-propynyl uracil, 6-azouracil, or 4-thiouracil.

Examples of other modified base variants known in the art, which may be present in a modified verion of an aptamer, e.g., a modified version of Antagonist include, without limitation, 4-acetylcytidine, 5-(carboxyhydroxylmethyl) uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N-6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, undine-5-oxyacetic acid methylester, uridine-5-oxy acetic acid, wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, wybutosine, 3-(3-amino-3-carboxypropyl)uridine.

Examples of modified nucleoside and nucleotide sugar backbone variants known in the art include, without limitation, those having, e.g., 2′-ribosyl substituents such as F, SH, SCH₃, OCN, Cl, Br, CN, CF₃, OCF₃, SOCH₃, SO₂, CH₃, ONO₂, NO₂, N₃, NH₂, OCH₂CH₂OCH₃, O(CH₂)₂ON(CH₃)₂, OCH₂OCH₂N(CH₃)₂, O(C₁₋₁₀ alkyl), O(C₂₋₁₀ alkenyl), O(C₂₋₁₀ alkynyl), S(C₁₋₁₀ alkyl), S(C₂₋₁₀ alkenyl), S(C₂₋₁₀ alkynyl), NH(C₁₋₁₀ alkyl), NH(C₂₋₁₀ alkenyl), NH(C₂₋₁₀ alkynyl), and O-alkyl-O-alkyl. Desirable 2′ ribosyl substituents include 2′-methoxy (2′-OCH₃), 2′-aminopropoxy (2′ OCH₂CH₂CH₂NH₂), 2′-allyl (2′-CH₂—CH═CH₂), 2′-O-allyl (2′-O—CH₂—CH═CH₂), 2′-amino (2′—NH₂), and 2′-fluoro (2′-F). The 2′-substituent may be in the arabino (up) position or ribo (down) position. These may be present in a modified form of Antagonist A.

Examples of modifications include: a purine substitution for a pyrimidine; a 2′-deoxy dihydrouridine substitution for a uridine; a 2′-deoxy-5-methyl cytidine for a cytidine; a 2-amino purine substitution for a purine; a phosphorothioate substituted for a phosphodiester; a phosphorodithioate substituted for a phosphodiester; a deoxynucleotide substituted for a 2′-OH nucleotide; a 2′-OMe nucleotide, a 2′-fluoro nucleotide or a 2′-O-methoxyethyl nucleotide substituted for a 2′-OH or deoxynucleotide; the addition of a PEG or PAG polymer; the addition of a large steric molecule; the addition of a 3′ cap; or any other modification known to block nuclease degradation. See, for example, U.S. Patent Publication No. 20090075342, which is incorporated by reference in its entirety.

Modified forms of an aptamer, e.g., modified forms of Antagonist A, may be made up of nucleotides or nucleotide analogs such as described herein, or a combination of both, or are oligonucleotide analogs. Modified forms of an aptamer, e.g., modified forms of Antagonist A, may contain nucleotide analogs at positions which do not affect the function of the oligomer, for example, to bind PDGF.

The anti-PDGF aptamers described herein can be linked with one or more non-physiologically active groups, such as a lipophilic compound (e.g., cholesterol); linked with one or more non-immunogenic high molecular weight compounds (e.g., polyalkylene glycol); or attached to or encapsulated in a complex comprising a lipophilic component (e.g., a liposome). In one embodiment, the linked aptamers enhance the cellular uptake of the aptamers by a cell for delivery of the aptamers to an intracellular target. U.S. Pat. No. 6,011,020, incorporated by reference herein in its entirety, describes a method for preparing aptamers linked with one or more lipophilic compounds or non-immunogenic, high molecular weight compounds.

The anti-PDGF aptamers described herein may be attached via a linker to one or more non-physiologically active groups, such as lipophilic or Non-immunogenic, High Molecular Weight compounds, in a diagnostic or therapeutic complex as described in U.S. Pat. No. 6,011,020. Aptamers that are attached via a linker to a Lipophilic Compound, such as diacyl glycerol or dialkyl glycerol, in a diagnostic or therapeutic complex are described in U.S. Pat. No. 5,859,228. Aptamers that are attached via a linker to a Lipophilic Compound, such as a glycerol lipid, or to a Non-immunogenic, High Molecular Weight Compound, such as polyalkylene glycol, are further described in U.S. Pat. No. 6,051,698. Aptamers that are attached via a linker to a Non-immunogenic, High Molecular Weight compound or to a lipophilic compound are also further described in PCT/US97/18944, filed Oct. 17, 1997, entitled “Vascular Endothelial Growth Factor (VEGF) Nucleic Acid Ligand Complexes.” Each of the herein described patents and patent applications are specifically incorporated by reference herein in its entirety.

One or more aptamers, e.g., Antagonist A, may be attached via a linker to a Non-Immunogenic, High Molecular Weight compound or lipophilic compound. A Non-Immunogenic, High Molecular Weight compound can be a linear or branched compound that has a molecular weight of about 100 Da to 1,000,000 Da, about 1000 Da to 500,000 Da, or about 1000 Da to 200,000 Da, that typically does not generate an immunogenic response. In one embodiment, the Non-Immunogenic, High Molecular Weight compound can be a polyalkylene glycol. In one embodiment, the Non-Immunogenic, High Molecular Weight compound comprises a polyalkylene glycol. In one embodiment, the Non-Immunogenic, High Molecular Weight compound comprises a plurality of polyalkylene glycols. In one embodiment, the Non-Immunogenic, High Molecular Weight compound comprises two polyalkylene glycols. In another embodiment, the polyalkylene glycol can be polyethylene glycol (PEG). In some embodiments, the PEG has a molecular weight of about 10 to about 80 kDa or a molecular weight of about 20 to about 45 kDa. In some embodiments, the plurality of PEGs has a combined molecular weight of about 10 to about 80 kDa or a molecular weight of about 20 to about 45 kDa. In other embodiments, the Non-Immunogenic, High Molecular Weight compound comprises two polyalkylene glycols, each of which has a molecular weight of about 20 kDa.

An aptamer, e.g., Antagonist A, may be attached via a linker to one or more lipophilic compounds. Lipophilic compounds are compounds that have the propensity to associate with or partition into lipid or other materials or phases having a low dielectric constant, including compounds based mostly on lipophilic components. Lipophilic compounds include lipids as well as non-lipid containing compounds that have the propensity to associate with lipids (or other materials or phases with low dielectric constants). Cholesterol, phospholipid, and glycerol lipids, such as dialkyl glycerol, diacyl glycerol, and glycerol amide lipids are further examples of lipophilic compounds. In one embodiment, the lipophilic compound is a glycerol lipid.

The Non-Immunogenic, High Molecular Weight compound or lipophilic compound can be covalently bound via a linker to a variety of positions on the aptamer, such as to an exocyclic amino group on a nucleotide's base, the 5-position of a pyrimidine nucleotide, the 8-position of a purine nucleotide, the hydroxyl group of a nucleotide's phosphate, or a hydroxyl group or other group at the 5′ or 3′ terminus of the aptamer. In some embodiments where the lipophilic compound is a glycerol lipid, or the Non-Immunogenic, High Molecular Weight compound is polyalkylene glycol or polyethylene glycol, the Non-Immunogenic, High Molecular Weight compound can be bonded via a linker to the 5′ or 3′ hydroxyl of the phosphate group thereof. In one embodiment, the lipophilic compound or Non-Immunogenic, High Molecular Weight compound is bonded via a linker to the 5′ phosphate group of the aptamer. Attachment of the Non-Immunogenic, High Molecular Weight compound or lipophilic compound to the aptamer can be done directly or with the utilization of one or more linkers that interpose between the aptamer and lipophilic compound or Non-Immunogenic, High Molecular Weight compound. When attachment is done directly, in some embodiments, no linker is present.

A linker is a molecular entity that connects two or more molecular entities through covalent bonds or non-covalent interactions, and can allow spatial separation of the molecular entities in a manner that preserves the functional properties of one or more of the molecular entities.

In one embodiment of the invention, the Non-Immunogenic, High Molecular Weight Compound is a polyalkylene glycol and has the structure R(O(CH₂)ₓ)ₙO—, where R is independently H or CH₃, x=2-5, and n˜MW of the Polyalkylene Glycol/(16+14x). In one embodiment of the present invention, the molecular weight of the Polyalkylene Glycol is about between 10-80 kDa. In another embodiment, the molecular weight of the Polyalkylene Glycol is about between 20-45 kDa. In yet another embodiment, x=2 and n=9×10². There can be one or more Polyalkylene Glycols attached via a linker to the same aptamer. In one embodiment, a plurality of Polyalkylene Glycols is attached via a linker to the same aptamer. In another embodiment, two Polyalkylene Glycols are attached via a linker to the same aptamer. In another embodiment, Polyalkylene Glycols is a polyethylene glycol that has a molecular weight of about 40 kDa.

In one embodiment, an anti-PDGF aptamer is attached via a linker to a Non-Immunogenic, High Molecular Weight Compound such as Polyalkylene Glycol or PEG, or to a plurality of Non-Immunogenic, High Molecular Weight Compounds. In this embodiment, the pharmacokinetic properties of the linked PDGF aptamer are improved relative to the anti-PDGF aptamer alone. The Polyalkylene Glycol or PEG can be covalently bound via a linker to a variety of positions on the PDGF aptamer. In embodiments where Polyalkylene Glycol or PEG are used, the anti-PDGF aptamer can be bonded via a linker through the 5′ hydroxyl group via a phosphodiester linkage.

In some embodiments, a plurality of aptamers can be associated with a single Non-Immunogenic, High Molecular Weight Compound, such as Polyalkylene Glycol or PEG, or a Lipophilic Compound, such as a glycerolipid. The aptamers can all be to one target or to different targets. In embodiments where a compound comprises more than one anti-PDGF aptamer, there can be an increase in avidity due to multiple binding interactions with the target, PDGF. In yet further embodiments, a plurality of one or more of Polyalkylene Glycol, PEG, and glycerol lipid molecules can be attached to each other, to the same linker, or to a plurality of linkers. In these embodiments, one or more aptamers can be associated with each Polyalkylene Glycol, PEG, or glycerol lipid. This can result in an increase in avidity of each aptamer to its target. In addition, in embodiments where there are aptamers to PDGF or aptamers to PDGF and different targets associated with Polyalkylene Glycol, PEG, or glycerol lipid, a drug can also be associated with, e.g., covalently bonded to, Polyalkylene Glycol, PEG, or glycerol lipid. Thus the compound would provide targeted delivery of the drug, with Polyalkylene Glycol, PEG, or glycerol lipid serving as a linker, optionally, with one or more additional linkers.

In particular embodiments, aptamers can be 5′-capped and/or 3′-capped with a 5′-5′ inverted nucleotide cap structure at the 5′ end and/or a 3′-3′ inverted nucleotide cap structure at the 3′ end. In certain embodiments, Antagonist A (or a modified form of Antagonist A) is 5′ or 3′ end-capped. In other embodiments, the nucleotide cap is an inverted thymidine.

VEGF Antagonists

VEGF antagonists useful in the compositions of the invention include, but are not limited to, ranibizumab, bevacizumab, aflibercept, and pharmaceutically acceptable salts thereof.

In certain embodiments, a VEGF antagonist is an antibody, or fragment thereof, that binds human VEGF, which may be a humanized or human anti-VEGF antibody. In particular embodiments, an anti-VEGF antibody heavy chain variable domain comprises the amino acid sequence:

EVQLVESGGGLVQPGGSLRLSCAASGYX₁FTX₂YGMNWVRQAPGKGLEWVGW INTYTGEPTYAADFKRRFTFSLDTSKSTAYLQMNSLRAEDTAVYYCAKYPX₃Y YG X₄SHWYFDNvVGQGTLVTVSS (SEQ ID NO:76), wherein X₁ is T or D; X₂ is N or H; X₃ is Y or H; and X₄ is S or T. In a particular embodiment, the heavy chain variable domain comprises the amino acid sequence: EVQLVESGGGLVQPGGSLRLSCAASGYTFTNYGMNWVRQAPGKGLEWVGWI NTYTGEPTYAADFKRRFTFSLDTSKSTAYLQMNSLRAEDTAVYYCAKYPHYY GSSHWYFDVWGQGTL (SEQ ID NO:77). These heavy chain variable domain sequences may be combined with the following light chain variable domain sequences or with other light chain variable domain sequences, provided that the antibody so produced binds human VEGF.

In certain embodiments, an anti-VEGF antibody light chain variable domain comprises hypervariable regions with the following amino acid sequences: CDRL1 (SASQDISNYLN [SEQ ID NO:78]), CDRL2 (FTSSLHS [SEQ ID NO:79]) and CDRL3 (QQYSTVPWT [SEQ ID NO:80]). In particular embodiment, the three light chain hypervariable regions are provided in a human framework region, e.g., as a contiguous sequence represented by the following formula: FR1-CDRL1-FR2-CDRL2-FR3-CDRL3-FR4. In one embodiment, an anti-VEGF antibody light chain variable domain comprises the amino acid sequence: DIQX₁TQSPSSLSASVGDRVTITCSASQDISNYLNWYQQKPGKAPKVLIYFTSSL HS GVPSRFSGSGSGTDFTLTISSLQPEDFATYYCQQYSTVPWTFGQGTKVEIKR (SEQ ID NO:81), wherein X₁ is M or L. In particular embodiments, the light chain variable domain comprises the amino acid sequence: DIQMTQSPSSLSASVGDRVTITCSASQDISNYLNWYQQKPGKAPKVLIYFTSSLH SGVPSRFSGSGSGTDFTLTISSLQPEDFATYYCQQYSTVPWTFGQGTKVEIKRTV (SEQ ID NO:82). These light chain variable domain sequences may be combined with the above-identified heavy chain variable domain sequences or with other heavy chain variable domain sequences, provided that the antibody so produced retains the ability to bind to human VEGF.

In one particular embodiment, the VEGF antagonist is the antibody bevacizumab or a pharmaceutically acceptable salt thereof, which includes the following heavy and light chain variable domain sequences, respectively: EVQLVESGGGLVQPGGSLRLSCAASGYTFTNYGMNWVRQAPGKGLEWVGWI NTYTGEPTYAADFKRRFTFSLDTSKSTAYLQMNSLRAEDTAVYYCAKYPHYY GSSHWYFDVWGQGTL (SEQ ID NO:77); and DIQMTQSPSSLSASVGDRVTITCSASQDISNYLNWYQQKPGKAPKVLIYFTSSLH SGVPSRFSGSGSGTDFTLTISSLQPEDFATYYCQQYSTVPWTFGQGTKVEIKRTV (SEQ ID NO:82). Bevacizumab is commercially available under the trademark Avastin® (Genentech, S. San Francisco, Calif.) and is also described in U.S. Pat. No. 6,054,297.

In certain embodiments, the VEGF antagonist is a variant of a parent anti-VEGF antibody (which parent is optionally a humanized or human anti-VEGF antibody), wherein the variant binds human VEGF and comprises an amino acid substitution in a hypervariable region of the heavy or light chain variable domain of the parent anti-VEGF antibody. In particular embodiments, the variant has one or more substitution(s) in one or more hypervariable region(s) of the anti-VEGF antibody. In more particular embodiments, the substitution(s) are in the heavy chain variable domain of the parent antibody. For example, the amino acid subsition(s) may be in the CDRH1 or CDRH3 of the heavy chain variable domain, or there may be substitutions in both these hypervariable regions. In certain embodiments, such “affinity matured” variants bind human VEGF more strongly than the parent anti-VEGF antibody from which they are generated, i.e., they have a K_{d} value which is significantly less than that of the parent anti-VEGF antibody. In certain embodiments, the variant has an ED50 value for inhibiting VEGF-induced proliferation of endothelial cells in vitro which is at least about 10 fold lower, at least about 20 fold lower, or at least about 50 fold lower, than that of the parent anti-VEGF antibody. In one embodiment, a variant has a CDRH1 comprising the amino acid sequence: GYDFTHYGMN (SEQ ID NO:83) and a CDRH3 comprising the amino acid sequence: YPYYYGTSHWYFDV (SEQ ID NO:84). These hypervariable regions and CDRH2 may be provided in a human framework region, e.g., resulting in a heavy chain variable domain comprising the following amino acid sequence: EVQLVESGGGLVQPGGSLRLSCAASGYDFTHYGMNWVRQAPGKGLEWVGWI NTYTGEPTYAADFKRRFTFSLDTSKSTAYLQMNSLRAEDTAVYYCAKYPYYY GTSHWYFDVWGQGTL (SEQ ID NO:77). Such heavy chain variable domain sequences are optionally combined with a light chain variable domain comprising the amino acid domain comprising the following amino acid sequence:

(SEQ ID NO: 82)
DIQLTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTV.

In one embodiment, the VEGF antagonist is the antibody fragment ranibizumab or a pharmaceutically acceptable salt thereof, which includes the following heavy and light chain variable domain sequences, respectively: EVQLVESGGGLVQPGGSLRLSCAASGYDFTHYGMNWVRQAPGKGLEWVGWI NTYTGEPTYAADFKRRFTFSLDTSKSTAYLQMNSLRAEDTAVYYCAKYPYYY GTSHWYFDVWGQGTL (SEQ ID NO:77); and DIQLTQSPSSLSASVGDRVTITCSASQDISNYLNWYQQKPGKAPKVLIYFTSSLH SGVPSRFSGSGSGTDFTLTISSLQPEDFATYYCQQYSTVPWTFGQGTKVEIKRTV (SEQ ID NO:82). Ranibizumab is commercially available under the trademark Lucentis®, in which it is formulated for intravitreal administration (Genentech, S. San Francisco, Calif.) and is also described in U.S. Pat. No. 7,060,269.

In another embodiment, the VEGF antagonist is a VEGF-Trap™, such as aflibercept or a pharmaceutically acceptable salt thereof (see Do et al. (2009) Br J. Ophthalmol. 93: 144-9, which is hereby incorporated by reference in its entirety). Aflibercept is also known by the name VEGF-Trap-Eye™ and is commercially available under the trademark Eylea™ (Regeneron Pharmaceuticals, Tarrytown, N.Y.). In particular embodiments, a VEGF-Trap™ comprises a dimeric fusion polypeptide comprising two fusion polypeptides, each fusion polypeptide comprising a VEGF receptor component consisting of an immunoglobulin-like (Ig) domain 2 of a first VEGF receptor human Flt1 and an Ig domain 3 of a second VEGF receptor human Flkl of human Flt4. Aflibercept is a fusion protein comprising Fc fragments of IgG fused to VEGF receptor 1 domain 2 and VEGF receptor 2 domain 3, which binds both VEGF-A and Placental Growth Factor (PlGF). Aflibercept is a dimeric glycoprotein with a protein molecular weight of 97 kilodaltons (kDa) and contains glycosylation, constituting an additional 15% of the total molecular mass, resulting in a total molecular weight of 115 kDa. Illustrative VEGF-Traps, including aflibercept, and methods of producing the same are described in U.S. Pat. Nos. 7,306,799, 7,531,173, 7,608,261, 7,070,959, 7,374,757, and 7,374,758. In particular embodiments, a VEGF-Trap™ is a polypeptide comprising or consisting of the following amino acid sequence:

(SEQ ID NO: 85)
MVSYWDTGVLLCALLSCLLLTGSSSGSDTGRPFVEMYSEIPEIIHMTEGR
ELVIPCRVTSPNITVTLKKFPLDTLIPDGKRIIWDSRKGFIISNATYKEI
GLLTCEATVNGHLYKTNYLTHRQTNTIIDVVLSPSHGIELSVGEKLVLNC
TARTELNVGIDFNWEYPSSKHQHKKLVNRDLKTQSGSEMKKFLSTLTIDG
VTRSDQGLYTCAASSGLMTKKNSTFVRVHEKDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPGK.

### Compositions

The present invention provides compositions, including pharmaceutical compositions, comprising an anti-PDGF aptamer and a VEGF antagonist. In some embodiments, the compositions provide stability to anti-PDGF aptamers or VEGF antagonists, or to both anti-PDGF aptamers and VEGF antagonists, including those useful in treating or preventing ophthalmological diseases. In particular embodiments, the one or more anti-PDGF aptamers present in the composition is the aptamer Antagonist A or a modified form thereof. In particular embodiments, the one or more VEGF antagonists present in the composition is one or more of ranibizumab, bevacizumab, and aflibercept. In particular embodiments, compositions of the invention comprise: (i) Antagonist A (or a modified form thereof) and ranibizumab; (ii) Antagonist A (or a modified form thereof) and bevacizumab; or (iii) Antagonist A (or a modified form thereof) and aflibercept. In certain embodiments, the compositions comprise a pharmaceutically acceptable salt of any of the anti-PDGF aptamers or VEGF antagonists. In particular embodiments, at least about 90% of the anti-PDGF aptamer or VEGF antagonist is chemically stable when the composition is stored at a temperature of from about 2.0° C. to about 8.0° C. for at least about twelve weeks.

The relative concentrations of the anti-PDGF aptamer and the VEGF antagonist present in a composition of the invention may be determined based on the strength and specificity of these antagonists, and the types and concentration of their binding targets. In one embodiment, the anti-PDGF aptamer and the VEGF antagonist are present in substantially equal concentration in the composition. In another embodiment, the anti-PDGF aptamer or the VEGF antagonist is present in a substantially higher concentration than the other, e.g, the ratio of the anti-PDGF aptamer:VEGF antagonist concentrations in a composition is about 1.5:1, about 2:1, about 2.5:1, about 3:1, about 4:1, or about 5:1, or the ratio of the VEGF antagonist:anti-PDGF aptamer concentrations in a composition is about 1.5:1, about 2:1, about 2.5:1, about 3:1, about 4:1, or about 5:1. In certain embodiments, the ratio of the anti-PDGF aptamer:VEGF antagonist concentration in a composition is in the range of about 1:1 to about 5:1, about 1.5:1 to about 5:1, or about 2.0:1 to about 5:1; in other embodiments, the ratio of the VEGF antagonist:anti-PDGF aptamer concentration in a composition is in the range of about 1:1 to about 5:1, about 1.5:1 to about 5:1, or about 2.0:1 to about 5:1. Unless otherwise indicated, the concentration of an aptamer is based solely on the molecular weight of the nucleic acid portion of the aptamer, which can optionally comprise a short-chain polyethylene glycol. Where the nucleic acid portion comprises a short chain polyethylene glycol, the molecular weight of the nucleic acid portion includes the molecular weight of all short chain polyethylene glycol residues.

In some embodiments, the anti-PDGF aptamer and the VEGF antagonist are each present in the composition of the invention at a concentration from about 0.1 mg/mL to about 200 mg/mL, about 1 to about 150 mg/mL, about 2 mg/mL to about 100 mg/mL, about 3 mg/mL to about 80 mg/mL, about 4 mg/mL to about 50 mg/mL, about 4 mg/mL to about 30 mg/mL, about 5 mg/mL to about 25 mg/mL, or about 5 mg/mL to about 20 mg/mL. In some embodiments, the anti-PDGF aptamer is present in the composition at a concentration from about 0.1 mg/mL to about 200 mg/mL, about 1 to about 150 mg/mL, about 2 mg/mL to about 100 mg/mL, about 3 mg/mL to about 80 mg/mL, about 4 mg/mL to about 50 mg/mL, about 4 mg/mL to about 30 mg/mL, about 5 mg/mL to about 25 mg/mL, or about 5 mg/mL to about 20 mg/mL. In some embodiments, the VEGF antagonist is present in the composition at a concentration from about 0.1 mg/mL to about 200 mg/mL, about 1 to about 150 mg/mL, about 2 mg/mL to about 100 mg/mL, about 3 mg/mL to about 80 mg/mL, about 4 mg/mL to about 50 mg/mL, about 4 mg/mL to about 30 mg/mL, about 5 mg/mL to about 25 mg/mL, about 10 mg/mL to about 25 mg/mL, or about 5 mg/mL to about 20 mg/mL. In some embodiments, the anti-PDGF aptamer and the VEGF antagonist are each present at a concentration of at least about 0.1 mg/mL, at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, at least about 10 mg/mL, at least about 15 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 120 mg/mL, at least about 150 mg/mL or at least about 200 mg/mL. In some embodiments, at least one of the anti-PDGF aptamer or VEGF antagonist is present at a concentration of at least about 0.1 mg/mL, at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, at least about 10 mg/mL, at least about 15 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 120 mg/mL, at least about 150 mg/mL or at least about 200 mg/mL.

Compositions of the invention may also comprise one or more excipients, buffers (i.e., buffering agents), cryoprotectants, tonicity agents (i.e., tonicity modifiers), liquids, stabilizers, surfactants (e.g., nonionic surfactants), lyoprotectants, antioxidants, amino acids, pH-adjusting agents or preservatives, such as any of those described herein. Suitable buffering agents include, but are not limited to, monobasic sodium phosphate, dibasic sodium phosphate, tris(hydroxymethyl)aminomethane (Tris) and sodium acetate. In certain embodiments, a buffer is capable of adjusting the pH of a composition to a desired pH or within a desired pH range, and/or is capable of achieving or maintaining the pH of a composition at a desired pH or within a desired pH range. Suitable nonionic surfactants include, but are not limited to, polyoxyethylene sorbitan fatty acid esters such as polysorbate 20 and polysorbate 80. Suitable preservatives include, but are not limited to, benzyl alcohol. Suitable tonicity agents include, but are not limited to sodium chloride, mannitol, and sorbitol. Suitable lyoprotectants include, but are not limited to, sucrose, trehalose, and mannitol. Suitable amino acids include, but are not limited to glycine and histidine. Suitable pH-adjusting agents (or agents capable of achieving or maintaining a desired pH or pH range) include, but are not limited to, hydrochloric acid, acetic acid, and sodium hydroxide. In one embodiment, the pH-adjusting agent or agents (or agent(s) capable of achieving or maintaining a desired pH or pH range) are present in an amount effective to provide a composition with a pH of about 3 to about 8, about 4.0 to about 8.0, about 4 to about 7, about 5 to about 6, about 6 to about 7, about 6 to about 8, or about 7 to about 7.5. Suitable excipients for a composition also include those described in U.S. Pat. No. 7,365,166, the contents of which are herein incorporated by reference in its entirety.

In particular embodiments, compositions of the invention comprise the following: (1) an anti-PDGF aptamer; (2) a VEGF antagonist; (3) a buffer; (4) a tonicity modifier; and, optionally, (5) a surfactant. In specific embodiments of such compositions, the buffer is acetate, phosphate, Tris or histidine, or a mixture thereof; the tonicity modifier is sodium chloride, mannitol, sorbitol, or trehalose, or a mixture thereof; and the surfactant is polysorbate 20. In various embodiments, the anti-PDGF aptamer is present in the composition of the invention at a concentration of about 0.1 mg/mL to about 200 mg/mL; the VEGF antagonist is present at a concentration of about 0.1 mg/mL to about 200 mg/mL; the buffer is present at a concentration of about 1 mM to about 200 mM; the tonicity modifier is present at a concentration of about 10 mM to about 200 mM (sodium chloride), about 1% to about 10% (w/v) (sorbitol), or about 1% to about 20% (w/v) (trehalose); and the surfactant, when present, is present at a concentration of about 0.005% to about 0.05% or a concentration of about 0.001% to about 0.05%.

The compositions of the invention are, in one useful aspect, administered parenterally (e.g., by intramuscular, intraperitoneal, intravenous, intraocular, intravitreal, retro-bulbar, subconjunctival, subtenon or subcutaneous injection or implant) or systemically. Compositions for parenteral or systemic administration may include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, gelatin, hydrogels, hydrogenated naphalenes, and injectable organic esters, such as ethyl oleate. Such compositions may also contain auxiliary substances, such as preserving, wetting, buffering, emulsifying, or dispersing agents. Biocompatible, biodegradable lactide polymer, lactide/glycolide copolymer, or polyoxyethylene-polyoxypropylene copolymers may be used to control the release of the active ingredients. In one embodiment, a composition comprising an anti-PDGF aptamer and a VEGF antagonist is in the form of an aqueous solution that is suitable for injection. In one embodiment, a composition comprises an anti-PDGF aptamer, a VEGF antagonist, a buffering agent, a pH-adjusting agent (or agent capable of achieving or maintaining a desired pH or pH range), and water for injection.

In some examples, the compositions of the invention can also be administered topically, for example, by patch or by direct application to a region, such as the epidermis or the eye, susceptible to or affected by a neovascular disorder, or by iontophoresis.

Compositions of the invention may be administered intraocularly by intravitreal injection into the eye as well as by subconjunctival and subtenon injections. Other routes of administration include transcleral, retrobulbar, intraperitoneal, intramuscular, and intravenous. Alternatively, compositions can be administered using a drug delivery device or an intraocular implant. Compositions useful for ophthalmic use include pharmaceutical compositions comprising an anti-PDGF aptamer and a VEGF antagonist in admixture with a pharmaceutically acceptable excipient, including those described herein. These excipients may be, for example, buffers, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

In particular embodiments, compositions of the invention confer physical or chemical stability to one or more of the anti-PDGF aptamers or VEGF antagonists present in the composition. In these embodiments, the compositions of the invention are physically or chemically stable compositions. For example, compositions of the invention may render the anti-PDGF aptamer(s) or VEGF antagonist(s) present in the composition physically or chemically stable during storage. Various analytical techniques useful for evaluating the stability of the anti-PDGF aptamer(s) and VEGF antagonist(s) are available in the art, including those described in the accompanying Examples, and those reviewed in Reubsaet et al. (1998) J. Pharm. Biomed. Anal. 17(6-7): 955-78 and Wang (1999) Int. J. Pharm. 185(2): 129-88, including visual inspection, SDS-PAGE, IEF, (high pressure) size exclusion chromatography (HPSEC), RFFIT, kappa/lambda ELISA. Methods described in the accompanying Examples include SE-HPLC, AEX-HPLC, and WCX-HPLC.

An anti-PDGF aptamer's or a VEGF antagonist's physical stability in a composition of the invention can be determined by, but not limited to, measuring the aptamer's or antagonist's state of physical integrityₛ determining whether it shows any sign of aggregation, precipitation or denaturation upon visual examination of color or clarity, or performing UV light scattering or by size exclusion chromatography (SEC) or differential scanning calorimetry (DSC). For example, micro-flow analysis can be used to measure the presence and size of subvisible particles in a composition, e.g., as described in Example 4.

An anti-PDGF aptamer's or a VEGF antagonist's chemical stability in a composition of the invention can be determined by, but not limited to, measuring its state of chemical integrity or determining whether it shows any sign of decomposition or modification resulting in formation of a new chemical entity. Chemical integrity can be assessed by detecting and quantifying chemically altered forms of the aptamer or antagonist. Chemical alteration may involve size modification (e.g., clipping) which can be evaluated using size exclusion chromatography, SDS-PAGE, size exclusion chromatography with HPLC (to determine the presence of LMW and HMW species) or matrix-assisted laser desorption ionization/time-of-flight mass spectrometry (MALDI/TOF MS), for example. Suitable systems for making such measurements are known in the art, e.g., HPLC systems (Waters, Milford, Mass.) and cation exchange-HPLC (CEX-HPLC to detect variants and monitor surface charge). In addition, the methods described in the accompanying Examples useful for measuring stability of anti-PDGF aptamers or VEGF antagonists may be used. These include SE-HPLC, WCX-HPLC, and AEX-HPLC. Other types of chemical alteration include charge alteration (e.g. occurring as a result of deamidation) which can be evaluated by ion-exchange chromatography, for example. Oxidation is another chemical modification that can be detected using methods disclosed herein or methods known to those skilled in the art.

In particular embodiments, a composition of the invention is physically stable if at least about 50%, at least about 60%, at least about 70%, at least about 80%, at least about 90%, a least about 95%, or at least about 99% of an anti-PDGF aptamer or a VEGF antagonist present in the composition shows no sign of aggregation, precipitation or denaturation upon visual examination of color or clarity, or as measured by UV light scattering or by size exclusion chromatography (SEC) or differential scanning calorimetry (DSC). In particular embodiments, a composition is physically stable if at least about 50%, at least about 60%, at least about 70%, at least about 80%, at least about 90%, at least about 95%, or at least about 99% of both the anti-PDGF aptamer(s) and the VEGF antagonist(s) present in the composition show no sign of aggregation, precipitation or denaturation upon visual examination of color or clarity, or as measured by UV light scattering or by size exclusion chromatography (SEC) or differential scanning calorimetry (DSC).

In certain embodiments, physical stability may be determined by micro-flow imaging, where a greater number of particles or greater size of particles detected generally correlates with reduced physical stability. In particular embodiments, a composition of the invention is physically stable if its particle count as determined by micro-flow imaging, e.g., as described in Example 4, e.g., is less than about 500,000, less than about 100,000, or less than about 50,000 total particles/mL, where the particles have an equivalent circular diameter in the range of 0 m to about 100 m or, in another embodiment, in the range of 0 m to about 25 m. In another embodiment, a composition of the invention is considered physically stable if its particle count as determined by micro-flow imaging, e.g., as described in Example 4, e.g., is less than about 100,000, less than about 50,000, less than about 20,000, less than about 10,000, less than about 5,000, less than about 2,500, less than about 1,000, or less than about 500 particles/mL, where the particles have an equivalent circular diameter in the range of about 1 m to about 2 m or, in another embodiment, in the range of about 1 m to about 5 m.

In particular embodiments, a composition of the invention is chemically stable when at least about 50%, at least about 60%, at least about 70%, at least about 80%, at least about 90%, a least about 95%, or at least about 99% of an anti-PDGF aptamer or a VEGF antagonist present in the composition shows no decomposition or modification resulting in formation of a new chemical entity.

In particular embodiments, an anti-PDGF aptamer or a VEGF antagonist is chemically stable when at least about 50%, at least about 60%, at least about 70%, at least about 80%, at least about 90%, a least about 95%, or at least about 99% of an anti-PDGF aptamer or a VEGF antagonist shows no decomposition or modification resulting in formation of a new chemical entity. In particular embodiments, a composition of the invention is chemically stable if at least about 50%, at least about 60%, at least about 70%, at least about 80%, at least about 90%, a least about 95%, or at least about 99% of both the anti-PDGF aptamer(s) and the VEGF antagonist(s) present in the composition show no decomposition or modification resulting in formation of a new chemical entity. In certain embodiments, the decomposition or modification is that which results in formation of a new chemical entity, for example, by chemical bond cleavage.

In particular embodiments, a composition of the invention is chemically stable when at least about 50%, at least about 60%, at least about 70%, at least about 80%, at least about 90%, a least about 95%, or at least about 99% of one or more of the anti-PDGF aptamers or VEGF antagonists in the composition show no sign of decomposition or modification resulting in formation of a new chemical entity, when stored at about room temperature for at least five days, at least seven days, at least 10 days, at least 14 day, at least 20 days, at least 30 days, at least two weeks, at least four weeks, at least eight weeks, at least twelve weeks, at least sixteen weeks, or at least 24 weeks, at least two months, at least three months, at least four months, at least six months, or at least about a year, or alternatively for at least about two years, or alternatively for at least about three years, or alternatively for at least about four years, or alternatively for at least about five years; or alternatively at a temperature from about 2.0° C. to about 8.0° C. for at least five days, at least seven days, at least 10 days, at least 14 day, at least 20 days, at least 30 days, at least 30 days, at least two weeks, at least four weeks, at least eight weeks, at least twelve weeks, at least sixteen weeks, at least 24 weeks, at least two months, at least three months, at least four months, at least six months, or at least about a year, or alternatively for at least about two years, or alternatively for at least about three years, or alternatively for at least about four years, or alternatively for at least about five years; or alternatively at a temperature of about 5.0° C. for at least two weeks, at least four weeks, at least eight weeks, at least twelve weeks, at least sixteen weeks, at least 24 weeks, at least about one year, or at least about two years, or alternatively for at least about three years, or alternatively for at least about four years, or alternatively for at least about five years. In particular embodiments, a composition is physically or chemically stable when the anti-PDGF aptamer(s) and VEGF antagonist(s) present in the composition are chemically stable. In some embodiments, the compositions of the invention are stable, i.e., physically or chemically stable, at about 40° C. for up to or at least one week, up to or at least two weeks, or up to or at least one month. In some embodiments, the compositions are stable at about −20° C. for up to or at least one year, or alternatively up to or least two years, three years, four years, or five years. In some embodiments, the compositions are stable at about −80° C. for up to or at least one year, of alternatively up to or at least two years, three years, four years, or five years. In certain embodiments, the compositions of the invention are physicallr or chemically stable if their particle count as determined by micro-flow imaging as described, e.g., in Example 4, e.g., is less than about 500,000, less than about 100,000, or less than about 50,000 total particles/mL, where the particles have an equivalent circular diameter in the range of 0 m to about 100 m or, in another embodiment, in the range of 0 m to about 25 m; or is less than about 100,000, less than about 50,000, less than about 20,000, less than about 10,000, less than about 5,000, less than about 2,500, less than about 1,000, or less than about 500 particles/mL, where the particles have an equivalent circular diameter in the range of 1 m to 2 m or, in another embodiment, in the range of 1 m to 5 m, after storage at about 5° C. or about 30° C. for about four hours.

In particular embodiments, the compositions of the invention are considered physically or chemically stable if after storage the average number of particles detected does not exceed about 50 particles/mL, where the particles have a diameter >about 10 m and does not exceed 5 particles/mL, where the particles have a diameter >25 m, as measured by the Light Obscuration Particle Count Test described in (788) Particulate Matter in Injections, Revised Bulletin, Official Oct. 1, 2011, The United States Pharmacopeial Convention. As described therein, this test is performed using a suitable apparatus based on a principle of light blockage that allows for an automatic determination of the size of particles and the number of particles according to size. The apparatus is calibrated using dispersions of spherical particles of known sizes from 10 m to 25 m. These standard particles are dispersed in particle-free water. Care is taken to avoid aggregation of particles during dispersion. The test is carried out under conditions that limit exposure to extraneous particulate matter, for example, in a laminar flow cabinet. Glassware and filtration equipment used, except for the membrane filters, are carefully washed with a warm detergent solution and rinsed with abundant amounts of water to remove all traces of detergent. Immediately before use, the equipment is rinsed from top to bottom, outside and then inside, with particle-free water. Care is taken to not introduce air bubbles into the sample to be measured, especially when fractions of the preparation are being transferred to the container in which the measurement is to be carried out. In order to check that the environment is suitable for the test, that the glassware is properly cleaned, and that the water to be used is particle-free, the particulate matter in 5 samples of particle-free water, each of 5 mL, is determined as immediately follows. If the number of particles of 10 m or greater exceeds 25 for the combined 25 mL, the precautions taken for the test are not sufficient. The preparatory steps are then repeated until the environment, glassware, and the water are suitable.

Once the environment, glassware, and water are suitable for the test, the test is conducted on the test sample. The contents of the sample are mixed by slowly inverting the sample's container 20 times successively. If necessary, the container's sealing closure, if any, is cautiously removed. The outer surfaces of the container are cleaned using a jet of particle-free water and the container's sealing closure, if any, is removed, avoiding any contamination of the contents. Gas bubbles are eliminated by appropriate measures such as allowing the container to stand for 2 minutes or sonicating.

For large-volume samples, 25 mL of greater of volume, single units are tested. For small-volume samples, less than 25 mL of volume, the contents of 10 or more units are combined in a cleaned container to obtain a volume of not less than 25 mL; the test solution may be prepared by mixing the contents of a suitable number of vials and diluting the resultant mixture to 25 mL with particle-free water or with an appropriate particle-free solvent when particle-free water is not suitable. Small-volume parenterals having a volume of 25 mL or more may be tested individually. Powders are reconstituted with particle-free water or with an appropriate particle-free solvent when particle-free water is not suitable. The number of test samples should be adequate to provide a statistically significant assessment. For large-volume samples or for small-volume samples having a volume of 25 mL or more, fewer than 10 units may be tested, using an appropriate sampling plan.

Four portions, not less than 5 mL each, are removed from each sample, and the number of particles equal to or greater than 10 μm or 25 μm are counted. The result obtained for the first portion is disregarded, and the mean number of particles for the preparation being examined is calculated.

For samples in containers having a nominal volume of more than 100 mL, the criteria of Test 1.A described herein should be considered.

For samples in containers having a nominal volume of 100 mL or less, the criteria of Test 1.B described herein should be considered.

If the average number of particles exceeds the test limits, the sample should be tested using the Microscopic Particle Count Test.

Test 1.A. The sample complies with the test limits if the average number of particles present in the sample containers tested does not exceed 25 per mL, where the particles have a diameter that is equal to or greater than 10 μm, or if the average number of particles present in the sample containers tested does not exceed 3 per mL, where the particles have a diameter that is equal to or greater than 25 μm.

Test 1.B. The sample complies with the test limits if the average number of particles present in the sample containers tested does not exceed 6000 per container, where the particles have a diameter that is equal to or greater than 10 μm, or if the average number of particles present in the sample containers does not exceed 600 per container, where the particles have a diameter that is equal to or greater than 25 μm.

In particular embodiments, the compositions are considered physically or chemically stable if after storage the average number of particles detected does not exceed 50 particles/mL, where the particles have a diameter >10 m; does not exceed 5 particles/mL, where the particles have a diameter >25 m; and does not exceed 2 particles/mL, where the particles have a diameter >50 m, as measured by the microscopic method particle count test described in (788) Particulate Matter in Injections, Revised Bulletin, Official Oct. 1, 2011, The United States Pharmacopeial Convention.

The Microscopic Particle Count Test is performed using a suitable binocular microscope, a filter assembly for retaining particulate matter, and a membrane filter for examination. The microscope is adjusted to 100±10 magnifications and is equipped with an ocular micrometer calibrated with an objective micrometer, a mechanical stage capable of holding and traversing the entire filtration area of the membrane filter, and two suitable illuminators to provide episcopic illumination in addition to oblique illumination. The ocular micrometer is a circular diameter graticule and consists of a large circle divided by crosshairs into quadrants, transparent and black reference circles 10 μm and 25 μm in diameter at 100 magnifications, and a linear scale graduated in 10 μm increments. It is calibrated using a stage micrometer that is certified by either a domestic or international standard institution. A relative error of the linear scale of the graticule within ±2% is acceptable. The large circle is designated the graticule field of view (GFOV). Two illuminators are used. One is an episcopic brightfield illuminator internal to the microscope, the other is an external, focusable auxiliary illuminator that can be adjusted to give reflected oblique illumination at an angle of 10° to 20°. The filter assembly for retaining particulate matter consists of a filter holder made of glass or other suitable material, and is equipped with a vacuum source and a suitable membrane filter. The membrane filter is of suitable size, black or dark gray in color, nongridded or gridded, and 1.0 μm or finer in nominal pore size.

The test is carried out under conditions that limit exposure to extraneous particulate matter, for example, in a laminar flow cabinet. The glassware and filter assembly used, except for the membrane filter, are carefully washed with a warm detergent solution, and rinsed with abundant amounts of water to remove all traces of detergent. Immediately before use, both sides of the membrane filter and the equipment are rinsed from top to bottom, outside and then inside, with particle-free water.

In order to check that the environment is suitable for the test, that the glassware and the membrane filter are properly cleaned, and that the water to be used is particle-free, the following test is carried out: the particulate matter of a 50-mL volume of particle-free water is determined according to the method immediately below. If more than 20 particles of 10 μm or larger in size or if more than 5 particles of 25 μm or larger in size are present within the filtration area, the precautions taken for the test are not sufficient. The preparatory steps are repeated until the environment, glassware membrane filter, and water are suitable for the test.

The contents of the samples are mixed by slowly inverting the sample's container 20 times successively. If necessary, the container's sealing closure, if any, is cautiously removed. The outer surfaces of the container opening are cleaned using a jet of particle-free water and the sealing closure, if any, is removed, avoiding any contamination of the contents.

For large-volume samples, single units are tested. For small-volume samples less than 25 mL in volume, the contents of 10 or more sample containers are combined in a cleaned container; the test solution may be prepared by mixing the contents of a suitable number of vials and diluting to 25 mL with particle-free water or with an appropriate particle-free solvent when particle-free water is not suitable. Small-volume samples having a volume of 25 mL or more may be tested individually. Powders for parenteral use are constituted with particle-free water or with an appropriate particle-free solvent when particle-free water is not suitable. The number of test samples should be adequate to provide a statistically significant assessment. For large-volume samples or for small-volume samples having a volume of 25 mL or more, fewer than 10 units may be tested, using an appropriate sampling plan.

The inside of the filter holder fitted with the membrane filter is wetted with several mL of particle-free water. The total volume of a solution pool or of a single sample container is transferred to a filtration funnel, and a vacuum is applied. If needed, a portion of the sample is added stepwise until the entire volume is filtered. After the last addition of sample, the inner walls of the filter holder are rinsed by using a jet of particle-free water. The vacuum is maintained until the surface of the membrane filter is free from liquid. The membrane filter is placed in a Petri dish, and the membrane filter is allowed to air-dry with the cover slightly ajar. After the membrane filter has been dried, the Petri dish is placed on the stage of the microscope, the entire membrane filter is scanned under the reflected light from the illuminating device, and the number of particles that are equal to or greater than 10 μm and the number of particles that are equal to or greater than 25 μm are counted. Alternatively, partial membrane filter count and determination of the total filter count by calculation can be performed. The mean number of particles for the preparation to be examined is determined.

The particle sizing process with the use of the circular diameter graticule is carried out by estimating the equivalent diameter of the particle in comparison with the 10 μm and 25 μm reference circles on the graticule. Thereby the particles are not moved from their initial locations within the graticule field of view and are not superimposed on the reference circles for comparison. The inner diameter of the transparent graticule reference circles is used to size white and transparent particles, while dark particles are sized by using the outer diameter of the black opaque graticule reference circles.

Amorphous, semiliquid, or otherwise morphologically indistinct materials that have the appearance of a stain or discoloration on the membrane filter should not be sized or counted because these materials show little or no surface relief and present a gelatinous or film-like appearance. In such cases, the interpretation of enumeration may be aided by testing a sample of the solution by the Light Obscuration Particle Count Test.

For samples in containers having a nominal volume of more than 100 mL, apply the criteria of Test 2.A.

For samples in containers having a nominal volume of 100 mL or less, apply the criteria of Test 2.B.

Test 2.A. The sample complies with the test limits if the average number of particles present in the sample containers tested does not exceed 12 per mL and the particles have a diameter that is equal to or greater than 10 μm, or the average number of particles present in the sample containers tested does not exceed 2 per mL and the particles have a diameter that is equal to or greater than 25 μm.

Test 2.B. The sample complies with the test limits if the average number of particles present in the sample containers tested does not exceed 3000 per container and the particles have a diameter that is equal to or greater than 10 μm, or the average number of particles present in the sample containers tested does not exceed 300 per container and the particles have a diameter that is equal to or greater than 25 μm.

In certain embodiments, the compositions of the invention are in lyophilized form.

Compositions Comprising Antagonist A and Ranibizumab

In certain embodiments, a composition of the invention comprises Antagonist A or a modified form thereof and ranibizumab. In particular embodiments, the ratio of the concentration (mass of Antagonist A less that of its —R group/volume of composition) of Antagonist A or modified form thereof to the concentration (mass/volume of composition) of ranibizumab present in the composition is less than 25.0, less than 10.0, less than 9.0, less than 8.0, less than 7.0, less than 6.0, less than 5.0, less than 4.0, less than 3.0, less than 2.0 or less than 1.0. In particular embodiments, the ratio of the concentration (mass of Antagonist A less that of its —R group/volume of composition) of Antagonist A or modified form thereof to the concentration (mass/volume of composition) of ranibizumab present in the composition is less than or equal to 25.0, less than or equal to 10.0, less than or equal to 9.0, less than or equal to 8.0, less than or equal to 7.0, less than or equal to 6.0, less than or equal to 5.0, less than or equal to 4.0, less than or equal to 3.0, less than or equal to 2.0 or less than or equal to 1.0. In particular embodiments, the ratio of the concentration (mass of Antagonist A less that of its —R group/volume of composition) of Antagonist A or modified form thereof to the concentration (mass/volume of composition) of ranibizumab present in the composition is in the range of about 1 to about 10, about 2 to about 5, about 3 about 4, or about 5.

Antagonist A's —R group is depicted in FIG. 78A.

In particular embodiments, a composition of the invention comprises Antagonist A or a modified form thereof and ranibizumab, and the composition is stable with respect to both active agents at a particular pH or suitable for parenteral administration. In certain embodiments, the composition comprises one or more of a tonicity modifier, a surfactant, and a buffer suitable to achieve or maintain the particular pH or be suitable for parenteral administration. Appropriate buffers include those described herein as well as others known in the art, such as, e.g., a Good's buffers, e.g., MES.

In certain embodiments, the concentration of Antagonist A or modified form thereof in the composition of the invention is less than or about 100 mg/mL, less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL. In certain embodiments, the concentration of Antagonist A or modified form thereof is about 0.3 mg/mL to about 100 mg/mL, about 0.3 mg/mL to about 50 mg/mL, about 0.3 mg/mL to about 40 mg/mL, about 0.3 mg/mL to about 30 mg/mL, about 0.3 to about 25 mg/mL, about 0.3 mg/mL to about 20 mg/mL, about 0.3 mg/mL to about 15 mg/mL, about 0.3 mg/mL to about 10 mg/mL, about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, about 1 mg/mL to about 5 mg/mL, about 5 mg/mL to about 100 mg/mL, or about 5 mg/mL to about 50 mg/mL. In certain embodiments, the concentration of Antagonist A or modified form thereof is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 24 mg/mL, about 25 mg/mL, about 30 mg/mL, about 40 mg/mL, or about 50 mg/mL.

In certain embodiments, the concentration of ranibizumab in the composition of the invention is about 0.5 mg/mL to about 50 mg/mL, about 0.5 mg/mL to about 20 mg/mL, about 1.0 mg/mL to about 50 mg/mL, about 1 mg/mL to about 20 mg/mL, about 2 mg/mL to about 10 mg/mL, or about 4 mg/mL, about 5 mg/mL, about 6 mg/mL, about 7 mg/mL, about 8 mg/mL, about 9 mg/mL, about 10 mg/mL, about 11 mg/mL, or about 12 mg/mL.

In certain embodiments, the concentration of ranibizumab in the composition of the invention is about 0.5 mg/mL to about 50 mg/mL, about 0.5 mg/mL to about 20 mg/mL, about 1.0 mg/mL to about 50 mg/mL, about 1 mg/mL to about 20 mg/mL, about 2 mg/mL to about 10 mg/mL, or about 4 mg/mL, about 5 mg/mL, about 6 mg/mL, about 7 mg/mL, about 8 mg/mL, about 9 mg/mL, about 10 mg/mL, about 11 mg/mL, or about 12 mg/mL, and the concentration of Antagonist A or modified form thereof in the composition is less than about 100 mg/mL, less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL.

In certain embodiments, the concentration of ranibizumab in the composition of the invention is about 0.5 mg/mL to about 50 mg/mL, about 0.5 mg/mL to about 20 mg/mL, about 1 mg/mL to about 50 mg/mL, about 1 mg/mL to about 20 mg/mL, about 2 mg/mL to about 10 mg/mL, or about 4 mg/mL, about 5 mg/mL, about 6 mg/mL, about 7 mg/mL, about 8 mg/mL, about 9 mg/mL, about 10 mg/mL, about 11 mg/mL, or about 12 mg/mL, and the concentration of Antagonist A or modified form thereof is about 0.3 mg/mL to about 100 mg/mL, 0.3 mg/mL to about 50 mg/mL, about 0.3 mg/mL to about 40 mg/mL, about 0.3 mg/mL to about 30 mg/mL, about 0.3 to about 25 mg/mL, about 0.3 mg/mL to about 20 mg/mL, about 0.3 mg/mL to about 15 mg/mL, about 0.3 mg/mL to about 10 mg/mL, about 1.0 mg/mL to about 100 mg/mL, about 1 mg/mL to about 50 mg/mL, about 1 mg/mL to about 40 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, about 1 mg/mL to about 5 mg/mL, about 5 mg/mL to about 100 mg/mL, or about 5 mg/mL to about 50 mg/mL.

In certain embodiments, the concentration of ranibizumab in the compositions of the invention is about 0.5 mg/mL to about 50 mg/mL, about 0.5 mg/mL to about 20 mg/mL, about 1 mg/mL to about 50 mg/mL, about 1 mg/mL to about 20 mg/mL, about 2 mg/mL to about 50 mg/mL, about 2 mg/mL to about 10 mg/mL, or about 4 mg/mL, about 5 mg/mL, about 6 mg/mL, about 7 mg/mL, about 8 mg/mL, about 9 mg/mL, or about 10 mg/mL, and the concentration of Antagonist A or modified form thereof is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 24 mg/mL, about 25 mg/mL, about 30 mg/mL, about 40 mg/mL, or about 50 mg/mL. In one embodiment, the concentration of Antagonist A or modified form thereof is about 3 mg/mL, and the concentration of ranibizumab is about 5 mg/mL. In one embodiment, the concentration of Antagonist A or modified form thereof is about 6 mg/mL, and the concentration of ranibizumab is about 10 mg/mL. In one embodiment, the concentration of Antagonist A or modified form thereof is about 15 mg/mL, and the concentration of ranizumab is about 5 mg/mL. In one embodiment, the concentration of Antagonist A or modified form thereof is about 24 mg/mL, and the concentration of ranizumab is about 8 mg/mL.

In certain embodiments of a composition comprising Antagonist A or modified form thereof and ranibizumab, the composition further comprises a tonicity modifier that is sorbitol or sodium chloride, or mixtures thereof. In particular embodiments, the tonicity modifier is sorbitol, and the pH of the composition is about 5.0 to about 8.0, about 5.0 to about 7.0, about 6.0 or about 7.0. In particular embodiments, the tonicity modifier is sodium chloride, and the pH of the composition is about 5.0 to about 8.0, about 5.0 to about 7.0, about 5.5 to about 7.5, about 6.0 to about 8.0, about 8.0, about 7.0, or about 6.0. In certain embodiments, the tonicity modifier is sorbitol at about 1% to about 10% (w/v), or about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In particular embodiments, the tonicity modifier is sodium chloride at a concentration of about 10 mM to about 200 mM, about 50 mM to 200 mM, about 75 mM to about 200 mM, about 50 mM to about 150 mM, about 100 mM, about 110 mM, about 120 mM, about 130 mM about 140 mM or about 150 mM. In one embodiment, the tonicity modifier is sodium chloride at a concentration of about 130 mM. In other embodiments, the tonicity modifier is sodium chloride at a concentration of about 75 mM or about 120 mM. With respect to tonicity modifier concentration, “mM” refers to milimoles of the tonicity modifier per liter of composition.

In certain embodiments of a composition of the invention comprising Antagonist A or a modified form thereof and ranibizumab, the composition further comprises a buffer capable of achieving or maintaining the pH of the composition within a desired range. In certain embodiments, the composition comprises histidine (e.g., L-histidine or a pharmaceutically acceptable salt thereof) or phosphate as a buffer, e.g., sodium phosphate of potassium phosphate (or both histidine and phosphate). In certain embodiments, the buffer is present at a concentration of about 1 mM to about 200 mM, about 1 mM to about 150 mM, about 1 mM to about 20 mM, about 1 mM to about 10 mM, about 2 mM to about 100 mM, about 2 mM to about 20 mM, about 5 mM to about 20 mM, or about 10 mM. In particular embodiments, the pH of the buffered composition is about 5.0 to about 8.0, about 5.0 to about 7.0, about 5.5 to about 7.5, about 5.5 to about 7.0, or about 6.0. In one embodiment, the buffered composition has a pH of about 5.5 to about 7.0. In certain embodiments, the buffer comprises histidine at a concentration of about 1 mM to about 200 mM, about 1 mM to about 150 mM, about 2 mM to about 100 mM, about 5 mM to about 20 mM, or about 10 mM, and the buffered composition has a pH of about 5.5 to about 7.0, or about 6.0. In one particular embodiment, the buffer comprises histidine at a concentration of about 10 mM and the pH of the histidine-buffered composition is about 6.0. With respect to buffer concentration, “mM” refers to millimoles of buffer (e.g., histidine) per liter of composition.

In certain embodiments of a composition comprising Antagonist A or a modified form thereof and ranibizumab, the buffer comprises phosphate, alone or in combination with histidine. The phosphate buffer may be, e.g., a sodium phosphate or a potassium phosphate buffer. In certain embodiments, the buffer comprises phosphate at a concentration of about 1 mM to about 200 mM, about 1 mM to about 50 mM, about 2 mM to about 200 mM, about 2 mM to about 50 mM, about 5 mM to about 200 mM, about 5 mM to about 100 mM, about 5 mM to about 50 mM, about 10 mM to about 150 mM, about 10 mM to about 100 mM, about 5 mM, about 10 mM, about 25 mM, or about 50 mM. In particular embodiments, the pH of the buffered composition is about 5.0 to about 8.0, about 6.0 to about 8.0, about 5.5 to about 7.5, about 5.5 to about 7.0, about 6.0, about 7.0, or about 8.0. In one embodiment, the buffer comprises phosphate, and the buffered composition has a pH of about 6.0 to about 8.0. In certain embodiments, the buffer comprises phosphate at a concentration of about 5 mM to about 200 mM, about 5 mM to about 150 mM, about 5 mM to about 100 mM, about 5 mM, about 8 mM, about 10 mM, about 25 mM, or about 50 mM, and the buffered composition has a pH of about 5.5 to about 7.5, about 5.5 to about 7.0, or about 6.0. In one particular embodiment, the buffer comprises phosphate at a concentration of about 10 mM, and the buffered composition has a pH of about 6.2.

In certain embodiments of a composition comprising Antagonist A or a modified form thereof and ranibizumab, the composition further comprises a surfactant. In particular embodiments, the surfactant is polysorbate 20 at a concentration of about 0.001% (w/v) to about 0.05% (w/v), about 0.002% (w/v) to about 0.05% (w/v), about 0.005% (w/v) to about 0.05% (w/v), about 0.01% (w/v) to about 0.05% (w/v), or about 0.02% (w/v).

In one embodiment, a composition comprises Antagonist A or a modified form thereof, ranibizumab, histidine, and NaCl. The composition may further comprise polysorbate.

In one particular embodiment, a composition of the invention comprises Antagonist A or a modified verion thereof and ranibizumab; the ratio of the concentration of Antagonist A (or modified form thereof) to the concentration of ranibizumab is less than 2; and the composition further comprises sodium chloride at a concentration of about 10 mM to about 200 mM, histidine at a concentration of about 1 mM to about 100 mM, and polysorbate (e.g., polysorbate 20) at a concentration of about 0.005% to about 0.05%, where the pH of the composition is about 5.5 to about 7.0.

In certain embodiments, the present invention provides compositions comprising Antagonist A or a modified form thereof, or a pharmaceutically acceptable salt thereof, and ranibizumab, or a pharmaceutically acceptable salt thereof. In certain embodiments, a composition of the invention comprises: (a) about 0.3 mg/mL to about 30 mg/mL Antagonist A or modified form thereof, or a pharmaceutically acceptable salt thereof; and (b) about 0.5 mg/mL to about 20 mg/mL ranibizumab or pharmaceutically acceptable salt thereof. In other embodiments, the compositions further comprise one or both of: (c) about 1 mM to about 20 mM L-histidine; and (d) about 10 mM to about 200 mM NaCl. In further embodiments, the compositions further comprise: (e) about 0.001% (w/v) to about 0.05% (w/v) surfactant, which is optionally polysorbate. In a particular embodiment, the compositions comprise: (a) about 0.3 mg/mL to about 30 mg/mL Antagonist A or modified form thereof, or pharmaceutically acceptable salt thereof; (b) about 0.5 mg/mL to about 20 mg/mL ranibizumab or pharmaceutically acceptable salt thereof; (c) about 1 mM to about 20 mM L-histidine; and (d) about 10 mM to about 200 mM NaCl, wherein the pH of the compositions is about pH 5.0 to about pH 7.0. In a further embodiment, the compositions comprise: (a) about 3 mg/mL Antagonist A or modified form thereof, or pharmaceutically acceptable salt thereof; (b) about 5 mg/mL ranibizumab or pharmaceutically acceptable salt thereof; (c) about 10 mM L-histidine; and (d) about 130 mM NaCl, wherein the pH of the compositions is about pH 6.0. In certain embodiments, the compositions further comprise: (e) about 0.01% (w/v) polysorbate 20.

In certain embodiments, compositions of the invention comprise: (a) about 1.0 mg/mL to about 100 mg/mL, or about 5.0 mg/mL to about 50 mg/mL, Antagonist A or modified form thereof, or a pharmaceutically acceptable salt thereof; and (b) about 1.0 mg/mL to about 50 mg/mL ranibizumab or pharmaceutically acceptable salt thereof. In other embodiments, the compositions further comprise one or both of (c) about 1 mM to about 20 mM L-histidine; and (d) about 10 mM to about 200 mM NaCl. In further embodiments, the compositions further comprise: (e) about 0.001% (w/v) to about 0.05% (w/v) surfactant, which is optionally polysorbate. In a particular embodiment, the compositions comprise: (a) about 5.0 mg/mL to about 50 mg/mL Antagonist A or modified form thereof, or pharmaceutically acceptable salt thereof; (b) about 1.0 mg/mL to about 50 mg/mL ranibizumab or pharmaceutically acceptable salt thereof; (c) about 1 mM to about 20 mM L-histidine; and (d) about 10 mM to about 200 mM NaCl, wherein the pH of the compositions is about pH 5.0 to about pH 8.0 or about pH 5.5 to about pH 7.5. In a further embodiment, the compositions comprise: (a) about 15 mg/mL Antagonist A or modified form thereof, or pharmaceutically acceptable salt thereof; (b) about 5 mg/mL ranibizumab or pharmaceutically acceptable salt thereof; (c) about 5 mM L-histidine; and (d) about 75 mM NaCl, wherein the pH of the compositions is about pH 5.5 to about pH 7.5 or about pH 6.0. In certain embodiments, the compositions further comprise: (e) about 0.005% (w/v) polysorbate 20. In a further embodiment, the compositions comprise: (a) about 24 mg/mL Antagonist A or modified form thereof, or pharmaceutically acceptable salt thereof; (b) about 8 mg/mL ranibizumab or pharmaceutically acceptable salt thereof; (c) about 2 mM L-histidine; and (d) about 120 mM NaCl, wherein the pH of the compositions is about pH 5.5 to about pH 7.5 or about pH 6.0. In certain embodiments, the compositions further comprise: (e) about 0.002% (w/v) polysorbate 20.

In certain embodiments, compositions of the invention comprise: (a) about 0.3 mg/mL to about 30 mg/mL Antagonist A or a modified form thereof, or a pharmaceutically acceptable salt thereof; (b) about 0.5 mg/mL to about 20 mg/mL ranibizumab; and one or both of (c) a buffer capable of achieving or maintaining the pH of the composition to about pH 5.0 to about pH 8.0; and (d) a tonicity modifier. In particular embodiments, the buffer, where present, is about 1 mM to about 20 mM L-histidine or about 1 mM to about 20 mM sodium phosphate; and the tonicity modifier, where present, is about 10 mM to about 200 mM NaCl, about 1% to about 20% (w/v) sorbitol, or about 1% to about 20% (w/v) trehalose. In certain embodiments, the buffer is about 1 mM to about 20 mM L-histidine; and the tonicity modifier is about 10 mM to about 200 mM NaCl, wherein the pH of the composition is about pH 5.0 to about pH 7.0.

Any of the compositions of the invention may also comprise a surfactant, e.g., about 0.001% (w/v) to about 0.05% (w/v) surfactant.

Examples of compositions of the invention include the compositions described in Table 1, Table 3 or Table 8. In other embodiments, the invention includes the compositions described in Table 1 but without the polysorbate.

In one embodiment, a composition of the invention comprises Antagonist A or a modified form thereof at a concentration of about 3 mg/mL, ranibizumab at a concentration of about 5 mg/mL, histidine at a concentration of about 10 mM, sodium chloride at a concentration of about 130 mM and polysorbate 20 at a concentration of about 0.02% (w/v), wherein the pH of the composition is about 6.0.

In one embodiment, a composition of the invention comprises about 3 mg/mL Antagonist A or modified form thereof, about 5 mg/mL ranibizumab, about 10 mM sodium phosphate, about 5% (w/v) sorbitol, and about 0.01% (w/v) polysorbate 20, wherein the pH of the composition is about pH 7.0.

In one embodiment, a composition of the invention comprises about 3 mg/mL Antagonist A or modified form thereof, about 5 mg/mL ranibizumab, about 10 mM sodium phosphate, about 130 mM NaCl, and about 0.01% (w/v) polysorbate 20, wherein the pH of the composition is about pH 7.0.

In one embodiment, a composition of the invention comprises about 3 mg/mL Antagonist A or modified form thereof, about 5 mg/mL ranibizumab, about 5 mM sodium phosphate, about 5 mM histidine HCl, about 75 mM NaCl, about 5% (w/v) trehalose, and about 0.005% (w/v) polysorbate 20, wherein the pH of the composition is about pH 6.5.

In certain embodiments the compositions of the invention comprise: (a) about 3 mg/mL to about 90 mg/mL Antagonist A or a modified form thereof; (b) about 1.0 mg/mL to about 30 mg/mL ranibizumab; and one or both of (c) a buffer capable of achieving or maintaining the pH of the composition to about pH 5.0 to about pH 8.0; and (d) a tonicity modifier. In particular embodiments, the buffer, where present, comprises about 1 mM to about 100 mM sodium phosphate or about 1.0 mM to about 10 mM histidine.HCl; and the tonicity modifier, where present, is about 0.5% (w/v) to about 10% (w/v) trehalose.

In one embodiment, a composition of the invention comprises Antagonist A or a modified form thereof at a concentration of about 15 mg/mL, ranibizumab at a concentration of about 5 mg/mL, histidine at a concentration of about 5 mM, sodium chloride at a concentration of about 75 mM and polysorbate 20 at a concentration of about 0.005% (w/v), wherein the pH of the composition is about 5.5 to about 7.5.

In one embodiment, a composition of the invention comprises Antagonist A or a modified form thereof at a concentration of about 24 mg/mL, ranibizumab at a concentration of about 8 mg/mL, histidine at a concentration of about 2 mM, sodium chloride at a concentration of about 120 mM and polysorbate 20 at a concentration of about 0.002% (w/v), wherein the pH of the composition is about 5.5 to about 7.5.

In particular embodiments, a composition comprising Antagonist A or a modified form thereof and ranibizumab is chemically stable for at least eight weeks or at least twelve weeks at 25° C. or for at least twelve weeks or at least sixteen weeks or at least 24 weeks at 4° C. In particular embodiments, at least 80% of each of Antagonist A and ranibizumab show no sign of decomposition or modification resulting in formation of a new chemical entity under at least one of these conditions.

Compositions Comprising Antagonist A and Bevacizumab

In certain embodiments, a composition of the invention comprises

Antagonist A or a modified form thereof and bevacizumab. In particular embodiments, the ratio of the concentration (mass of Antagonist A less that of its —R group/volume of composition) of Antagonist A (or modified form thereof) to the concentration (mass/volume of composition) of bevacizumab present in the composition is less than 25.0, less than 10.0, less than 9.0, less than 8.0, less than 7.0, less than 6.0, less than 5.0, less than 4.0, less than 3.0, less than 2.0, less than 1.0, or less than 0.5.

Antagonist A's —R group is depicted in FIG. 78A.

In particular embodiments, a composition of the invention comprises Antagonist A or a modified form thereof and bevacizumab, and the composition is stable with respect to both active agents at a particular pH suitable for parenteral administration. In certain embodiments, the composition comprises one or more tonicity modifier, surfactant, and buffer suitable to achieve or maintain the particular pH or be suitable for parenteral administration. Appropriate buffers include those described herein as well as others known in the art, such as, e.g., a Good's buffers, e.g., MES.

In certain embodiments, the concentration of Antagonist A or modified form thereof in the composition is less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL. In certain embodiments, the concentration of Antagonist A or modified form thereof is about 0.3 mg/mL to about 50 mg/mL, about 0.3 mg/mL to about 40 mg/mL, about 0.3 mg/mL to about 30 mg/mL, about 0.3 to about 25 mg/mL, about 0.3 mg/mL to about 20 mg/mL, about 0.3 mg/mL to about 15 mg/mL, about 0.3 mg/mL to about 10 mg/mL, about 1 mg/mL to about 50 mg/mL, about 1 mg/mL to about 40 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, or about 1 mg/mL to about 5 mg/mL. In certain embodiments, the concentration of Antagonist A or modified form thereof is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 40 mg/mL, or about 50 mg/mL.

In certain embodiments, the concentration of bevacizumab is about 0.5 mg/mL to about 50 mg/mL, about 0.5 mg/mL to about 25 mg/mL, about 1 mg/mL to about 50 mg/mL, about 1.0 to about 25 mg/mL, about 1.0 to about 20 mg/mL, about 5 mg/mL to about 50 mg/mL, about 5 mg/mL to about 25 mg/mL, about 5 mg/mL to about 25 mg/mL, about 5 mg/mL to about 20 mg/mL, about 12.5 mg/mL, about 25 mg/mL, or about 50 mg/mL.

In certain embodiments, the concentration of bevacizumab is about 0.5 mg/mL to about 50 mg/mL, about 0.5 mg/mL to about 25 mg/mL, about 1 mg/mL to about 50 mg/mL, about 1.0 to about 25 mg/mL, about 1.0 to about 20 mg/mL, about 5 mg/mL to about 50 mg/mL, about 5 mg/mL to about 25 mg/mL, about 5 mg/mL to about 25 mg/mL, about 5 mg/mL to about 20 mg/mL, about 12.5 mg/mL, about 25 mg/mL, or about 50 mg/mL, and the concentration of Antagonist A or modified form thereof is less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL.

In certain embodiments, the concentration of bevacizumab is about 0.5 mg/mL to about 50 mg/mL, about 0.5 mg/mL to about 25 mg/mL, about 1 mg/mL to about 50 mg/mL, about 1.0 to about 25 mg/mL, about 1.0 to about 20 mg/mL, about 5 mg/mL to about 50 mg/mL, about 5 mg/mL to about 25 mg/mL, about 5 mg/mL to about 25 mg/mL, about 5 mg/mL to about 20 mg/mL, about 12.5 mg/mL, about 25 mg/mL, or about 50 mg/mL, and the concentration of Antagonist A or modified form thereof is about 0.3 mg/mL to about 50 mg/mL, about 0.3 mg/mL to about 40 mg/mL, about 0.3 mg/mL to about 30 mg/mL, about 0.3 to about 25 mg/mL, about 0.3 mg/mL to about 20 mg/mL, about 0.3 mg/mL to about 15 mg/mL, about 0.3 mg/mL to about 10 mg/mL, about 1 mg/mL to about 50 mg/mL, about 1 mg/mL to about 40 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, or about 1 mg/mL to about 5 mg/mL.

In certain embodiments, the concentration of bevacizumab is about 0.5 mg/mL to about 50 mg/mL, about 0.5 mg/mL to about 25 mg/mL, about 1 mg/mL to about 50 mg/mL, about 1.0 to about 25 mg/mL, about 1.0 to about 20 mg/mL, about 5 mg/mL to about 50 mg/mL, about 5 mg/mL to about 25 mg/mL, about 5 mg/mL to about 25 mg/mL, about 5 mg/mL to about 20 mg/mL, about 12.5 mg/mL, about 25 mg/mL, or about 50 mg/mL, and the concentration of Antagonist A or modified form thereof is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 40 mg/mL, or about 50 mg/mL. In one embodiment, the concentration of Antagonist A or modified form thereof is about 3 mg/mL and the concentration of bevacizumab is about 12.5 mg/mL. In another embodiment, the concentration of Antagonist A or modified form thereof is about 6 mg/mL, and the concentration of bevacizumab is about 25 mg/mL or about 50 mg/mL.

In certain embodiments of a composition comprising Antagonist A or a modified form thereof and bevacizumab, the composition further comprises a tonicity modifier selected from sorbitol, sodium chloride and trehalose. In other embodiments, the composition comprises both sorbitol and sodium chloride, both sodium chloride and trehalose, or both sorbitol and trehalose. In particular embodiments, the composition comprises sorbitol, and the pH of the composition is about 7.0 to about 8.0. In particular embodiments, the composition comprises sodium chloride, and the pH of the composition is about 6.0 to about 8.0. In certain embodiments, the composition comprises trehalose, and the pH of the composition is about 6.0 to about 7.0. In certain embodiments, the composition comprises sorbitol at about 1% to about 10% (w/v), or about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In particular embodiments, the composition comprises sodium chloride at a concentration of about 10 mM to about 200 mM, about 50 mM to 200 mM, about 75 mM to about 200 mM, about 50 mM to about 150 mM, about 100 mM, about 110 mM, about 120 mM, about 130 mM about 140 mM or about 150 mM. In one embodiment, the composition comprises sodium chloride at a concentration of about 130 mM. In certain embodiments, the composition comprises trehalose at about 1% to about 10% (w/v), or about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

In certain embodiments of a composition comprising Antagonist A or a modified form thereof and bevacizumab, the composition further comprises a buffer capable of achieving or maintaining the pH of the composition within a desired range. In certain embodiments, the composition comprises one or more of acetate, phosphate, and Tris as a buffer. In certain embodiments, the buffer comprises phosphate at a concentration of about 5 mM to about 200 mM, about 5 mM to about 100 mM, about 10 mM to about 150 mM, about 10 mM to about 100 mM, about 25 mM to about 100 mM, or about 50 mM. The phosphate buffer may be, e.g., a sodium phosphate buffer or a potassium phosphate buffer. In particular embodiments, the pH of the buffered composition is about 5.0 to about 8.0, about 6.0 to about 8.0, about 5.5 to about 7.0, about 6.0, about 7.0, or about 8.0. In one embodiment, the buffer comprises phosphate, and the pH of the buffered composition is about 5.5 to about 7.0. In certain embodiments, the buffer comprises phosphate at a concentration of about 5 mM to about 200 mM, about 10 mM to about 150 mM, about 25 mM to about 100 mM, or about 50 mM, and the buffered composition has a pH of about 5.5 to about 7.0, or about 6.0. In one particular embodiment, the buffer comprises phosphate at a concentration of about 50 mM, and the buffered composition has a pH of about 6.0.

In certain embodiments of a composition comprising Antagonist A or a modified form thereof and bevacizumab, the composition further comprises a surfactant. In particular embodiments, the surfactant is polysorbate 20 at a concentration of about 0.005% (w/v) to about 0.05% (w/v), about 0.01% (w/v) to about 0.05% (w/v), or about 0.02% (w/v).

In one embodiment, a composition comprising Antagonist A or a modified form thereof and bevacizumab comprises Antagonist A, bevacizumab, sodium chloride, and phosphate. The composition may further comprise polysorbate.

In one particular embodiment: a composition comprises Antagonist A or a modified form thereof and bevacizumab; the ratio of the concentration of Antagonist A (or modified form thereof) to the concentration of bevacizumab is less than 1.5, less than 1.2 or less than 1; and the composition further comprises sodium chloride at a concentration of about 10 mM to about 200 mM, phosphate at a concentration of about 5 mM to about 200 mM, and polysorbate (e.g., polysorbate 20) at a concentration of about 0.005% to about 0.05%, wherein the pH of the composition is about 5.5 to about 7.0.

In certain embodiments, the present invention provides compositions comprising Antagonist A or a modified form thereof, or a pharmaceutically acceptable salt thereof, and bevacizumab, or a pharmaceutically acceptable salt thereof. In certain embodiments, a composition of the invention comprises: (a) about 0.3 mg/mL to about 30 mg/mL Antagonist A or a modified form thereof, or pharmaceutically acceptable salt thereof; and (b) about 0.5 mg/mL to about 25 mg/mL bevacizumab or pharmaceutically acceptable salt thereof. In other embodiments, the composition further comprises one or both of (c) about 5 mM to about 200 mM phophate buffer; and (d) about 10 mM NaCl to about 200 mM NaCl. In other embodiments, the composition comprises: (a) about 0.3 mg/mL to about 30 mg/mL Antagonist A or modified form thereof, or pharmaceutically acceptable salt thereof; (b) about 0.5 mg/mL to about 25 mg/mL bevacizumab or pharmaceutically acceptable salt thereof; (c) about 5 mM to about 200 mM phosphate buffer, (e.g., about 5 mM to about 200 mM sodium phosphate); and (d) about 10 mM NaCl to about 200 mM NaCl, wherein the pH of the composition is about pH 5.0 to about pH 7.0. In particular embodiments of compositions comprising bevacizumab, the composition further comprises: (e) about 0.001% (w/v) to about 0.05% (w/v) surfactant, which is optionally polysorbate. In a particular embodiment, the composition comprises: (a) about 3 mg/mL Antagonist A or modified form thereof, or pharmaceutically acceptable salt thereof; (b) about 12.5 mg/mL bevacizumab or pharmaceutically acceptable salt thereof; (c) about 50 mM phosphate buffer; and (d) about 130 mM NaCl, wherein the pH of the composition is about pH 6.0. In another embodiment, the composition further comprises: (e) about 0.01% (w/v) polysorbate 20.

In certain embodiments, the compositions of the invention comprise: (a) about 0.3 mg/mL to about 30 mg/mL Antagonist A or a modified form thereof; (b) about 0.5 mg/mL to about 25 mg/mL bevacizumab; and one or both of (c) a buffer capable of achieving or maintaining the pH of the composition to about pH 5.0 to about pH 8.0; and (d) a tonicity modifier. In particular embodiments, the buffer is about 5 mM to about 200 mM sodium phosphate or about 5 mM to about 200 mM Tris.HCl; and the tonicity modifier is about 10 mM to about 200 mM NaCl, about 1% to about 20% (w/v) sorbitol, or about 1% to about 20% (w/v) trehalose. In certain embodiments, the buffer is about 5 mM to about 200 mM sodium phosphate; and the tonicity agent is about 10 mM to about 200 mM NaCl, wherein the pH of the composition is about pH 5.0 to about pH 7.0. In particular embodiments, compositions of the invention comprise a surfactant, e.g., about 0.001% (w/v) to about 0.05% (w/v) surfactant.

Examples of compositions of the invention include the compositions described in Table 3, as well as these compositions absent the surfactant.

In one embodiment, a composition comprises Antagonist A or a modified form thereof at a concentration of about 3 mg/mL, bevacizumab at a concentration of about 12.5 mg/mL, sodium phosphate at a concentration of about 50 mM, sodium chloride at a concentration of about 130 mM and polysorbate 20 at a concentration of about 0.02% (w/v), wherein the pH of the composition is about 6.0.

In one embodiment, a composition of the invention comprises about 3 mg/mL Antagonist A or modified form thereof, about 12.5 mg/mL bevacizumab, about 50 mM sodium phosphate, about 5% (w/v) sorbitol, and about 0.02% (w/v) polysorbate 20, wherein the pH of the composition is about pH 6.0.

In one embodiment, a composition of the invention comprises about 3 mg/mL Antagonist A, or modified form thereof, about 12.5 mg/mL bevacizumab, about 50 mM sodium phosphate, about 5% (w/v) sorbitol, and about 0.02% (w/v) polysorbate 20, wherein the pH of the composition is about pH 7.0.

In one embodiment, a composition of the invention comprises about 3 mg/mL Antagonist A, or modified form thereof, about 12.5 mg/mL bevacizumab, about 50 mM sodium phosphate, about 150 mM NaCl, and about 0.02% (w/v) polysorbate 20, wherein the pH of the composition is about pH 7.0.

In one embodiment, a composition of the invention comprises about 3 mg/mL Antagonist A or modified form thereof, about 12.5 mg/mL bevacizumab, about 50 mM Tris.HCl, about 130 mM NaCl, and about 0.02% (w/v) polysorbate 20, wherein the pH of the composition is about pH 8.0.

In one embodiment, a composition of the invention comprises about 15 mg/mL Antagonist A, or modified form thereof, about 12.5 mg/mL bevacizumab, about 30 mM sodium phosphate, about 75 mM NaCl, about 3% (w/v) trehalose, and about 0.02% (w/v) polysorbate 20, wherein the pH of the composition is about pH 6.3.

In one embodiment, a composition of the invention comprises about 3 mg/mL Antagonist A, or modified form thereof, about 12.5 mg/mL bevacizumab or a pharmaceutically acceptable salt thereof, about 30 mM sodium phosphate, about 75 mM NaCl, about 3% (w/v) trehalose, and about 0.02% (w/v) polysorbate 20, wherein the pH of the composition is about pH 6.3.

In particular embodiments, a composition comprising Antagonist A or a modified form thereof and bevacizumab is chemically stable for at least four weeks or at least eight weeks at 25° C. or for at least twelve weeks or at least 24 weeks at 4° C. In particular embodiments, at least 70% of each of Antagonist A or modified form thereof and bevacizumab show no sign of decomposition or modification resulting in formation of a new chemical entity under these conditions.

Compositions Comprising Antagonist A and Aflibercept

In certain embodiments, a composition comprises Antagonist A or a modified form thereof and aflibercept. In particular embodiments, the ratio of the concentration (mass of Antagonist A less that of its —R group/volume of composition) of Antagonist A to the concentration (mass/volume of composition) of aflibercept present in the composition is less than 25.0, less than 10.0, less than 9.0, less than 8.0, less than 7.0, less than 6.0, less than 5.0, less than 4.0, less than 3.0, less than 2.0, less than 1.0, less than 0.5, or less than 0.25.

Antagonist A's —R Group is Depicted in FIG. 78A.

In particular embodiments, a composition comprises Antagonist A or a modified form thereof and aflibercept, and the composition is stable with respect to both active agents at a particular pH or suitable for parenteral administration. In certain embodiments, the composition comprises one or more tonicity modifier, surfactant, and buffer suitable to achieve or maintain the particular pH or be suitable for parenteral administration. Appropriate buffers include those described herein as well as others known in the art, such as, e.g., a Good's buffers, e.g., MES.

In certain embodiments, the concentration of Antagonist A or modified form thereof in the composition is less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL. In certain embodiments, the concentration of Antagonist A or modified form thereof is about 0.3 mg/mL to about 50 mg/mL, about 0.3 mg/mL to about 40 mg/mL, about 0.3 mg/mL to about 30 mg/mL, about 0.3 to about 25 mg/mL, about 0.3 mg/mL to about 20 mg/mL, about 0.3 mg/mL to about 15 mg/mL, about 0.3 mg/mL to about 10 mg/mL, about 1 mg/mL to about 50 mg/mL, about 1 mg/mL to about 40 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, or about 1 mg/mL to about 5 mg/mL. In certain embodiments, the concentration of Antagonist A or modified form thereof is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 40 mg/mL, or about 50 mg/mL.

In certain embodiments, the concentration of aflibercept is about 5 mg/mL to about 100 mg/mL, about 5 mg/mL to about 50 mg/mL, about 5 mg/mL to about 40 mg/mL, about 10 mg/mL to about 100 mg/mL, about 10 mg/mL to about 50 mg/mL, about 10 mg/mL to about 40 mg/mL, about 20 mg/mL to about 40 mg/mL, about 30 mg/mL, about 50 mg/mL, or about 40 mg/mL.

In certain embodiments, the concentration of aflibercept is about 5 mg/mL to about 100 mg/mL, about 5 mg/mL to about 50 mg/mL, about 5 mg/mL to about 40 mg/mL, about 10 mg/mL to about 100 mg/mL, about 10 mg/mL to about 50 mg/mL, about 10 mg/mL to about 40 mg/mL, about 20 mg/mL to about 40 mg/mL, about 30 mg/mL, about 50 mg/mL, or about 40 mg/mL, and the concentration of Antagonist A or modified form thereof is less than about 50 mg/mL, less than about 40 mg/mL, less than about 30 mg/mL, less than about 25 mg/mL, less than about 20 mg/mL, less than about 15 mg/mL, less than about 10 mg/mL, or less than about 5 mg/mL.

In certain embodiments, the concentration of aflibercept is about 5 mg/mL to about 100 mg/mL, about 5 mg/mL to about 50 mg/mL, about 5 mg/mL to about 40 mg/mL, about 10 mg/mL to about 100 mg/mL, about 10 mg/mL to about 50 mg/mL, about 10 mg/mL to about 40 mg/mL, about 20 mg/mL to about 40 mg/mL, about 30 mg/mL, about 50 mg/mL, or about 40 mg/mL, about 1 mg/mL to about 10 mg/mL, or about 1 mg/mL to about 5 mg/mL, and the concentration of Antagonist A or modified form thereof is about 0.3 mg/mL to about 50 mg/mL, about 0.3 mg/mL to about 40 mg/mL, about 0.3 mg/mL to about 30 mg/mL, about 0.3 to about 25 mg/mL, about 0.3 mg/mL to about 20 mg/mL, about 0.3 mg/mL to about 15 mg/mL, about 0.3 mg/mL to about 10 mg/mL, about 1 mg/mL to about 50 mg/mL, about 1 mg/mL to about 40 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, or about 1 mg/mL to about 5 mg/mL.

In certain embodiments, the concentration of aflibercept is about 5 mg/mL to about 100 mg/mL, about 5 mg/mL to about 50 mg/mL, about 5 mg/mL to about 40 mg/mL, about 10 mg/mL to about 100 mg/mL, about 10 mg/mL to about 50 mg/mL, about 10 mg/mL to about 40 mg/mL, about 20 mg/mL to about 40 mg/mL, about 30 mg/mL, about 50 mg/mL, or about 40 mg/mL, about 1 mg/mL to about 10 mg/mL, or about 1 mg/mL to about 5 mg/mL, and the concentration of Antagonist A or modified form thereof is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 40 mg/mL, or about 50 mg/mL. In one embodiment, the concentration of Antagonist A is about 3 mg/mL, and the concentration of aflibercept is about 20 mg/mL. In one embodiment, the concentration of Antagonist A is about 6 mg/mL, and the concentration of aflibercept is about 40 mg/mL. In another embodiment, the concentration of Antagonist A or modified form thereof is about 12 mg/mL, and the concentration of alfibercept is about 80 mg/mL.

In certain embodiments of a composition comprising Antagonist A or a modified form thereof and aflibercept, the composition further comprises one or more tonicity modifier(s) selected from sorbitol and sodium chloride. In particular embodiments, the tonicity modifier comprises sorbitol, and the pH of the composition is about 6.0 to about 8.0. In particular embodiments, the tonicity modifier comprises sodium chloride, and the pH of the composition is about 6.0 to about 8.0. In certain embodiments, the tonicity modifier comprises sorbitol at about 1% to about 10% (w/v), or about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In particular embodiments, the tonicity modifier is sodium chloride at a concentration of about 10 mM to about 200 mM, about 50 mM to 200 mM, about 75 mM to about 200 mM, about 25 mM to about 150 mM, about 50 mM to about 150 mM, about 20 mM, about 30 mM, about 40 mM, about 50 mM, about 60 mM, about 70 mM, about 80 mM, about 90 mM, about 100 mM, about 110 mM, about 120 mM, about 130 mM about 140 mM or about 150 mM. In one embodiment, the tonicity modifier is sodium chloride at a concentration of about 40 mM.

In certain embodiments of a composition comprising Antagonist A or a modified form thereof and aflibercept, the composition further comprises a buffer capable of achieving or maintaining the pH within a desired range. In certain embodiments, the composition comprises one or more buffer(s) selected from acetate, phosphate, histidine and Tris. In certain embodiments, the buffer comprises phosphate at a concentration of about 1 mM to about 200 mM, about 1 mM to about 50 mM, about 5 mM to about 200 mM, about 5 mM to about 100 mM, about 5 mM to about 50 mM, about 10 mM to about 150 mM, about 10 mM to about 100 mM, about 5 mM, about 10 mM, about 25 mM, or about 50 mM. In certain embodiments, the phosphate buffer is sodium phosphate or potassium phosphate. In particular embodiments, the pH of the buffered composition is about 5.0 to about 8.0, about 6.0 to about 8.0, about 5.5 to about 7.0, about 6.0, about 7.0, or about 8.0. In one embodiment, the buffer comprises phosphate, and the buffered composition has a pH of about 6.0 to about 8.0. In certain embodiments, the buffer comprises phosphate at a concentration of about 5 mM to about 200 mM, about 5 mM to about 150 mM, about 5 mM to about 100 mM, about 5 mM, about 10 mM, about 25 mM, or about 50 mM, and the buffered composition has a pH of about 5.5 to about 7.0, or about 6.0. In one particular embodiment, the buffer comprises phosphate at a concentration of about 10 mM, and the buffered composition has a pH of about 6.2.

In certain embodiments of a composition comprising Antagonist A or a modified form thereof and aflibercept, the composition further comprises sucrose. In particular embodiments, sucrose is present in the composition at a concentration of about 0% (w/v) to about 10% (w/v), about 1% (w/v) to about 10% (w/v), about 2% (w/v) to about 10% (w/v), or about 5% (w/v).

In certain embodiments of a composition comprising Antagonist A or a modified form thereof and aflibercept, the composition further comprises a surfactant. In particular embodiments, the surfactant is polysorbate 20 at a concentration of about 0.005% (w/v) to about 0.05% (w/v), about 0.01% (w/v) to about 0.05% (w/v), about 0.03% (w/v), or about 0.02% (w/v).

In one embodiment, a composition comprising Antagonist A or a modified form thereof and aflibercept comprises Antagonist A or modified form thereof, aflibercept, sodium chloride, and phosphate. The composition may further comprise polysorbate or sucrose (or both).

In one particular embodiment a composition comprises Antagonist A or a modified form thereof and aflibercept; the ratio of the concentration of Antagonist A or modified form thereof to the concentration of aflibercept is less than 1; and the composition further comprises sodium chloride at a concentration of about 10 mM to about 200 mM, phosphate at a concentration of about 5 mM to about 50 mM, sucrose at a concentration of about 0% (w/v) to about 10% (w/v), and polysorbate (e.g., polysorbate 20) at a concentration of about 0.005% to about 0.05%, wherein the pH of the composition is about 6.0 to about 8.0.

In certain embodiments, the compositions comprise: (a) about 0.3 mg/mL to about 30 mg/mL Antagonist A or a modified form thereof, or pharmaceutically acceptable salt thereof; and (b) about 5 mg/mL to about 40 mg/mL aflibercept or pharmaceutically acceptable salt thereof. In particular embodiments, the compositions further comprise one or both of (c) about 5 mM to about 50 mM phosphate buffer (e.g., about 5 mM to about 50 mM sodium phosphate); and (d) about 10 mM to about 200 mM NaCl. In further embodiments, the compositions further comprise: (e) 0 to about 10% (w/v) sucrose. In certain embodiments, the compositions comprise: (a) about 0.3 mg/mL to about 30 mg/mL Antagonist A or modified form thereof, or pharmaceutically acceptable salt thereof; (b) about 5 mg/mL to about 40 mg/mL aflibercept or pharmaceutically acceptable salt thereof; (c) about 5 mM to about 50 mM phosphate buffer; (d) about 10 mM to about 200 mM NaCl; and (e) 0 to about 10% (w/v) sucrose, wherein the pH of the composition is about pH 6.0 to about pH 8.0. In another embodiment, the compositions further comprise: (f) about 0.001% (w/v) to about 0.05% (w/v) polysorbate. In one particular embodiment, the compositions comprise: (a) about 6 mg/mL Antagonist A or modified form thereof or pharmaceutically acceptable salt thereof; (b) about 40 mg/mL aflibercept or pharmaceutically acceptable salt thereof; (c) about 10 mM phosphate buffer; (d) about 40 mM NaCl; and (e) about 5% (w/v) sucrose, wherein the pH of the composition is about pH 6.2. In a further embodiment, the compositions further comprise: (f) about 0.03% (w/v) polysorbate 20.

In certain embodiments of a composition of the invention comprises: (a) about 0.3 mg/mL to about 30 mg/mL Antagonist A, or a modified form thereof; (b) about mg/mL to about 40 mg/mL aflibercept; and one or more of (c) a buffer capable of achieving or maintaining the pH of the composition to about pH 5.0 to about pH 8.0; (d) a tonicity modifier; and (e) 0 to about 10% (w/v) sucrose. In particular embodiments, the buffer, where present, is about 5 mM to about 50 mM phosphate, and the tonicity modifier, where present, is about 10 mM to about 200 mM NaCl.

In particular embodiments, a composition of the invention comprises (a) about 0.3 mg/mL to about 30 mg/mL Antagonist A, or a modified form thereof; (b) about 5 mg/mL to about 40 mg/mL aflibercept; (c) about 5 mM to about 50 mM phosphate; (d) about 10 mM to about 200 mM NaCl; (e) 0 to about 10% (w/v) sucrose; and (f) about 0.001% (w/v) to about 0.05% (w/v) surfactant, wherein the pH of the composition is about pH 6.0 to about pH 8.0.

Compositions of the invention also include any of the compositions described herein absent the surfactant.

In one embodiment, a composition of the invention comprises Antagonist A or a modified form thereof at a concentration of about 6 mg/mL, aflibercept at a concentration of about 40 mg/mL, phosphate at a concentration of about 10 mM, sodium chloride at a concentration of about 40 mM and polysorbate 20 at a concentration of about 0.03% (w/v), and the composition has a pH about 6.2.

In another embodiment, a composition of the invention comprises Antagonist A or a modified form thereof at a concentration of about 3 mg/mL, aflibercept at a concentration of about 20 mg/mL, phosphate at a concentration of about 10 mM, sodium chloride at a concentration of about 40 mM and polysorbate 20 at a concentration of about 0.03% (w/v), and the composition has a pH about 6.2.

In particular embodiments, a composition comprising Antagonist A and aflibercept is chemically stable for at least four weeks or at least eight weeks at 25° C. or for at least twelve weeks or at least 24 weeks at 4° C. In particular embodiments, at least 70% of both antagonists show no sign of decomposition or modification resulting in formation of a new chemical entity under these conditions.

Methods for Making Compositions of the Invention

Compositions of the invention, including those described herein, may be prepared by a method comprising, consisting essentially of, or consisting of, admixing the antagonists (e.g., one or more anti-PDGF aptamers and one or more VEGF antagonists) and an effective amount of a buffer, e.g., a histidine, phosphate, acetate or Tris buffer, and optionally adjusting the pH of the resulting mixture to a pH of about 5.5 to about 8.0 and variations in between as described herein.

In some embodiments, the method further comprises, consists essentially of, or consists of admixing the anti-PDGF aptamer and the VEGF antagonist and an effective amount of a tonicity agent. In a particular aspect, the tonicity agent is sodium chloride or sorbitol.

In some embodiments, the method further comprises, consists essentially of, or consists of admixing the anti-PDGF aptamer and VEGF antagonist and an effective amount of a surfactant. In particular aspects, the surfactant is a polysorbate, e.g., Tween 20 or Tween 80.

In some embodiments, the method further comprises, consists essentially of, or consists of admixing the anti-PDGF aptamer and VEGF antagonist and an effective amount of a stabilizer, cryoprotectant, or lyoprotectant. The stabilizer can be at least one a sugar, an amino acid, a polyol, a surfactant, an antioxidant, a preservative, a cyclodextrine, a polyethyleneglycol, albumin or a salt.

In particular aspects of the method, the compositions are prepared by admixing the anti-PDGF aptamer and the VEGF antagonist and various excipients present in the various compositions described herein and in the range of concentrations described herein, including each the specific compositions described above that comprise Antagonist A or a modified form thereof in combination with either bevacizumab, ranibizumab, or aflibercept.

Thus, in one embodiment, a composition of the invention is prepared by admixing the following: Antagonist A or a modified form thereof to a final concentration of about 3 mg/mL, bevacizumab to a final concentration of about 12.5 mg/mL, phosphate to a final concentration of about 50 mM, sodium chloride to a final concentration of about 130 mM, and polysorbate 20 to a final concentration of about 0.02% (w/v). In another embodiment, a composition of the invention is prepared by admixing the following: Antagonist A or a modified form thereof to a final concentration of about 6 mg/mL, bevacizumab to a final concentration of about 25 mg/mL, phosphate to a final concentration of about 50 mM, sodium chloride to a final concentration of about 130 mM, and polysorbate 20 to a final concentration of about 0.02% (w/v). In certain embodiments, the pH of the composition is adjusted to about 6.0.

In another embodiment, a composition is prepared by admixing the following: Antagonist A or a modified form thereof to a final concentration of about 3 mg/mL, ranibizumab to a final concentration of about 5 mg/mL, histidine to a final concentration of about 10 mM, sodium chloride to a final concentration of about 130 mM and polysorbate 20 to a final concentration of about 0.02% (w/v). In another embodiment, a composition is prepared by admixing the following: Antagonist A or a modified form thereof to a final concentration of about 6 mg/mL, ranibizumab to a final concentration of about 10 mg/mL, histidine to a final concentration of about 10 mM, sodium chloride to a final concentration of about 130 mM and polysorbate 20 to a final concentration of about 0.02% (w/v). In certain embodiments, the pH of the composition is adjusted to about 6.0.

In another embodiment, a composition is prepared by admixing the following: Antagonist A or a modified form thereof to a final concentration of about 6 mg/mL, aflibercept to a final concentration of about 40 mg/mL, phosphate to a final concentration of about 10 mM, sodium chloride to a final concentration of about 40 mM, sucrose to a final concentration of about 5% (w/v) and polysorbate 20 to a final concentration of about 0.03% (w/v). In another embodiment, a composition is prepared by admixing the following: Antagonist A or a modified form thereof to a final concentration of about 3 mg/mL, aflibercept to a final concentration of about 20 mg/mL, phosphate to a final concentration of about 10 mM, sodium chloride to a final concentration of about 40 mM, sucrose to a final concentration of about 5% (w/v) and polysorbate 20 to a final concentration of about 0.03% (w/v). In certain embodiments, the pH of the composition is adjusted to about 6.2.

In certain embodiments, the compositions are admixed in a glass vial or syringe or are stored after admixing in a glass viable or a syringe.

### Methods of Treating or Preventing Opthalmological Diseases

Compositions of the invention are useful for treating or preventing a variety of ophthalmological diseases. In some embodiments, the ophthalmological disease is a neovascular disorder. In other embodiments, the ophthalmological disease results in retinal edema. Illustrative ophthalmological diseases that can be treated or prevented by the present invention are described herein.

In certain embodiments, the invention provides methods for treating or preventing an ophthalmological disease, comprising administering to a mammal in need thereof a composition of the invention. In particular embodiments, an anti-PDGF aptamer present in the composition is Antagonist A or a modified form thereof. In particular embodiments, a VEGF antagonist present in the composition is ranibizumab, bevacizumab, or aflibercept. In particular embodiments, therapeutic agents present in compositions of the invention comprise an effective amount of: (i) Antagonist A or a modified form thereof and ranibizumab; (ii) a Antagonist A or a modified form thereof and bevacizumab; or (iii) Antagonist A or a modified form thereof and aflibercept.

In one embodiment, a composition of the invention comprises Antagonist A or a modified form thereof, ranibizumab, histidine, and sodium chloride. The composition may further comprise polysorbate.

In one particular embodiment, the composition of the invention comprises Antagonist A or modified form thereof and ranibizumab at a ratio of the concentration of Antagonist A or modified form thereof to the concentration of bevacizumab of less than 2, sodium chloride at a concentration of about 10 mM to about 200 mM, histidine at a concentration of about 1 mM to about 100 mM, and polysorbate (e.g., polysorbate 20) at a concentration of about 0.005% to about 0.05% or 0.001% to about 0.05%, wherein the pH of the composition is about 5.5 to about 7.0.

In one embodiment, the composition of the invention comprises Antagonist A or a modified form thereof at a concentration of about 3 mg/mL, ranibizumab at a concentration of about 5 mg/mL, histidine at a concentration of about 10 mM, sodium chloride at a concentration of about 130 mM and polysorbate 20 at a concentration of about 0.02% (w/v), wherein the pH of the composition is about 6.0. In a further embodiment, the composition comprises Antagonist A or a modified form thereof at a concentration of about 6 mg/mL, ranibizumab at a concentration of about 10 mg/mL, histidine at a concentration of about 10 mM, sodium chloride at a concentration of about 130 mM and polysorbate 20 at a concentration of about 0.02% (w/v), wherein the pH of the composition is about 6.0.

In one embodiment, a composition of the invention comprises Antagonist A or a modified form thereof, bevacizumab, sodium chloride, phosphate, and polysorbate. The composition may further comprise polysorbate.

In one particular embodiment, the composition of the invention comprises Antagonist A or modified form thereof and bevacizumab at a ratio of the concentration of Antagonist A or modified form thereof to the concentration of bevacizumab of less than 1, sodium chloride at a concentration of about 10 mM to about 200 mM, phosphate at a concentration of about 5 mM to about 200 mM, and polysorbate (e.g., polysorbate 20) at a concentration of about 0.005% to about 0.05%, wherein the pH of the composition is about 5.5 to about 7.0.

In one embodiment, the composition of the invention comprises Antagonist A or a modified form thereof at a concentration of about 3 mg/mL, bevacizumab at a concentration of about 12.5 mg/mL, phosphate at a concentration of about 50 mM, sodium chloride at a concentration of about 130 mM and polysorbate 20 at a concentration of about 0.02% (w/v), wherein the pH of the composition is about 6.0. In another embodiment, the composition comprises Antagonist A or a modified form thereof at a concentration of about 6 mg/mL, bevacizumab at a concentration of about 25 mg/mL, phosphate at a concentration of about 50 mM, sodium chloride at a concentration of about 130 mM and polysorbate 20 at a concentration of about 0.02% (w/v), wherein the pH of the composition is about 6.0.

In one embodiment, a composition of the invention comprises Antagonist A or a modified form thereof and aflibercept, sodium chloride, and phosphate. The composition may further comprise polysorbate or sucrose (or both).

In one particular embodiment, the composition of the invention comprises Antagonist A or a modified form thereof and aflibercept at a ratio of the concentration of Antagonist A to the concentration of aflibercept of less than 1, sodium chloride at a concentration of about 10 mM to about 200 mM, phosphate at a concentration of about 5 mM to about 50 mM, sucrose at a concentration of about 0% (w/v) to about 10% (w/v), and polysorbate (e.g., polysorbate 20) at a concentration of about 0.005% to about 0.05%, wherein the composition has a pH of about 6.0 to about 8.0.

In one embodiment, the composition of the invention comprises Antagonist A or a modified form thereof at a concentration of about 6 mg/mL, aflibercept at a concentration of about 40 mg/mL, phosphate at a concentration of about 10 mM, sodium chloride at a concentration of about 40 mM and polysorbate 20 at a concentration of about 0.03% (w/v), wherein the composition has a pH of about 6.2.

Ophthalmological Diseases

In certain embodiments, the ophthalmological disease is age-related macular degeneration. Examples of age-related macular degeneration are normeovascular (also known as “Dry”) and neovascular (also known as “Wet”) macular degeneration. In one embodiment, the dry age-related macular degeneration is associated with the formation of drusen. In some embodiments, treating or preventing dry macular degeneration encompasses treating or preventing an abnormality of the retinal pigment epithelium. Examples of abnormalities of the retinal pigment epithelium include geographic atrophy, non-geographic atrophy, focal hypopigmentation, and focal hyperpigmentation. In some embodiments, treating or preventing wet age-related macular degeneration encompasses treating or preventing choroidal neovascularization or pigment epithelial detachment.

In other embodiments, the ophthalmological disease is polypoidal choroidal vasculopathy. Polypoidal choroidal vasculopathy is characterized by a lesion from an inner choroidal vascular network of vessels ending in an aneurysmal bulge or outward projection (Ciardella et al (2004) Surv Ophthalmol 49 25-37).

In certain embodiments, the ophthalmological disease is a condition associated with choroidal neovascularization. Examples of conditions associated with choroidal neovascularization include a degenerative, inflammatory, traumatic or idiopathic condition. In some embodiments, treating or preventing a degenerative disorder associated with choroidal neovascularization encompasses treating or preventing a heredodegenerative disorder. Examples of heredodegenerative disorders include vitelliform macular dystrophy, fundus flavimaculatus and optic nerve head drusen. Examples of degenerative conditions associated with choroidal neovascularization include myopic degeneration or angioid streaks. In other embodiments, treating or preventing an inflammatory disorder associated with choroidal neovascularization encompasses treating or preventing ocular histoplasmosis syndrome, multifocal choroiditis, serpimnous choroiditis, toxoplasmosis, toxocariasis, rubella, Vogt-Koyanagi-Harada syndrome, Behcet syndrome or sympathetic ophthalmia. In still other embodiments, treating or preventing a traumatic disorder associated with choroidal neovascularization encompasses treating or preventing choroidal rupture or a traumatic condition caused by intense photocoagulation.

In other embodiments, the ophthalmological disease is hypertensive retinopathy or sicle cell retinopathy.

In one embodiment, the ophthalmological disease is diabetic retinopathy. Diabetic retinopathy can be nonproliferative or proliferative diabetic retinopathy. Examples of nonproliferative diabetic retinopathy include macular edema and macular ischemia.

In particular embodiments, the ophthalmological disease is a condition associated with peripheral retinal neovascularization. Examples of conditions associated with peripheral retinal neovascularization include ischemic vascular disease, inflammatory disease with possible ischemia, incontinentia pigmenti, retinitis pigmentosa, retinoschisis or chronic retinal detachment. Examples of ischemic vascular disease include proliferative diabetic retinopathy, branch retinal vein occlusion, branch retinal arteriolar occlusion, carotid cavernous fistula, sickling hemoglobinopathy, non-sickling hemoglobinopathy, IRVAN syndrome (retinal vasculitic disorder characterized by idiopathic retinal vasculitis, an aneurysm, and neuroretinitis), retinal embolization, retinopathy of prematurity, familial exudative vitreoretinopathy, hyperviscosity syndrome, aortic arch syndrome or Eales disease. Examples of sickling hemoglobinopathy include SS hemoglobinopathy and SC hemoglobinopathy. Examples of non-sickling hemoglobinopathy include AC hemoglobinopathy and AS hemoglobinopathy. Examples of hyperviscosity syndrome include leukemia, Waldenstrom macroglobulinemia, multiple myeloma, polycythemia or myeloproliferative disorder.

In some embodiments, treating or preventing an inflammatory disease with possible ischemia encompasses treating or preventing retinal vasculitis associated with systemic disease, retinal vasculitis associated with an infectious agent, uveitis or birdshot retinopathy. Examples of systemic diseases include systemic lupus erythematosis, Behcet's disease, inflammatory bowel disease, sarcoidosis, multiple sclerosis, Wegener's granulomatosis and polyarteritis nodosa. Examples of infectious agents include a bacterial agent that is the causative agent for syphilis, tuberculosis, Lyme disease or cat-scratch disease, a virus such as herpesvirus, or a parasite such as Toxocara canis or Toxoplasma gondii. Examples of uveitis include pars planitis or Fuchs uveitis syndrome.

In certain embodiments, the ophthalmological disease is retinopathy of prematurity. Retinopathy of prematurity can result from abnormal growth of blood vessels in the vascular bed supporting the developing retina (Pollan C (2009) Neonatal Netw. 28:93-101).

In other embodiments, the ophthalmological disease is venous occlusive disease or arterial occlusive disease. Examples of venous occlusive disease include branch retinal vein occlusion and central retinal vein occlusion. A branch retinal vein occlusion can be a blockage of the portion of the circulation that drains the retina of blood. The blockage can cause back-up pressure in the capillaries, which can lead to hemorrhages and also to leakage of fluid and other constituents of blood. Examples of arterial occlusive disease include branch retinal artery occlusion, central retinal artery occlusion or ocular ischemic syndrome. A branch retinal artery occlusion (BRAO) can occur when one of the branches of the arterial supply to the retina becomes occluded.

In particular embodiments, the ophthalmological disease is central serous chorioretinopathy (CSC). In one embodiment, CSC is characterized by leakage of fluid in the central macula

In one embodiment, the ophthalmological disease is cystoid macular edema (CME) In certain embodiments, CME affects the central retina or macula. In another embodiment, CME occurs after cataract surgery.

In other embodiments, the ophthalmological disease is retinal telangiectasia. In one embodiment, retinal telangiectasia is characterized by dilation and tortuosity of retinal vessels and formation of multiple aneurysms. Idiopathic JXT, Leber's miliary aneurysms, and Coats' disease are three types of retinal telangiectasias

In one embodiment, the ophthalmological disease is arterial macroaneurysm.

In one embodiment, the ophthalmological disease is retinal angiomatosis. In one embodiment, retinal angiomatosis occurs when the ocular vessels form multiple angiomas

In one embodiment, the ophthalmological disease is radiation-induced retinopathy (RIRP). In one embodiment, RIRP may display symptoms such as macular edema and nonproliferative and proliferative retinopathy

In certain embodiments, the ophthalmological disease is rubeosis iridis. In one embodiment, rubeosis iridis results in the formation of neovascular glaucoma. In another embodiment, rubeosis iridis is caused by diabetic retinopathy, central retinal vein occlusion, ocular ischemic syndrome, or chronic retinal detachment.

In certain embodiments, the ophthalmological disease is a neoplasm. Examples of neoplams include an eyelid tumor, a conjunctival tumor, a choroidal tumor, an iris tumor, an optic nerve tumor, a retinal tumor, an infiltrative intraocular tumor or an orbital tumor. Examples of an eyelid tumor include basal cell carcinoma, squamous carcinoma, sebaceous carcinoma, malignant melanoma, capillary hemangioma, hydrocystoma, nevus or seborrheic keratosis. Examples of a conjunctival tumor include conjunctival Kaposi's sarcoma, squamous carcinoma, intraepithelial neoplasia of the conjunctiva, epibular dermoid, lymphoma of the conjunctiva, melanoma, pingueculum, or pterygium. Examples of a choroidal tumor include choroidal nevus, choroidal hemangioma, metastatic choroidal tumor, choroidal osteoma, choroidal melanoma, ciliary body melanoma or nevus of Ota. Examples of an iris tumor include anterior uveal metastasis, iris cyst, iris melanocytoma, iris melanoma, or pearl cyst of the iris. Examples of an optic nerve tumor include optic nerve melanocytoma, optic nerve sheath meningioma, choroidal melanoma affecting the optic nerve, or circumpapillary metastasis with optic neuropathy. Examples of a retinal tumor include retinal pigment epithelial (RPE) hypertrophy, RPE adenoma, RPE carcinoma, retinoblastoma, hamartoma of the RPE, or von Hippel angioma. Examples of an infiltrative intraocular tumor include chronic lymphocytic leukemia, infiltrative choroidopathy, or intraocular lymphoma. Examples of an orbital tumor include adenoid cystic carcinoma of the lacrimal gland, cavernous hemangioma of the orbit, lymphangioma of the orbit, orbital mucocele, orbital pseudotumor, orbital rhabdomyosarcoma, periocular hemangioma of childhood, or sclerosing orbital pseudotumor.

The compositions of the invention can be administered alone or in conjunction with another therapy and can be provided at home, a doctor's office, a clinic, a hospital's outpatient department, or a hospital. The duration of the administration can depend on the type of ophthalmological disease being treated or prevented, the age and condition of the mammal, the stage and type of the mammal's disease, and how the mammal responds to the treatment. In particular embodiments, the mammal is a human. Additionally, a mammal having a greater risk of developing an ophthalmological disease (e.g., a diabetic patient) can receive treatment to inhibit or delay the onset of symptoms. In one embodiment, the present methods or compositions allow for the administration of a relatively lower dose of one or more of the anti-PDGF aptamer(s) and VEGF antagonist(s) present in the composition, as compared to the dose utilized when the therapeutic agent is used alone.

Administration of the composition of the invention may be by any suitable means that results in an amount of anti-PDGF aptamer and VEGF antagonist that is effective for the treatment or prevention of an ophthalmological disease. In one embodiment, the composition is administered in an amount effective to treat or prevent an ophthalmological disease.

The dosage of composition administered can depend on several factors including the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Furthermore, the exact individual dosages can be adjusted somewhat depending on a variety of factors, including the specific combination of therapeutic agents present in the composition, the time of administration, the route of administration, the nature of the composition, the rate of excretion, the particular ophthalmological disease being treated, the severity of the disorder, and the anatomical location of the disorder. The amount of each antagonist that is admixed with the carrier materials to produce a single dosage can vary depending upon the mammal being treated and the particular mode of administration.

For administration of compositions by parenteral injection, the dosage of each of the anti-PDGF aptamer and VEGF antagonist is typically 0.1 mg to 250 mg per day, 1 mg to 20 mg per day, or 3 mg to 5 mg per day. Injections may be given up to four times daily. Generally, when parenterally administered, the dosage of an anti-PDGF aptamer or VEGF antagonist for use in the present invention is typically 0.1 mg to 1500 mg per day, or 0.5 mg to 10 mg per day, or 0.5 mg to 5 mg per day. A dosage of at least up to 3000 mg per day can be administered.

When ophthalmologically administered to a human, for example intravitreally, the dosage of each of the anti-PDGF aptamer and VEGF antagonist present in the composition of the invention is typically 0.003 mg to 5.0 mg per eye per administration, or 0.03 mg to 3.0 mg per eye per administration, or 0.1 mg to 1.0 mg per eye per administration In one embodiment, the dosage of one or more anti-PDGF aptamer in the composition is 0.03 mg, 0.3 mg, 1.5 mg or 3.0 mg per eye. In another embodiment, the dosage of VEGF antagonist in the composition is about 0.5 mg per eye. The dosage can range from 0.01 mL to 0.2 mL administered per eye, or 0.03 mL to 0.15 mL administered per eye, or 0.05 mL to 0.10 mL administered per eye. For example, in certain embodiments, the anti-PDGF aptamer Antagonist A is delivered intravitreally at up to 30 mg/ml with injection volumes up to 100 μL.

Administration of the composition of the invention may be one to four times daily or one to four times per month or one to six times per year or once every two, three, four or five years. Administration can be for the duration of one day or one month, two months, three months, six months, one year, two years, three years, and may even be for the life of the patient. In one embodiment, the administration is performed once a month for three months. Chronic, long-term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

In addition to treating pre-existing ophthalmological diseases, the compositions can be administered prophylactically in order to prevent or slow the onset of these disorders. In prophylactic applications, the composition can be administered to a mammal susceptible to or otherwise at risk of a particular ophthalmological disease.

In one embodiment, the compositions of the invention are administered to a mammal in need of treatment thereof, typically in the form of an injectable pharmaceutical composition. The administration can be by injection, for example by intraocular injection, or by using a drug delivery device. Parenteral, systemic, or transdermal administration is also within the scope of the invention.

Compositions may be formulated to release the anti-PDGF aptamer or VEGF antagonist substantially immediately upon administration or at any predetermined time period after administration, using controlled release compositions. For example, a composition can be provided in sustained-release form. The use of immediate or sustained-release compositions depends on the nature of the condition being treated. For example, if the condition consists of an acute disorder, treatment with an immediate release form may be used over a prolonged release composition. For certain preventative or long-term treatments, a sustained released composition can also be used.

Many strategies can be pursued to obtain controlled release in which the rate of release outweighs the rate of degradation or metabolism of the therapeutic agents. For example, controlled release can be obtained by the appropriate selection of composition parameters and ingredients, including, e.g., appropriate controlled release compositions and coatings. Examples include oil solutions, suspensions, emulsions, microcapsules, microspheres, nanoparticles, patches, and liposomes. Depot formulations may also be used, e.g., in the form of microparticles, implants, or solid boluses that form in situ. Depot formulations may comprise a biodegradable polymer excipient that controls the rate of drug release and resorb during or after drug release. One class of biodegradable polymers is lactide/glycolide polymers. These resorbable polymers are biocompatible and are believed to resorb by hydrolysis, initially to lactic acid and glycolic acid, and eventually to carbon dioxide and water.

Compositions of the invention can also be delivered using a drug-delivery device such as an implant. Such implants can be biodegradable or biocompatible, or can be non-biodegradable. The implants can be permeable to the anti-PDGF aptamer or VEGF antagonist or deliver the agents by bioerosion. Ophthalmic drug delivery devices can be inserted into a chamber of the eye, such as the anterior or posterior chamber or can be implanted in or on the sclera, choroidal space, or an avascularized region exterior to the vitreous. In one embodiment, the implant can be positioned over an avascular region, such as on the sclera, so as to allow for transcleral diffusion of the anti-PDGF aptamer and VEGF antagonist to the desired site of treatment, e.g., the intraocular space and macula of the eye. Furthermore, the site of transcleral diffusion can be proximal to a site of neovascularization such as a site proximal to the macula. Suitable drug delivery devices are described, for example, in U.S. Publication Nos. 2008/0286334; 2008/0145406; 2007/0184089; 2006/0233860; 2005/0244500; 2005/0244471; and 2005/0244462, and U.S. Pat. Nos. 6,808,719 and 5,322,691, the contents of each of which is herein incorporated by reference in its entirety.

In one embodiment, the implant comprises a composition of the inventon dispersed in a biodegradable polymer matrix. The matrix can comprise PLGA (polylactic acid-polyglycolic acid copolymer), an ester-end capped polymer, an acid end-capped polymer, or a mixture thereof. In another embodiment, the implant comprises a composition comprising an anti-PDGF aptamer and a VEGF antagonist, a surfactant, and lipophilic compound. The lipophilic compound can be present in an amount of about 80-99% by weight of the implant. Suitable lipophilic compounds include, but are not limited to, glyceryl palmitostearate, diethylene glycol monostearate, propylene glycol monostearate, glyceryl monostearate, glyceryl monolinoleate, glyceryl monooleate, glyceryl monopalmitate, glyceryl monolaurate, glyceryl dilaurate, glyceryl monomyristate, glyceryl dimyristate, glyceryl monopalmitate, glyceryl dipalmitate, glyceryl monostearate, glyceryl distearate, glyceryl monooleate, glyceryl dioleate, glyceryl monolinoleate, glyceryl dilinoleate, glyceryl monoarachidate, glyceryl diarachidate, glyceryl monobehenate, glyceryl dibehenate, and mixtures thereof.

In another embodiment, the implant comprises a composition of the invention housed within a hollow sleeve. The composition comprising the anti-PDGF aptamer and VEGF antagonist are delivered to the eye by inserting the sleeve into the eye, releasing the implant from the sleeve into the eye, and then removing the sleeve from the eye. An example of this delivery device is described in U.S. Publication No. 2005/0244462, which is hereby incorporated by reference in its entirety.

In one embodiment, the implant is a flexible ocular insert device adapted for the controlled sustained release of an anti-PDGF aptamer and a VEGF antagonist into the eye. In one embodiment, the device includes an elongated body of a polymeric material in the form of a rod or tube containing a composition comprising an anti-PDGF aptamer and a VEGF antagonist, and with at least two anchoring protrusions extending radially outwardly from the body. The device may have a length of at least 8 mm and the diameter of its body portion including the protrusions does not exceed 1.9 mm. The sustained release mechanism can, for example, be by diffusion or by osmosis or bioerosion. The insert device can be inserted into the upper or lower formix of the eye so as to be independent of movement of the eye by virtue of the formix anatomy. The protrusions can be of various shapes such as, for example, ribs, screw threads, dimples or bumps, truncated cone-shaped segments or winding braid segments. In a further embodiment, the polymeric material for the body is selected as one which swells in a liquid environment. Thus a device of smaller initial size can be employed. The insert device can be of a size and configuration such that, upon insertion into the upper or lower formix, the device remains out of the field of vision so as to be well retained in place and imperceptible by a recipient over a prolonged period of use. The device can be retained in the upper or lower formix for 7 to 14 days or longer. An example of this device is described in U.S. Pat. No. 5,322,691, which is hereby incorporated by reference in its entirety.

In certain embodiments, compositions of the invention can also be delivered using a drug-delivery device such as an exoplant, e.g., an episcleral oxplant, such as one described in Pontes de Carvalho, R. A. et al., Invest Ophthalmol V is Sci. 2006, 47(1):4532-9, incorporated by reference in its entirety. Such exoplants can be biodegradable or biocompatible, or can be non-biodegradable.

In other embodiments, compositions of the invention can also be delivered using a drug-delivery device such as a refillable intraocular depot.

Dosing is generally dependent on severity and responsiveness of the condition to be treated, with course of treatment lasting from several days to several months or until a cure is effected or a diminution of disease state is achieved. Optimal dosing schedules can be calculated from measurements of drug accumulation in the body or at a localized site or based upon a patient's response. Persons of ordinary skill can optimize dosages, dosing methodologies, and repetition rates. Optimum dosages may vary depending on the potency of anti-PDGF agonists and VEGF antagonists, and may also be estimated based on EC50's in in vitro and in vivo animal studies.

### EXAMPLES

### Example 1

### Stability of Compositions Comprising Antagonist A and Ranibizumab

The composition stability of Antagonist A and ranibizumab, commercially available as Lucentis® from Genentech (S. San Francisco, Calif.), in various compositions was examined under a range of conditions. Various pHs (5.0-8.0) and tonicity modifiers (sodium chloride, sorbitol, and trehalose) were used to optimize the composition stability at various storage conditions (4° C., 25° C., and 37° C.) and under a physical stress (agitation). The composition stability of Antagonist A and ranibizumab was characterized by visual observation, pH measurement, and various HPLC methods (anion exchange [AEX-HPLC], weak cation exchange [WCX-HPLC], and size exclusion [SE-HPLC]).

Throughout the 16 weeks of the study, it was determined that among the compositions examined a composition comprising Antagonist A at 3 mg/mL and ranibizumab at 5 mg/mL in 10 mM L-histidine at pH 6.0, 130 mM NaCl, 0.01% (w/v) polysorbate 20 (F6) was the most stable and provided the greatest protection against the degradation of Antagonist A and ranibizumab. A more detailed description of the experiments performed is provided herein.

### Composition Parameters

The following composition parameters were examined:
(1) pH: 4.0, 5.0, 6.0, 6.5, 7.0, 7.3, 8.0
(2) Buffers: Acetate, Phosphate, Histidine and 2-Amino-2-hydroxymethyl-propane-1,3-diol (“Tris”)
(3) Tonicity Modifiers: Sodium Chloride, Sorbitol, and Trehalose
(4) Surfactants: Polysorbate 20 [0.01% and 0.005% (% w/v)]

The following parameters were fixed:
(1) Fill volume was 300 μL in modified 3 cc vials provided by Ophthotech Corp. (obtained from Mglas AG, Munnerstadt, Germany)
(2) The concentration of ranibizumab was 5 mg/mL
(3) The concentration of Antagonist A was fixed at 3 mg/mL

Table 1 below summarizes the composition matrix used in this study.

TABLE 1
Composition Matrix

| | | | | [Ant. A] | [ran.] | Polysorbate |
|---|---|---|---|---|---|---|
| Comp. | Buffer | pH | Tonicity Modifier | (mg/mL) | (mg/mL) | 20 (% w/v) |
| F1 | 10 mM Sodium | 7.3 | 150 mM NaCl | 3 | 0 | 0% |
| | Phosphate | | | | | |
| F2 | 10 mM Sodium | 5.0 | 5% (w/v) Sorbitol | 3 | 5 | 0.01% |
| | Acetate | | | | | |
| F3 | 10 mM Sodium | 5.0 | 130 mM NaCl | 3 | 5 | 0.01% |
| | Acetate | | | | | |
| F4 | 10 mM Histidine•HCl | 5.5 | 10% (w/v) | 0 | 5 | 0.01% |
| | | | Trehalose | | | |
| F5 | 10 mM Histidine•HCl | 6.0 | 5% (w/v) Sorbitol | 3 | 5 | 0.01% |
| F6 | 10 mM Histidine•HCl | 6.0 | 130 mM NaCl | 3 | 5 | 0.01% |
| F7 | 10 mM Sodium | 7.0 | 5% (w/v) Sorbitol | 3 | 5 | 0.01% |
| | Phosphate | | | | | |
| F8 | 10 mM Sodium | 7.0 | 130 mM NaCl | 3 | 5 | 0.01% |
| | Phosphate | | | | | |
| F9 | 10 mM Tris•HCl | 8.0 | 5% (w/v) Sorbitol | 3 | 5 | 0.01% |
| F10 | 10 mM Tris•HCl | 8.0 | 130 mM NaCl | 3 | 5 | 0.01% |
| F11 | 5 mM Sodium | 6.5 | 75 mM NaCl + | 3 | 5 | 0.005% |
| | Phosphate + 5 mM | | 5% (w/v) | | | |
| | Histidine | | Trehalose | | | |

“Ant. A” is Antagonist A; “ran.” is ranibizumab

### Sample Preparation

In order to obtain a 3 mg/mL concentration of Antagonist A in the composition, an Antagonist A stock solution was prepared at 6 mg/mL in 10 mM phosphate, 150 mM NaCl, and pH 7.3. The resulting stock solution was admixed 1:1 with a diluted form of commercial Lucentis® (10 mg/mL), resulting in final concentrations of 3 mg/mL Antagonist A and 5 mg/mL ranibizumab (F11). The composition was placed in 10 kDa molecular weight cutoff dialysis cassettes and dialyzed ˜1,000,000-fold against the various composition buffers listed in Table 1 (Comp. Nos. F2-F3, F5-F 10).

### Composition Studies

The compositions were tested under the following conditions (although certain compositions were not tested at all time points due to degradation at earlier time points):

TABLE 2
Test Conditions

| Conditions | Timepoints |
|---|---|
| 4° C. | 0, 2, 4, 8, 12, and 16 weeks |
| 25° C. | 2, 4, 8, and 12 weeks |
| 37° C. | 2, 4, 8, and 12 weeks |
| Agitation | 4 hours |

### Analytical Methods

In order to measure the concentration of any degradation products generated under stress in the various compositions, the following stability-indicating assays were used:

(1) SE-HPLC (Analysis of Antagonist A and Ranibizumab)
Mobile Phase: 50 mM phosphate buffer, 100 mM sodium chloride, pH 7.0
Column. Tosoh TSKgel G3000SWXL 7.8 mm×300 mm, 5 nm particles
Column Temperature: Ambient
Flow Rate: 1.0 mL/min
Wavelength: Signal, 280 nm; Reference, 360 nm
Injection volume: 5 μL
Sample Preparation: No dilution
Percent purity reported based on integrated area percent of main peaks identified for both Antagonist A and ranibizumab.

(2) WCX-HPLC (Analysis of Ranibizumab)
Mobile Phase A: 10 mM phosphate buffer, pH 7.0
Mobile Phase B: 10 mM phosphate buffer, 500 mM sodium chloride, pH 7.0
Column: Dionex ProPac WCX-10, 4×250 mm
Column Temperature: Ambient
Flow Rate: 1.0 mL/min
Wavelength: Signal, 214 nm; Reference, 360 nm
Injection volume: 5 μL
Sample Preparation: No dilution
Percent purity reported based on integrated area percent of main peaks identified for both Antagonist A and ranibizumab.

(3) AEX-HPLC (Analysis of Antagonist A)
Mobile Phase A: 10 mM phosphate buffer, pH 7.0
Mobile Phase B: 10 mM phosphate buffer, 500 mM sodium chloride, pH 7.0
Column: Dionex DNA Pac PA-100, 4×250 mm
Column Temperature: 40° C.
Flow Rate: 1.2 mL/min
Wavelength: Signal, 258 nm; Reference, 360 nm
Injection volume: 5 μL
Sample Preparation: No dilution
Percent purity reported based on integrated area percent of main peaks identified for both Antagonist A and ranibizumab.

(4) pH
VWR symphony SB70P

(5) Visual Observation
Photos taken from Sony Cyber-shot DSC-H9 Digital Still Camera (8.1 Mega pixels)

(6) Osmolarity
Advanced Instruments, Inc. The Advanced Osmometer Model 3D3

### Stability Overview

The effects of both agitation (4 hours) and the various storage temperatures (4° C., 25° C., and 37° C.) on various Antagonist A and ranibizumab compositions were analyzed. Throughout the study, all of the compositions tested were able to maintain their target pH values, i.e., titrated initial pH, through all storage and stress conditions.

### Stability Indicating Assays

Composition F2 developed visible precipitation during storage at 37° C. after two weeks (data not shown). No other assays were performed for quantitative measurement of the precipitation.

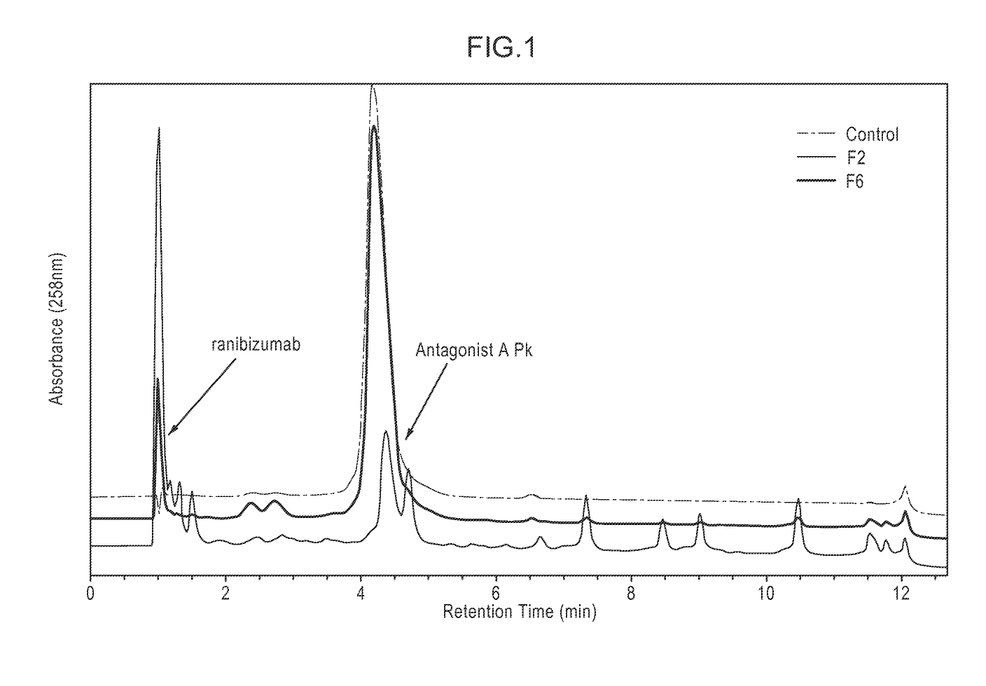

The degradation of Antagonist A during storage was effectively analyzed by AEX-HPLC (FIG. 1). The formation of pre-peaks and post-peaks was observed when samples were incubated at elevated temperature (FIG. 1). In composition F2, the AEX-HPLC purity of Antagonist A decreased by nearly 20% during storage for 8 weeks at 37° C.

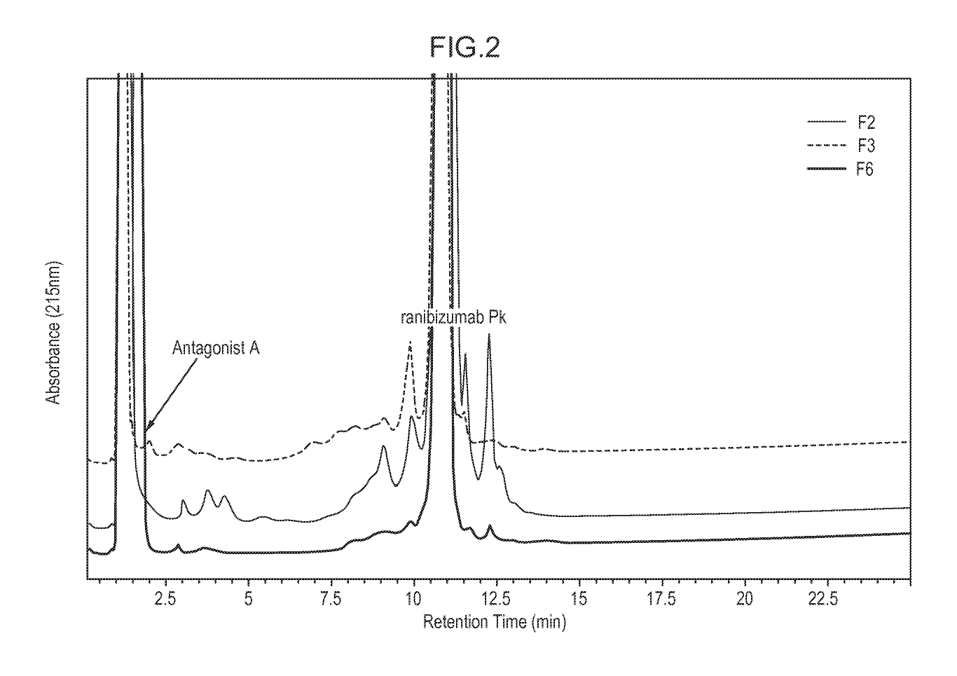

WCX-HPLC was also effective at characterizing the degradation of ranibizumab during storage (FIG. 2). The formation of both pre-peaks and post-peaks was observed when ranibizumab was incubated at elevated temperature (FIG. 2).

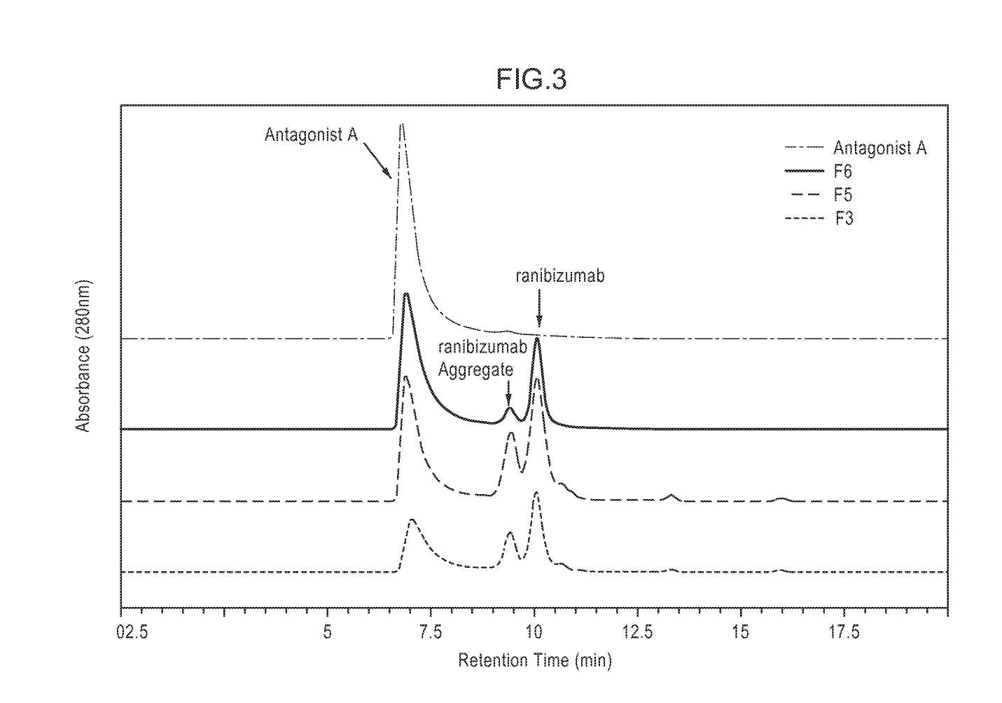

The SE-HPLC assay was useful for characterizing soluble aggregation or fragmentation of ranibizumab (FIG. 3). Antagonist A did not show significant changes by SE-HPLC, although the resolution of its aggregated form may be beyond the capacity of Tosoh TSKgel G3000SWXL column. Ranibizumab underwent aggregation or fragmentation in composition F3 and composition F5 during storage at 37° C. (FIG. 3).

Effect of Agitation on Stability

All compositions listed in Table 1 underwent 4 hours of agitation, with a set of non-agitated control compositions left at room temperature. No differences were seen between control and agitated samples on any analytical methods (data not shown).

Effect of Storage Temperature on Stability

Storage at 37° C. produced significant albeit varying levels of degradation of both Antagonist A and ranibizumab in the various compositions investigated. By two weeks, composition F2 developed precipitation (data not shown). All other compositions remained clear through eight weeks, and up to 12 weeks for several compositions (Compositions F1, F4, F6, F8, and F11).

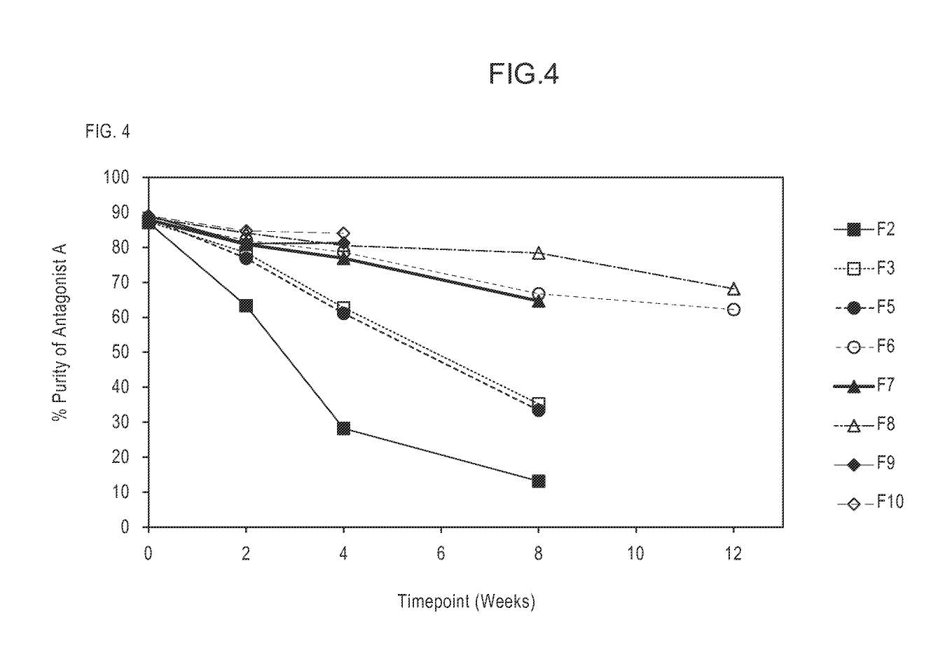

After two weeks at 37° C., Antagonist A purity in composition F2 had decreased by nearly 20% based on AEX-HPLC. Compositions F3 and F5 also revealed increased Antagonist A degradation after four weeks under the same storage conditions (FIG. 4). By eight weeks, it appeared that composition F8 offered greater protection to Antagonist A than did F6 and F7. By 12 weeks, F8 continued to display higher purity of Antagonist A (FIG. 4).

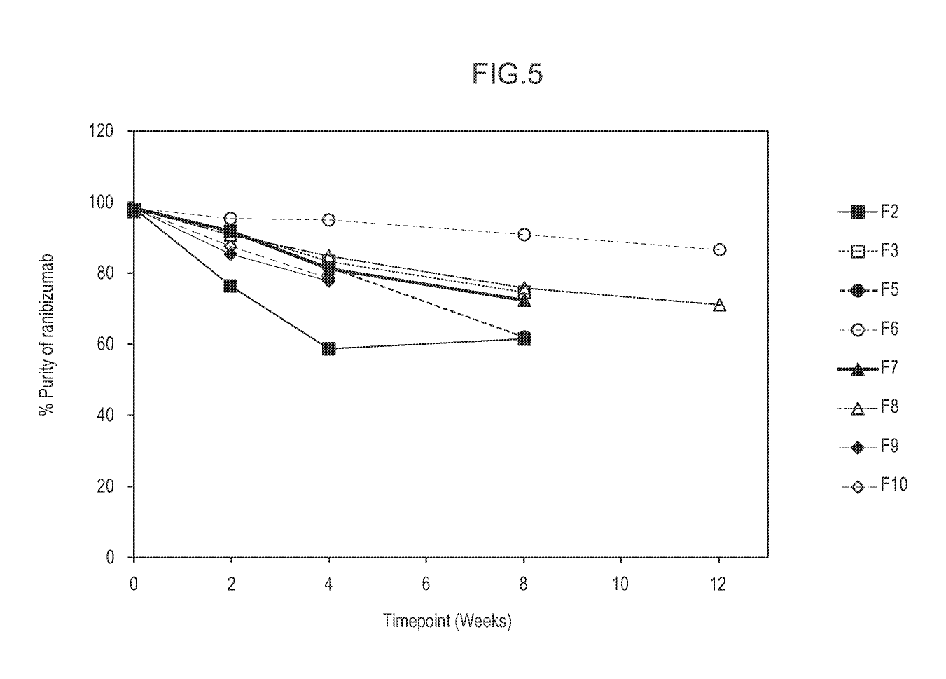

Composition F2 also could not prevent degradation of ranibizumab, as WCX-HPLC detected nearly 20% loss of purity by 2 weeks (FIG. 5). By the fourth week, many compositions (F3, F5, F7, F8, F9, and F10) exhibited significant degradation of ranibizumab in comparison to F6 (FIG. 5). Composition F6 maintained the best purity of ranibizumab for up to 12 weeks (FIG. 5).

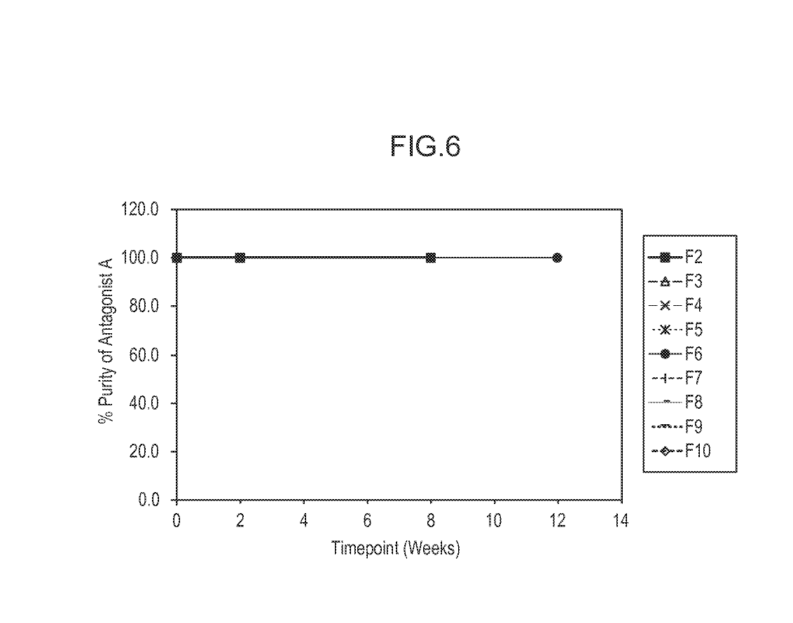

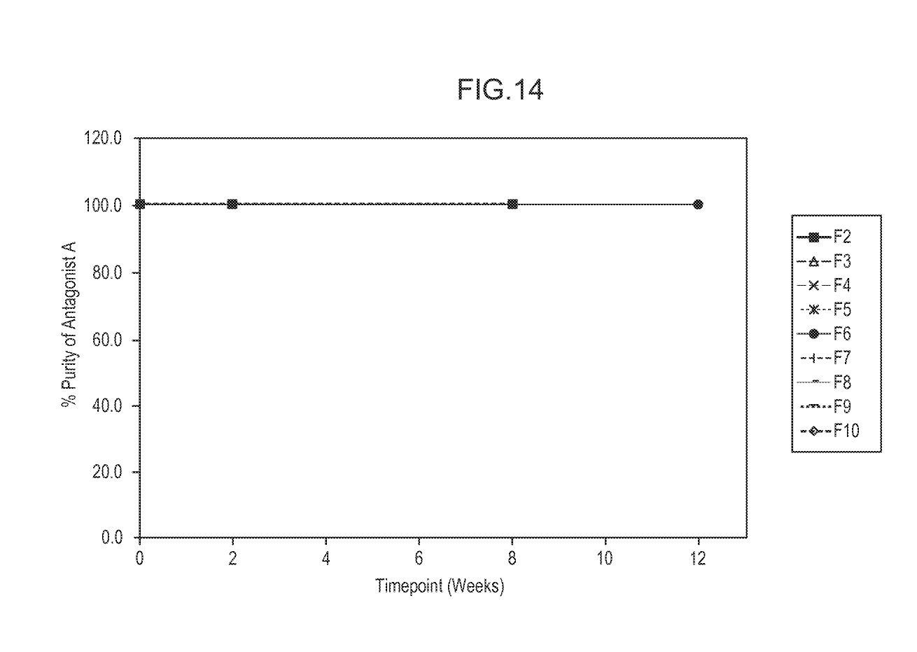

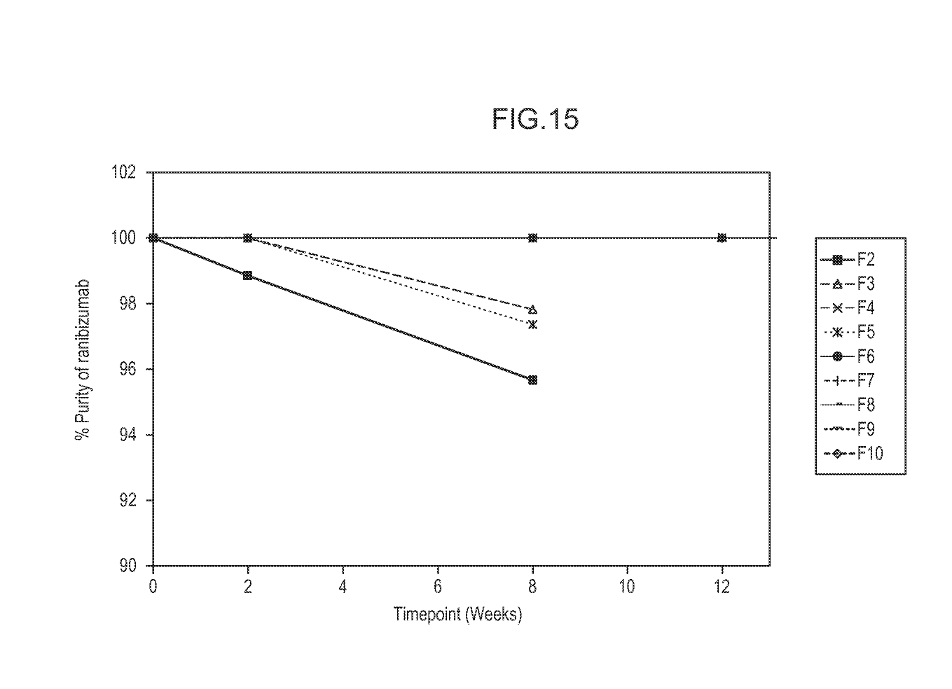

Based on the results from 2, 8, and 12 weeks at 4° C., which showed a single peak for the native form of ranibizumab, all compositions displayed similar purity profiles of Antagonist A and ranibizumab for up to 4 weeks by SE-HPLC (FIG. 14 and FIG. 15). No significant change was observed for Antagonist A at all storage conditions, including the 12 week storage at 37° C. (FIG. 6). However, ranibizumab underwent aggregation during storage at 25° C. and 37° C. No significant aggregation was observed with a diluted form of commercial Lucentis® (F4) under the same storage condition (FIG. 7).

All compositions showed better visual stability during storage at 25° C. than at 37° C. Over 8 weeks, all compositions remained clear. Two compositions (F6 and F8) remained clear at the additional 12 week time point.

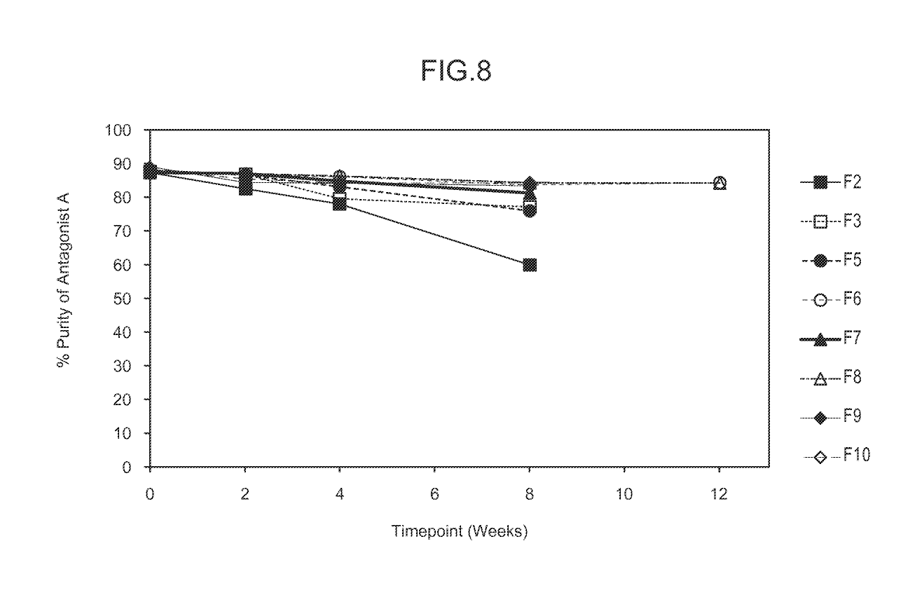

For the first four weeks at 25° C., all compositions maintained comparable Antagonist A purity as characterized by AEX-HPLC (F1G. 8). Composition F2 underwent a significant increase in Antagonist A degradation by 8 weeks (FIG. 8). Also, compositions F3 and F5 displayed considerable decreases in purity over the same timeframe (FIG. 8). Compositions F6, F7, and F8 were able to maintain the purity of Antagonist A for up to 12 weeks (FIG. 8).

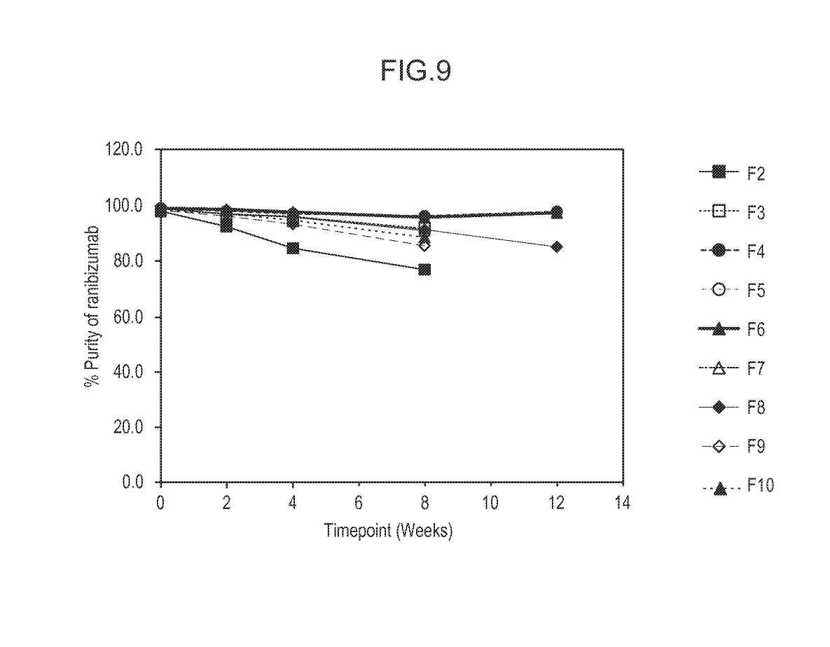

WCX-HPLC analysis of ranibizumab displayed subtle yet distinctive changes in purity profiles between the compositions. After two weeks of storage at 25° C., composition F2 developed considerable degradation of ranibizumab (FIG. 9). The remaining compositions maintained comparable purity of ranibizumab until eight weeks, when the pH 8.0 compositions (F9 and F 10) revealed a considerable decrease in purity of ranibizumab (FIG. 9). Composition F6 was able to prevent degradation of ranibizumab at 25° C. as determined by WCX-HPLC analysis (FIG. 9).

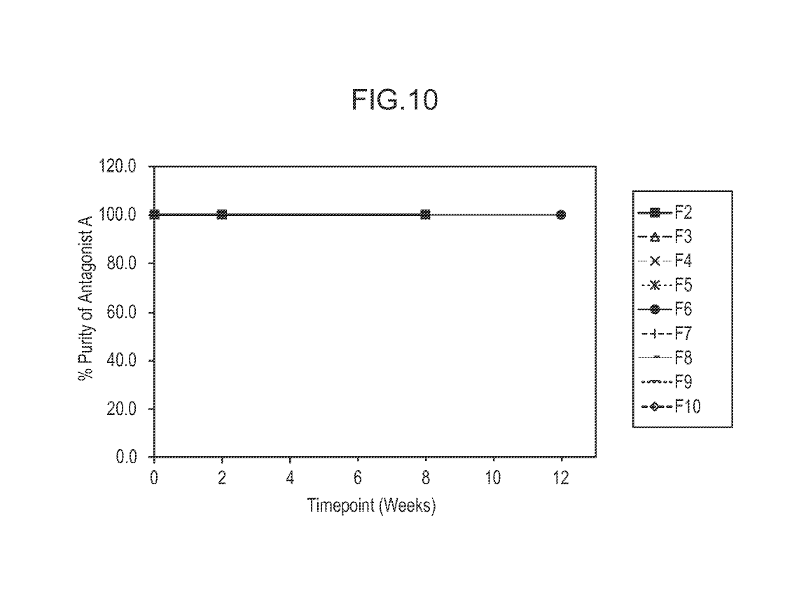

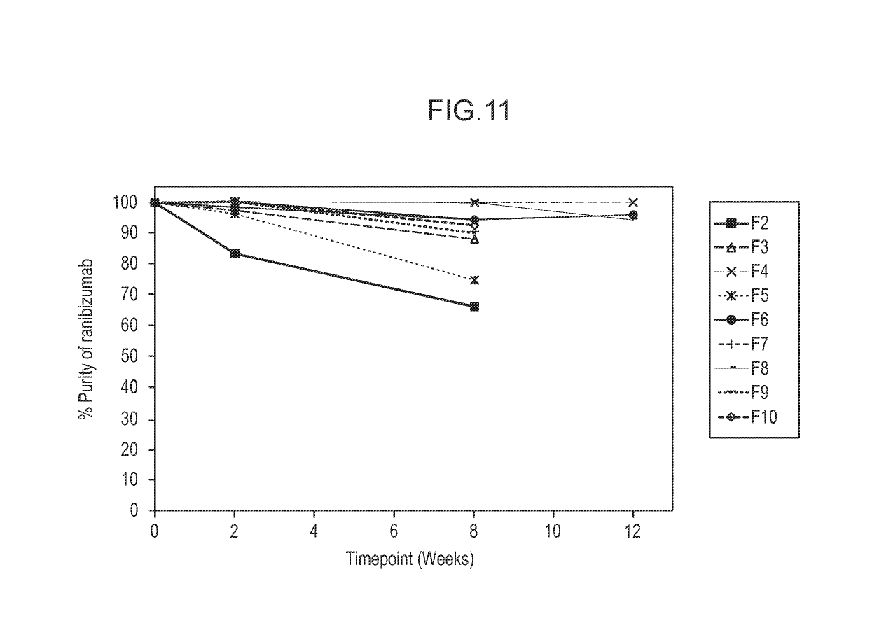

Other than its inherent variability, the SE-HPLC assay showed no significant change of Antagonist A profile during storage at 25° C. (FIG. 10). In general, all compositions appeared to prevent aggregation or fragmentation of Antagonist A over eight weeks, and over twelve weeks for compositions F6 and F8 (FIG. 10). Compositions F8 and F6 maintained good ranibizumab purity for twelve weeks at 25° C. (FIG. 11).

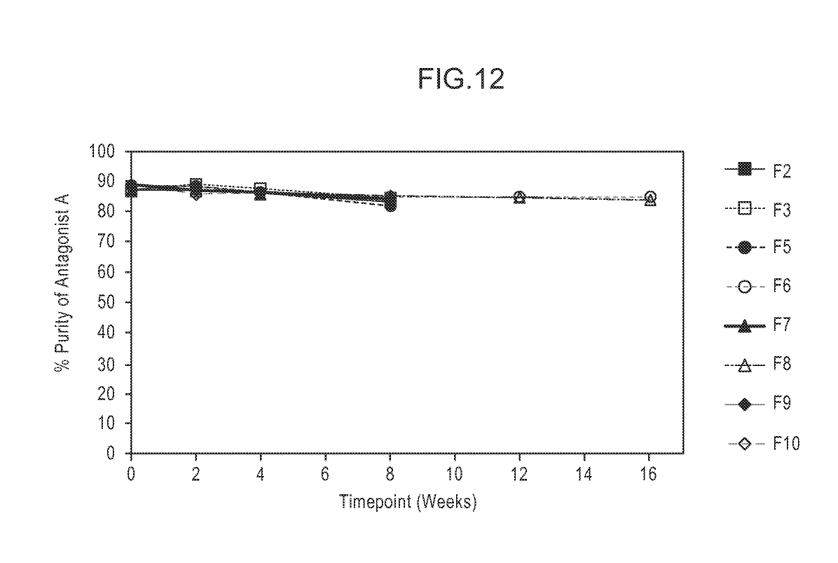

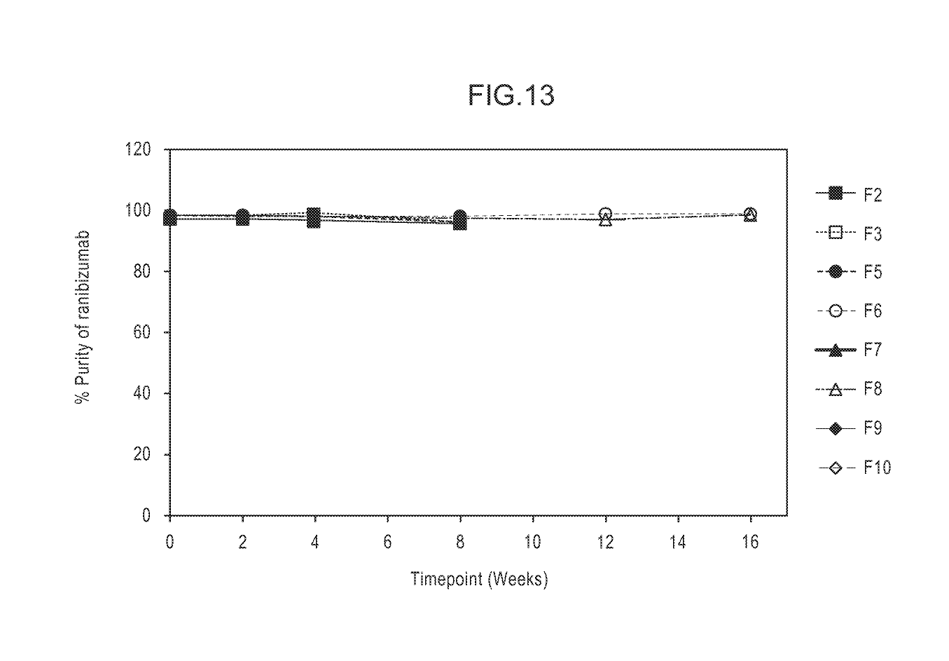

Antagonist A and ranibizumab remained stable in most compositions at 4° C. All compositions remained clear by visual inspection. Furthermore, most compositions maintained comparable purity to the starting material by all HPLC methods (FIG. 12-15), except for F2, F3, and F5, which yielded substantial amounts of soluble aggregates of ranibizumab (FIG. 15).

### Effect of Composition Characteristics/Components on Stability

To determine the effect that pH and different composition components have on the stability of Antagonist A and ranibizumab, Antagonist A and ranibizumab were coformulated at various pH levels (5.0-8.0) and with different tonicity modifiers (sodium chloride and sorbitol). This section describes the effects of pH and composition components on the stability of one or both of Antagonist A and ranibizumab when stored at various temperatures.

Effect of pH on Stability

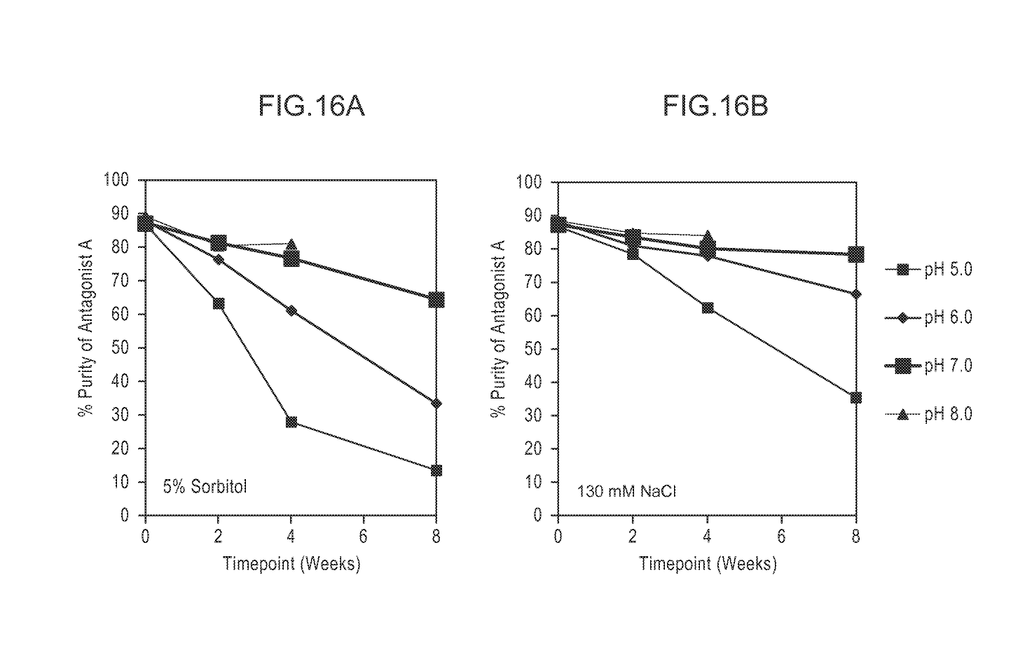

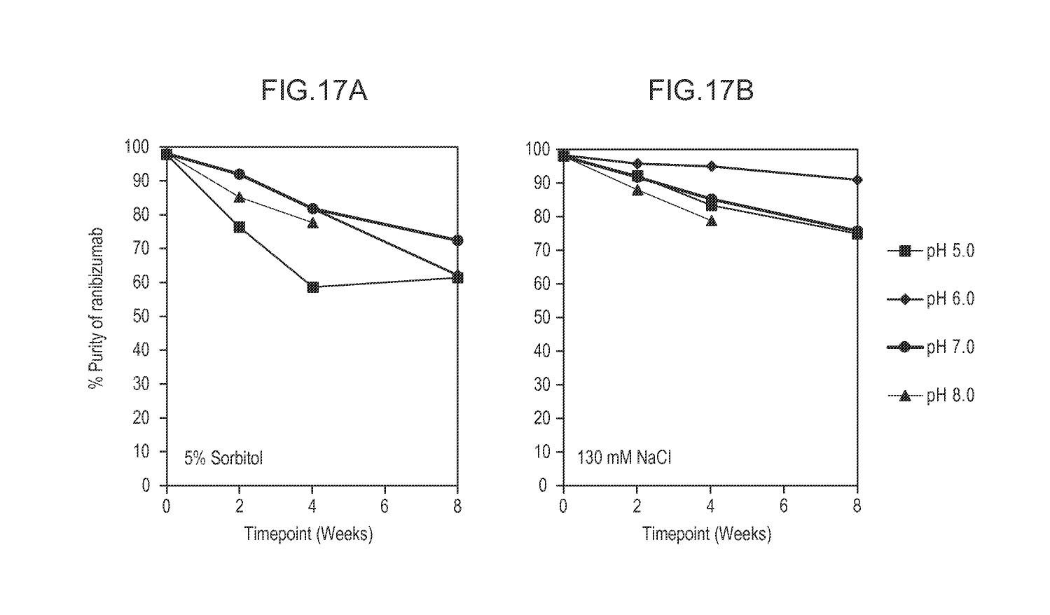

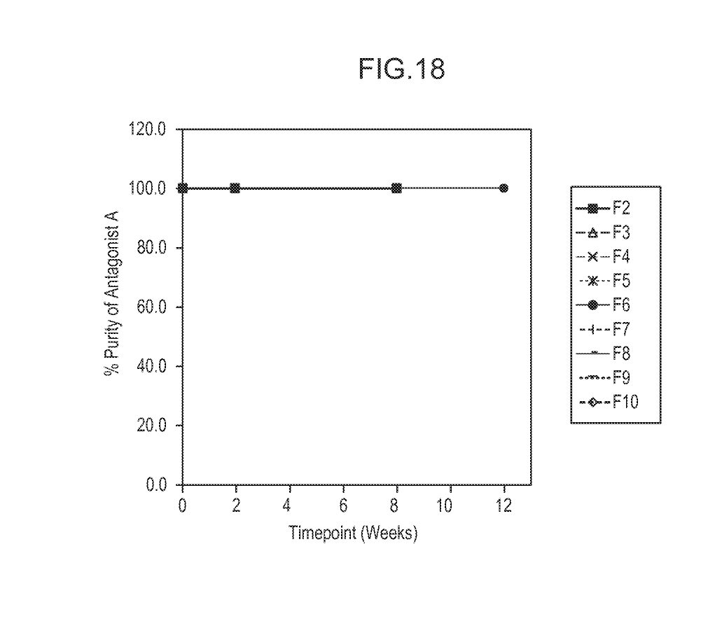

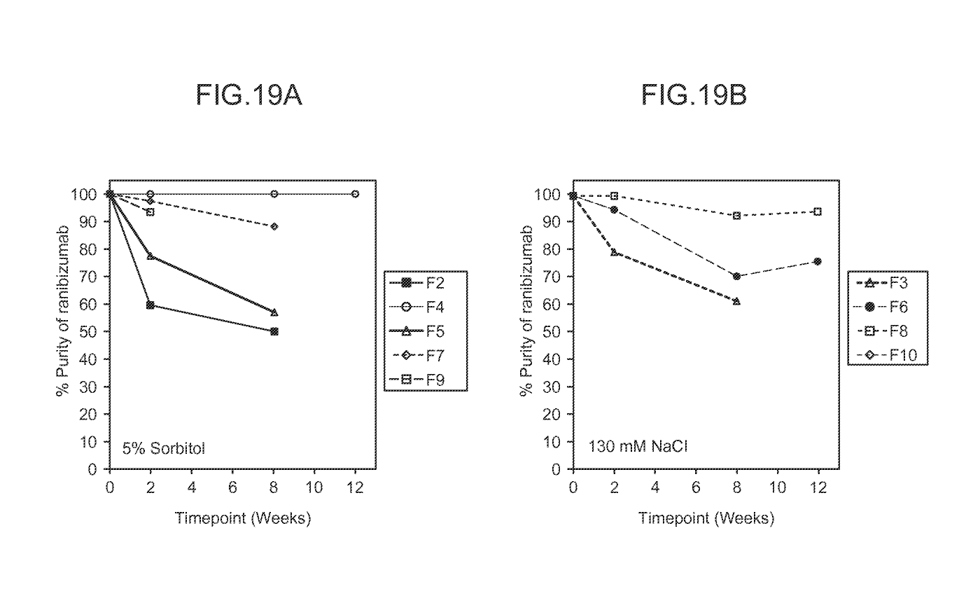

The effect of pH on the stability of Antagonist A and ranibizumab was best differentiated by storage at 37° C. in both sorbitol-containing and NaCl containing compositions (FIG. 16). Based on AEX-HPLC, degradation of Antagonist A was inversely correlated with pH, with the greatest degradation at pH 5.0 (FIG. 16). Changes in pH caused less significant changes to the purity profile of ranibizumab in sorbitol-containing compositions. Based on WCX-HPLC, formulating at pH 5.0 yielded faster degradation of ranibizumab after four weeks at 37° C., but yielded similar degradation to the pH 6.0 compositions after eight weeks at 37° C. (FIG. 17). Ranibizumab was least degraded at pH 6.0 among the NaCl-containing compositions, while pH 7.0 was the best among the sorbitol-containing compositions. Using SE-HPLC for evaluation of both Antagonist A (FIG. 18) and ranibizumab (FIG. 19), the aggregation rate of ranibizumab was slowest in compositions at pH 7.0, while no changes were observed for Antagonist A degradation.

Effect of Tonicity Modifier on Stability

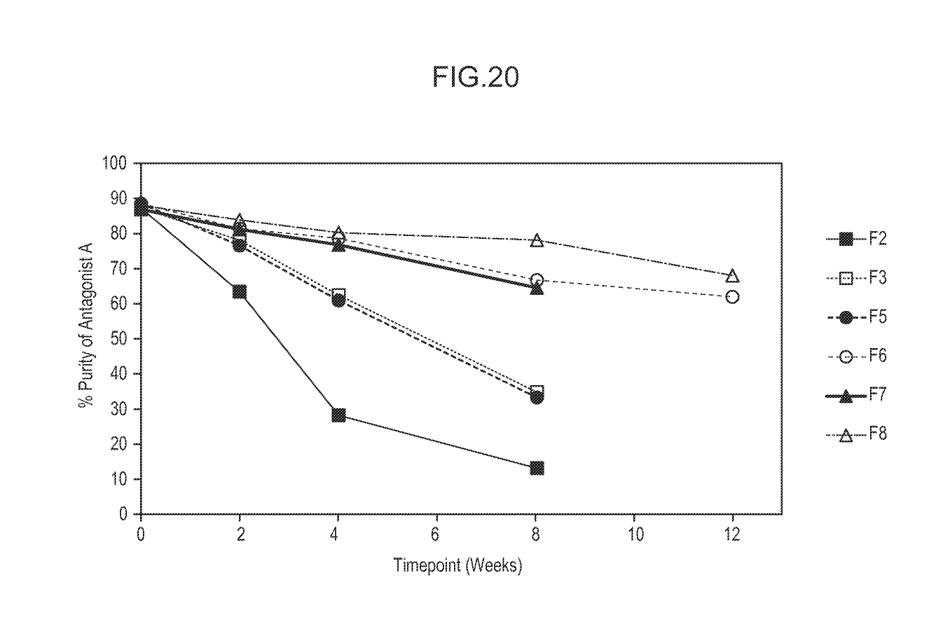

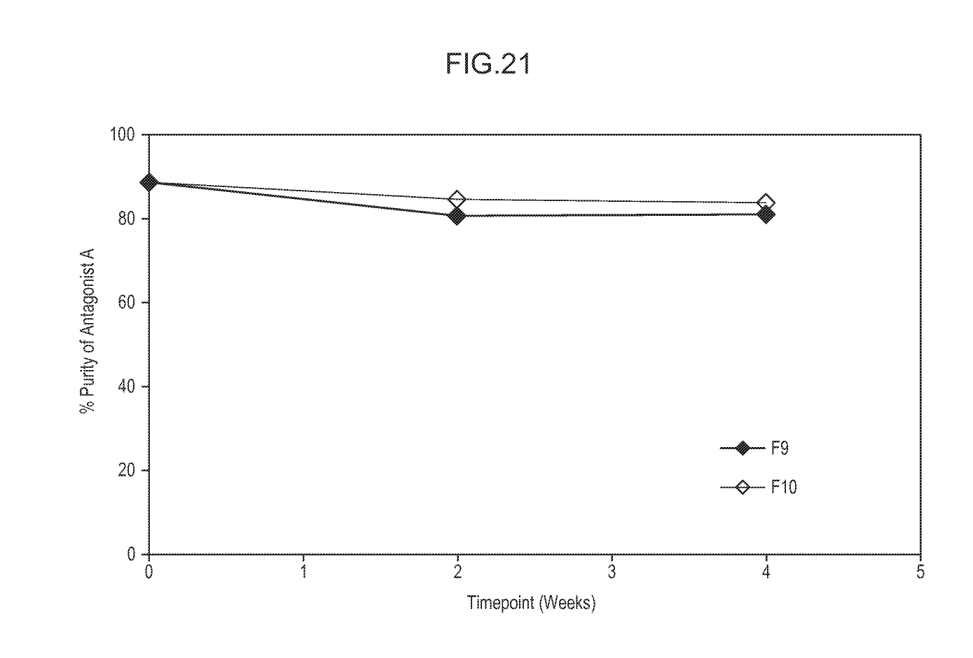

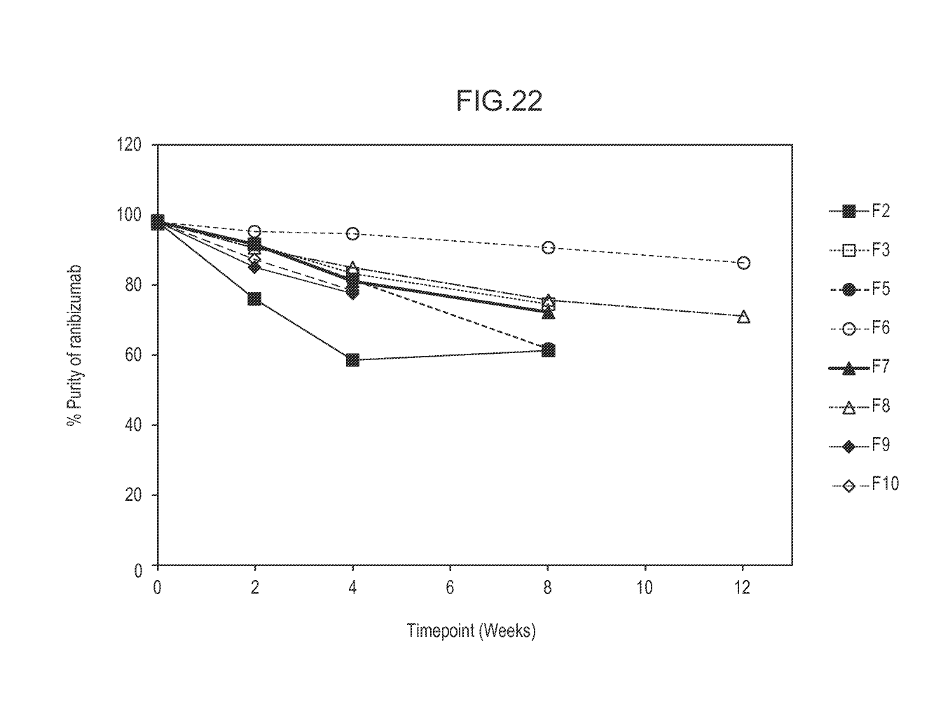

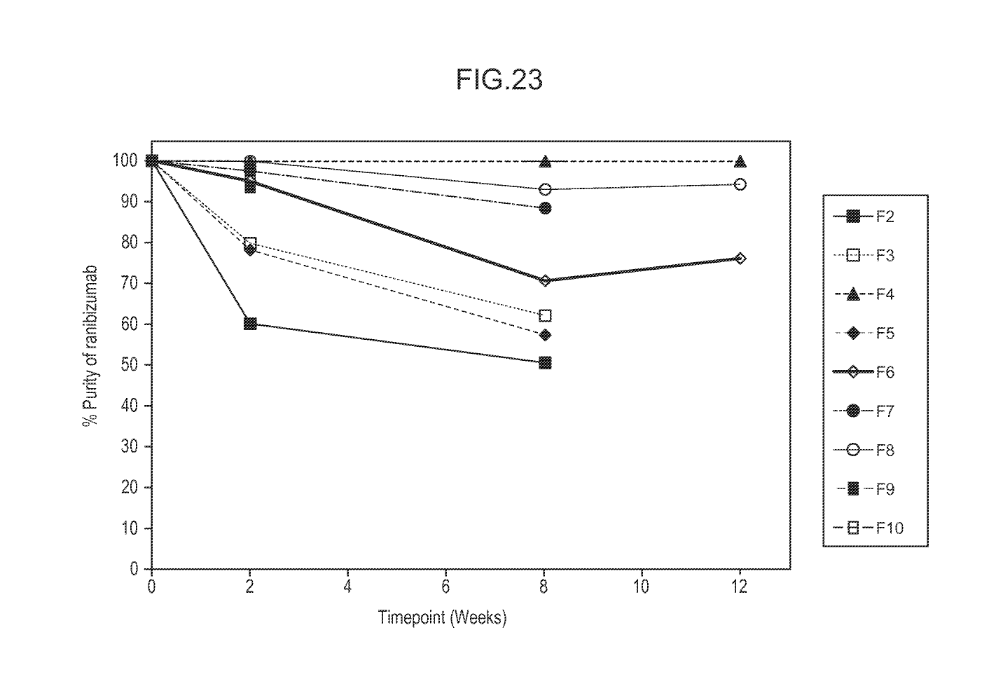

The effect of tonicity modifiers on the stability of Antagonist A and ranibizumab was differentiated by comparing the results from 37° C. storage. As characterized by AEX-HPLC, Antagonist A remained more stable in NaCl compositions than in sorbitol compositions at pH 5.0-7.0 over 8 weeks (FIG. 20). At pH 8.0, no discernable difference could be made between compositions containing sodium chloride or sorbitol over 4 weeks (FIG. 21). For ranibizumab compositions, as characterized by WCX-HPLC, sodium chloride compositions outperformed sorbitol compositions across the pH range tested (pH 5.0-8.0) (FIG. 22). The superior performance of sodium chloride compositions in stabilizing ranibizumab was also revealed by SE-HPLC (FIG. 23). For compositions with both tonicity modifiers, the level of soluble aggregation was lowest at pH 7.0 and highest at pH 5.0 (FIG. 23).

Stability of 1:1 Mixture of Antagonist A and Lucentis®

Another aspect of the study involved characterizing the effect of admixing Antagonist A and commercial Lucentis®. To accomplish this, Antagonist A was diluted to 6 mg/mL from its original concentration of 30 mg/mL in a composition of 10 mM sodium phosphate and 150 mM NaCl, pH 7.3, followed by combining the resulting composition with an equal volume (1:1) of commerical Lucentis® (10 mg/mL). Stability of the 1:1 mixture (F11) was examined by storage at 37° C., and was compared to F1 and F4 alone at similar concentrations and storage temperatures.

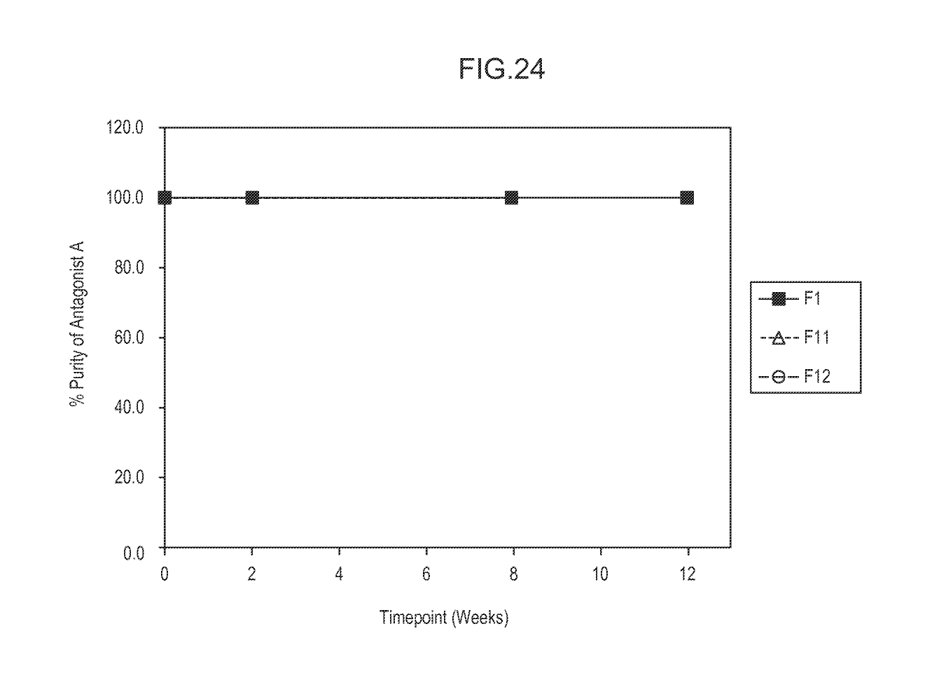

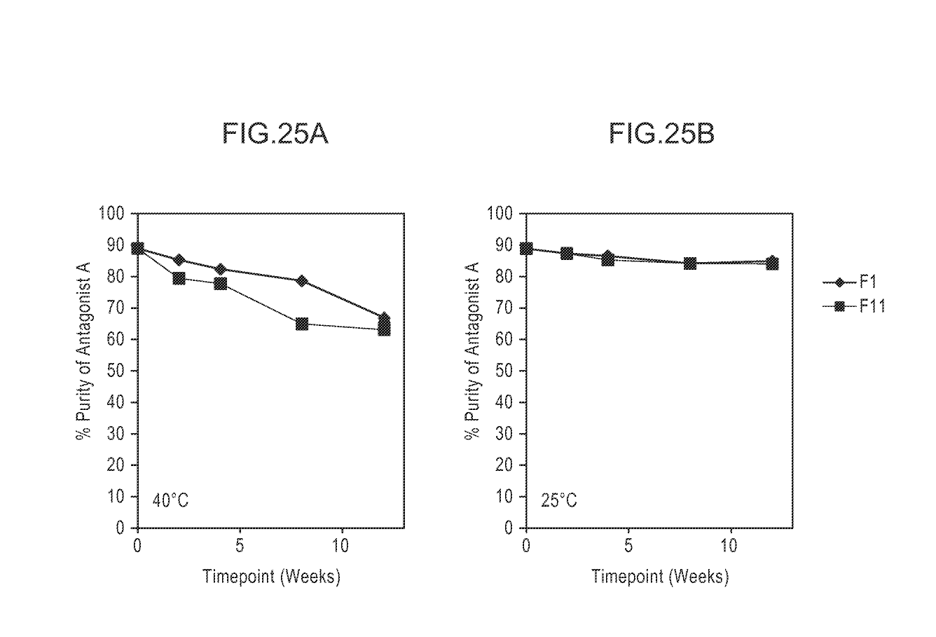

For Antagonist A, SE-HPLC analysis indicated that the stability of Antagonist A in the 1:1 mixture, F11, is comparable to F1 alone over twelve weeks at 37° C. (FIG. 24). Although it appeared by AEX-HPLC that Antagonist A underwent faster degradation in the 1:1 mixture at earlier time points during storage at 37° C., both F1 and the 1:1 mixture (F11) displayed comparable purity by 12 weeks (FIG. 25). No difference in the AEX-HPLC purity profile was observed when the samples were stored at 25° C. (FIG. 25).

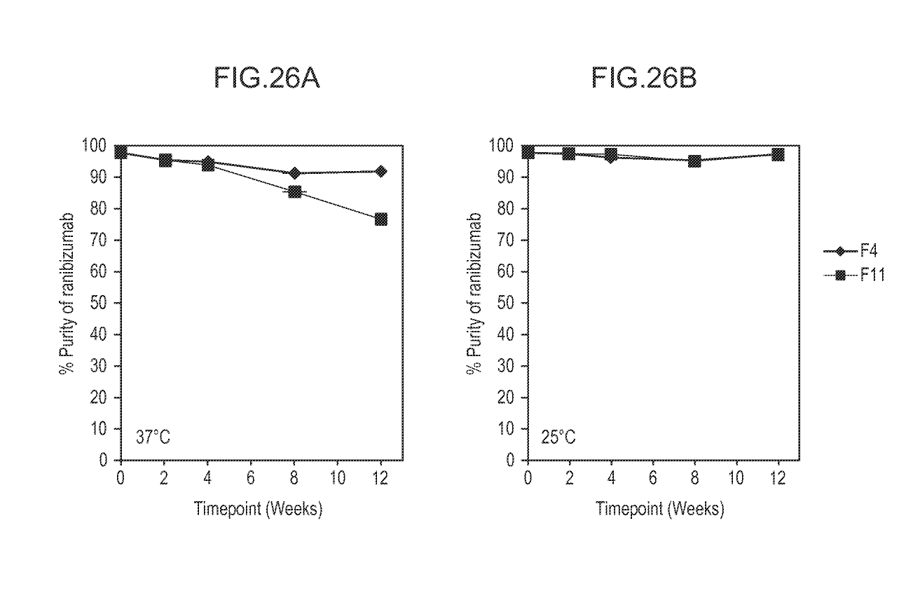

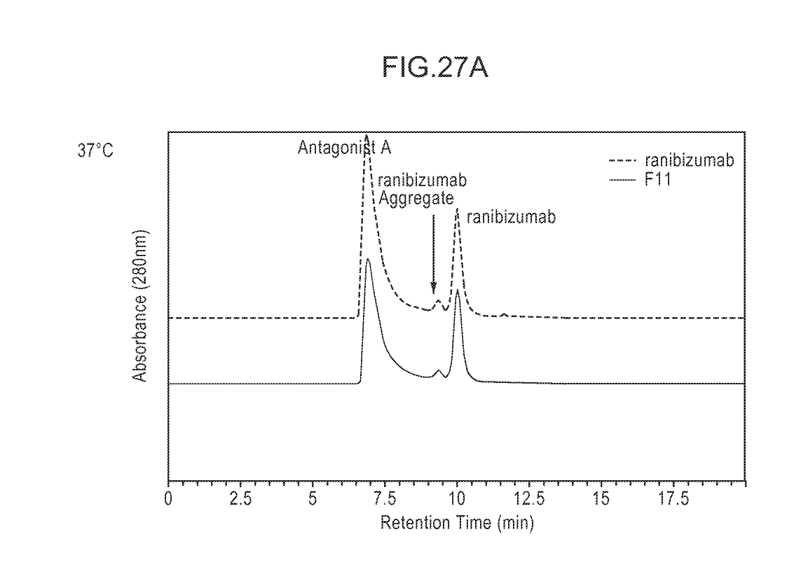

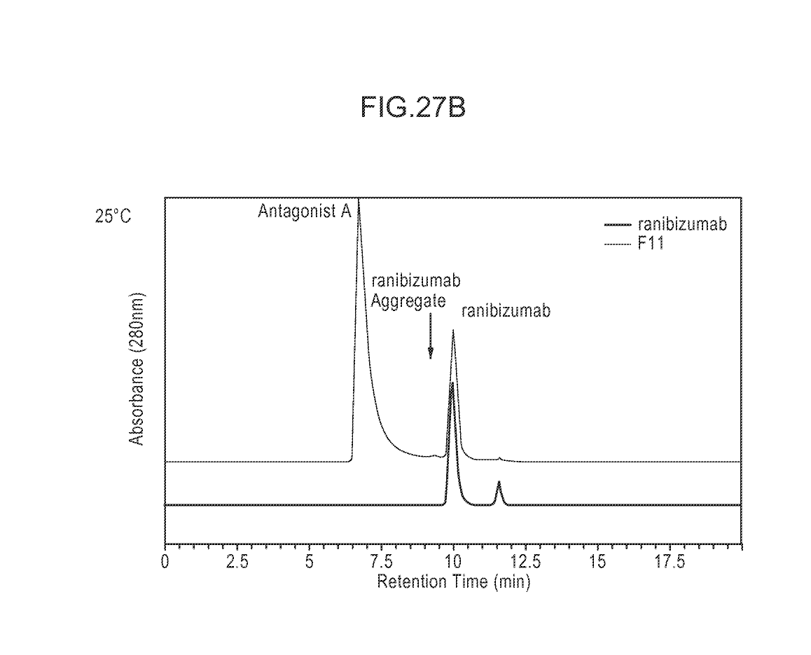

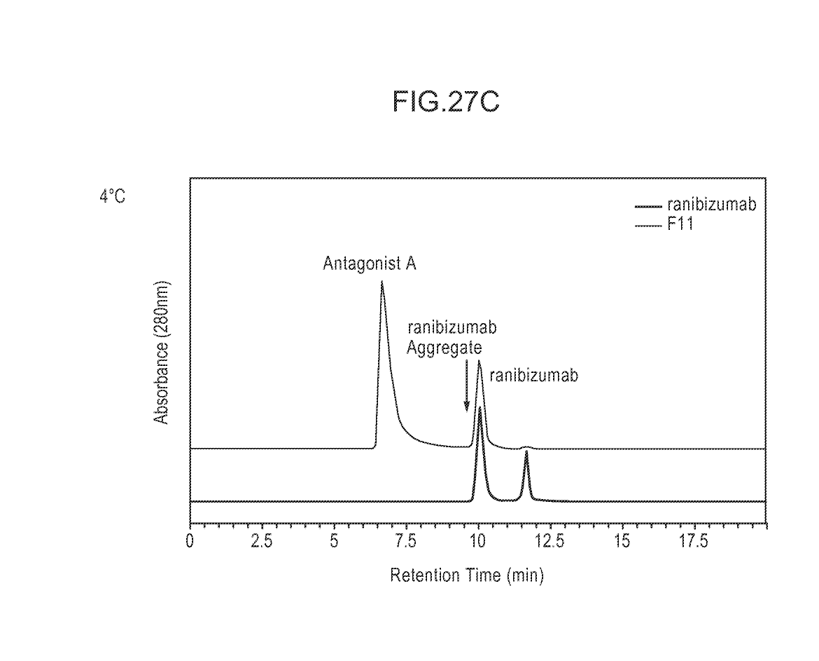

Ranibizumab encountered more stability issues in the 1:1 mixture than Antagonist A did at similar storage conditions. Ranibizumab in F11 maintained a comparable WCX-HPLC profile to ranibizumab in F4 up to 4 weeks storage at 37° C., after which ranibizumab underwent faster degradation in the mixture (F 11) (FIG. 26). Ranibizumab, however, remained fairly stable in the mixture when the samples were stored at 25° C. (FIG. 26). No significant difference in the WCX-HPLC purity profile of ranibizumab was observed between the mixture and F4 at 25° C. SE-HPLC revealed a noticeable increase in aggregated ranibizumab in the 1:1 mixture after 8 weeks of storage at 37° C. compared to F4 (FIG. 27a). The aggregation in the mixture was substantially lower when stored at 25° C., and was not observed at 4° C. (FIG. 27b-c).

Stability of Composition F6

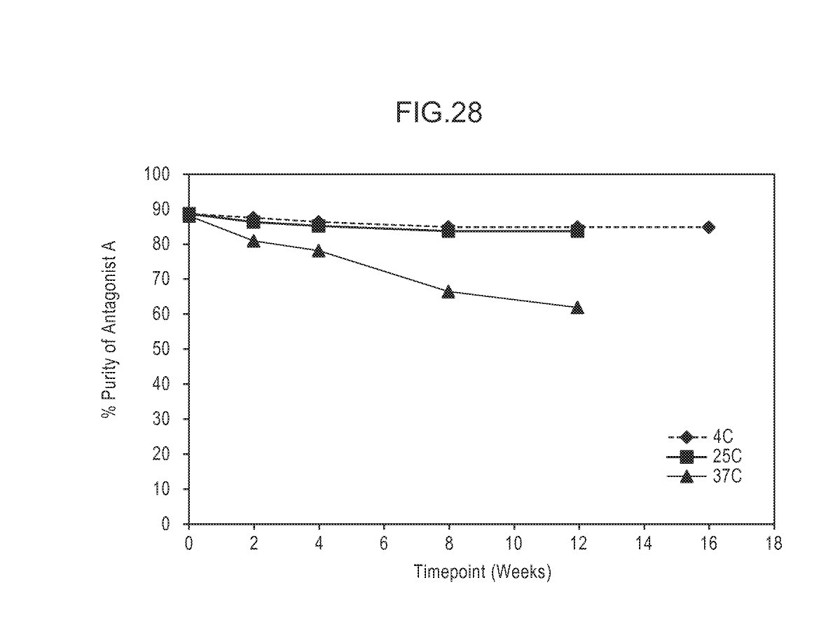

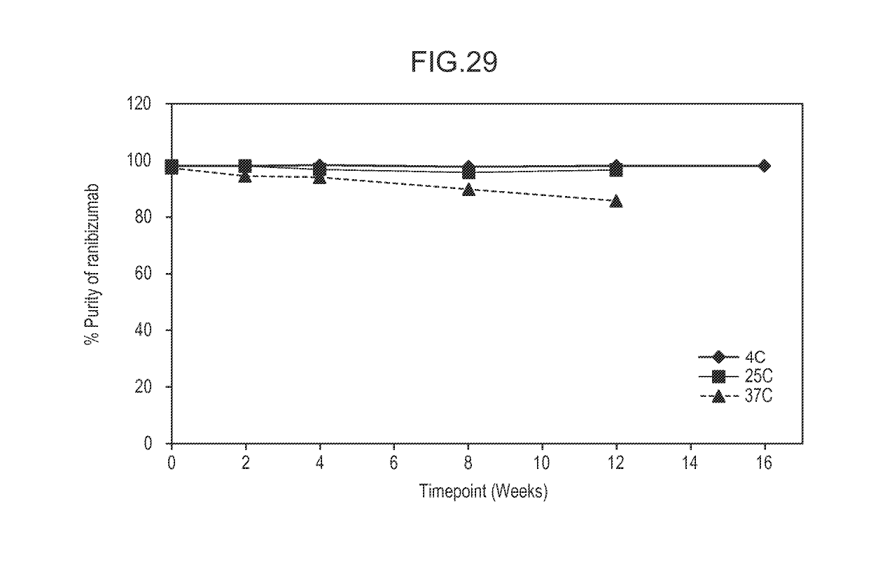

AEX-HPLC analysis of Antagonist A indicated that the F6 composition maintained the purity of Antagonist A up to twelve weeks at 25° C., and up to at least sixteen weeks (the latest timepoint tested) at 4° C. (FIG. 28). At 37° C., Antagonist A degradation was observed as early as two weeks (FIG. 28). Formulating with F6 helped protect ranibizumab from degradation at 37° C. for up to four weeks before a significant decrease in purity by WCX-HPLC developed by eight weeks (FIG. 29). However, ranibizumab was stable in composition F6 for up to at least twelve weeks at 25° C., and for up to at least sixteen weeks at 4° C., without any substantial loss in purity by WCX-HPLC (FIG. 29).

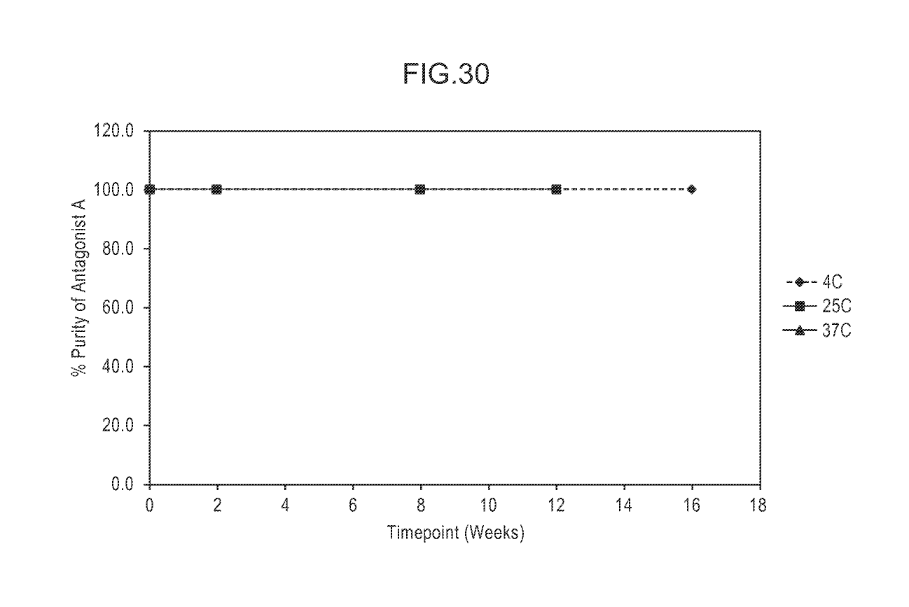

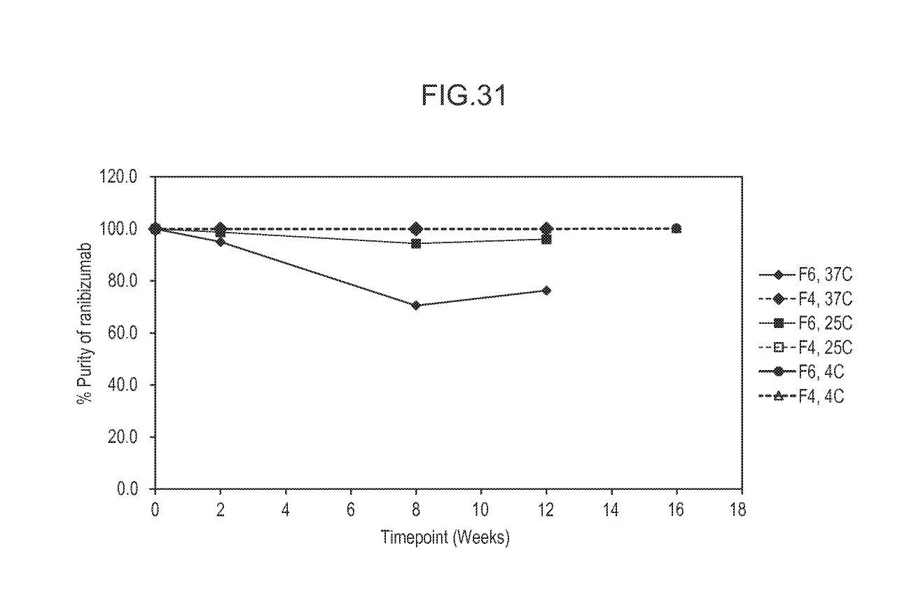

SE-HPLC results indicated that Antagonist A remains stable over sixteen weeks at all storage conditions (FIG. 30). For ranibizumab, no significant aggregation was observed when incubated for twelve weeks at 37° C. in the F6 composition (FIG. 31). The F6 composition performed better when stored at either 4° C. or 25° C., with comparable purity over eight weeks at both temperatures. Aggregation of ranibizumab at 25° C. and 37° C. was faster in the F6 composition than in F4.

From these ranging studies, composition F6, on average, demonstrated the best stability over all storage temperatures and analaysis methods employed for this study.

### Example 2

### Stability of Compositions Comprising Antagonist A and Bevacizumab

The stability of Antagonist A in a composition that also includes the anti-VEGF monoclonal antibody (mAb) bevacizumab was examined under a range of conditions. Various pHs (4.0-8.0) and tonicity modifiers (sodium chloride, sorbitol, and trehalose) were used to optimize the composition stability of Antagonist A and bevacizumab when stored at various temperatures (4° C., 25° C., and 37° C.) and against a physical stress (agitation). The stability of Antagonist A and bevacizumab was characterized by visual observation, pH measurement, and various HPLC methods (anion exchange [AEX-HPLC], weak cation exchange [WCX-HPLC], and size exclusion [SE-HPLC]).

Antagonist A was compatible with bevacizumab with no discernable stability issue when both were combined together in certain of the compositions tested. Based on the results from a 24 week stability study, the best stability was observed with Composition F 19. In the F 19 composition, both Antagonist A and bevacizumab remained stable throughout 24 weeks at 4° C., and for up to at least 4 weeks at 25° C.

### Composition Parameters

The following composition parameters were examined:
(1) pH: 4.0, 5.0, 6.0, 6.2, 6.3, 7.0, 7.3, 8.0
(2) Buffers: Acetate, Phosphate, and Tris
(3) Tonicity Modifiers: Sodium Chloride, Sorbitol, and Trehalose
(4) Surfactants: Polysorbate 20
(5) Antagonist A Concentration: 30 mg/mL, 15 mg/mL, and 3 mg/mL

The following parameters were fixed:
(1) Fill volume was 300 μL in modified 3 cc vials provided by Ophthotech Corp. (obtained from Mglas AG, Munnerstadt, Germany)
(2) The concentration of bevacizumab was 12.5 mg/mL.

Table 3 below summarizes the composition matrix used in this study.

TABLE 3
Composition Matrix for Antagonist A: Bevacizumab Compositions

| | | | | Antagonist A | Bevacizumab | |
|---|---|---|---|---|---|---|
| | | | | Concentration | Concentration | |
| Comp. | Buffer | pH | Tonicity Modifier | (mg/mL, oligo wt.) | (mg/mL) | Surfactant |
| F12 | 10 mM | 7.3 | 150 mM Sodium | 30 | 0.0 | 0% |
| | Phosphate | | Chloride | | | |
| F13 | 50 mM | 4 | 5% (w/v) Sorbitol | 3 | 12.5 | 0.02% |
| | Acetate | | | | | Polysorbate |
| | | | | | | 20 |
| F14 | 50 mM | 4 | 130 mM Sodium | 3 | 12.5 | 0.02% |
| | Acetate | | Chloride | | | Polysorbate |
| | | | | | | 20 |
| F15 | 50 mM | 5 | 5% (w/v) Sorbitol | 3 | 12.5 | 0.02% |
| | Acetate | | | | | Polysorbate |
| | | | | | | 20 |
| F16 | 50 mM | 5 | 130 mM Sodium | 3 | 12.5 | 0.02% |
| | Acetate | | Chloride | | | Polysorbate |
| | | | | | | 20 |
| F17 | 50 mM | 6 | 5% (w/v) Sorbitol | 3 | 12.5 | 0.02% |
| | Phosphate | | | | | Polysorbate |
| | | | | | | 20 |
| F18 | 50 mM | 6.2 | 6% (w/v) | 0 | 12.5 | 0.02% |
| | Phosphate | | Trehalose | | | Polysorbate |
| | | | | | | 20 |
| F19 | 50 mM | 6 | 130 mM Sodium | 3 | 12.5 | 0.02% |
| | Phosphate | | Chloride | | | Polysorbate |
| | | | | | | 20 |
| F20 | 50 mM | 7 | 5% (w/v) Sorbitol | 3 | 12.5 | 0.02% |
| | Phosphate | | | | | Polysorbate |
| | | | | | | 20 |
| F21 | 50 mM | 7 | 130 mM Sodium | 3 | 12.5 | 0.02% |
| | Phosphate | | Chloride | | | Polysorbate |
| | | | | | | 20 |
| F22 | 50 mM | 8 | 5% (w/v) Sorbitol | 3 | 12.5 | 0.02% |
| | Tris | | | | | Polysorbate |
| | | | | | | 20 |
| F23 | 50 mM | 8 | 130 mM Sodium | 3 | 12.5 | 0.02% |
| | Tris | | Chloride | | | Polysorbate |
| | | | | | | 20 |
| F24 | 30 mM | 6.3 | 75 mM sodium | 15 | 12.5 | 0.02% |
| | Phosphate | | Chloride + 3% | | | Polysorbate |
| | | | (w/v) Trehalose | | | 20 |
| F25 | 10 mM | 7.3 | 150 mM Sodium | 3 | 0.0 | 0% |
| | Phosphate | | Chloride | | | |
| F26 | 30 mM | 6.3 | 75 mM sodium | 3 | 12.5 | 0.02% |
| | Phosphate | | Chloride + 3% | | | Polysorbate |
| | | | (w/v) Trehalose | | | 20 |

### Sample Preparation

An Antagonist A stock solution was prepared at 6 mg/mL in 10 mM phosphate, 150 mM NaCl, and pH 7.3. The resulting stock solution was admixed 1:1 with commercial Avastin® (25 mg/mL), resulting in final concentrations of 3 mg/mL Antagonist A and 12.5 mg/mL bevacizumab (Composition F26). The composition was placed in 10 kDa molecular weight cutoff dialysis cassettes and dialyzed ˜1,000,000-fold against the various composition buffers listed in Table 3 (Comp. Nos. F13-F17, F19-F23). Exceptions include the following:
Composition F12 needed no additional dilution or dialysis.
Commercial Avastin® was diluted 1:1 with 50 mM phosphate buffer (pH 6.2) containing 6% (w/v) trehalose and 0.02% (w/v) polysorbate 20 to provide Composition F18.
Composition F24 was made by admixing 1:1 of composition F12 with commercial Avastin®.
Composition F25 was created with 10× dilution of Composition F12 with 10 mM phosphate buffer (pH 7.3) containing 150 mM NaCl.

### Stress Studies

The compositions of Table 3 were tested under the following stress conditions:

TABLE 4
Stress Conditions

| Stress Conditions | | Time points |
|---|---|---|
| Storage | 4° C. | 0, 2, 4, 8, 12, and 24 weeks |
| | 25° C. | 2, 4, 8, 12, and 24 weeks |
| | 37° C. | 2, 4, 8, and 12 weeks |

| | |
|---|---|
| Agitation | 4 hours |

### Analytical Methods

In order to analyze degradation products generated under stress, the following stability-indicating assays were developed and used in this study.

(1) SE-HPLC (Analysis of Antagonist A and Bevacizumab)
Mobile Phase: 50 mM phosphate buffer, 100 mM sodium chloride, pH 7.0
Column: TOSOH TSKgel G3000SW_{XL}
Column Temperature: Ambient
Flow Rate: 1.0 mL/min
Wavelength: Signal, 214 nm; Reference, 360 nm
Injection volume: 1 μL
Sample Preparation:
   10× dilution in Milli-Q water for 30 mg/mL aptamer samples
   No dilution for other samples
Percent purity reported based on integrated area percent of main peaks identified for both Antagonist A and bevacizumab

(2) WCX-HPLC (Analysis of Bevacizumab)
Mobile Phase A: 10 mM phosphate buffer, pH 7.0
Mobile Phase B: 10 mM phosphate buffer, 500 mM sodium chloride, pH 7.0
Column: Dionex ProPac WCX-10, 4×250 mm
Column Temperature: Ambient
Flow Rate: 1.0 mL/min
Wavelength: Signal, 214 nm; Reference, 360 nm
Injection volume: 10 μL
Sample Preparation: 10× dilution in Milli-Q water
Percent purity reported based on integrated area percent of the main peak identified for bevacizumab

(3) AEX-HPLC (Analysis of Antagonist A)
Mobile Phase A: 10 mM phosphate buffer, pH 7.0
Mobile Phase B: 10 mM phosphate buffer, 500 mM sodium chloride, pH 7.0
Column: Dionex DNA Pac PA-100, 4×250 mm
Column Temperature: 40° C.
Flow Rate: 1.2 mL/min
Wavelength: Signal, 258 nm; Reference, 360 nm
Injection volume: 5 μL
Sample Preparation:
   10× dilution in Milli-Q water for 3.0 mg/mL aptamer samples
   50× dilution in Milli-Q water for 15 mg/mL aptamer samples
   100× dilution in Milli-Q water for 30 mg/mL aptamer samples
Percent purity reported based on integrated area percent of the main peak identified for Antagonist A

(4) pH
VWR symphony SB70P

(5) Visual Observation
Photos taken from Sony Cyber-shot DSC-H9 Digital Still Camera (8.1 Mega pixels)

(6) Osmolarity (at Time Point Zero)
Advanced Instruments, Inc. Advanced Osmometer Model 3D3

### Stability Overview

This section describes the effect of both agitation (4 hours) and the storage at various temperatures (4° C., 25° C., and 37° C.) on Antagonist A and bevacizumab. Throughout the study, each composition was able to maintain targeted pH values through all physical stresses.

Stability Indicating Assays

By visual observation, it was noted that compositions F15, F16, and F24 developed precipitation during 2 weeks of storage at 37° C. (data not shown). Due to the limited volumes available for the study, no other assay was performed for quantitative measurement of precipitation.

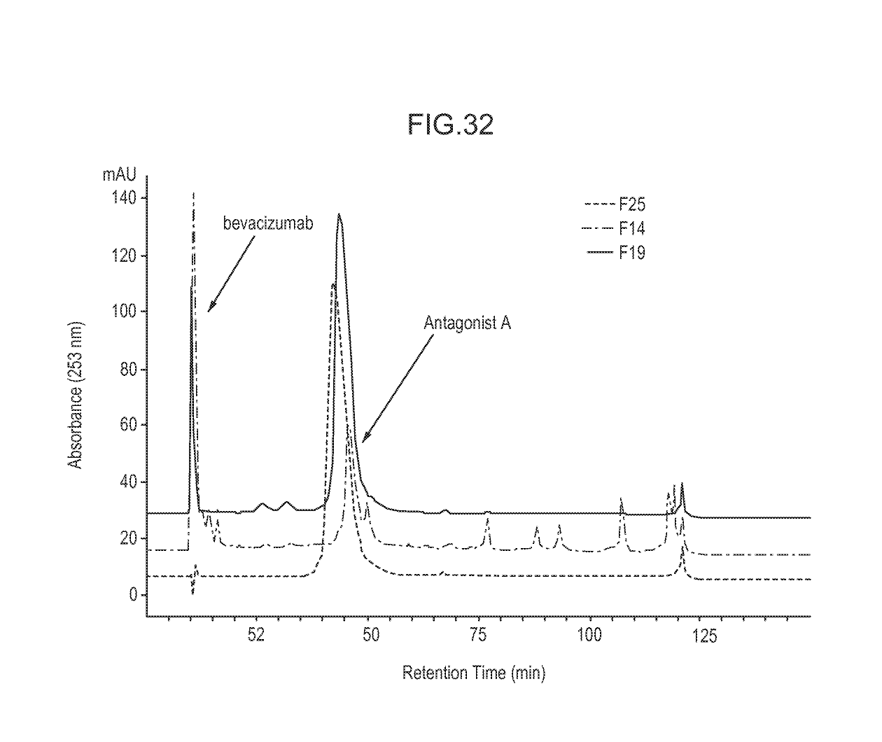

The stability of Antagonist A during storage was effectively analyzed by AEX-HPLC. The formation of both pre-peaks and post peaks was observed when Antagonist A in certain compositions was incubated at elevated temperature of 37° C. For example, in Composition F14, the AEX-HPLC purity of Antagonist A had decreased by nearly 50% during storage for 2 weeks at 37° C. (FIG. 32).

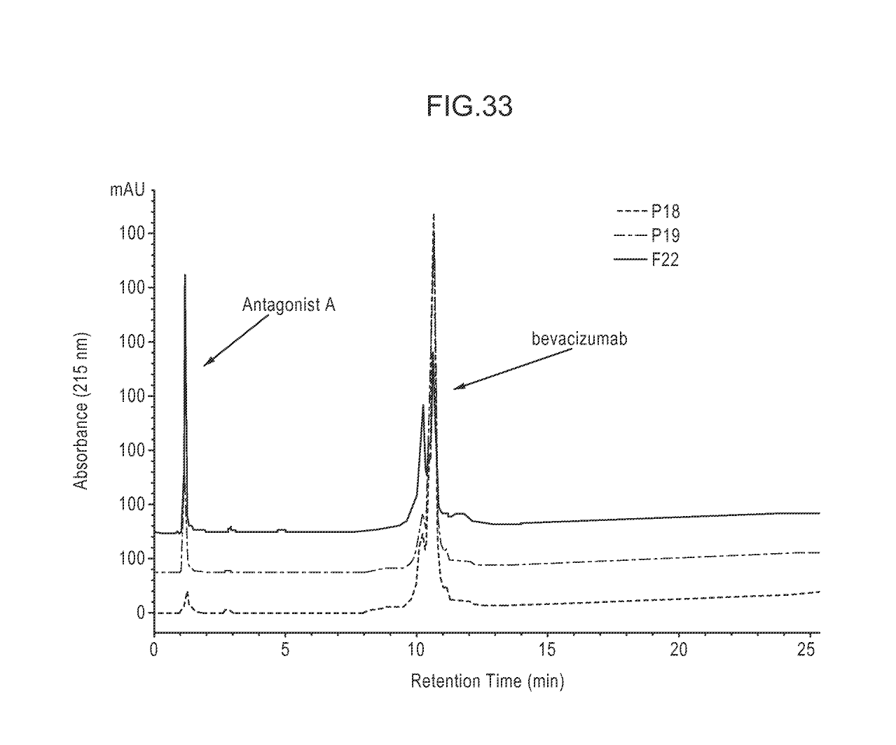

WCX-HPLC was also effective in characterizing the stability of bevacizumab. The formation of both pre-peaks and post peaks was observed when bevacizumab in certain compositions was incubated at a temperature of 25° C. For example, in Composition F22, bevacizumab purity decreased nearly 30% during 8 weeks of storage at 25° C. (FIG. 33).

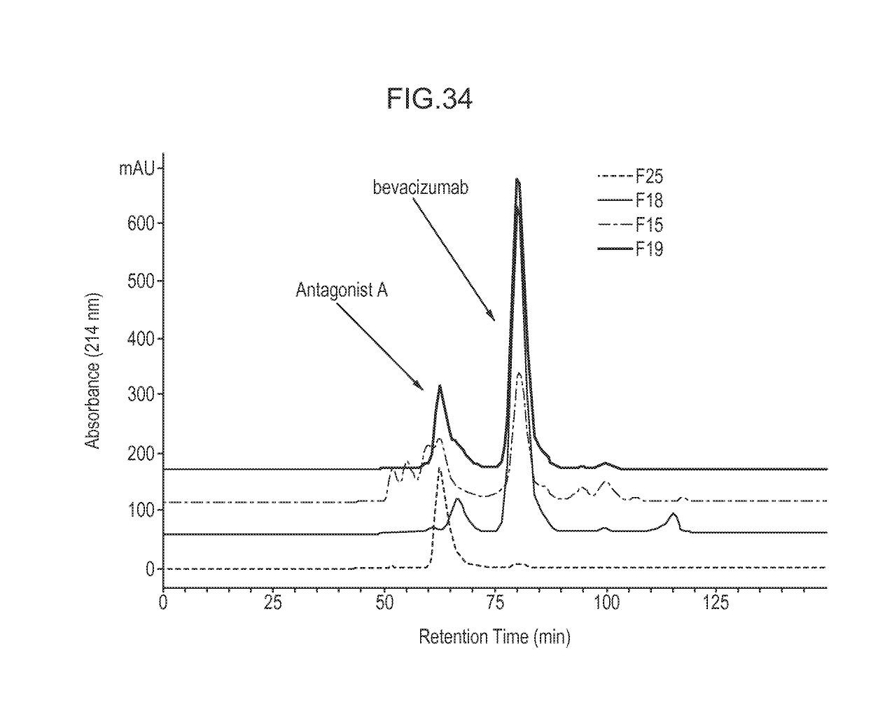

SE-HPLC proved useful for characterizing soluble aggregation or fragmentation of bevacizumab. Antagonist A did not show significant changes by SE-HPLC, although the resolution of its aggregated form may be beyond the capacity of TSKgel G3000SWXL column due to assay sensitivity to stability. Degradation of bevacizumab was seen in composition F15 after 8 weeks of storage at 37° C. (FIG. 34).

Effect of Agitation on Stability

The effect of agitation on one or both of Antagonist A and bevacizumab was assesed. The compositions listed in Table 3 were agitated for 4 hours with an in-house agitator, while a control set of compositions was left unagitated at room temperature. No differences in visual observation, pH, AEX-HPLC, and WCX-HPLC were observed between agitated samples and controls (data not shown). However, SE-HPLC, which assesses aggregation or fragmentation of both Antagonist A and bevacizumab, displayed slight variations between agitated and control samples in F23 and F24 samples (Table 5 and Table 6). After 4 hours of agitation, more soluble aggregates (pre-Antagonist A peak and pre-bevacizumab peak) formed in the F23 samples and the direct 1:1 mixture of 30 mg/mL Antagonist A and 25 mg/mL Avastin® (F24). This suggests that formulating at pH 8.0 with sodium chloride, or having a higher concentration of Antagonist A coformulated with bevacizumab, leads to Antagonist A or bevacizumab forming soluble aggregates or fragments during shear stress. The other compositions appeared to maintain the integrity of Antagonist A and bevacizumab as determined by SE-HPLC. These results suggest that except under the conditions noted above, no apparent degradation of coformulated Antagonist A or bevacizumab was induced by agitation.

TABLE 5
SE-HPLC results for samples before agitation

| | | | | | bevacizumab | | |
|---|---|---|---|---|---|---|---|
| | | | Antagonist A | | Area (%) | | |
| | | | Area (%) | | | | Post- |
| | Conc. (mg/mL) | | Pre- Ant. | Ant. A | Pre- bev. | bev. | bev. |
| Comp. | Ant. A | bev. | A Peak | Peak | Pk | Peak | Peak |
| F12 | 30.0 | 0.0 | 0.3 | 99.7 | NA | NA | NA |
| F13 | 3.0 | 12.5 | 4.6 | 95.4 | 4.6 | 89.3 | 6.1 |
| F14 | 3.0 | 12.5 | 6.2 | 93.8 | 4.0 | 89.6 | 6.4 |
| F15 | 3.0 | 12.5 | 5.6 | 94.4 | 3.9 | 90.1 | 6.0 |
| F16 | 3.0 | 12.5 | 4.2 | 95.8 | 4.5 | 89.4 | 6.1 |
| F17 | 3.0 | 12.5 | 3.6 | 96.4 | 4.0 | 90.7 | 5.3 |
| F18 | 0.0 | 12.5 | NA | NA | 4.7 | 90.1 | 5.3 |
| F19 | 3.0 | 12.5 | 2.5 | 97.5 | 4.4 | 91.1 | 4.5 |
| F20 | 3.0 | 12.5 | 2.4 | 97.6 | 9.5 | 86.1 | 4.4 |
| F21 | 3.0 | 12.5 | 2.6 | 97.4 | 13.5 | 82.0 | 4.5 |
| F22 | 3.0 | 12.5 | 3.0 | 97.0 | 13.8 | 81.6 | 4.6 |
| F23 | 3.0 | 12.5 | 3.1 | 96.9 | 16.7 | 78.0 | 5.2 |
| F24 | 15.0 | 12.5 | 2.3 | 97.7 | 16.8 | 76.1 | 7.0 |
| F25 | 3.0 | 0.0 | 0.7 | 99.3 | NA | NA | NA |
| F26 | 3.0 | 12.5 | 1.7 | 98.3 | 7.2 | 88.0 | 4.8 |

“Ant. A” is Antagonist A; “bev.” is bevacizumab; “NA” means not applicable

TABLE 6
SE-HPLC results for samples after 4 hours agitation

| | | | | | bevacizumab | | |
|---|---|---|---|---|---|---|---|
| | | | Antagonist A | | Area (%) | | |
| | | | Area (%) | | | | Post- |
| | Conc. (mg/mL) | | Pre- Ant. | Ant. A | Pre- bev. | bev. | bev. |
| Comp. | Ant. A | bev. | A Peak | Peak | Peak | Peak | Peak |
| F12 | 30.0 | 0.0 | 0.3 | 99.7 | NA | NA | NA |
| F13 | 3.0 | 12.5 | 5.4 | 94.6 | 4.0 | 90.0 | 6.0 |
| F14 | 3.0 | 12.5 | 6.0 | 94.0 | 3.9 | 90.1 | 6.0 |
| F15 | 3.0 | 12.5 | 3.4 | 96.6 | 3.2 | 91.6 | 5.1 |
| F16 | 3.0 | 12.5 | 3.4 | 96.6 | 4.0 | 89.9 | 6.2 |
| F17 | 3.0 | 12.5 | 2.4 | 97.6 | 4.2 | 90.5 | 5.4 |
| F18 | 0.0 | 12.5 | NA | NA | 4.1 | 90.8 | 5.2 |
| F19 | 3.0 | 12.5 | 1.7 | 98.3 | 3.9 | 91.7 | 4.4 |
| F20 | 3.0 | 12.5 | 2.7 | 97.3 | 9.2 | 86.2 | 4.7 |
| F21 | 3.0 | 12.5 | 4.4 | 95.6 | 13.3 | 82.2 | 4.5 |
| F22 | 3.0 | 12.5 | 4.8 | 95.2 | 13.4 | 82.0 | 4.6 |
| F23 | 3.0 | 12.5 | 7.1 | 92.9 | 16.0 | 78.7 | 5.3 |
| F24 | 15.0 | 12.5 | 5.4 | 94.6 | 20.7 | 74.6 | 4.7 |
| F25 | 3.0 | 0.0 | 1.1 | 98.9 | NA | NA | NA |
| F26 | 3.0 | 12.5 | 2.3 | 97.7 | 7.3 | 88.4 | 4.3 |

“Ant. A” is Antagonist A; “bev.” is bevacizumab; “NA” means not applicable

Effect of Storage Temperature on Stability

During the 24-week study, the compositions listed in Table 3 were placed in 4° C., 25° C., and 37° C. stability chambers to study the effects of temperature on one or both of Antagonist A and bevacizumab stability. Both Antagonist A and bevacizumab exhibited greater degradation with increasing storage temperature, based on the chromatographic assays.

Storage at 37° C. induced significantly elevated levels of degradation of Antagonist A and bevacizumab. By 2 weeks, precipitation of Antagonist A or bevacizumab was seen in F15, F16, and F24 (data not shown). By 4 weeks, F14 also began showing insoluble aggregation of Antagonist A or bevacizumab (data not shown). All other composition remained clear throughout 12 weeks.

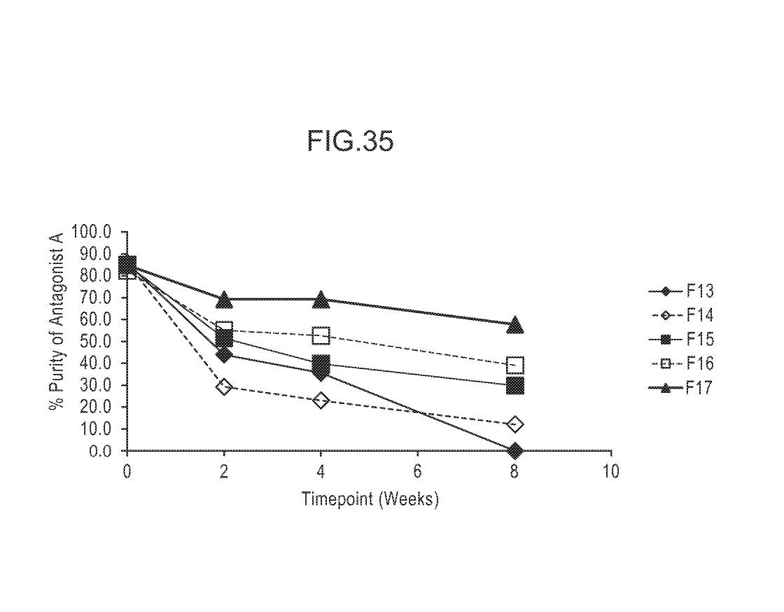

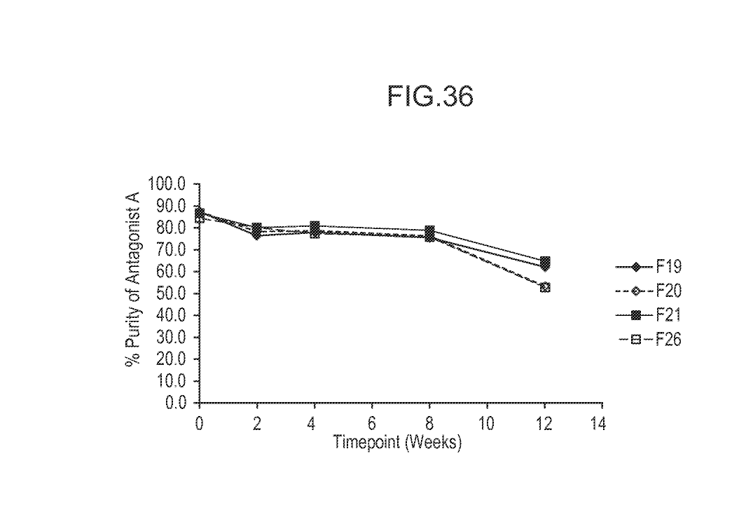

AEX-HPLC revealed significant Antagonist A degradation in composition samples at pH 4.0 and 5.0 (F13, F14, F15, and F16), while coformulated samples in F17 displayed better stability (FIG. 35). Antagonist A maintained comparable purity at pH 6.0-7.0 through 12 weeks of storage at 37° C., with the exception of F20 and F26, where decreases in Antagonist A purity were observed at 12 weeks (FIG. 36).

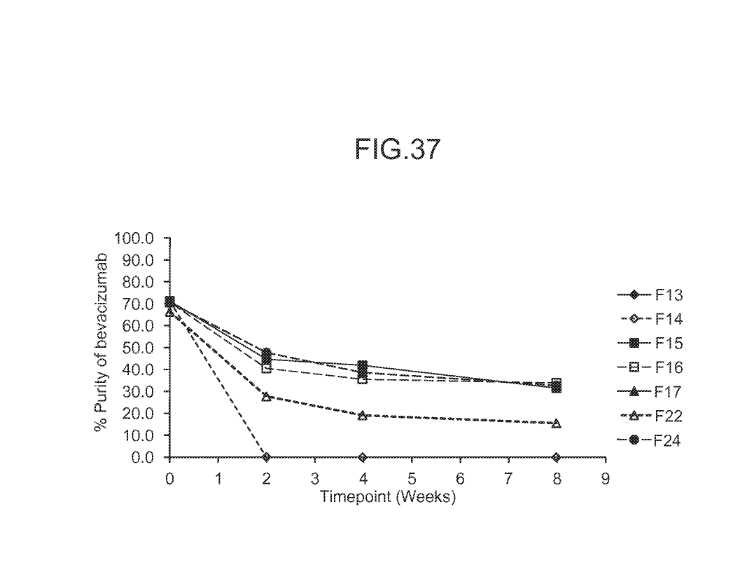

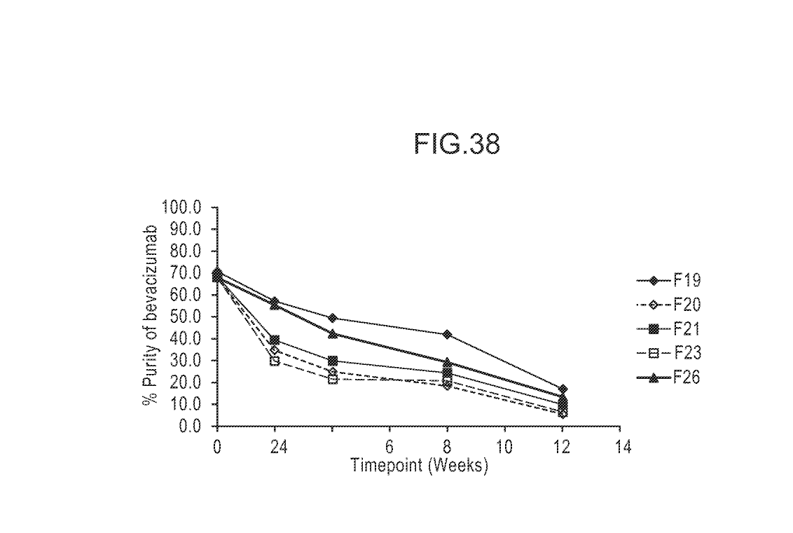

After 2 weeks of storage at 37° C., WCX-HPLC revealed significant decreases in bevacizumab purity in pH 4.0 composition (F13 and F14), displaying low to no intact bevacizumab remaining (FIG. 37). Accelerated degradation was observed through 12 weeks of storage at 37° C. in all other composition except for F19, which consistently revealed slower degradation than other composition (FIG. 38).

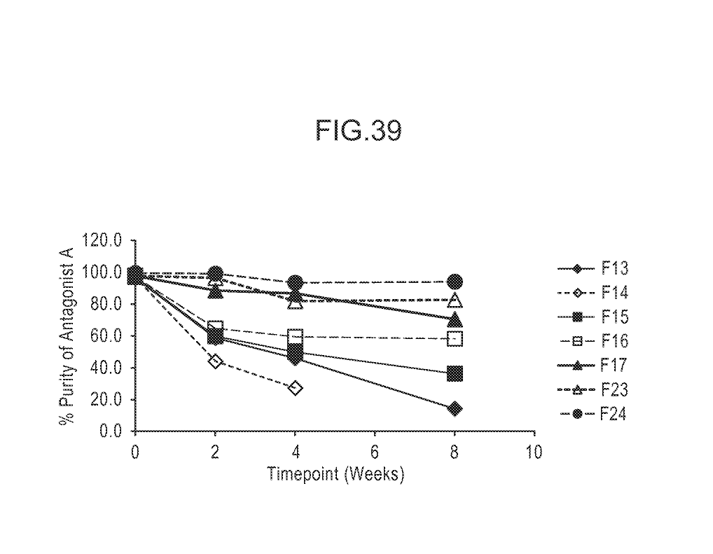

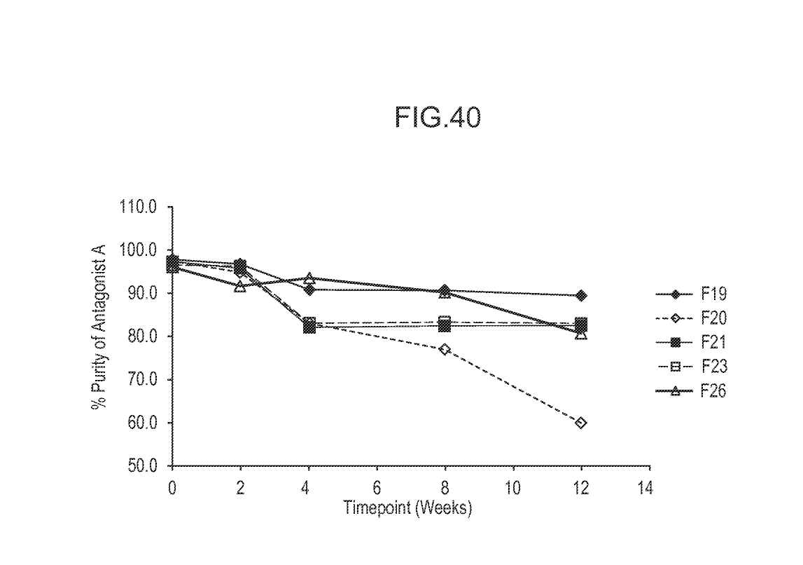

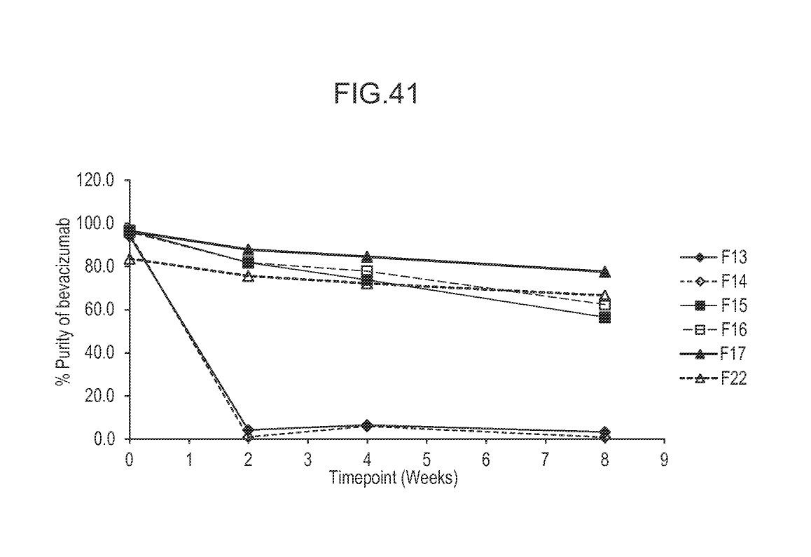

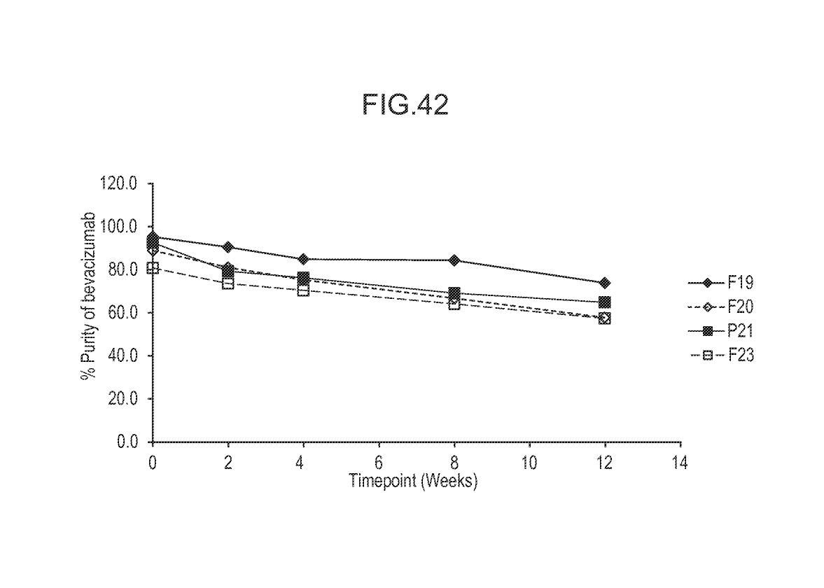

SE-HPLC revealed the formation of soluble aggregates in the stressed samples. For Antagonist A, 2 weeks of storage at 37° C. caused composition at pH 4.0-5.0 to rapidly form soluble aggregates (FIG. 39). Antagonist A formulated in F17 also showed soluble aggregation but at a lower rate (FIG. 39). By the fourth week, most of the Antagonist A compositions displayed lower Antagonist A purity, with the exception of F19 and the two 1:1 mixtures (F24 and F26), which were able to maintain high Antagonist A purity (FIG. 40). This trend was maintained until Week 12, when F26 revealed slightly reduced Antagonist A purity, leaving F19 as the composition of choice for Antagonist A with respect to stability. For bevacizumab, formulating outside of pH 6.0 caused a significant decrease in the mAb purity (FIG. 41). This trend continued throughout 12 weeks of storage at 37° C., leaving F19 as the composition providing bevacizumab with the greatest stability (FIG. 42).

Some compositions provided better stability during storage at 25° C. All of the compositions remained clear after 24 weeks at 25° C. except for F14, in which precipitation was observed at 8 weeks (data not shown).

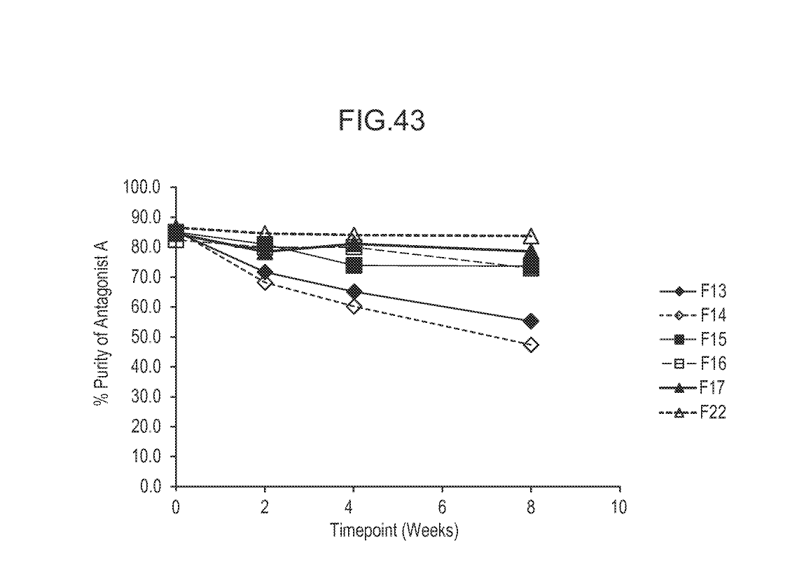

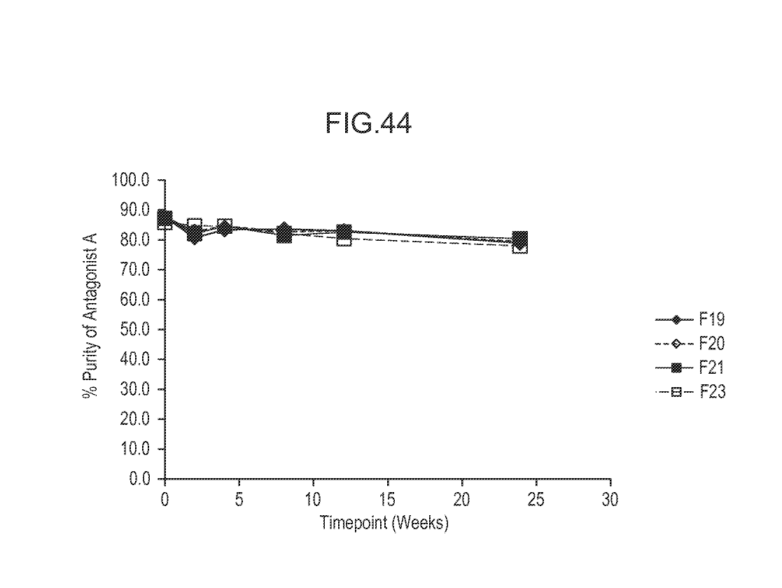

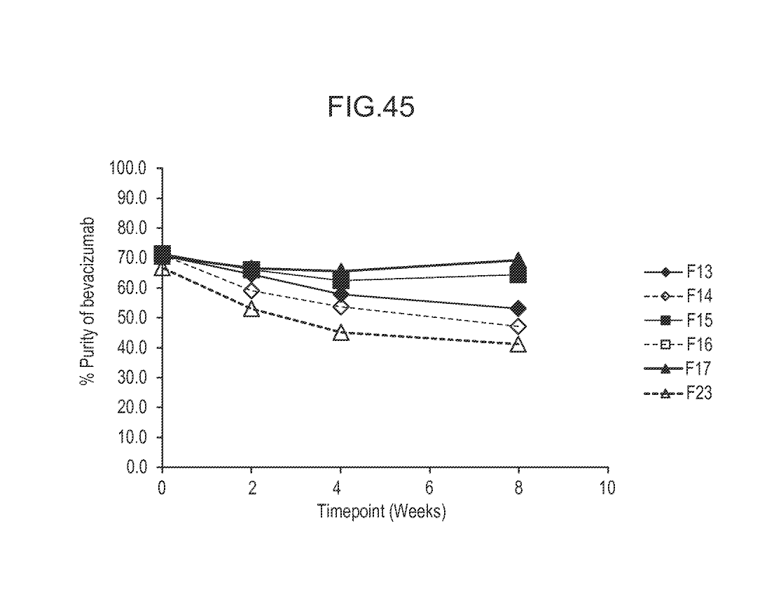

Based on AEX-HPLC, formulating Antagonist A at pH 4.0 (F13 and F14) caused degradation of the aptamer after just 2 weeks of storage (FIG. 43). F15 revealed degradation at 4 weeks of 25° C. storage; however, F16 exhibited improved stability up to 8 weeks (FIG. 43). Formulating Antagonist A at pH 6.0-8.0 maintained comparable stability through 8 weeks of storage at 25° C., and up to at least 24 weeks storage at 4° C. with compositions F19, F20, F21, and F23 (FIG. 44).

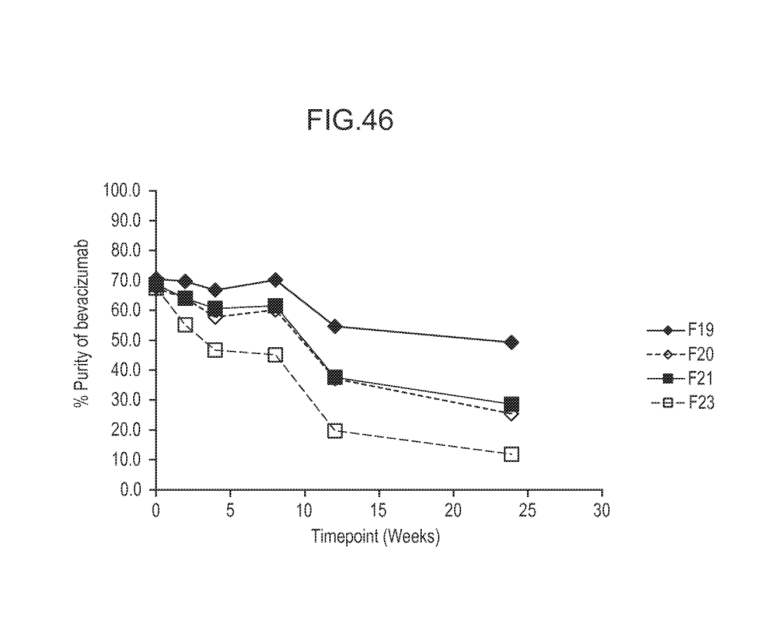

WCX-HPLC indicated that pH had the opposite effect on the stability of bevacizumab compared to Antagonist A. After 2 weeks at 25° C., pH 8.0 samples revealed substantial degradation of bevacizumab (FIG. 45 and FIG. 46). By 4 weeks at 25° C., pH 4.0 and pH 7.0 compositions began displaying signs of bevacizumab degradation (FIG. 45 and FIG. 46). Compositions at pH 5.0-6.0 provided comparable stability of bevacizumab up to 12 weeks at 25° C., at which time all leading candidates displayed accelerated signs of degradation. However, the F19 composition, at pH 6.0, did not undergo further accelerated degradation of bevacizumab from 12 to 24 weeks of storage at 25° C. (FIG. 45 and FIG. 46).

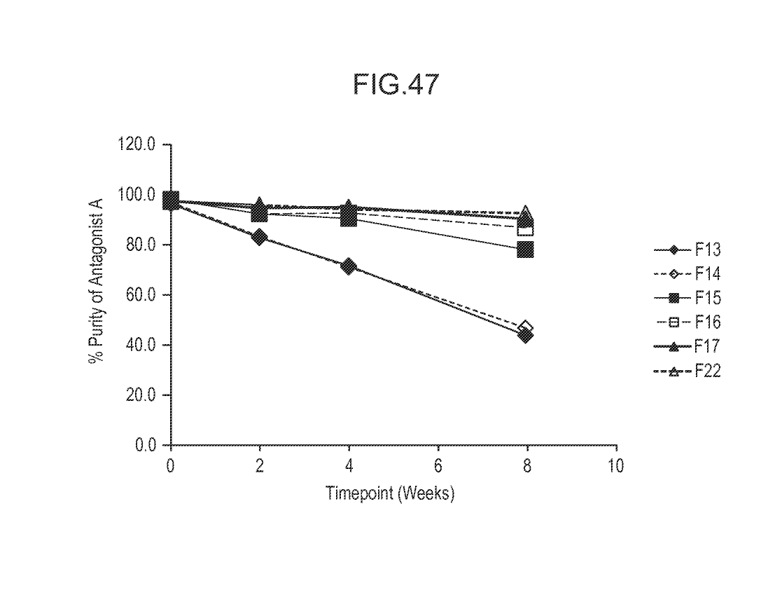

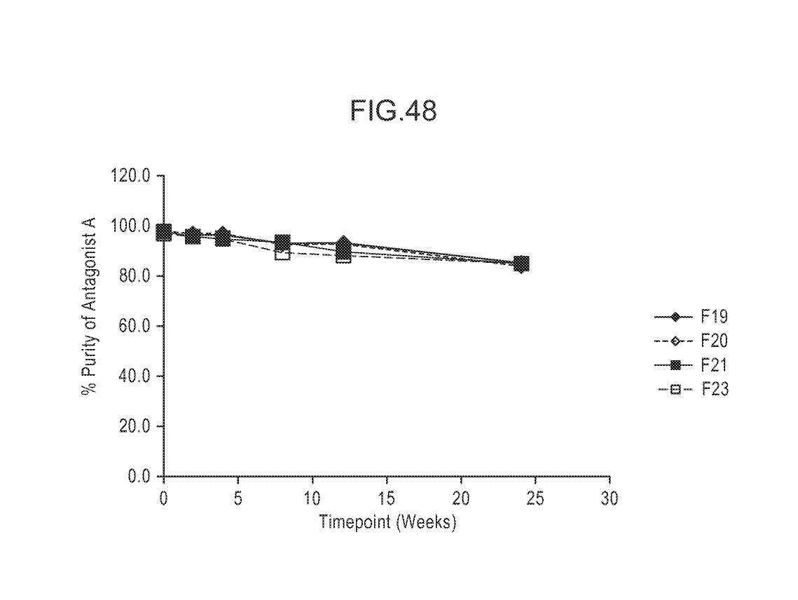

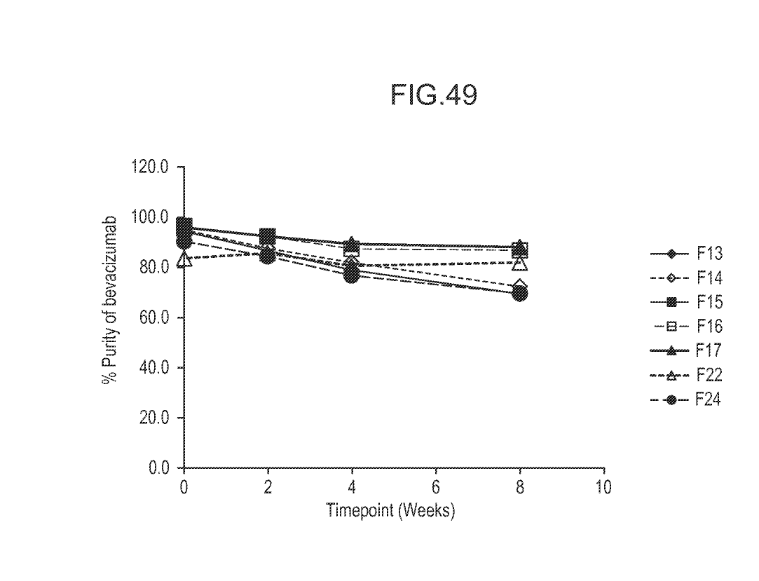

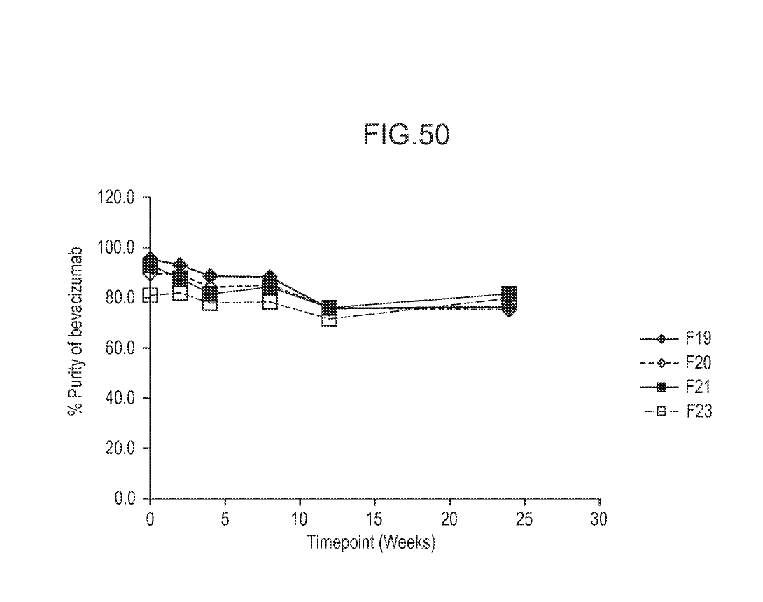

Similar degradation trends seen in AEX-HPLC and WCX-HPLC were observed by SE-HPLC. Antagonist A formulated at pH 4.0 was unable to maintain Antagonist A purity when stored at 25° C. (FIG. 47). By 8 weeks, Antagonist A formulated at pH 5.0 underwent significant aggregation or fragmentation (FIG. 47). Formulating Antagonist A in the pH range of 6.0-8.0 provided for comparable purity through up to at least 24 weeks of 25° C. storage (FIG. 21). The purity of bevacizumab depended on the pH of the composition and the concentration of Antagonist A in the composition. After 4 weeks of storage at 25° C., formulating at pH 4.0 and pH 8.0 caused an accelerated decrease in the purity of bevacizumab (FIG. 49 and FIG. 50). Under the same time and storage conditions, Antagonist A coformulated at 15 mg/mL appeared to adversely affect the purity of bevacizumab (FIG. 49). Compositions at pH 5.0-7.0 provided for better stability at 25° C. over 8 weeks (FIG. 49 and FIG. 50). Further time points revealed that leading compositions (pH 6.0 and 7.0) were able to maintain comparable purity (FIG. 50).

Storage at 4° C. provided the best stability for most compositions during this study. Visual observation revealed no insoluble aggregation during 4° C. storage for up to at least 24 weeks for compositions F19, F20, F21, and F23.

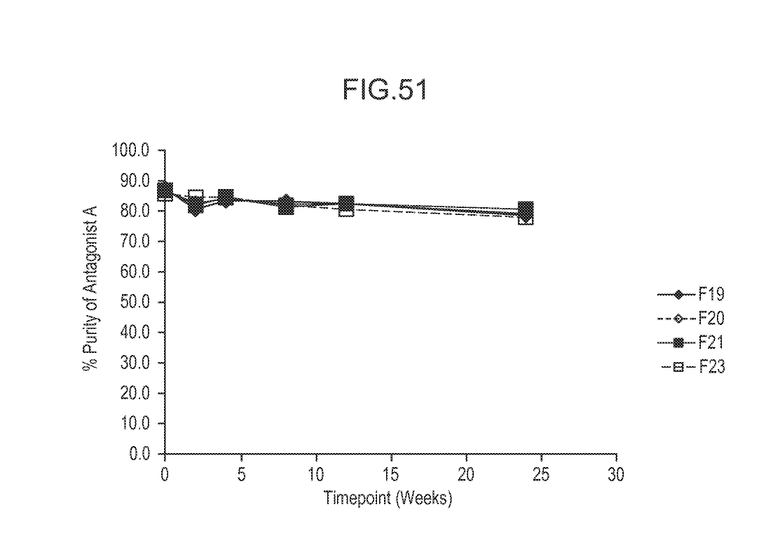

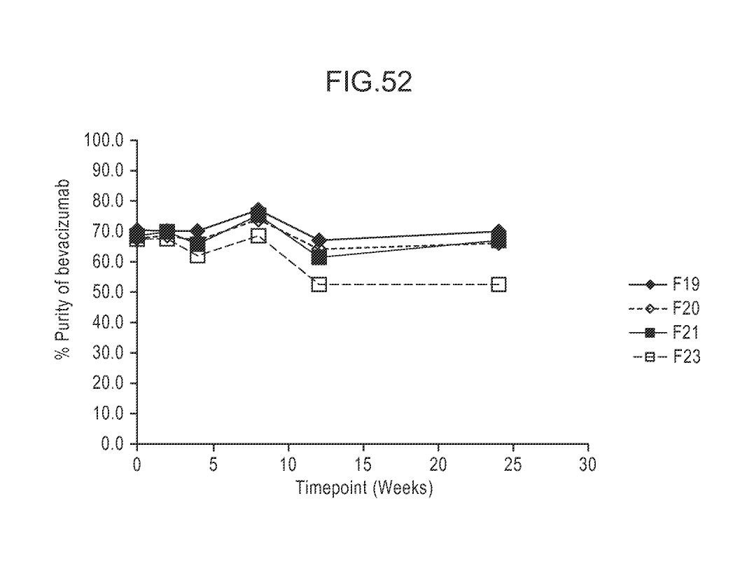

For Antagonist A, all compositions maintained comparable purity by AEX-HPLC after eight weeks of storage, and through 24 weeks at 4° C. with compositions F19, F20, F21, and F23 (FIG. 51). However, as observed by WCX-HPLC, formulating bevacizumab at pH 8.0 caused a considerable increase in degradation after eight weeks at 4° C., a trend which carried on through 24 weeks (FIG. 52).

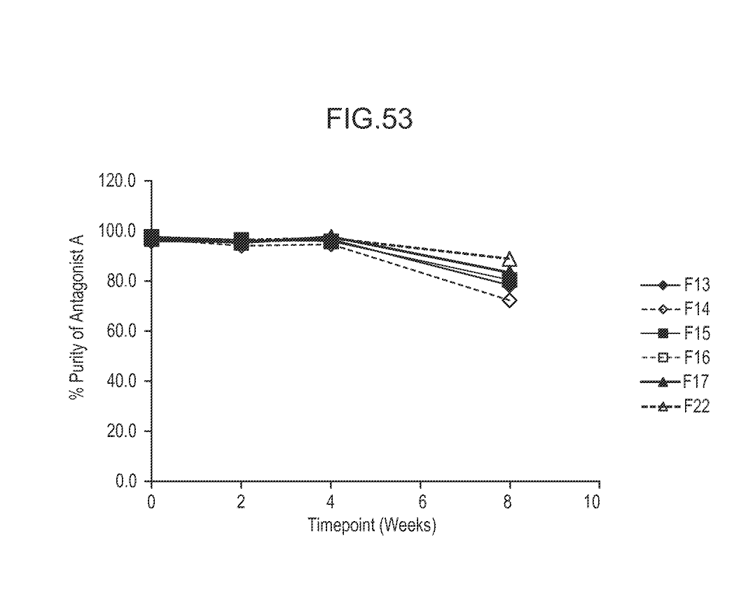

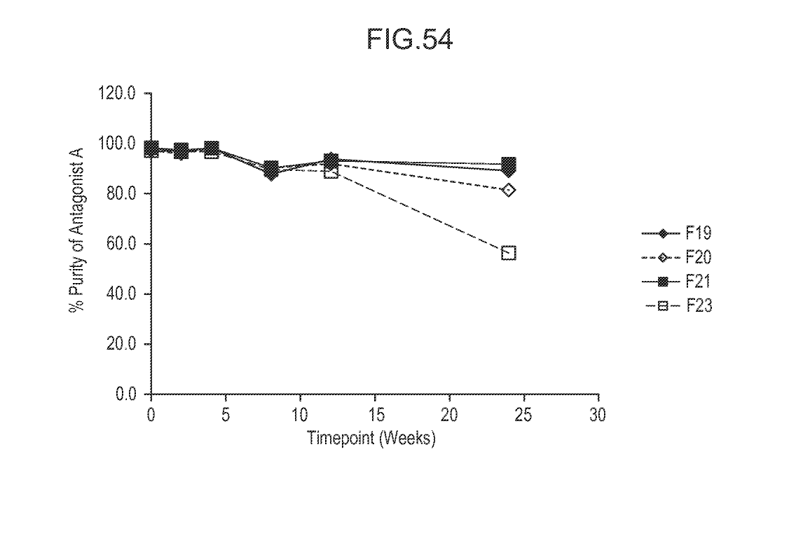

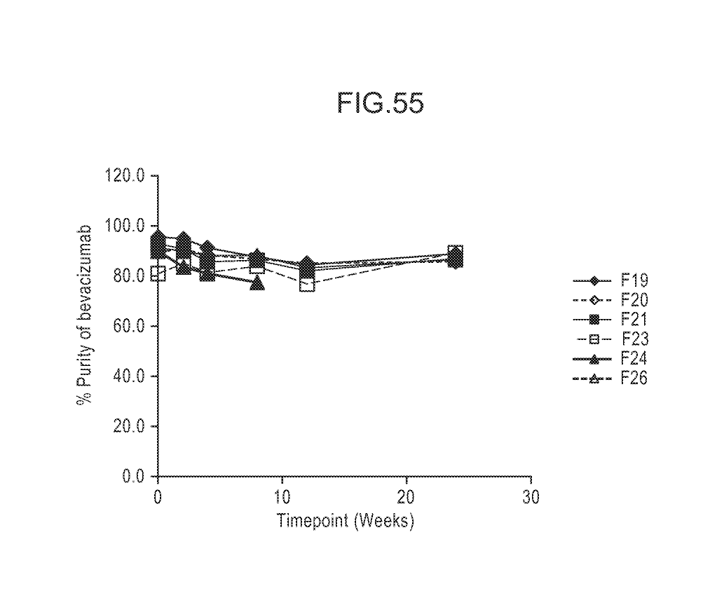

SE-HPLC revealed some fragmentation of Antagonist A or aggregation of bevacizumab in a few compositions. For Antagonist A, most compositions maintained their purity up to 8 weeks at 4° C., while compositions at pH 4.0-5.0 revealed significant losses of purity (FIG. 53). Compositions F19, F20, F21 and F23 maintained comparable Antagonist A purity up to 12 weeks of 4° C. storage; however, after 12 and 24 weeks, Antagonist A purity in F23 decreased substantially, while that of the other three selected compositions remained similarly elevated (FIG. 54). Formulating at pH 8.0 caused formation of soluble aggregates of bevacizumab during initial dialysis; however, storage at 4° C. maintained the purity of bevacizumab through at least eight weeks, similar to the other compositions (FIG. 55). The one exception was composition F24, where the concentration of Antagonist A at 15 mg/mL affected the purity of bevacizumab over the eight weeks of storage (FIG. 55).

### Effect of Composition Characteristics/Components on Stability

Antagonist A and bevacizumab were coformulated at varying pH and with different tonicity modifiers in order to determine the effects of these factors on stability. This section describes the effects of the composition on the stability of one or both of Antagonist A and bevacizumab.

Effect of pH on Stability

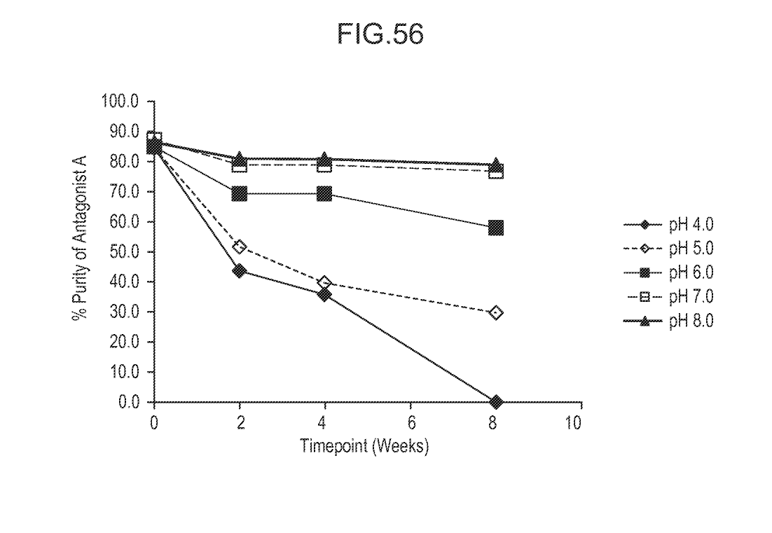

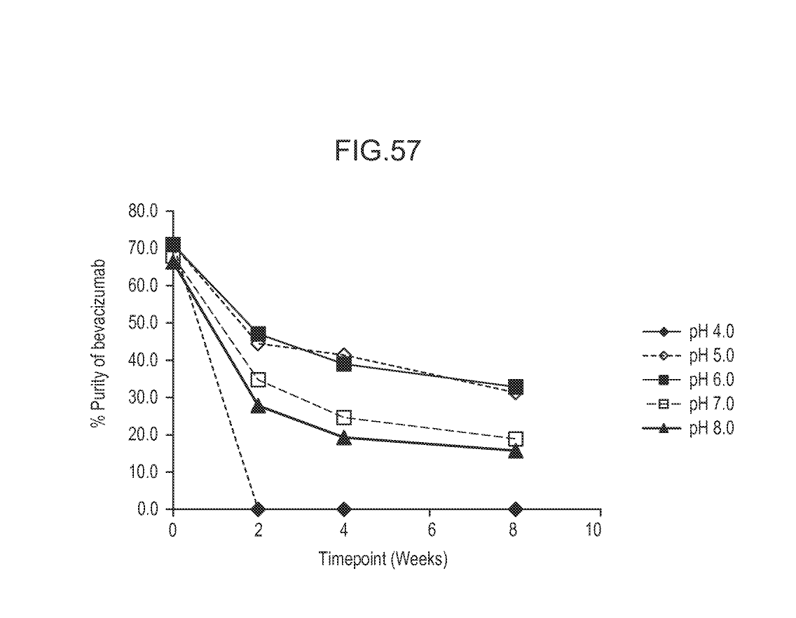

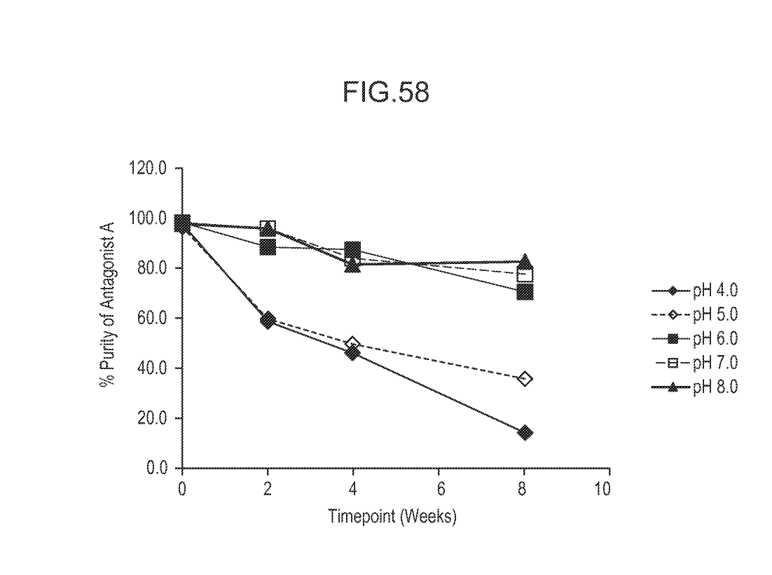

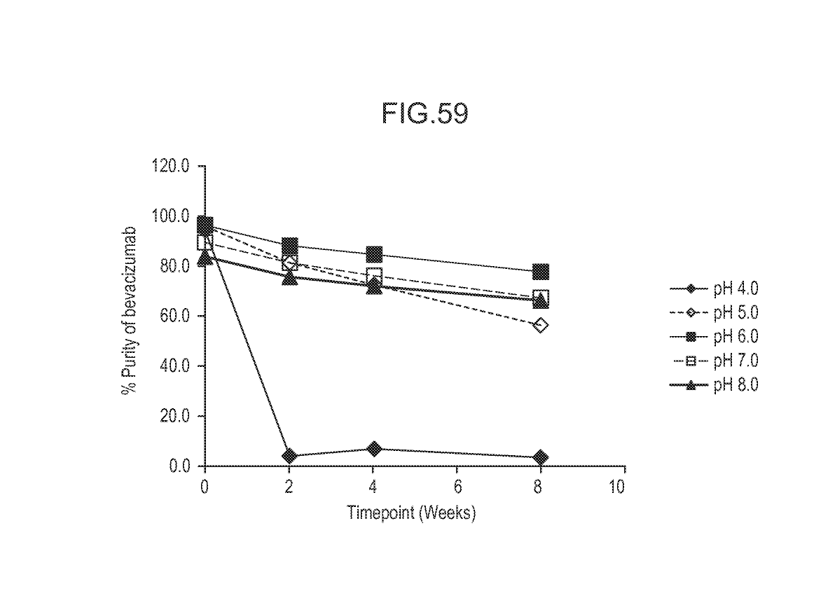

The effects of pH on stability of Antagonist A and bevacizumab were differentiated by storage at 37° C. As observed by AEX-HPLC, Antagonist A was stable at 37° C. in the pH 7.0 and pH 8.0 sorbitol-containing compositions F20 and F22 in contrast to the pH 4.0-6.0 sorbitol-containing compositions F13, F15, and F17, where accelerated degradation occurred (FIG. 56). For bevacizumab, as observed by WCX-HPLC, sorbitol-containing compositions outside of pH 5.0-6.0 (F13, F20, and F22 exhibited accelerated degradation of bevacizumab at 37° C. (FIG. 57). Similar to the AEX-HPLC results, SE-HPLC revealed that Antagonist A in sorbitol-containing compositions F13 and F15 (pH 4.0-5.0) underwent fragmentation or aggregation at 37° C. (FIG. 58). However, despite the degradation seen by WCX-HPLC for sorbitol-containing compositions outside the range of pH 5.0-6.0, SE-HPLC revealed that bevacizumab underwent slower aggregation or fragmentation in sorbitol-containing compositions at pH 5.0-8.0 when stored at 37° C. (FIG. 59). SE-HPLC of the pH 4.0 sorbitol-containing composition F13 stored at 37° C. revealed substantial degradation of bevacizumab. Formulating at pH 6.0 (F 17) appeared to maintain the purity of bevacizumab better than the other pH levels assayed for sorbitol-containing compositions (FIG. 58 and FIG. 59).

Effect of Tonicity Modifier on Stability

The effect of tonicity modifiers on the stability of Antagonist A and bevacizumab was differentiated by storage at 37° C. The benefits of either sorbitol or sodium chloride depended on pH of the composition.

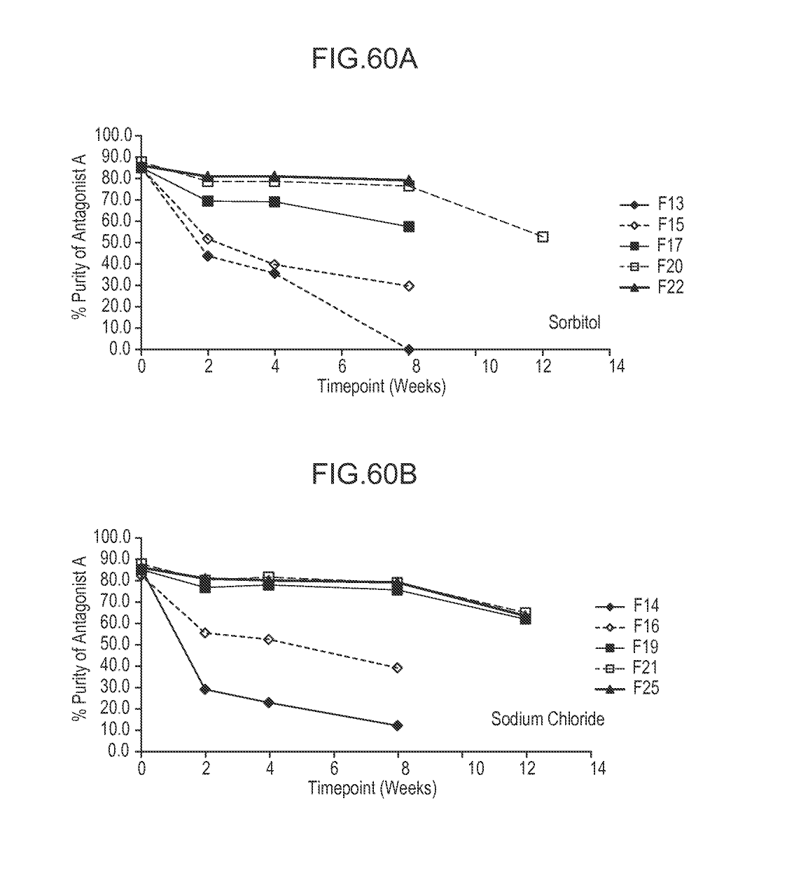

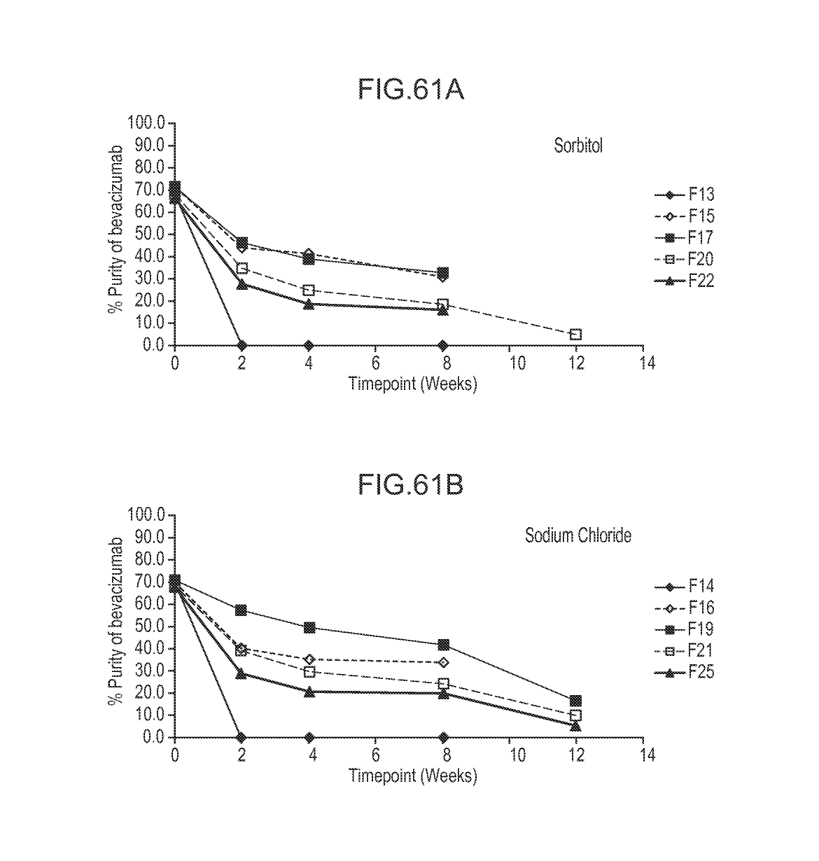

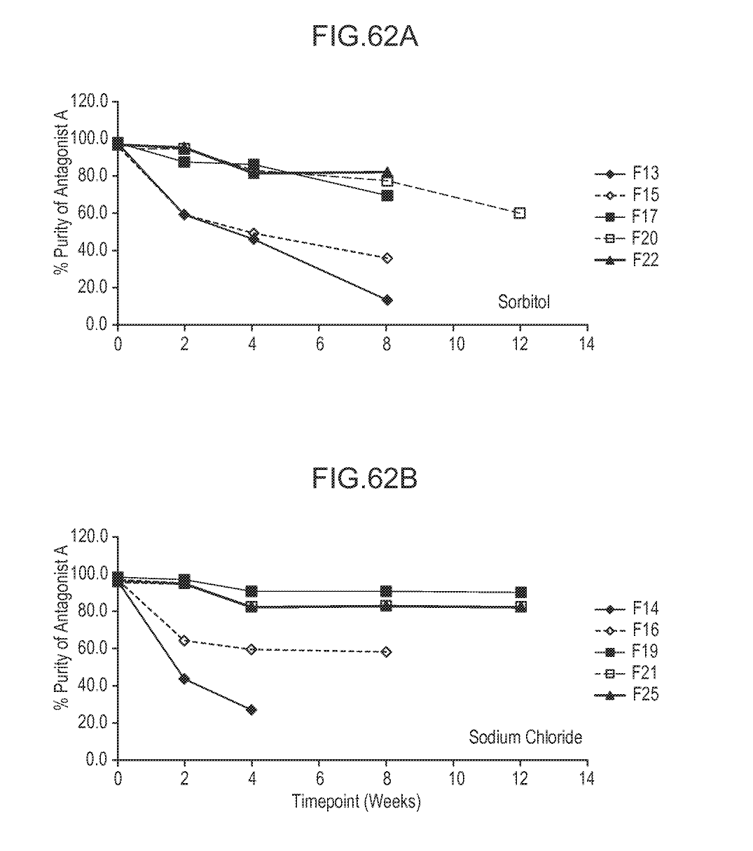

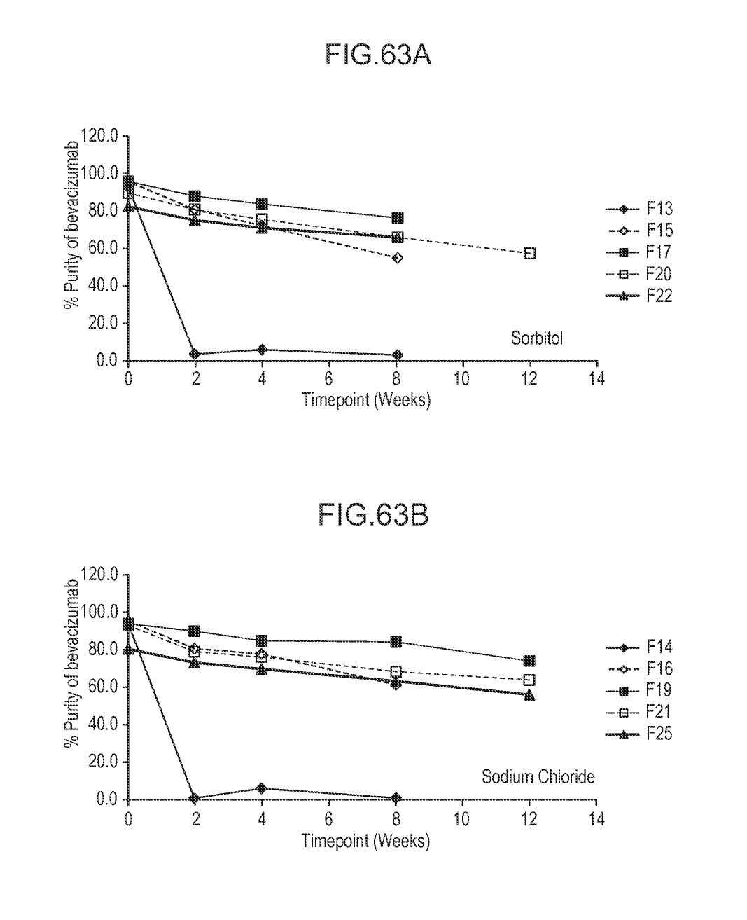

At pH 5.0 and 6.0, Antagonist A underwent degradation in sorbitol compositions (F 15 and F 17) throughout the eight week study as observed by AEX-HPLC (FIG. 60). However, compositions at these pH levels with sodium chloride as tonicity modifier (F16 and F19) did not undergo such degradation (FIG. 60). The pH 4.0 composition containing sodium chloride (F 14) proved to have reduced stability after 4 weeks of accelerated stress, resulting in sorbitol being the superior tonicity modifier at pH 4.0 (FIG. 60). At pH 7.0 and pH 8.0, compositions with either sodium chloride or sorbitol as tonicity modifier (F20, F21, F22, and F23) maintained comparable stability. Analysis of bevacizumab by WCX-HPLC revealed that formulating with sodium chloride from pH 6.0-7.0 improved stability relative to sorbitol (FIG. 61). However, the opposite was true for pH 5.0 compositions, where sorbitol limited degradation relative to sodium chloride for 4 weeks storage at 37° C. (FIG. 61). By SE-HPLC, Antagonist A stability was impacted by the presence of sodium chloride or sorbitol, while the stability of bevacizumab remained comparable between both tonicity modifiers. For pH 5.0-6.0 compositions, the presence of sodium chloride protected Antagonist A from aggregation or fragmentation better than sorbitol (FIG. 62). With the other pHs assayed, Antagonist A displayed lower purity at pH 4.0 with sorbitol (FIG. 62). Antagonist A formulated at pH 7.0 and pH 8.0 (FIG. 62) and bevacizumab formulated at pH 4.0, pH 7.0, and pH 8.0 (FIG. 63) maintained comparable purity with either sorbitol or sodium chloride as tonicity modifier.

Effect of 1:1 Mixture on Stability

Another parameter analyzed was the effect of mixing Antagonist A and commercial bevacizumab. Also, compositions containing different concentrations of Antagonist A with a fixed concentration of bevacizumab (1:1 Mix (F24) and 1:1 Mix (F26) were analyzed. Also, stressing the compositions at 37° C. provided information on the degradation of both Antagonist A and bevacizumab.

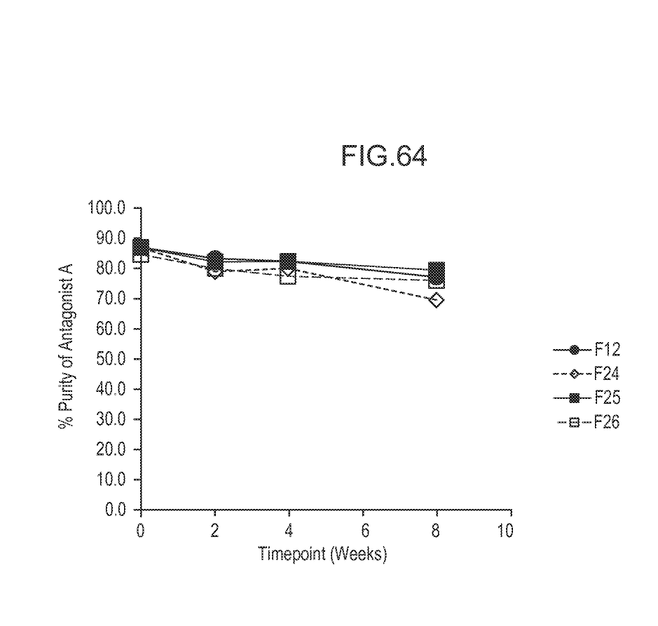

For Antagonist A alone, formulating at 30 mg/mL (F12) or 3 mg/mL (F25) produced no difference in stability profiles by AEX-HPLC and SE-HPLC. Upon mixing commercial Avastin® with varying Antagonist A concentrations (15 mg/mL and 3 mg/mL), Antagonist A in both compositions maintained comparable stability up to 8 weeks at 37° C., whereas formulating Antagonist A at 15 mg/mL with 12.5 mg/mL of bevacizumab produced slight degradation as observed by AEX-HPLC (FIG. 64).

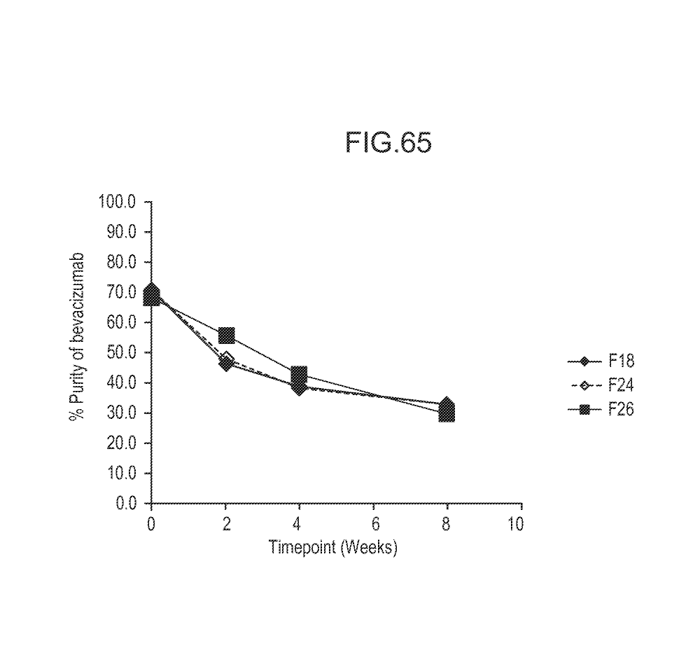

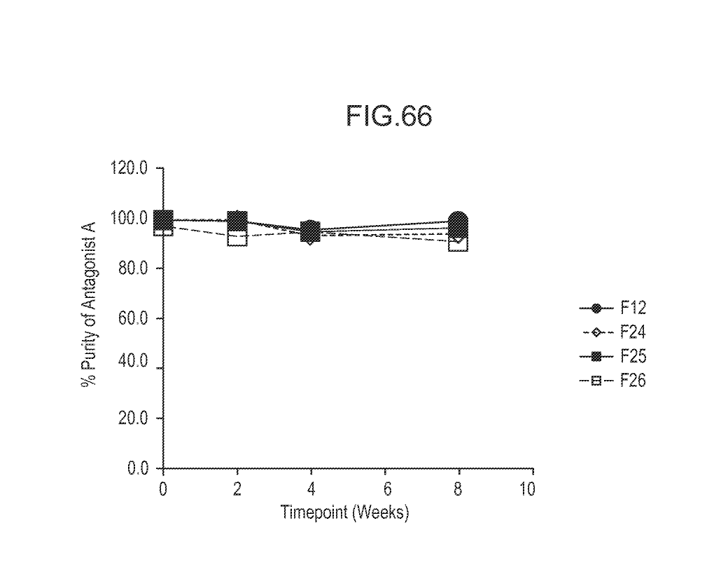

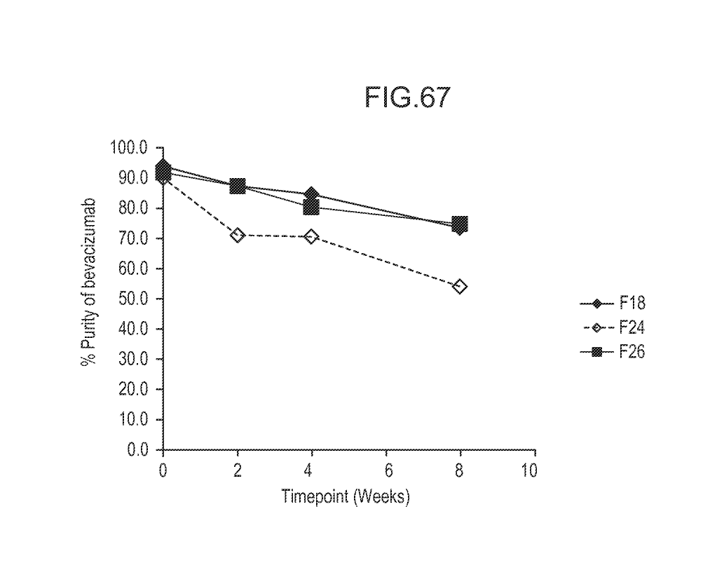

Even though the concentration of bevacizumab was constant in all of the compositions in this study, the varying concentrations of Antagonist A affected the stability of bevacizumab. After 8 weeks storage at 37° C., WCX-HPLC revealed minor differences in the degradation profile of bevacizumab when formulated with either 3 mg/mL Antagonist A (F26) or 15 mg/mL Antagonist A (F24) (FIG. 65). By SE-HPLC, no significant differences in purity profiles were seen between Antagonist A at 30 mg/mL and 3 mg/mL compared to direct 1:1 mixing at two concentrations (F24 and F26) (FIG. 66). However, for bevacizumab, compositions with 15 mg/mL Antagonist A (F24) produced more soluble aggregation and fragmentation of the bevacizumab compared to the 3 mg/mL composition 1:1 Mix (F26) and to a diluted form of commercial Avastin® (F18; FIG. 67).

### Stability of the F19 Composition

Throughout the 24-week study, composition F19 displayed the best stability among all of the compositions assayed. Throughout the study, all F19 compositions remained visually clear and maintained targeted pH values. This section highlights the stability profile of this composition.

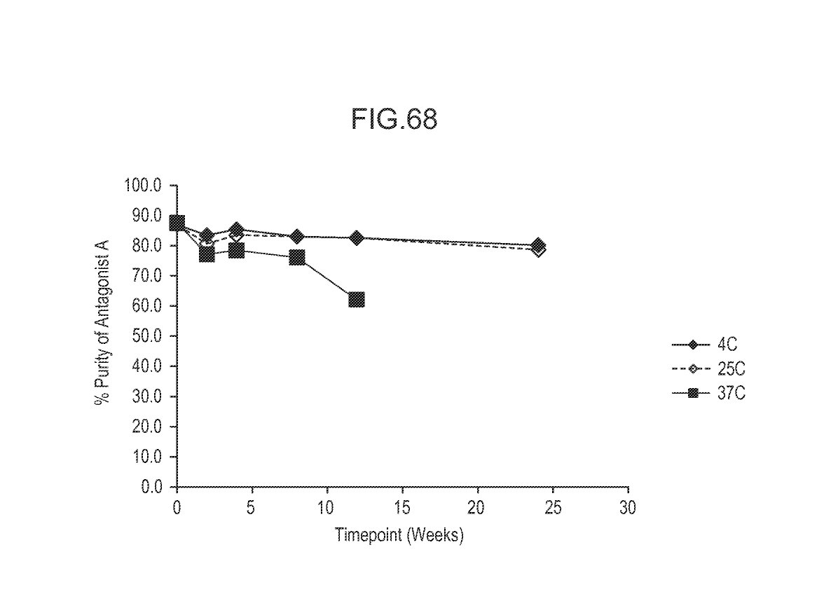

By AEX-HPLC analysis, the F19 composition maintained comparable Antagonist A purity throughout 24 weeks at both 4° C. and 25° C. (FIG. 68). However, when stored at 37° C., the purity of Antagonist A was approximately 5% lower by the second week (FIG. 68). This trend at 37° C. continued over the next 12 weeks, as Antagonist A purity dropped by approximately 20% compared to the other storage conditions for Antagonist A (FIG. 68).

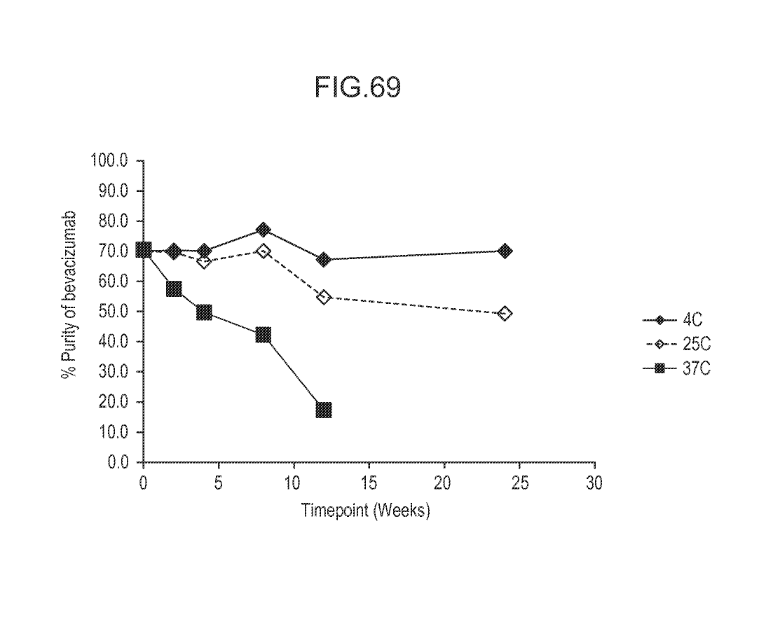

WCX-HPLC analysis revealed a correlation between the storage temperature and the rate of bevacizumab degradation in the F19 composition. After 2 weeks at 37° C., bevacizumab purity dropped approximately 10% compared to 4° C. samples (FIG. 69). This trend continued up to 12 weeks, where the purity of the bevacizumab stored at 37° C. dropped approximately 50% compared to 4° C. (FIG. 69). Storing at 25° C. maintained comparable purity to 4° C. up to 4 weeks (FIG. 42). However, by the eighth week, the 25° C. samples suffered a 7% drop in purity relative to the 4° C. samples (FIG. 69). The increased degradation of bevacizumab stored at 25° C. continued for the rest of the 24 weeks of the study, where at the end of which bevacizumab purity was approximately 20% lower than samples stored at 4° C. (FIG. 69). Storage at 4° C. appeared to maintain comparable purity to starting values throughout the 24 weeks of the study (FIG. 69).

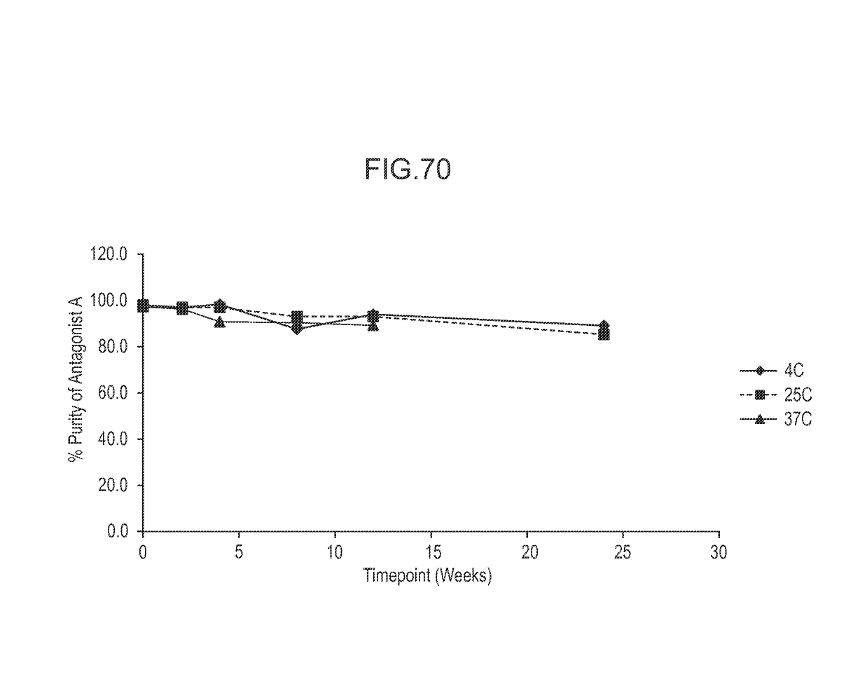

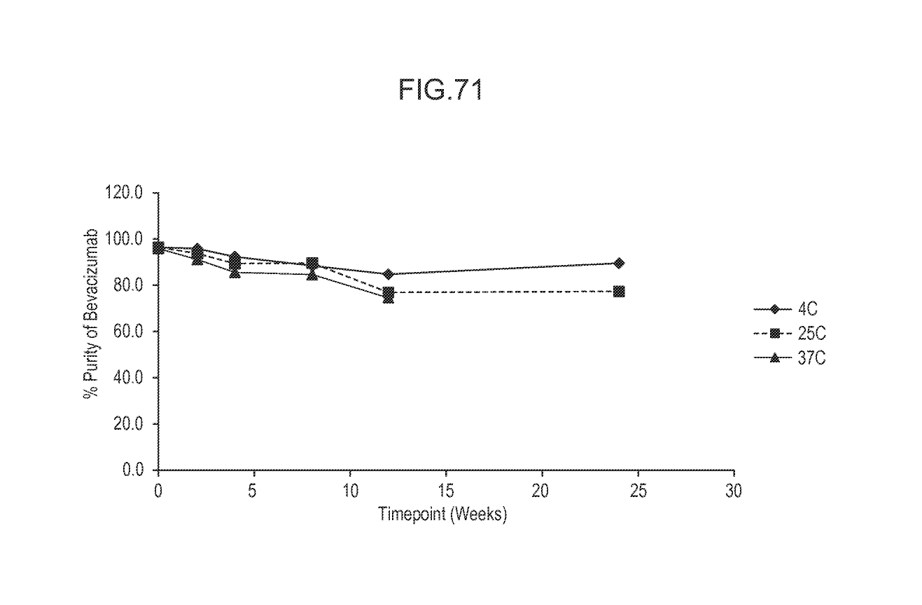

Composition F19 prevented additional soluble aggregation or fragmentation of Antagonist A comparable to starting values by SE-HPLC (FIG. 70). Bevacizumab in F19 stored at 37° C. maintained comparable purity to storage at 4° C. and 25° C. for up to 2 weeks, after which soluble aggregation developed by 4 weeks (FIG. 71). Bevacizumab purity was maintained for up to 8 weeks at 25° C. before significant soluble aggregation developed by 12 weeks (FIG. 71). At 4° C., bevacizumab maintained purity values comparable to the initial time point for 24 weeks (FIG. 71).

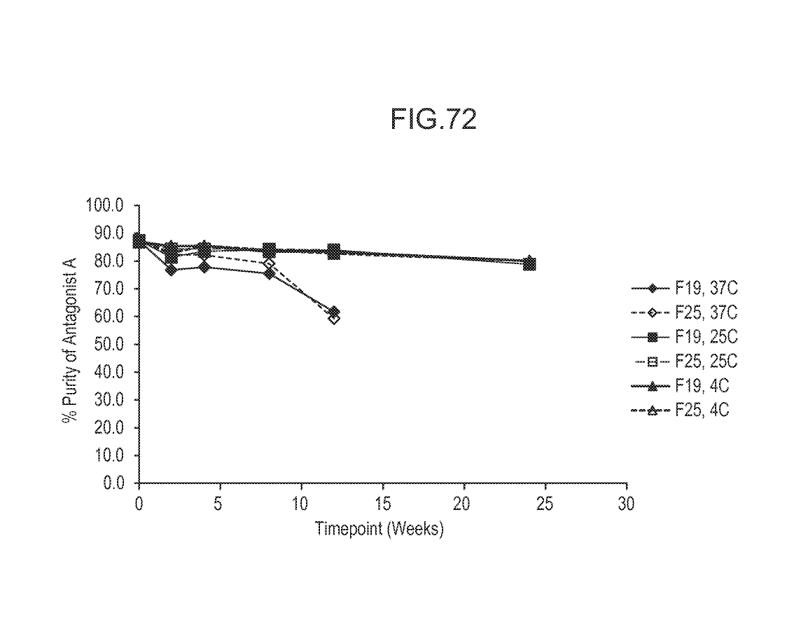

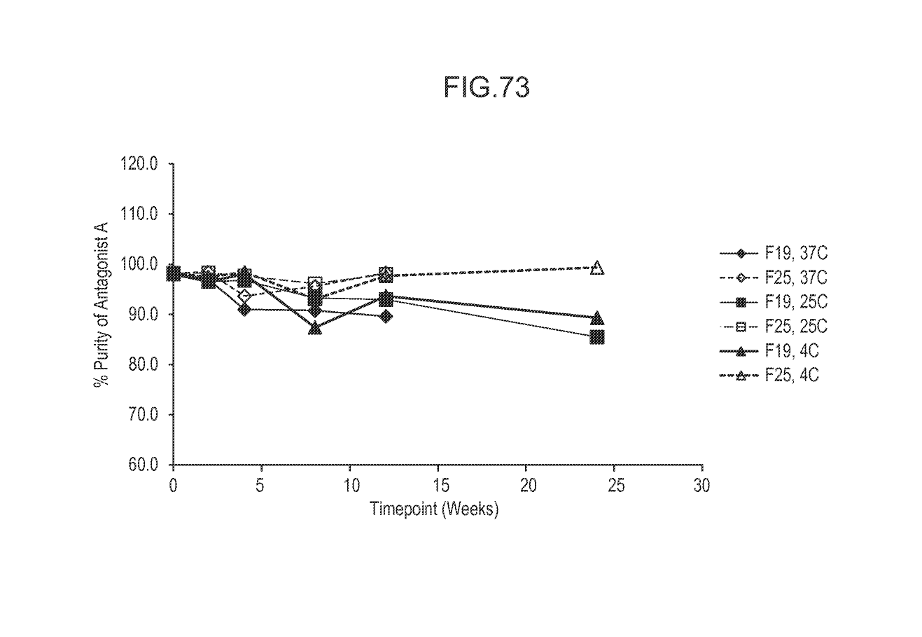

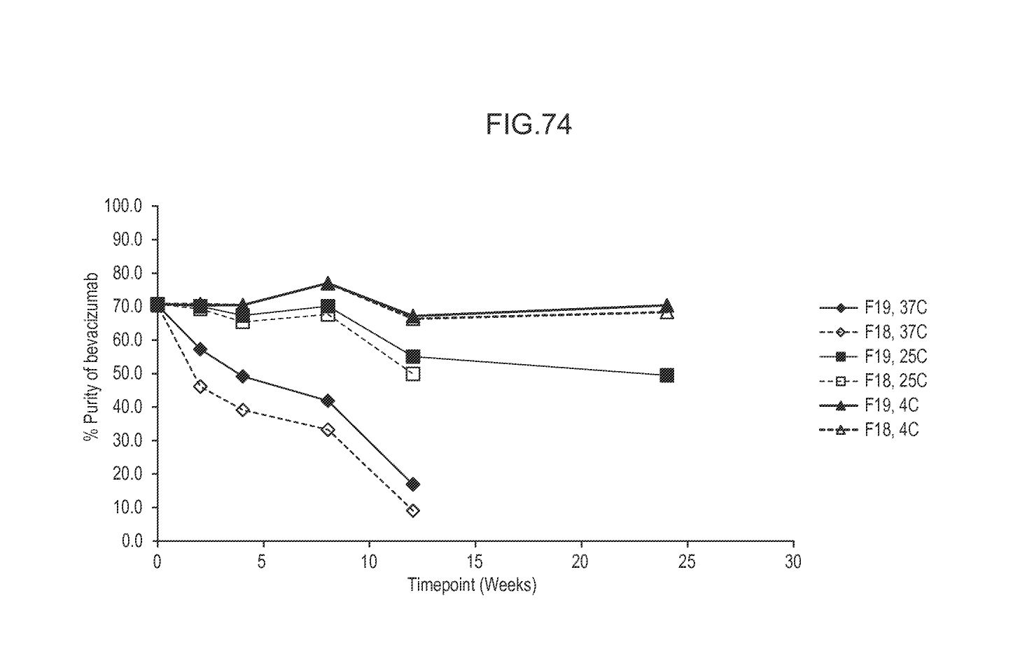

Contrasts of purity between Antagonist A and bevacizumab were seen when comparing composition F19 to compositions comprising only Antagonist A or bevacizuimab. From 2 to 8 weeks at 37° C., Composition F25 maintained 5-8% higher Antagonist A purity than F19 by AEX-HPLC analysis. However, by Week 12, both compositions dropped to similar purity levels (FIG. 72). Furthermore, at 4° C. and 25° C., both compositions maintained comparable purity levels (FIG. 72). By SE-HPLC, the composition F12 appeared better than F19 at each storage condition with the greatest difference seen at 4° C., although some assay variability was observed (FIG. 73).

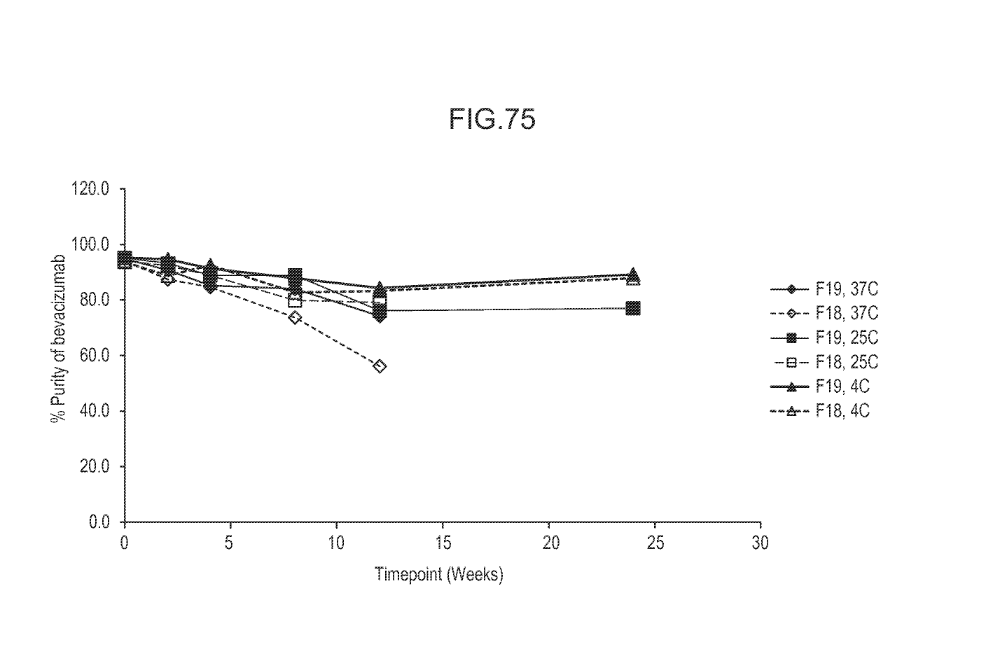

Formulating bevacizumab in F19 provided better stability compared to a diluted form of commercial Avastin® (F18). Based on WCX-HPLC, F19 stabilized bevacizumab better than F18 at 25° C. and especially at 37° C., revealing an 8%-11% improvement from 2-12 weeks (FIG. 74). Similarly, SE-HPLC analysis showed better prevention of aggregation or fragmentation of bevacizumab compared to F 18 stored at 37° C. (FIG. 75).

Based on the data collected over the 24 weeks of stability testing, it was determined that F19 is the most stable composition of Antagonist A and bevacizumab. Among the compositions tested, F 19 helped stabilize both the 3 mg/mL Antagonist A and 12.5 mg/mL bevacizumab when stored at 4° C. for up to at least 24 weeks. Also, the purity of both Antagonist A and bevacizumab in the F19 composition was maintained for up to at least 4 weeks at 25° C.

### Example 3

### Biological Activity of Composition Comprising Ranibizumab and Antagonist A

The purpose of this study was to evaluate the biological activity of a composition comprising both ranibizumab and Antagonist A, as compared to Lucentis® and Antagonist A alone. The activity was measured via the level of gene expression, using real-time PCR, as a function of inhibition of VEGF and PDGF-BB binding to their respective cellular receptors. Three different ranibizumab/Antagonist A compositions were analyzed: F6, F8, and F11 (see Example 1). The compositions had been stored at 4° C. for 12 months prior to their use in this study.

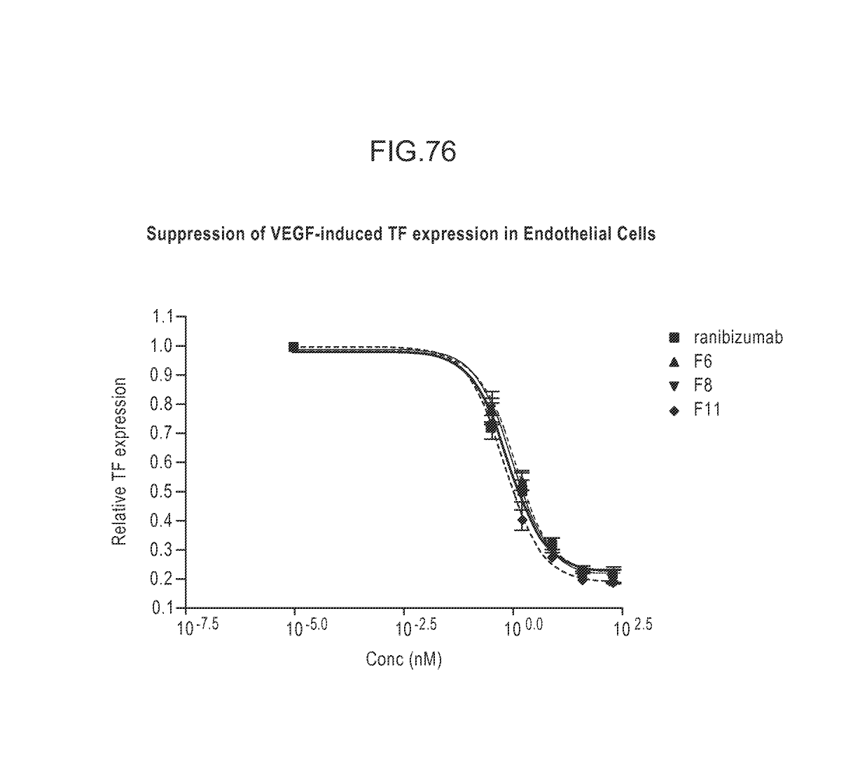

Ranibizumab anti-VEGF activity, alone or present in a composition also comprising Antagonist A, was determined by its ability to inhibit VEGF induction of the Tissue Factor (TF) gene in human umbilical vein endothelial cells (HUVEC). The samples were analyzed in triplicate and all data normalized to that obtained for the VEGF only treatment. As shown in FIG. 76, the anti-VEGF EC50 (nM) values determined for the all compostions and for Lucentis® alone were identical within a 95% confidence interval.

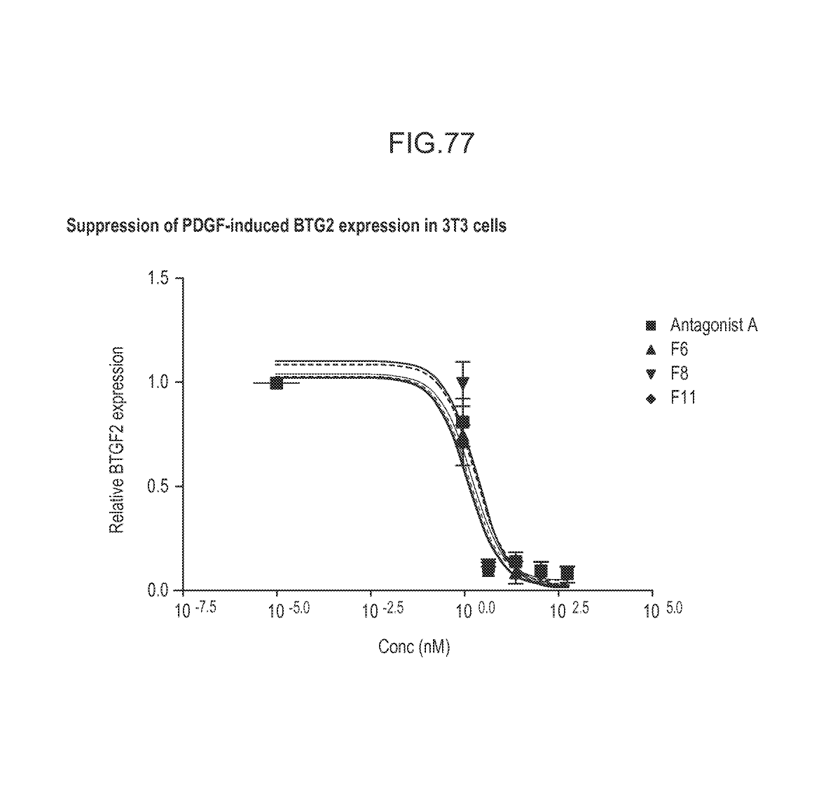

Antagonist A anti-PDGF activity, alone or present in a composition also comprising ranibizumab, was determined by its ability to inhibit PDGF-BB induction of BTG2 gene expression in 3T3 fibroblast cell. The samples were analyzed in duplicate and all data normalized to that obtained for the PDGF-BB only treatment. As shown in FIG. 77, the anti-PDGF EC50 (nM) values determined for all compositions and for Antagonist A alone were identical within a 95% confidence interval. These results demonstrate that a composition comprising both ranibizumab and Antagonist A shows activity for each agent for at least 12 months when stored at 4° C.

### Example 4

### Effect of Storage Conditions on the Stability of Compositions Comprising Antagonist A and Ranibizumab

The stability of Antagonist A and ranibizumab in various compositions was examined using subvisible particle analysis to evaluate the effects of different storage temperature and different storage containers. Subvisible particle analysis was performed for Antagonist A (30 mg/mL), ranibizumab (10 mg/mL and 40 mg/mL), and various combinations of Antagonist A and ranibizumab by micro-flow imaging (MFI). A total of five separate compositions were analyzed following different storage conditions to evaluate the effects of storage temperature (5° C. and 30° C. for 4 hours) and storage container (2 cc vials and 1 mL syringes) on the subvisible particle count for each formulation. The MFI results for each sample were presented in particular particle size ranges (including total, ≧2 μm, ≧5 μm, ≧10 μm and ≧25 μm). Some relative correlation of particle counts was observed for different samples stored under the same conditions.

### Materials

The following Antagonist A and ranibizumab compositions were used in the study:
(1) 30 vials containing 0.23 mL of 30 mg/mL Antagonist A in 10 mM sodium phosphate and 150 mM sodium chloride, pH 7.3 (Composition F27).
(2) 9 vials containing 0.5 mL of 10 mg/mL ranibizumab in 10 mM histidine HCl, 10% α,α-trehalose and 0.01% polysorbate 20, pH 5.5 (Composition F28; Genentech, South San Francisco, Calif.).
(3) 7 vials containing 0.5 mL of 40 mg/mL ranibizumab in 10 mM histidine HCl, 10% α,α-trehalose and 0.01% polysorbate 20, pH 5.5 (Composition F29; Genentech, South San Francisco, Calif.).

The container materials used for composition preparation are listed in Table 7.

TABLE 7
Container materials used in the sample preparations

| Item | Description | Vendor | Cat # |
|---|---|---|---|
| 5 cc vials^{†} | Type 1 borosilicate glass, | Schott | 68000344 |
| | 20 mm finish | | |
| 2 cc vials^{†} | Type 1 borosilicate glass, | Schott | 68000314 |
| | 13 mm finish | | |
| 13 mm vial | FluroTec coated 13 mm | West | 19700004 |
| stoppers | serum stopper | | |
| 13 mm | Aluminum crimp seal with | West | 54130229 |
| Aluminum seal | Flip-Off cap | | |
| 1 mL syringe | Luer-Lok Tip Sterile Syringe | BD | 309628 |
| Syringe stopper | Bromobutyl formulation, 4023/50 | West | |
| | Gray | | |
| 1000 μL | pre-sterile, natural polypropylene | Neptune | BT1000 |
| Barrier tip* | 1000 μL tips | | |
| 25G 11/2 | precisionGlide needle | BD | 305127 |
| needles | | | |

^{†}Vials rinsed with Milli-Q water and dried before use
*Recommended for use with MFI instrument by Protein Simple

### Composition Preparation

In order to prepare the compositions examined in this study, vials of the same sample, i.e., Antagonist A or ranibizumab, were pooled together. In this process, 30 vials of 30 mg/mL Antagonist A (0.20 mL/vial) were pooled into a 5 cc glass vial, 7 vials of 10 mg/mL ranibizumab (0.5 mL/vial) were pooled into a separate 5 cc glass vial, and 7 vials of 40 mg/mL ranibizumab (0.5 mL/vial) were pooled into a third 5 cc glass vial. Although the vials of 30 mg/mL Antagonist A were intended to contain 0.23 mL, only ˜0.2 mL was recovered per each vial when pooling. Pooling was performed by removing the cap from each vial and transferring the contents via pipette in an aseptic manner. Two additional samples were prepared in clean glass vials with various combinations of the pooled materials. Table 8 details the contents for each of the five samples prepared for this study. To ensure sample cleanliness and to prevent particle contamination, all pooling and sample preparations were performed in a class 100 Biological Safety Cabinet (Nuaire NU-425-600).

TABLE 8
Composition matrix for MFI analysis

| | Sample | Containers | Fill volume per |
|---|---|---|---|
| Composition | Description | filled | container |
| F27 | 30 mg/mL Ant. A | 3 vials and | 0.5 mL |
| | | 2 syringes | |
| F28 | 10 mg/mL | 3 vials and | 0.5 mL |
| | Ranibizumab | 2 syringes | |
| F29 | 40 mg/mL | 3 vials and | 0.5 mL |
| | Ranibizumab | 2 syringes | |
| F30 | 50% F27 and | 3 vials and | 0.5 mL |
| | 50% F28 | 2 syringes | |
| | (by volume) | | |
| F31 | 80% F27 and | 2 vials and | 0.5 mL |
| | 20% F29 | 2 syringes* | |
| | (by volume) | | |

*Not enough volume of F31 was available to fill one vial; thus only two vials were filled for this formulation.
“Ant. A” is Antagonist A

In this process, each sample was prepared in a total of two 1 mL syringes and three 2 cc glass vials at a 0.5 mL fill volume, except for F31, which was prepared in two syringes and two vials. The various compositions were prepared individually to allow for precise time point analyses on the MFI instrument. After preparation, each container was fitted with a stopper, and the samples were subjected to stability-study conditions.

### Storage Conditions

Samples of each composition were stored at either 5° C. or 30° C. for 4 hours, in either vials or syringes, to determine the affects of storage temperature and container type on the levels of subvisible particles. T=0 analysis was performed on samples in glass vials immediately after filling. The temperature conditions and analysis time points for this study are shown in Table 9.

TABLE 9
Temperature conditions and analysis time points

| Sample Type | Storage Temperature | Time Point(s) | |
|---|---|---|---|
| Compositions in vials | 5° C. and 30° C. | 0 and 4 | hours |
| Compositions in | 5° C. and 30° C. | 4 | hours |
| syringes | | | |

### Analytical Analysis and Data Processing

Size measurements and subvisible particle counts were collected using an MFI instrument from Brightwell Technologies, model # DPA-4200. 0.5 mL of each sample was directly applied using a pipette tip via an inlet port mounted at top of the flow cell for analysis. In this process, the flow cell was purged with 0.17 mL of sample, thus affording approximately 0.30 mL for particle evaluation.

Subtractions were applied to the MFI data to reduce the number of air bubbles and non-proteinaceous particles included in the total particle count. In this process, stuck particles, slow moving particles, and bubble-like particles with a high circularity were removed from the data in an attempt to isolate and evaluate the oligonucleotide or proteinaceous particles in each sample. Edge particles were also removed in this subtraction, so that the properties of each particle could be properly screened.

Results from MFI analysis were obtained as particle counts per sample. These data were converted to units of particles per mL of sample by dividing the acquired particle count by the exact volume analyzed (approximately 0.30 mL). The values of particles per mL of sample were rounded to the nearest integer.

### Results and Discussion

Table 10 summarizes the results of MFI analysis for the five compositions, F27 to F31, analyzed in this study. Both the raw and subtracted MFI data for the compositions under each storage condition are presented in terms of total particle count/mL, as well particle counts/mL at particle sizes of ≧2 μm, ≧5 μm, ≧8 μm, ≧10 μm and ≧25 μm. Various results were observed during different temperature and container conditions.

TABLE 10
MFI results of Compositions F27 to F31 stored at varying conditions

| | | Raw MFI data (particles/mL) | | | | | Subtracted MFI data (particles/mL) | | | | |
|---|---|---|---|---|---|---|---|---|---|---|---|
| Comp. | Condition | Total | ≧2 μm | ≧5 μm | ≧10 μm | ≧25 μm | Total | ≧2 μm | ≧5 μm | ≧10 μm | ≧25 μm |
| water | None | 498 | 239 | 39 | 13 | 0 | 318 | 141 | 23 | 7 | 0 |
| F27 | T = 0 | 30482 | 5046 | 531 | 75 | 20 | 27185 | 4552 | 406 | 69 | 16 |
| | 5° C. in vial | 29050 | 4466 | 518 | 49 | 3 | 25380 | 3749 | 200 | 23 | 0 |
| | (4 hrs) | | | | | | | | | | |
| | 30° C. in vial | 29748 | 4322 | 321 | 46 | 3 | 26192 | 3870 | 262 | 46 | 3 |
| | (4 hrs) | | | | | | | | | | |
| | 5° C. in syringe | 59955 | 15995 | 2835 | 226 | 3 | 51408 | 12865 | 1380 | 72 | 3 |
| | (4 hrs) | | | | | | | | | | |
| | 30° C. in syringe | 60351 | 16181 | 2638 | 265 | 3 | 51041 | 12305 | 983 | 36 | 0 |
| | (4 hrs) | | | | | | | | | | |
| F28 | T = 0 | 22788 | 4231 | 193 | 23 | 3 | 20399 | 3788 | 167 | 20 | 3 |
| | 5° C. in vial | 29902 | 5990 | 623 | 151 | 26 | 26989 | 5155 | 406 | 102 | 20 |
| | (4 hrs) | | | | | | | | | | |
| | 30° C. in vial | 26553 | 5322 | 531 | 102 | 13 | 24207 | 4811 | 416 | 82 | 10 |
| | (4 hrs) | | | | | | | | | | |
| | 5° C. in syringe | 20648 | 3490 | 210 | 43 | 13 | 18649 | 3188 | 197 | 43 | 13 |
| | (4 hrs) | | | | | | | | | | |
| | 30° C. in syringe | 54082 | 11554 | 1583 | 292 | 52 | 48908 | 10555 | 1265 | 249 | 49 |
| | (4 hrs) | | | | | | | | | | |
| F29 | T = 0 | 17889 | 3598 | 426 | 69 | 7 | 16398 | 3342 | 383 | 49 | 3 |
| | 5° C. in vial | 37190 | 7956 | 1268 | 216 | 36 | 33965 | 7396 | 1222 | 213 | 33 |
| | (4 hrs) | | | | | | | | | | |
| | 30° C. in vial | 68393 | 14068 | 2212 | 210 | 23 | 62373 | 13442 | 2127 | 206 | 23 |
| | (4 hrs) | | | | | | | | | | |
| | 5° C. in syringe | 57917 | 12489 | 2066 | 380 | 39 | 51027 | 10416 | 1774 | 338 | 36 |
| | (4 hrs) | | | | | | | | | | |
| | 30° C. in syringe | 42190 | 9405 | 1462 | 220 | 13 | 38753 | 8923 | 1396 | 216 | 13 |
| | (4 hrs) | | | | | | | | | | |
| F30 | T = 0 | 58241 | 14759 | 2320 | 383 | 36 | 49908 | 11708 | 1065 | 236 | 36 |
| | 5° C. in vial | 80979 | 19770 | 2681 | 315 | 49 | 79210 | 18000 | 1616 | 197 | 33 |
| | (4 hrs) | | | | | | | | | | |
| | 30° C. in vial | 76893 | 17905 | 1891 | 151 | 13 | 67033 | 15123 | 1167 | 111 | 13 |
| | (4 hrs) | | | | | | | | | | |
| | 5° C. in syringe | 90128 | 22820 | 2972 | 279 | 29 | 76392 | 17708 | 1104 | 72 | 13 |
| | (4 hrs) | | | | | | | | | | |
| | 30° C. in syringe | 84961 | 20723 | 2123 | 141 | 26 | 73583 | 16994 | 1232 | 111 | 20 |
| | (4 hrs) | | | | | | | | | | |
| F31 | T = 0 | 69978 | 19046 | 3161 | 310 | 27 | 53929 | 9712 | 722 | 78 | 17 |
| | 5° C. in vial | N/A | N/A | N/A | N/A | N/A | N/A | N/A | N/A | N/A | N/A |
| | (4 hrs)* | | | | | | | | | | |
| | 30° C. in vial | 401875 | 132486 | 25652 | 3883 | 239 | 324874 | 107470 | 16198 | 3277 | 233 |
| | (4 hrs)^{Δ} | | | | | | | | | | |
| | 5° C. in syringe | 103463 | 28893 | 4832 | 683 | 89 | 87777 | 22848 | 2578 | 290 | 41 |
| | (4 hrs) | | | | | | | | | | |
| | 30° C. in syringe | 104495 | 26232 | 3628 | 288 | 23 | 90518 | 22565 | 2048 | 111 | 23 |
| | (4 hrs) | | | | | | | | | | |

*Not enough volume of F31 was available to analyze this particular condition.
^{Δ}Due the low volume for F31, this sample was prepared from a portion of F31 that was initially drawn into a syringe.

The temperature at T=0 was room temperature.

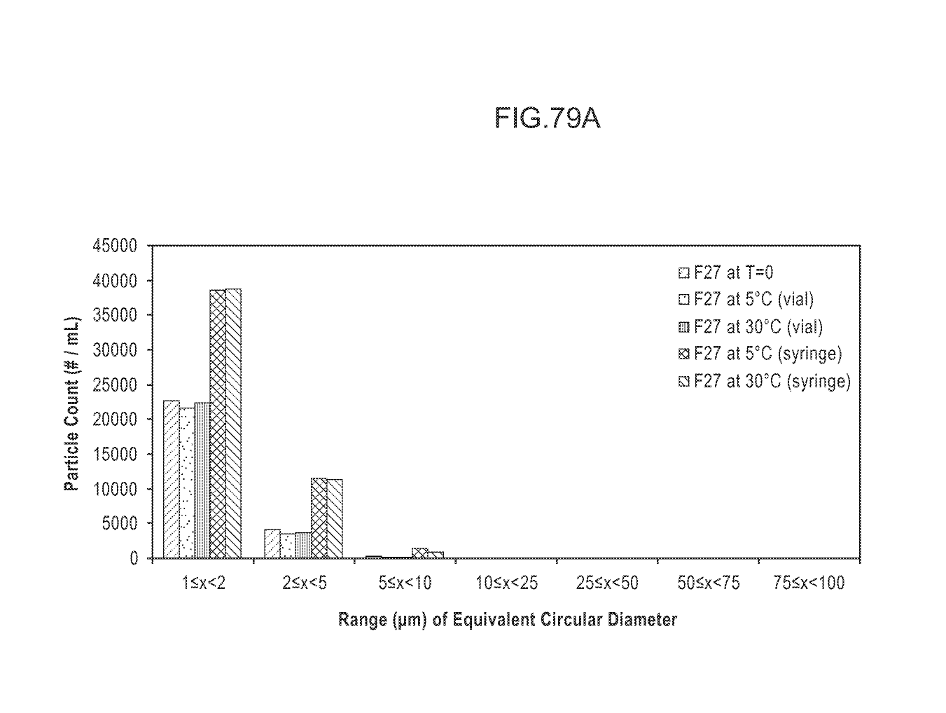

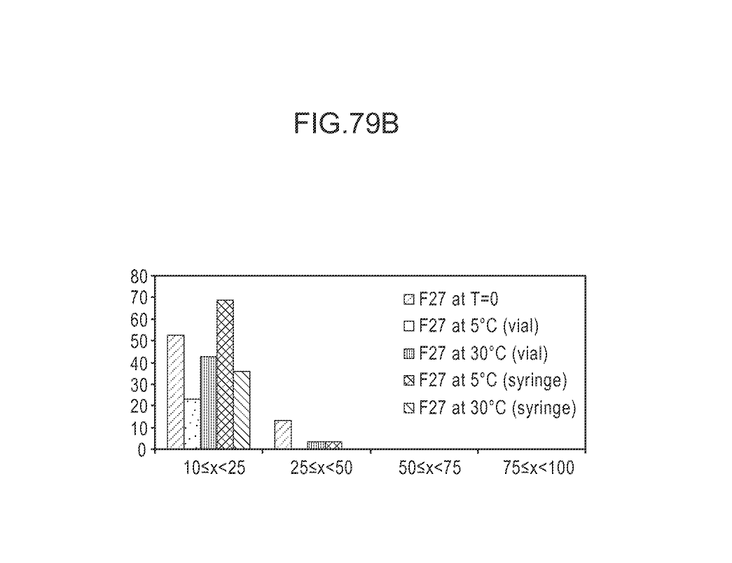

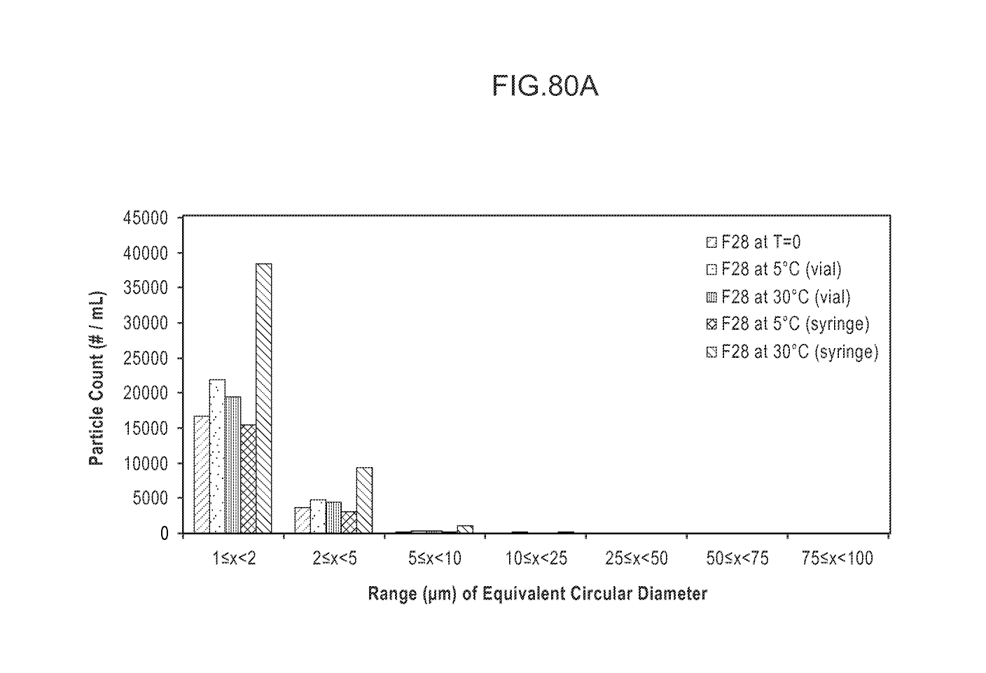

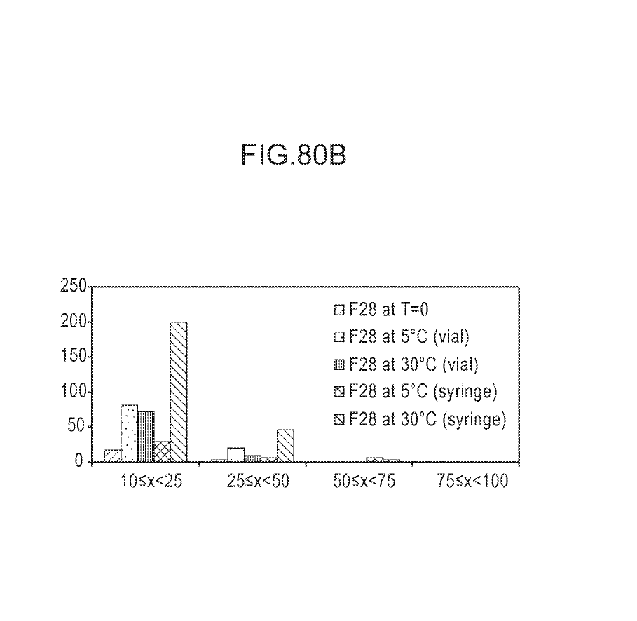

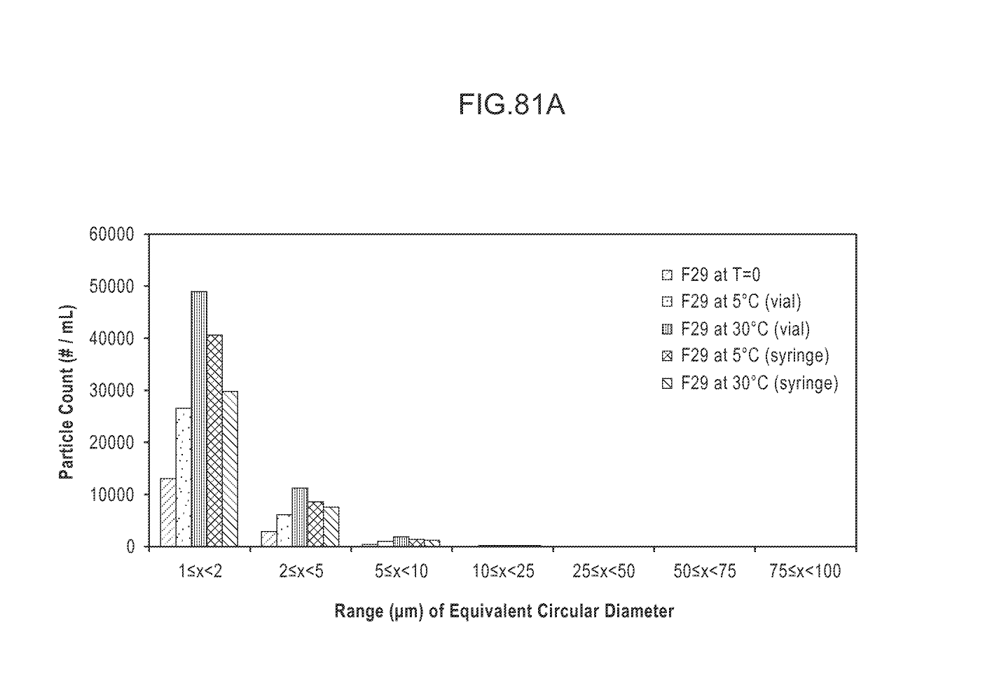

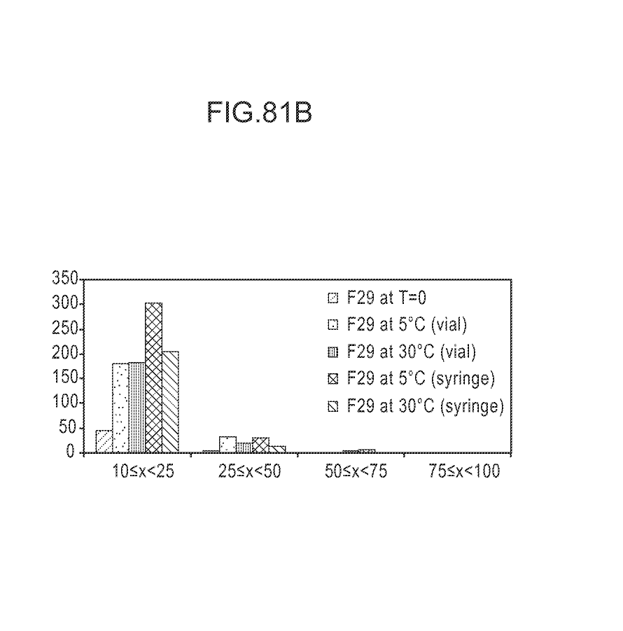

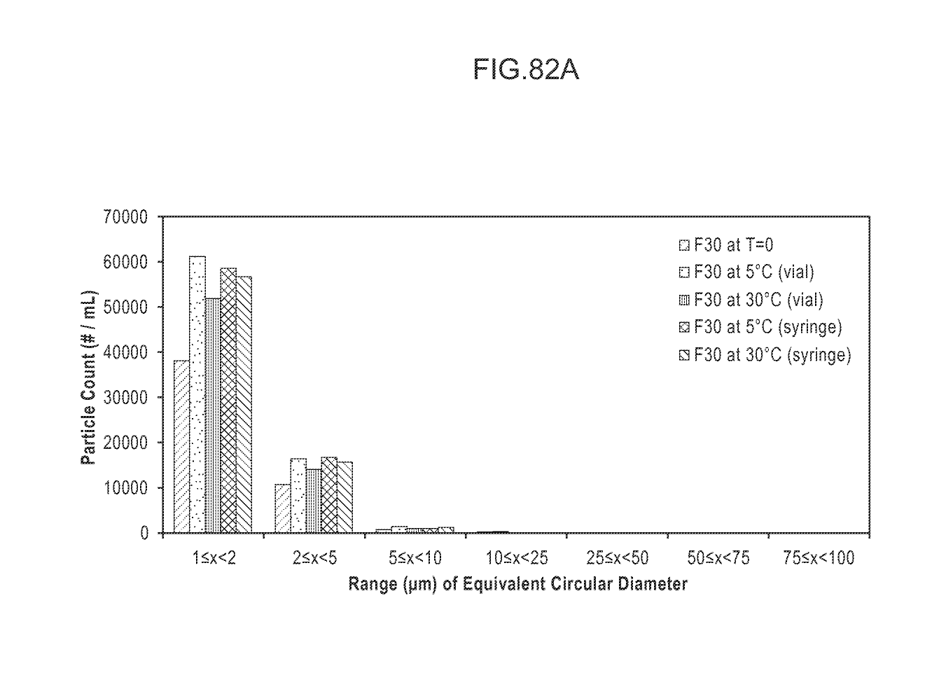

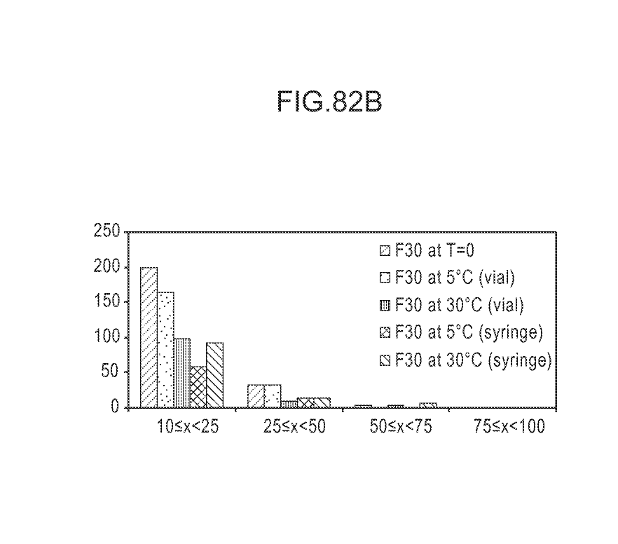

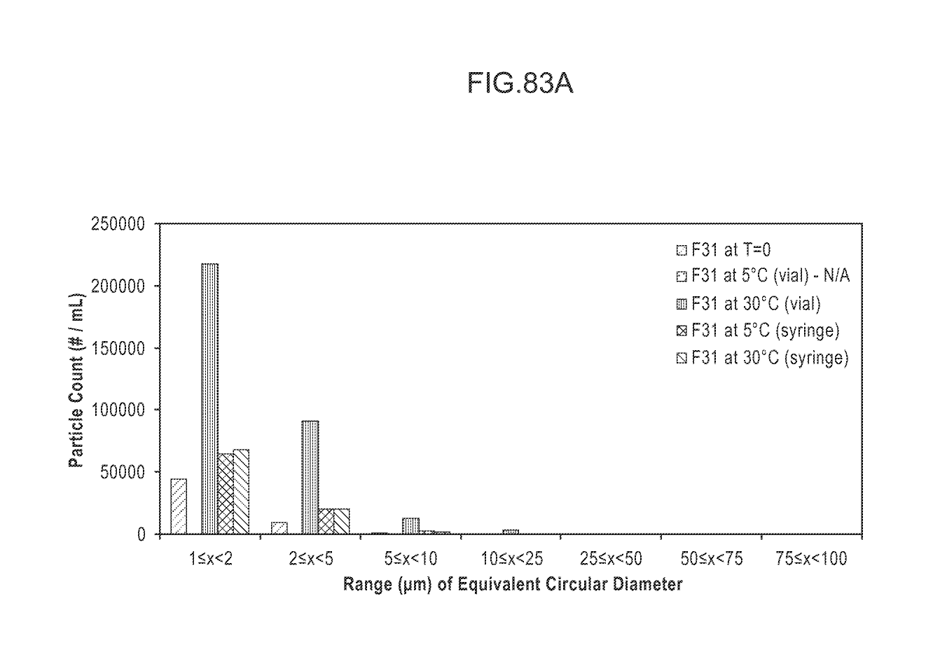

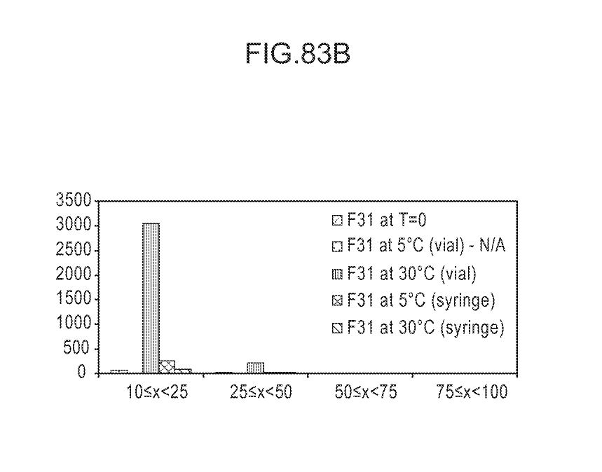

The subtracted MFI results for Compositions F27 to F31, after the different storage conditions, are graphically displayed as histograms in FIGS. 79 to 83, respectively. These histograms present the particle counts for each sample at various size ranges, including 1 to 2 μm, 2 to 5 μm, 5 to 10 μm, 10 to 25 μm, 25 to 50 μm, 50 to 75 μm and 75 to 100 μm. These Figs. also show varying results for the compositions following different storage conditions.

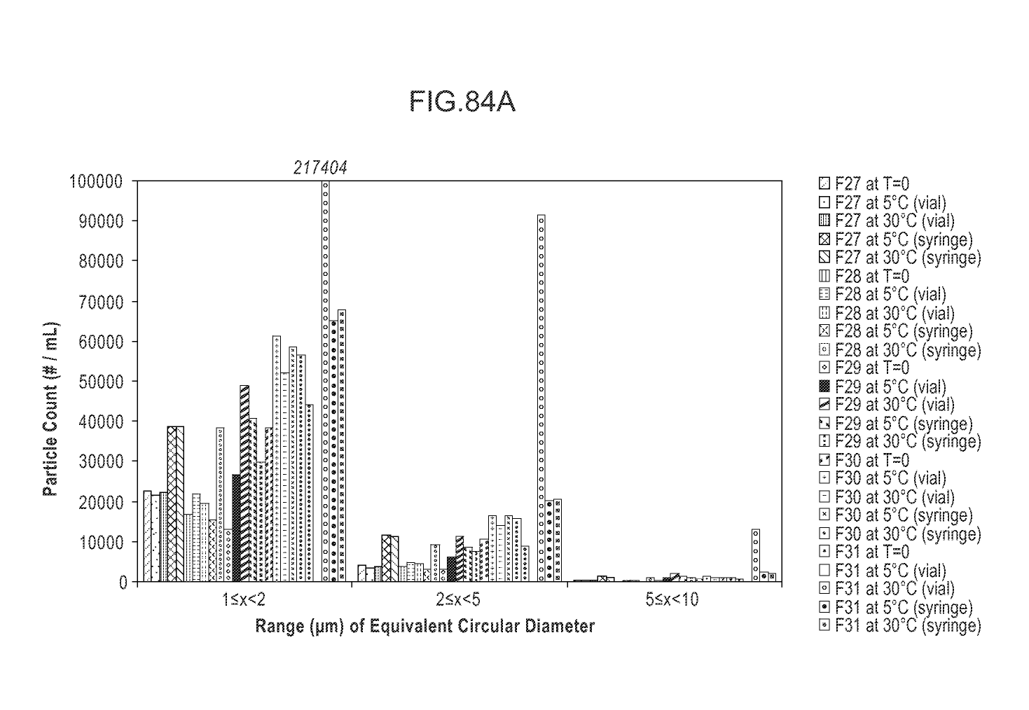

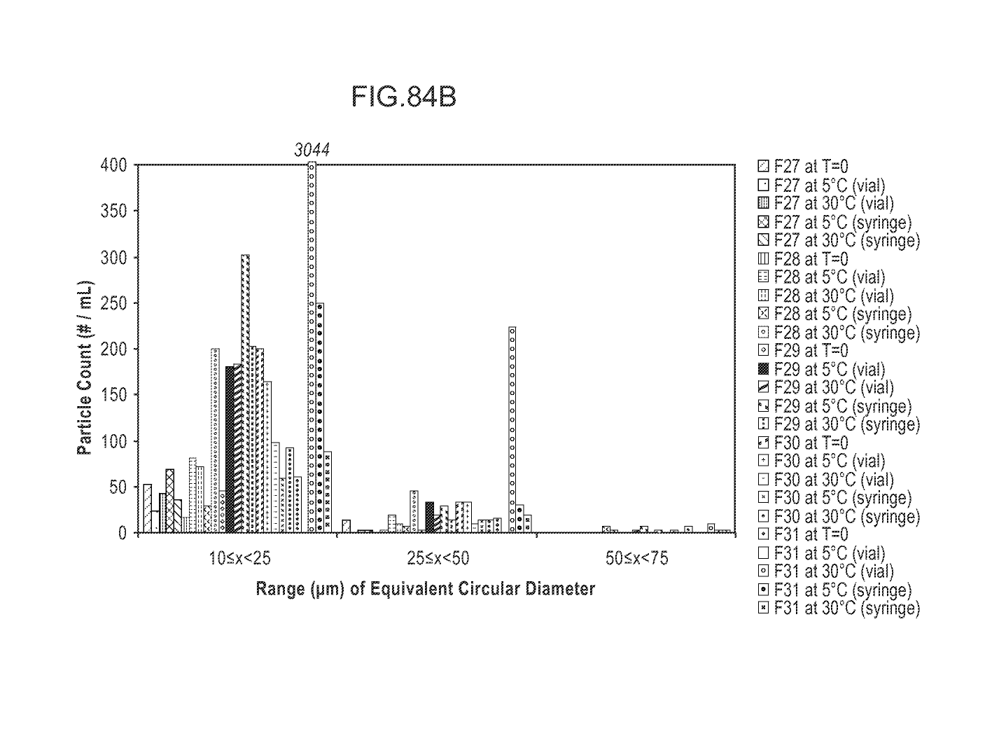

FIG. 84 compares the subtracted MFI results for each sample at the different storage conditions. In this Fig., particle counts were evaluated at 1 to 2 μm, 2 to 5 μm, 5 to 10 μm, 10 to 25 μm, 25 to 50 μm and 50 to 75 μm. The high particle counts observed for F31 in a glass vial following 4 hours at 30° C. may result from sample handling issues. However, additional sample was not available for re-analysis.

### CONCLUSION

The execution of particle analysis for Antagonist A, ranibizumab and various combinations of Antagonist A and ranibizumab was performed by MFI. A total of 24 different samples of 5 compositions were analyzed in this study following 4 hours of storage at 5° C. and 30° C. in either 2 cc glass vials or 1 mL syringes. The results for each sample were presented in particular particle size ranges including ≧2 μm, ≧5 μm, ≧10 μm, ≧25 μm and total particle counts. No considerable differences were observed; however, higher particle counts were detected for F31 in a glass vial following 30° C. storage.

### Example 5

### Synthesis of Antagonist A

An iterative chemical synthesis of the 32-mer oligonucleotide of Antagonist A was performed on a solid phase inverted deoxyribothymidine controlled pore glass (CPG) support using a flow through reactor design. The oligonucleotide synthesis process was comprised of four chemical reactions carried out in the following sequence: (a) deblocking of the dimethyoxytrityl (DMT) protected nucleoside or nascent oligonucleotide (detritylation); (b) activation and coupling of the incoming phosphoramidite (amidite); (c) oxidation of the resultant phosphite triester to the pentavalent phosphate linkage; and (d) capping of oligonucleotide chains that failed to successfully couple.

Starting with an inverted thymidine CPG support (3′-DMT-5′-dT-CPG), the four steps above were repeated to add phosphoramidites in the order of the sequence until the desired oligonucleotide, terminating in the hexylamino linker, was synthesized. The internal hexaethylene glycol spacers were coupled in the same manner as the other phosphoramidites.

The first step in the cycle involved removal of the dimethyoxytrityl protecting group on the terminal hydroxyl group of the nascent oligonucleotide chain. This was achieved by treating the DMT protected oligonucleotide on CPG with a solution of dichloroacetic acid in dichloromethane. This reaction produced the unprotected terminal hydroxyl group. The cleaved DMT group was removed with the dichloroacetic acid/dichloromethane (DCA/DCM) solvent. The CPG was then washed with acetonitrile (ACN).

The second step involved activation of the incoming phosphoramidite with ethylthiotetrazole (ETT) to produce a species that would quickly couple with the terminal hydroxyl group produced in the previous step. The resultant phosphite triester was washed with ACN to remove activator and unreacted phosphoramidite

The third step was oxidation of the newly formed phosphite triester to the pentavalent phosphate. This was accomplished by reacting the phosphite triester with a mixture of iodine and pyridine in water. Unused oxidant was washed from the CPG with ACN.

The fourth step involved capping of any unreacted hydroxyls that had failed to couple. The CPG was treated with a mixture of CAP NMI (N-methylimidazole in ACN) and CAP ALA (acetic anhydride, 2,6-lutidine, ACN). These reagents were washed from the CPG with ACN.

This cycle of four reactions was repeated until an oligonucleotide of the correct length and sequence was assembled on the solid support. The last phosphoramidite (hexylamino linker at the 5′ terminus of the oligonucleotide) was reacted in the same fashion as the other phosphoramidites used in the synthesis; however, this linker was not capped.

The oligonucleotide was deprotected and cleaved by treating the solid support, containing the crude synthesized oligonucleotide, with a t-butyl amine/ammonium hydroxide solution. The CPG was separated from the deprotected and cleaved oligonucleotide. The purity of the crude fully deprotected oligonucleotide was determined by analytical anion exchange chromatography and met a specification of greater than 50%.

The resultant oligonucleotide was diafiltered against sodium chloride to remove amine salts

A covalent bond was then formed between the primary amine at the 5′end of the oligonucleotide and the pegylation reagent (mPEG₂-NHS ester). The reaction was conducted at pH 9 in sodium borate buffer. The reaction has been demonstrated to be site specific to the hexylamino linker at the 5′ end of the oligonucleotide using the pegylation conditions described.

The pegylated oligonucleotide was purified from unconjugated PEG reagent, unpegylated aptamer, and other by-products by preparative anion exchange chromatography (AX HPLC). The individual fractions were analyzed by analytical AX HPLC. Selected fractions of full length pegylated oligonucleotide were pooled and the resultant pool was desalted, concentrated, and filtered.

The resultant Antagonist A was vacuum freeze dried to reduce the water content.

All of the U.S. patents, U.S. patent application publications, U.S. patent applications, foreign patents, foreign patent applications and non-patent publications referred to in this specification or listed in any Application Data Sheet are incorporated herein by reference in their entirety.

From the foregoing it will be appreciated that, although specific embodiments of the invention have been described herein for purposes of illustration, various modifications may be made without deviating from the spirit and scope of the invention.

```
<160> NUMBER OF SEQ ID NOS: 85
<210> SEQ ID NO 1
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (6)..(6)
<223> OTHER INFORMATION: uridine is 2′-fluoro
<220> FEATURE:
<221> NAME/KEY: modified_base
<222> LOCATION: (8)..(8)
<223> OTHER INFORMATION: cytidine is 2′-fluoro
<220> FEATURE:
<221> NAME/KEY: modified_base
<222> LOCATION: (9)..(9)
<223> OTHER INFORMATION: gm
<400> SEQUENCE: 1
caggcuacg 9
<210> SEQ ID NO 2
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: gm
<220> FEATURE:
<221> NAME/KEY: modified_base
<222> LOCATION: (7)..(7)
<223> OTHER INFORMATION: gm
<220> FEATURE:
<221> NAME/KEY: modified_base
<222> LOCATION: (10)..(10)
<223> OTHER INFORMATION: uridine is 2′-fluoro
<220> FEATURE:
<221> NAME/KEY: modified_base
<222> LOCATION: (11)..(11)
<223> OTHER INFORMATION: cytidine is 2′-fluoro
<220> FEATURE:
<221> NAME/KEY: modified_base
<222> LOCATION: (12)..(12)
<223> OTHER INFORMATION: adenosine is 2′-O-Methyl
<400> SEQUENCE: 2
cgtagagcau ca 12
<210> SEQ ID NO 3
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(6)
<223> OTHER INFORMATION: cytidine is 2′-fluoro
<220> FEATURE:
<221> NAME/KEY: modified_base
<222> LOCATION: (7)..(7)
<223> OTHER INFORMATION: uridine is 2′-fluoro
<220> FEATURE:
<221> NAME/KEY: modified_base
<222> LOCATION: (8)..(8)
<223> OTHER INFORMATION: gm
<400> SEQUENCE: 3
tgatccug 8
<210> SEQ ID NO 4
<211> LENGTH: 56
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (10)..(32)
<223> OTHER INFORMATION: From nucleotides 10 to 32, all purines
            (A and G) and all pyrimdines (C and U) are 2′-O-Methyl
<400> SEQUENCE: 4
caggctacga ugcaguuuga gaagucgcgc aucgtagagc atcagaaatg atcctg 56
<210> SEQ ID NO 5
<211> LENGTH: 54
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (10)..(30)
<223> OTHER INFORMATION: From nucleotides 10 to 30, all purines
            (A and G) and all pyrimdines (C and U) are 2′-O-Methyl
<400> SEQUENCE: 5
caggctacgu gcaguuugag aagucgcgca cgtagagcat cagaaatgat cctg 54
<210> SEQ ID NO 6
<211> LENGTH: 39
<212> TYPE: DNA
<213> ORGANISM: artificial
<220> FEATURE:
<223> OTHER INFORMATION: synthetic aptamer
<400> SEQUENCE: 6
cacaggctac ggcacgtaga gcatcaccat gatcctgtg 39
<210> SEQ ID NO 7
<211> LENGTH: 22
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(22)
<223> OTHER INFORMATION: phosphorothioate backbone
<400> SEQUENCE: 7
tgactgtgaa cgttcgagat ga 22
<210> SEQ ID NO 8
<211> LENGTH: 14
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(14)
<223> OTHER INFORMATION: phosphorothioate backbone
<400> SEQUENCE: 8
tgaacgttcg agat 14
<210> SEQ ID NO 9
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(12)
<223> OTHER INFORMATION: phosphorothioate backbone
<400> SEQUENCE: 9
aacgttcgag at 12
<210> SEQ ID NO 10
<211> LENGTH: 10
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(10)
<223> OTHER INFORMATION: phosphorothioate backbone
<400> SEQUENCE: 10
aacgttcgag 10
<210> SEQ ID NO 11
<211> LENGTH: 13
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(13)
<223> OTHER INFORMATION: phosphorothioate backbone
<400> SEQUENCE: 11
gtgaacgttc gag 13
<210> SEQ ID NO 12
<211> LENGTH: 24
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(24)
<223> OTHER INFORMATION: phosphorothioate backbone
<400> SEQUENCE: 12
tcgtcgtttt gtcgttttgt cgtt 24
<210> SEQ ID NO 13
<211> LENGTH: 18
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(18)
<223> OTHER INFORMATION: phosphorothioate backbone
<400> SEQUENCE: 13
gtcgttttgt cgttttgt 18
<210> SEQ ID NO 14
<211> LENGTH: 14
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(14)
<223> OTHER INFORMATION: phosphorothioate backbone
<400> SEQUENCE: 14
gtcgttttgt cgtt 14
<210> SEQ ID NO 15
<211> LENGTH: 46
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(11)
<223> OTHER INFORMATION: phosphorothiate backbone between nucleotides
            1-11
<220> FEATURE:
<221> NAME/KEY: misc_feature
<222> LOCATION: (44)..(46)
<223> OTHER INFORMATION: phosphorothiate backbone between nucleotides
            44-46
<400> SEQUENCE: 15
aacgttcgag caggctacgg cacgtagagc atcaccatga tcctgc 46
<210> SEQ ID NO 16
<211> LENGTH: 49
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(14)
<223> OTHER INFORMATION: phosphorothioate backbone between nucleotides
            1-14
<220> FEATURE:
<221> NAME/KEY: misc_feature
<222> LOCATION: (47)..(49)
<223> OTHER INFORMATION: phosphorothioate backbone between nucleotides
            47-49
<400> SEQUENCE: 16
gtgaacgttc gagcaggcta cggcacgtag agcatcacca tgatcctgc 49
<210> SEQ ID NO 17
<211> LENGTH: 64
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(23)
<223> OTHER INFORMATION: phoshporothiate backbone between nucleotides
            1-23
<220> FEATURE:
<221> NAME/KEY: misc_feature
<222> LOCATION: (56)..(64)
<223> OTHER INFORMATION: phoshporothiate backbone between nucleotides
            56-64
<400> SEQUENCE: 17
tgactgtgaa cgttcgagat gacaggctac ggcacgtaga gcatcaccat gatcctgttt 60
tttt 64
<210> SEQ ID NO 18
<211> LENGTH: 34
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(13)
<223> OTHER INFORMATION: phosphorothioate backbone between nucleotides
            8-13
<220> FEATURE:
<221> NAME/KEY: misc_feature
<222> LOCATION: (31)..(34)
<223> OTHER INFORMATION: phosphorothioate backbone between nucleotides
            31-34
<400> SEQUENCE: 18
caggctacgt tcgtagagca tcaccatgat cctg 34
<210> SEQ ID NO 19
<211> LENGTH: 35
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(14)
<223> OTHER INFORMATION: phosphorothioate backbone between nucleotides
            8-14
<220> FEATURE:
<221> NAME/KEY: misc_feature
<222> LOCATION: (32)..(35)
<223> OTHER INFORMATION: phosphorothioate backbone between nucleotides
            32-35
<400> SEQUENCE: 19
caggctacgt ttcgtagagc atcaccatga tcctg 35
<210> SEQ ID NO 20
<211> LENGTH: 35
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(14)
<223> OTHER INFORMATION: phosphorothioate backbone between nucleotides
            8-14
<220> FEATURE:
<221> NAME/KEY: misc_feature
<222> LOCATION: (32)..(35)
<223> OTHER INFORMATION: phosphorothioate backbone between nucleotides
            32-35
<400> SEQUENCE: 20
caggcaacgt ttcgttgagc atcaccatga tcctg 35
<210> SEQ ID NO 21
<211> LENGTH: 34
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(13)
<223> OTHER INFORMATION: phosphorothioate backbone between nucleotides
            8-13
<220> FEATURE:
<221> NAME/KEY: misc_feature
<222> LOCATION: (31)..(34)
<223> OTHER INFORMATION: phosphorothioate backbone between nucleotides
            31-34
<400> SEQUENCE: 21
caggcaacgt tcgttgagca tcaccatgat cctg 34
<210> SEQ ID NO 22
<211> LENGTH: 36
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(15)
<223> OTHER INFORMATION: phosphorothioate backbone between nucleotides
            8-15
<220> FEATURE:
<221> NAME/KEY: misc_feature
<222> LOCATION: (33)..(36)
<223> OTHER INFORMATION: phosphorothioate backbone between nucleotides
            33-36
<400> SEQUENCE: 22
caggcaacgt tttcgttgag catcaccatg atcctg 36
<210> SEQ ID NO 23
<211> LENGTH: 35
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (32)..(35)
<223> OTHER INFORMATION: phosphorothioate backbone between nucleotides
            32-35
<400> SEQUENCE: 23
caggctacgt ttcgtagagc atcaccatga tcctg 35
<210> SEQ ID NO 24
<211> LENGTH: 35
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(3)
<223> OTHER INFORMATION: phosphorothioate backbone between nucleotides
            1-3
<220> FEATURE:
<221> NAME/KEY: misc_feature
<222> LOCATION: (33)..(35)
<223> OTHER INFORMATION: phosphorothioate backbone between nucleotides
            33-35
<400> SEQUENCE: 24
caggctacgt ttcgtagagc atcaccatga tcctg 35
<210> SEQ ID NO 25
<211> LENGTH: 36
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosporothioate backbone between nucleotides
            1-4
<220> FEATURE:
<221> NAME/KEY: misc_feature
<222> LOCATION: (33)..(36)
<223> OTHER INFORMATION: phosporothioate backbone between nucleotides
            33-36
<400> SEQUENCE: 25
caggcgtcgt tttcgacgag catcaccatg atcctg 36
<210> SEQ ID NO 26
<211> LENGTH: 36
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (33)..(36)
<223> OTHER INFORMATION: phosphorothioate backbone between nucleotides
            33-36
<400> SEQUENCE: 26
caggcgtcgt cgtcgacgag catcaccatg atcctg 36
<210> SEQ ID NO 27
<211> LENGTH: 36
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (33)..(36)
<223> OTHER INFORMATION: phosphorothioate backbone between nucleotides
            33-36
<400> SEQUENCE: 27
caggcttcgt cgtcgaagag catcaccatg atcctg 36
<210> SEQ ID NO 28
<211> LENGTH: 36
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (33)..(36)
<223> OTHER INFORMATION: phosphorothioate backbone between nucleotides
            33-36
<400> SEQUENCE: 28
caggctacgt cgtcgtagag catcaccatg atcctg 36
<210> SEQ ID NO 29
<211> LENGTH: 35
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(14)
<223> OTHER INFORMATION: phosphorothioate backbone between nucleotides
            8-14
<220> FEATURE:
<221> NAME/KEY: misc_feature
<222> LOCATION: (32)..(35)
<223> OTHER INFORMATION: phosphorothioate backbone between nucleotides
            32-35
<400> SEQUENCE: 29
caggcaagct ttgcttgagc atcaccatga tcctg 35
<210> SEQ ID NO 30
<211> LENGTH: 36
<212> TYPE: DNA
<213> ORGANISM: artificial
<220> FEATURE:
<223> OTHER INFORMATION: synthetic aptamer
<220> FEATURE:
<221> NAME/KEY: misc_feature
<222> LOCATION: (1)..(4)
<223> OTHER INFORMATION: phosphorothioate backbone between nucleotides
            1-4
<220> FEATURE:
<221> NAME/KEY: misc_feature
<222> LOCATION: (8)..(15)
<223> OTHER INFORMATION: phosphorothioate backbone between nucleotides
            8-15
<220> FEATURE:
<221> NAME/KEY: misc_feature
<222> LOCATION: (33)..(36)
<223> OTHER INFORMATION: phosphorothioate backbone between nucleotides
            33-36
<400> SEQUENCE: 30
caggcaagct tttgcttgag catcaccatg atcctg 36
<210> SEQ ID NO 31
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (9)..(9)
<223> OTHER INFORMATION: gm
<400> SEQUENCE: 31
caggctacg 9
<210> SEQ ID NO 32
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: gm
<220> FEATURE:
<221> NAME/KEY: modified_base
<222> LOCATION: (7)..(7)
<223> OTHER INFORMATION: gm
<220> FEATURE:
<221> NAME/KEY: modified_base
<222> LOCATION: (12)..(12)
<223> OTHER INFORMATION: adenosine is 2′-O-Methyl
<400> SEQUENCE: 32
cgtagagcat ca 12
<210> SEQ ID NO 33
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (8)..(8)
<223> OTHER INFORMATION: gm
<400> SEQUENCE: 33
tgatcctg 8
<210> SEQ ID NO 34
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (6)..(6)
<223> OTHER INFORMATION: um
<220> FEATURE:
<221> NAME/KEY: modified_base
<222> LOCATION: (8)..(8)
<223> OTHER INFORMATION: cm
<220> FEATURE:
<221> NAME/KEY: modified_base
<222> LOCATION: (9)..(9)
<223> OTHER INFORMATION: gm
<400> SEQUENCE: 34
caggcuacg 9
<210> SEQ ID NO 35
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: gm
<220> FEATURE:
<221> NAME/KEY: modified_base
<222> LOCATION: (7)..(7)
<223> OTHER INFORMATION: gm
<220> FEATURE:
<221> NAME/KEY: modified_base
<222> LOCATION: (10)..(10)
<223> OTHER INFORMATION: um
<220> FEATURE:
<221> NAME/KEY: modified_base
<222> LOCATION: (11)..(11)
<223> OTHER INFORMATION: cm
<220> FEATURE:
<221> NAME/KEY: modified_base
<222> LOCATION: (12)..(12)
<223> OTHER INFORMATION: adenosine is 2′-O-Methyl
<400> SEQUENCE: 35
cgtagagcau ca 12
<210> SEQ ID NO 36
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(6)
<223> OTHER INFORMATION: cm
<220> FEATURE:
<221> NAME/KEY: modified_base
<222> LOCATION: (7)..(7)
<223> OTHER INFORMATION: um
<220> FEATURE:
<221> NAME/KEY: modified_base
<222> LOCATION: (8)..(8)
<223> OTHER INFORMATION: gm
<400> SEQUENCE: 36
tgatccug 8
<210> SEQ ID NO 37
<211> LENGTH: 9
<212> TYPE: DNA
<213> ORGANISM: artificial
<220> FEATURE:
<223> OTHER INFORMATION: synthetic aptamer
<400> SEQUENCE: 37
caggctacg 9
<210> SEQ ID NO 38
<211> LENGTH: 12
<212> TYPE: DNA
<213> ORGANISM: artificial
<220> FEATURE:
<223> OTHER INFORMATION: synthetic aptamer
<400> SEQUENCE: 38
cgtagagcat ca 12
<210> SEQ ID NO 39
<211> LENGTH: 8
<212> TYPE: DNA
<213> ORGANISM: artificial
<220> FEATURE:
<223> OTHER INFORMATION: synthetic aptamer
<400> SEQUENCE: 39
tgatcctg 8
<210> SEQ ID NO 40
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(9)
<223> OTHER INFORMATION: All purines (A and G) and all pyrimidines
            (C and U) are 2′-O-Methyl
<400> SEQUENCE: 40
caggcuacg 9
<210> SEQ ID NO 41
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(12)
<223> OTHER INFORMATION: All purines (A and G) and all pyrimidines
            (C and U) are 2′-O-Methyl
<400> SEQUENCE: 41
cguagagcau ca 12
<210> SEQ ID NO 42
<211> LENGTH: 8
<212> TYPE: RNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(8)
<223> OTHER INFORMATION: All purines (A and G) and all pyrimidines
            (C and U) are 2′-O-Methyl
<400> SEQUENCE: 42
ugauccug 8
<210> SEQ ID NO 43
<211> LENGTH: 10
<212> TYPE: DNA
<213> ORGANISM: artificial
<220> FEATURE:
<223> OTHER INFORMATION: synthetic aptamer
<400> SEQUENCE: 43
acaggctacg 10
<210> SEQ ID NO 44
<211> LENGTH: 9
<212> TYPE: DNA
<213> ORGANISM: artificial
<220> FEATURE:
<223> OTHER INFORMATION: synthetic aptamer
<400> SEQUENCE: 44
tgatcctgt 9
<210> SEQ ID NO 45
<211> LENGTH: 11
<212> TYPE: DNA
<213> ORGANISM: artificial
<220> FEATURE:
<223> OTHER INFORMATION: synthetic aptamer
<400> SEQUENCE: 45
cacaggctac g 11
<210> SEQ ID NO 46
<211> LENGTH: 10
<212> TYPE: DNA
<213> ORGANISM: artificial
<220> FEATURE:
<223> OTHER INFORMATION: synthetic aptamer
<400> SEQUENCE: 46
tgatcctgtg 10
<210> SEQ ID NO 47
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(1)
<223> OTHER INFORMATION: cm
<220> FEATURE:
<221> NAME/KEY: modified_base
<222> LOCATION: (2)..(2)
<223> OTHER INFORMATION: adenosine is 2′-O-Methyl
<220> FEATURE:
<221> NAME/KEY: modified_base
<222> LOCATION: (3)..(4)
<223> OTHER INFORMATION: gm
<400> SEQUENCE: 47
caggctacg 9
<210> SEQ ID NO 48
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(6)
<223> OTHER INFORMATION: cm
<220> FEATURE:
<221> NAME/KEY: modified_base
<222> LOCATION: (7)..(7)
<223> OTHER INFORMATION: um
<220> FEATURE:
<221> NAME/KEY: modified_base
<222> LOCATION: (8)..(8)
<223> OTHER INFORMATION: gm
<220> FEATURE:
<221> NAME/KEY: modified_base
<222> LOCATION: (9)..(9)
<223> OTHER INFORMATION: um
<400> SEQUENCE: 48
tgatccugu 9
<210> SEQ ID NO 49
<211> LENGTH: 10
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(6)
<223> OTHER INFORMATION: cm
<220> FEATURE:
<221> NAME/KEY: modified_base
<222> LOCATION: (7)..(7)
<223> OTHER INFORMATION: um
<220> FEATURE:
<221> NAME/KEY: modified_base
<222> LOCATION: (8)..(8)
<223> OTHER INFORMATION: gm
<220> FEATURE:
<221> NAME/KEY: modified_base
<222> LOCATION: (9)..(9)
<223> OTHER INFORMATION: um
<220> FEATURE:
<221> NAME/KEY: modified_base
<222> LOCATION: (10)..(10)
<223> OTHER INFORMATION: gm
<400> SEQUENCE: 49
tgatccugug 10
<210> SEQ ID NO 50
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (8)..(8)
<223> OTHER INFORMATION: cm
<220> FEATURE:
<221> NAME/KEY: modified_base
<222> LOCATION: (9)..(9)
<223> OTHER INFORMATION: gm
<400> SEQUENCE: 50
caggctacg 9
<210> SEQ ID NO 51
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (6)..(6)
<223> OTHER INFORMATION: um
<220> FEATURE:
<221> NAME/KEY: modified_base
<222> LOCATION: (9)..(9)
<223> OTHER INFORMATION: gm
<400> SEQUENCE: 51
caggcuacg 9
<210> SEQ ID NO 52
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: gm
<220> FEATURE:
<221> NAME/KEY: modified_base
<222> LOCATION: (7)..(7)
<223> OTHER INFORMATION: gm
<220> FEATURE:
<221> NAME/KEY: modified_base
<222> LOCATION: (11)..(11)
<223> OTHER INFORMATION: cm
<220> FEATURE:
<221> NAME/KEY: modified_base
<222> LOCATION: (12)..(12)
<223> OTHER INFORMATION: 2′-O-Methyl adenosine
<400> SEQUENCE: 52
cgtagagcat ca 12
<210> SEQ ID NO 53
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: gm
<220> FEATURE:
<221> NAME/KEY: modified_base
<222> LOCATION: (7)..(7)
<223> OTHER INFORMATION: gm
<220> FEATURE:
<221> NAME/KEY: modified_base
<222> LOCATION: (10)..(10)
<223> OTHER INFORMATION: um
<220> FEATURE:
<221> NAME/KEY: modified_base
<222> LOCATION: (12)..(12)
<223> OTHER INFORMATION: 2′-O-Methyl adenosine
<400> SEQUENCE: 53
cgtagagcau ca 12
<210> SEQ ID NO 54
<211> LENGTH: 9
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: cm
<220> FEATURE:
<221> NAME/KEY: modified_base
<222> LOCATION: (6)..(6)
<223> OTHER INFORMATION: um
<220> FEATURE:
<221> NAME/KEY: modified_base
<222> LOCATION: (8)..(8)
<223> OTHER INFORMATION: cm
<400> SEQUENCE: 54
caggcuacg 9
<210> SEQ ID NO 55
<211> LENGTH: 12
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(1)
<223> OTHER INFORMATION: cm
<220> FEATURE:
<221> NAME/KEY: modified_base
<222> LOCATION: (3)..(3)
<223> OTHER INFORMATION: um
<220> FEATURE:
<221> NAME/KEY: modified_base
<222> LOCATION: (8)..(8)
<223> OTHER INFORMATION: cm
<220> FEATURE:
<221> NAME/KEY: modified_base
<222> LOCATION: (10)..(10)
<223> OTHER INFORMATION: um
<220> FEATURE:
<221> NAME/KEY: modified_base
<222> LOCATION: (11)..(11)
<223> OTHER INFORMATION: cm
<400> SEQUENCE: 55
cguagagcau ca 12
<210> SEQ ID NO 56
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(1)
<223> OTHER INFORMATION: um
<220> FEATURE:
<221> NAME/KEY: modified_base
<222> LOCATION: (4)..(4)
<223> OTHER INFORMATION: um
<220> FEATURE:
<221> NAME/KEY: modified_base
<222> LOCATION: (5)..(6)
<223> OTHER INFORMATION: cm
<220> FEATURE:
<221> NAME/KEY: modified_base
<222> LOCATION: (7)..(7)
<223> OTHER INFORMATION: um
<400> SEQUENCE: 56
ugauccug 8
<210> SEQ ID NO 57
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (6)..(6)
<223> OTHER INFORMATION: cm
<220> FEATURE:
<221> NAME/KEY: modified_base
<222> LOCATION: (7)..(7)
<223> OTHER INFORMATION: um
<220> FEATURE:
<221> NAME/KEY: modified_base
<222> LOCATION: (8)..(8)
<223> OTHER INFORMATION: gm
<400> SEQUENCE: 57
tgatccug 8
<210> SEQ ID NO 58
<211> LENGTH: 11
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (11)..(11)
<223> OTHER INFORMATION: gm
<400> SEQUENCE: 58
cacaggctac g 11
<210> SEQ ID NO 59
<211> LENGTH: 10
<212> TYPE: DNA
<213> ORGANISM: artificial
<220> FEATURE:
<223> OTHER INFORMATION: synthetic aptamer
<400> SEQUENCE: 59
tgatcctgtg 10
<210> SEQ ID NO 60
<211> LENGTH: 10
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (6)..(6)
<223> OTHER INFORMATION: cm
<220> FEATURE:
<221> NAME/KEY: modified_base
<222> LOCATION: (7)..(7)
<223> OTHER INFORMATION: um
<220> FEATURE:
<221> NAME/KEY: modified_base
<222> LOCATION: (8)..(8)
<223> OTHER INFORMATION: gm
<220> FEATURE:
<221> NAME/KEY: modified_base
<222> LOCATION: (9)..(9)
<223> OTHER INFORMATION: um
<220> FEATURE:
<221> NAME/KEY: modified_base
<222> LOCATION: (10)..(10)
<223> OTHER INFORMATION: gm
<400> SEQUENCE: 60
tgatccugug 10
<210> SEQ ID NO 61
<211> LENGTH: 11
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (8)..(8)
<223> OTHER INFORMATION: um
<220> FEATURE:
<221> NAME/KEY: modified_base
<222> LOCATION: (10)..(10)
<223> OTHER INFORMATION: cm
<220> FEATURE:
<221> NAME/KEY: modified_base
<222> LOCATION: (11)..(11)
<223> OTHER INFORMATION: gm
<400> SEQUENCE: 61
cacaggcuac g 11
<210> SEQ ID NO 62
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: cm
<220> FEATURE:
<221> NAME/KEY: modified_base
<222> LOCATION: (7)..(7)
<223> OTHER INFORMATION: um
<220> FEATURE:
<221> NAME/KEY: modified_base
<222> LOCATION: (8)..(8)
<223> OTHER INFORMATION: gm
<400> SEQUENCE: 62
tgatccug 8
<210> SEQ ID NO 63
<211> LENGTH: 8
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(6)
<223> OTHER INFORMATION: cm
<220> FEATURE:
<221> NAME/KEY: modified_base
<222> LOCATION: (8)..(8)
<223> OTHER INFORMATION: gm
<400> SEQUENCE: 63
tgatcctg 8
<210> SEQ ID NO 64
<211> LENGTH: 11
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (11)..(11)
<223> OTHER INFORMATION: gm
<400> SEQUENCE: 64
cccaggctac g 11
<210> SEQ ID NO 65
<211> LENGTH: 10
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: cm
<220> FEATURE:
<221> NAME/KEY: modified_base
<222> LOCATION: (8)..(10)
<223> OTHER INFORMATION: gm
<400> SEQUENCE: 65
tgatcctggg 10
<210> SEQ ID NO 66
<211> LENGTH: 10
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (8)..(10)
<223> OTHER INFORMATION: gm
<400> SEQUENCE: 66
tgatcctggg 10
<210> SEQ ID NO 67
<211> LENGTH: 10
<212> TYPE: DNA
<213> ORGANISM: artificial
<220> FEATURE:
<223> OTHER INFORMATION: synthetic aptamer
<400> SEQUENCE: 67
tgatcctggg 10
<210> SEQ ID NO 68
<211> LENGTH: 10
<212> TYPE: DNA
<213> ORGANISM: artificial
<220> FEATURE:
<223> OTHER INFORMATION: synthetic aptamer
<220> FEATURE:
<221> NAME/KEY: modified_base
<222> LOCATION: (5)..(5)
<223> OTHER INFORMATION: cm
<400> SEQUENCE: 68
tgatcctggg 10
<210> SEQ ID NO 69
<211> LENGTH: 10
<212> TYPE: DNA
<213> ORGANISM: artificial
<220> FEATURE:
<223> OTHER INFORMATION: synthetic
<400> SEQUENCE: 69
aacgttcgag 10
<210> SEQ ID NO 70
<211> LENGTH: 87
<212> TYPE: RNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(87)
<223> OTHER INFORMATION: all pyrimidines (C and U) are 2′-fluoro
<400> SEQUENCE: 70
gggaaaagcg aaucauacac aagaucgcca ggagcaaagu cacggaggag uggggguacg 60
aaugcuccgc cagagaccaa ccgagaa 87
<210> SEQ ID NO 71
<211> LENGTH: 88
<212> TYPE: RNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(88)
<223> OTHER INFORMATION: all pyrimidines (C and U) are 2′-fluoro
<400> SEQUENCE: 71
gggaaaagcg aaucauacac aagaccggga acucggauuc uucgcaugug gaugcgauca 60
guaugcuccg ccagagacca accgagaa 88
<210> SEQ ID NO 72
<211> LENGTH: 88
<212> TYPE: RNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(88)
<223> OTHER INFORMATION: all pyrimidines (C and U) are 2′-fluoro
<400> SEQUENCE: 72
gggaaaagcg aaucauacac aagaccggga acucggauuc uucacaugug gaugugauca 60
guaugcuccg ccagagacca accgagaa 88
<210> SEQ ID NO 73
<211> LENGTH: 88
<212> TYPE: RNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(88)
<223> OTHER INFORMATION: all pyrimidines (C and U) are 2′-fluoro
<400> SEQUENCE: 73
gggaaaagcg aaucauacac aagaccggaa acucggauuc uucgcaugug gaugcgauca 60
guaugcuccg ccagagacca accgagaa 88
<210> SEQ ID NO 74
<211> LENGTH: 88
<212> TYPE: RNA
<213> ORGANISM: artificial
<220> FEATURE:
<223> OTHER INFORMATION: synthetic aptamer
<220> FEATURE:
<221> NAME/KEY: modified_base
<222> LOCATION: (1)..(88)
<223> OTHER INFORMATION: all pyrimidines (C and U) are 2′-fluoro
<400> SEQUENCE: 74
gggaaaagcg aaucauacac aagagagugg aggagguaug uaugguuugu gcgucuggug 60
cggugcuccg ccagagacca accgagaa 88
<210> SEQ ID NO 75
<211> LENGTH: 30
<212> TYPE: DNA
<213> ORGANISM: Artificial Sequence
<220> FEATURE:
<223> OTHER INFORMATION: anti-PDGF aptamer
<220> FEATURE:
<221> NAME/KEY: modified_base
<222> LOCATION: (30)..(30)
<223> OTHER INFORMATION: Inverted thymidine nucleotide that is attached
            to the 3′ end of the oligonucleotide at the 3′ position on the
            ribose sugar
<400> SEQUENCE: 75
caggctacgc gtagagcatc atgatcctgt 30
<210> SEQ ID NO 76
<211> LENGTH: 124
<212> TYPE: PRT
<213> ORGANISM: Artificial Sequence
<220> FEATURE:
<223> OTHER INFORMATION: Humanized anti-VEGF antibody heavy chain
            variable domain
<220> FEATURE:
<221> NAME/KEY: VARIANT
<222> LOCATION: (28)..(28)
<223> OTHER INFORMATION: Xaa = Thr or Asp
<220> FEATURE:
<221> NAME/KEY: VARIANT
<222> LOCATION: (31)..(31)
<223> OTHER INFORMATION: Xaa = Asn or His
<220> FEATURE:
<221> NAME/KEY: VARIANT
<222> LOCATION: (101)..(101)
<223> OTHER INFORMATION: Xaa = Tyr or His
<220> FEATURE:
<221> NAME/KEY: VARIANT
<222> LOCATION: (105)..(105)
<223> OTHER INFORMATION: Xaa = Ser or Thr
<400> SEQUENCE: 76
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Xaa Phe Thr Xaa Tyr
                        20 25 30
Gly Met Asn Trp Val Arg Gln Ala Pro Gly Lys Gly Leu Glu Trp Val
                35 40 45
Gly Trp Ile Asn Thr Tyr Thr Gly Glu Pro Thr Tyr Ala Ala Asp Phe
        50 55 60
Lys Arg Arg Phe Thr Phe Ser Leu Asp Thr Ser Lys Ser Thr Ala Tyr
65 70 75 80
Leu Gln Met Asn Ser Leu Arg Ala Glu Asp Thr Ala Val Tyr Tyr Cys
                                85 90 95
Ala Lys Tyr Pro Xaa Tyr Tyr Gly Xaa Ser His Trp Tyr Phe Asp Asn
                        100 105 110
Val Val Gly Gln Gly Thr Leu Val Thr Val Ser Ser
                115 120
<210> SEQ ID NO 77
<211> LENGTH: 118
<212> TYPE: PRT
<213> ORGANISM: Artificial Sequence
<220> FEATURE:
<223> OTHER INFORMATION: Humanized anti-VEGF antibody heavy chain
            variable domain
<400> SEQUENCE: 77
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Thr Phe Thr Asn Tyr
                        20 25 30
Gly Met Asn Trp Val Arg Gln Ala Pro Gly Lys Gly Leu Glu Trp Val
                35 40 45
Gly Trp Ile Asn Thr Tyr Thr Gly Glu Pro Thr Tyr Ala Ala Asp Phe
        50 55 60
Lys Arg Arg Phe Thr Phe Ser Leu Asp Thr Ser Lys Ser Thr Ala Tyr
65 70 75 80
Leu Gln Met Asn Ser Leu Arg Ala Glu Asp Thr Ala Val Tyr Tyr Cys
                                85 90 95
Ala Lys Tyr Pro His Tyr Tyr Gly Ser Ser His Trp Tyr Phe Asp Val
                        100 105 110
Trp Gly Gln Gly Thr Leu
                115
<210> SEQ ID NO 78
<211> LENGTH: 11
<212> TYPE: PRT
<213> ORGANISM: Mus musculus
<400> SEQUENCE: 78
Ser Ala Ser Gln Asp Ile Ser Asn Tyr Leu Asn
1 5 10
<210> SEQ ID NO 79
<211> LENGTH: 7
<212> TYPE: PRT
<213> ORGANISM: Mus musculus
<400> SEQUENCE: 79
Phe Thr Ser Ser Leu His Ser
1 5
<210> SEQ ID NO 80
<211> LENGTH: 9
<212> TYPE: PRT
<213> ORGANISM: Mus musculus
<400> SEQUENCE: 80
Gln Gln Tyr Ser Thr Val Pro Trp Thr
1 5
<210> SEQ ID NO 81
<211> LENGTH: 108
<212> TYPE: PRT
<213> ORGANISM: Artificial Sequence
<220> FEATURE:
<223> OTHER INFORMATION: Humanized anti-VEGF antibody light chain
            variable domain
<220> FEATURE:
<221> NAME/KEY: VARIANT
<222> LOCATION: (4)..(4)
<223> OTHER INFORMATION: Xaa = Met or Leu
<400> SEQUENCE: 81
Asp Ile Gln Xaa Thr Gln Ser Pro Ser Ser Leu Ser Ala Ser Val Gly
1 5 10 15
Asp Arg Val Thr Ile Thr Cys Ser Ala Ser Gln Asp Ile Ser Asn Tyr
                        20 25 30
Leu Asn Trp Tyr Gln Gln Lys Pro Gly Lys Ala Pro Lys Val Leu Ile
                35 40 45
Tyr Phe Thr Ser Ser Leu His Ser Gly Val Pro Ser Arg Phe Ser Gly
        50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Tyr Ser Thr Val Pro Trp
                                85 90 95
Thr Phe Gly Gln Gly Thr Lys Val Glu Ile Lys Arg
                        100 105
<210> SEQ ID NO 82
<211> LENGTH: 110
<212> TYPE: PRT
<213> ORGANISM: Artificial Sequence
<220> FEATURE:
<223> OTHER INFORMATION: Humanized anti-VEGF antibody light chain
            variable domain
<400> SEQUENCE: 82
Asp Ile Gln Met Thr Gln Ser Pro Ser Ser Leu Ser Ala Ser Val Gly
1 5 10 15
Asp Arg Val Thr Ile Thr Cys Ser Ala Ser Gln Asp Ile Ser Asn Tyr
                        20 25 30
Leu Asn Trp Tyr Gln Gln Lys Pro Gly Lys Ala Pro Lys Val Leu Ile
                35 40 45
Tyr Phe Thr Ser Ser Leu His Ser Gly Val Pro Ser Arg Phe Ser Gly
        50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Tyr Ser Thr Val Pro Trp
                                85 90 95
Thr Phe Gly Gln Gly Thr Lys Val Glu Ile Lys Arg Thr Val
                        100 105 110
<210> SEQ ID NO 83
<211> LENGTH: 10
<212> TYPE: PRT
<213> ORGANISM: Artificial Sequence
<220> FEATURE:
<223> OTHER INFORMATION: Variant hypervariable region of CDRH1
<400> SEQUENCE: 83
Gly Tyr Asp Phe Thr His Tyr Gly Met Asn
1 5 10
<210> SEQ ID NO 84
<211> LENGTH: 14
<212> TYPE: PRT
<213> ORGANISM: Artificial Sequence
<220> FEATURE:
<223> OTHER INFORMATION: Variant hypervariable region of CDRH3
<400> SEQUENCE: 84
Tyr Pro Tyr Tyr Tyr Gly Thr Ser His Trp Tyr Phe Asp Val
1 5 10
<210> SEQ ID NO 85
<211> LENGTH: 458
<212> TYPE: PRT
<213> ORGANISM: Artificial Sequence
<220> FEATURE:
<223> OTHER INFORMATION: VEGF-Trap - dimeric fusion polypeptide
            comprising two fusion polypeptides
<400> SEQUENCE: 85
Met Val Ser Tyr Trp Asp Thr Gly Val Leu Leu Cys Ala Leu Leu Ser
1 5 10 15
Cys Leu Leu Leu Thr Gly Ser Ser Ser Gly Ser Asp Thr Gly Arg Pro
                        20 25 30
Phe Val Glu Met Tyr Ser Glu Ile Pro Glu Ile Ile His Met Thr Glu
                35 40 45
Gly Arg Glu Leu Val Ile Pro Cys Arg Val Thr Ser Pro Asn Ile Thr
        50 55 60
Val Thr Leu Lys Lys Phe Pro Leu Asp Thr Leu Ile Pro Asp Gly Lys
65 70 75 80
Arg Ile Ile Trp Asp Ser Arg Lys Gly Phe Ile Ile Ser Asn Ala Thr
                                85 90 95
Tyr Lys Glu Ile Gly Leu Leu Thr Cys Glu Ala Thr Val Asn Gly His
                        100 105 110
Leu Tyr Lys Thr Asn Tyr Leu Thr His Arg Gln Thr Asn Thr Ile Ile
                115 120 125
Asp Val Val Leu Ser Pro Ser His Gly Ile Glu Leu Ser Val Gly Glu
        130 135 140
Lys Leu Val Leu Asn Cys Thr Ala Arg Thr Glu Leu Asn Val Gly Ile
145 150 155 160
Asp Phe Asn Trp Glu Tyr Pro Ser Ser Lys His Gln His Lys Lys Leu
                                165 170 175
Val Asn Arg Asp Leu Lys Thr Gln Ser Gly Ser Glu Met Lys Lys Phe
                        180 185 190
Leu Ser Thr Leu Thr Ile Asp Gly Val Thr Arg Ser Asp Gln Gly Leu
                195 200 205
Tyr Thr Cys Ala Ala Ser Ser Gly Leu Met Thr Lys Lys Asn Ser Thr
        210 215 220
Phe Val Arg Val His Glu Lys Asp Lys Thr His Thr Cys Pro Pro Cys
225 230 235 240
Pro Ala Pro Glu Leu Leu Gly Gly Pro Ser Val Phe Leu Phe Pro Pro
                                245 250 255
Lys Pro Lys Asp Thr Leu Met Ile Ser Arg Thr Pro Glu Val Thr Cys
                        260 265 270
Val Val Val Asp Val Ser His Glu Asp Pro Glu Val Lys Phe Asn Trp
                275 280 285
Tyr Val Asp Gly Val Glu Val His Asn Ala Lys Thr Lys Pro Arg Glu
        290 295 300
Glu Gln Tyr Asn Ser Thr Tyr Arg Val Val Ser Val Leu Thr Val Leu
305 310 315 320
His Gln Asp Trp Leu Asn Gly Lys Glu Tyr Lys Cys Lys Val Ser Asn
                                325 330 335
Lys Ala Leu Pro Ala Pro Ile Glu Lys Thr Ile Ser Lys Ala Lys Gly
                        340 345 350
Gln Pro Arg Glu Pro Gln Val Tyr Thr Leu Pro Pro Ser Arg Asp Glu
                355 360 365
Leu Thr Lys Asn Gln Val Ser Leu Thr Cys Leu Val Lys Gly Phe Tyr
        370 375 380
Pro Ser Asp Ile Ala Val Glu Trp Glu Ser Asn Gly Gln Pro Glu Asn
385 390 395 400
Asn Tyr Lys Thr Thr Pro Pro Val Leu Asp Ser Asp Gly Ser Phe Phe
                                405 410 415
Leu Tyr Ser Lys Leu Thr Val Asp Lys Ser Arg Trp Gln Gln Gly Asn
                        420 425 430
Val Phe Ser Cys Ser Val Met His Glu Ala Leu His Asn His Tyr Thr
                435 440 445
Gln Lys Ser Leu Ser Leu Ser Pro Gly Lys
        450 455
```

1. A composition comprising an effective amount of:
(a) about 0.3 mg/mL to about 30 mg/mL Antagonist A or a pharmaceutically acceptable salt thereof;
(b) about 0.5 mg/mL to about 20 mg/mL ranibizumab or a pharmaceutically acceptable salt thereof; and one or both of:
(c) a buffer capable of achieving or maintaining the pH of the composition at about pH 5.0 to about pH 8.0; and
(d) a tonicity modifier.

2. The composition of claim 1, wherein-the buffer is about 1 mM to about 20 mM L-histidine or about 1 mM to about 20 mM sodium phosphate; and
the tonicity modifier is about 10 mM to about 200 mM NaCl, about 1% to about 20% (w/v) sorbitol, or about 1% to about 20% (w/v) trehalose.

3. (canceled)

4. The composition of claim 1, further comprising:
(e) about 0.001% (w/v) to about 0.05% (w/v) surfactant.

5. The composition of claim 2, wherein the composition comprises:
(a) about 3 mg/mL Antagonist A or a pharmaceutically acceptable salt thereof;
(b) about 5 mg/mL ranibizumab or a pharmaceutically acceptable salt thereof;
(c) about 10 mM L-histidine; and
(d) about 130 mM NaCl,
wherein the pH of the composition is about pH 6.0.

6. (canceled)

7. The composition of claim 2, wherein the composition comprises:
(a) about 3 mg/mL Antagonist A or a pharmaceutically acceptable salt thereof;
(b) about 5 mg/mL ranibizumab or a pharmaceutically acceptable salt thereof;
(c) about 10 mM sodium phosphate; and
(d) about 5% (w/v) sorbitol,
wherein the pH of the composition is about pH 7.0.

8. (canceled)

9. The composition of claim 2, wherein the composition comprises:
(a) about 3 mg/mL Antagonist A or a pharmaceutically acceptable salt thereof;
(b) about 5 mg/mL ranibizumab or a pharmaceutically acceptable salt thereof;
(c) about 10 mM sodium phosphate; and
(d) about 130 mM NaCl,
wherein the pH of the composition is about pH 7.0.

10. (canceled)

11. The composition of claim 2, wherein the composition comprises:
(a) about 3 mg/mL Antagonist A or a pharmaceutically acceptable salt thereof;
(b) about 5 mg/mL ranibizumab or a pharmaceutically acceptable salt thereof;
(c) about 5 mM sodium phosphate;
(d) about 5 mM histidine HCl;
(e) about 75 mM NaCl; and
(f) about 5% (w/v) trehalose),
wherein the pH of the composition is about pH 6.5.

12. (canceled)

13. A composition comprising an effective amount of:
(a) about 0.3 mg/mL to about 30 mg/mL Antagonist A or a pharmaceutically acceptable salt thereof; and
(b) about 0.5 mg/mL to about 25 mg/mL bevacizumab or a pharmaceutically acceptable salt thereof; and one or both of:
(c) a buffer capable of achieving or maintaining the pH of the composition at about pH 5.0 to about pH 8.0; and
(d) a tonicity modifier.

14. The composition of claim 13, wherein the buffer is about 5 mM to about 200 mM sodium phosphate or about 5 mM to about 200 mM Tris.HCl; and
the tonicity modifier is about 10 mM to about 200 mM NaCl, about 1% to about 20% (w/v) sorbitol, or about 1% to about 20% (w/v) trehalose.

15. (canceled)

16. The composition of claim 13, further comprising:
(e) about 0.001% (w/v) to about 0.05% (w/v) surfactant.

17. The composition of claim 14, wherein the composition comprises:
(a) about 3 mg/mL Antagonist A or a pharmaceutically acceptable salt thereof;
(b) about 12.5 mg/mL bevacizumab or a pharmaceutically acceptable salt thereof;
(c) about 50 mM sodium phosphate; and
(d) about 130 mM NaCl,
wherein the pH of the composition is about pH 6.0.

18. (canceled)

19. The composition of claim 14, wherein the composition comprises:
(a) about 3 mg/mL Antagonist A or a pharmaceutically acceptable salt thereof;
(b) about 12.5 mg/mL bevacizumab or a pharmaceutically acceptable salt thereof;
(c) about 50 mM sodium phosphate; and
(d) about 5% (w/v) sorbitol,
wherein the pH of the composition is about pH 6.0.

20. (canceled)

21. The composition of claim 14, wherein the composition comprises:
(a) about 3 mg/mL Antagonist A or a pharmaceutically acceptable salt thereof;
(b) about 12.5 mg/mL bevacizumab or a pharmaceutically acceptable salt thereof;
(c) about 50 mM sodium phosphate; and
(d) about 5% (w/v) sorbitol,
wherein the pH of the composition is about pH 7.0.

22. (canceled)

23. The composition of claim 14, wherein the composition comprises:
(a) about 3 mg/mL Antagonist A or a pharmaceutically acceptable salt thereof;
(b) about 12.5 mg/mL bevacizumab or a pharmaceutically acceptable salt thereof;
(c) about 50 mM sodium phosphate; and
(d) about 150 mM NaCl,
wherein the pH of the composition is about pH 7.0.

24. (canceled)

25. The composition of claim 14, wherein the composition comprises:
(a) about 3 mg/mL Antagonist A or a pharmaceutically acceptable salt thereof;
(b) about 12.5 mg/mL bevacizumab or a pharmaceutically acceptable salt thereof;
(c) about 50 mM Tris.HCl; and
(d) about 130 mM NaCl,
wherein the pH of the composition is about pH 8.0.

26. (canceled)

27. The composition of claim 14, wherein the composition comprises:
(a) about 15 mg/mL Antagonist A or a pharmaceutically acceptable salt thereof;
(b) about 12.5 mg/mL bevacizumab or a pharmaceutically acceptable salt thereof;
(c) about 30 mM sodium phosphate;
(d) about 75 mM NaCl; and
(e) about 3% (w/v) trehalose,
wherein the pH of the composition is about pH 6.3.

28. (canceled)

29. The composition of claim 14, wherein the composition comprises:
(a) about 3 mg/mL Antagonist A or a pharmaceutically acceptable salt thereof;
(b) about 12.5 mg/mL bevacizumab or a pharmaceutically acceptable salt thereof;
(c) about 30 mM sodium phosphate;
(d) about 75 mM NaCl; and
(e) about 3% (w/v) trehalose,
wherein the pH of the composition is about pH 6.3.

30. (canceled)

31. A composition comprising an effective amount of:
(a) about 0.3 mg/mL to about 30 mg/mL Antagonist A or a pharmaceutically acceptable salt thereof;
(b) about 5 mg/mL to about 40 mg/mL aflibercept or a pharmaceutically acceptable salt thereof; and one or more of:
(c) a buffer capable of achieving or maintaining the pH of the composition at about pH 5.0 to about pH 8.0;
(d) a tonicity modifier; and
(e) 0 to about 10% (w/v) sucrose.

32. The composition of claim 31, wherein the buffer is about 5 mM to about 50 mM phosphate; and
the tonicity modifier is about 10 mM to about 200 mM NaCl.

33. (canceled)

34. The composition of claim 31, further comprising:
(f) about 0.001% (w/v) to about 0.05% (w/v) surfactant.

35. The composition of claim 32, wherein the composition comprises:
(a) about 6 mg/mL Antagonist A or a pharmaceutically acceptable salt thereof;
(b) about 40 mg/mL aflibercept or a pharmaceutically acceptable salt thereof;
(c) about 10 mM phosphate;
(d) about 40 mM NaCl; and
(e) about 5% (w/v) sucrose,
wherein the pH of the composition is about pH 6.2.

36. (canceled)

37. A composition comprising an effective amount of:
(a) about 3 mg/mL to about 90 mg/mL Antagonist A or a pharmaceutically acceptable salt thereof;
(b) about 1.0 mg/mL to about 30 mg/mL ranibizumab or a pharmaceutically acceptable salt thereof; and one or both of:
(c) a buffer capable of achieving or maintaining the pH of the composition at about pH 5.0 to about pH 8.0; and
(d) a tonicity modifier.

38. The composition of claim 37, wherein the buffer comprises about 1 mM to about 100 mM sodium phosphate or about 1.0 mM to about 10 mM histidine.HCl; and
the tonicity modifier is about 0.5% (w/v) to about 10% (w/v) trehalose.

39. The composition of claim 38, wherein the composition comprises:
(a) about 15 mg/mL Antagonist A or a pharmaceutically acceptable salt thereof;
(b) about 5 mg/mL ranibizumab or a pharmaceutically acceptable salt thereof;
(c) about 5 mM phosphate;
(d) about 75 mM NaCl;
(e) about 5 mM histidine.HCl; and
(f) about 5% (w/v) trehalose.

40. (canceled)

41. The composition of claim 38, wherein the composition comprises:
(a) about 24 mg/mL Antagonist A or a pharmaceutically acceptable salt thereof;
(b) about 8 mg/mL ranibizumab or a pharmaceutically acceptable salt thereof;
(c) about 8 mM phosphate;
(d) about 120 mM NaCl;
(e) about 2 mM histidine.HCl; and
(f) about 2% (w/v) trehalose.

42. (canceled)

43. A method for treating or preventing an ophthalmological disease, comprising administering to a mammal in need thereof the composition of any one of claims 1, 13, 31 and 37.

44. The method of claim 43, wherein the ophthalmological disease is age-related macular degeneration, polypoidal choroidal vasculopathy, condition associated with choroidal neovascularization, hypertensive retinopathy, diabetic retinopathy, sickle cell retinopathy, condition associated with peripheral retinal neovascularization, retinopathy of prematurity, venous occlusive disease, arterial occlusive disease, central serous chorioretinopathy, cystoid macular edema, retinal telangiectasia, arterial macroaneurysm, retinal angiomatosis, radiation-induced retinopathy, rubeosis iridis, or a neoplasm.

45.-49. (canceled)
